(19) United States
(12) Patent Application Publication
(10) Pub. No.: US 2021/0115519 A1
(43) Pub. Date: Apr. 22, 2021

(54) METHODS AND KITS FOR DIAGNOSIS AND TRIAGE OF PATIENTS WITH COLORECTAL LIVER METASTASES

(71) Applicant: The University of Chicago, Chicago, IL (US)

(72) Inventors: Sean P. PITRODA, Chicago, IL (US); Nikolai N. KHODAREV, Chicago, IL (US); Ralph R. WEICHSELBAUM, Chicago, IL (US)

(21) Appl. No.: 17/048,791

(22) PCT Filed: Apr. 18, 2019
(86) PCT No.: PCT/US19/28071

§ 371 Date: Oct. 19, 2020

Related U.S. Application Data
(60) Provisional application No. 62/659,936, filed on Apr. 19, 2018.

(51) Int. Cl. C12Q 1/6886

(52) U.S. Cl. CPC C12Q 1/6886; C12Q 2600/178

(57) ABSTRACT

Methods, assays, and compositions for identifying molecular subtypes of metastatic cancer are disclosed. Methods include determining expression levels of genes and/or miRNAs in a sample of metastatic tissue and identifying the molecular subtype of the metastasis based on the determined expression levels. Methods may further include providing a prognosis and making treatment decision based on the molecular subtype of the metastasis.

### CROSS-REFERENCE TO RELATED APPLICATIONS

This application claims the benefit of priority of U.S. Provisional Patent Application No. 62/659,936 filed Apr. 19, 2018, which is hereby incorporated by reference in its entirety.

### BACKGROUND OF THE INVENTION

### 1. Field of the Invention

The current disclosure relates generally to molecular biology and medicine. Particularly it concerns the field of oncology. More particularly, the disclosure relates to methods, compositions and kits involving diagnosis and treatment of metastatic cancer, including metastatic colorectal cancer.

### 2. Technical Background

Metastases are the leading cause of cancer-related deaths and are frequently widely disseminated, which has led to the prevailing view that metastases are always widespread. The oligometastasis hypothesis suggests that metastatic spread is a spectrum of virulence where some metastases are limited both in number and organ involvement and potentially curable with surgical resection or other loco-regional therapies^{1,2}. This paradigm is in stark contrast to the outcomes of patients with solid tumors where widespread metastases are largely fatal despite recent advances in systemic therapy. To date, the oligometastasis concept has been challenged, in large part, due to the lack of supporting molecular data to identify metastases associated with restricted spread^{3,4}.

Limited metastasis is relatively common. Data from clinical trials and single institution analyses of lung, breast, colorectal, prostate and renal cancers suggest that as many as 40-60% of patients with metastasis present with or develop limited disease⁵⁻⁸. Patients with limited liver metastases from colorectal cancer (CRC) have been consistently demonstrated to achieve prolonged survival after hepatic resection^{9,10} and provide an opportunity to investigate the molecular basis for oligometastasis. While there have been extensive investigations into the molecular subtypes of primary human cancers, little is known regarding molecular subtypes of metastasis and their relation to clinical outcomes.

There is a need for the identification of a molecular basis for differing outcomes among metastatic cancer patients, for the ability to identify molecular subtypes of metastatic cancer that are predictive of the clinical outcome, and for the identification of integrated molecular patterns in liver metastases that are associated with long-term survival. There is also a need for methods to differentially identify patients with potentially curable oligometastatic disease from those whose few metastases are a part of a large cascade of widespread disease. The identification of these subtypes of metastatic disease can help in deciding on an appropriate treatment plan for a particular patient.

### SUMMARY OF THE INVENTION

The inventors have discovered a molecular basis for oligometastasis that is predictive of clinical outcome and have developed methods of diagnosis, prognosis, and treatment that use the molecular classification of metastatic tissue to identify curable metastatic cancer and otherwise guide treatment decisions. Using integrated analysis of gene and miRNA expression data in metastatic tissue samples, the inventors identified three molecular subtypes of colorectal cancer metastases. The three subtypes correlate with different clinical outcomes, and knowing the subtype of the metastasis informs treatment decisions and helps provide an accurate assessment of patient prognosis. This discovery applies in metastatic cancers beyond only colorectal liver cancer—methods disclosed herein can be used to identify molecular subtypes of other metastatic cancers and to guide prognosis and treatment decisions for patients having such cancers.

Disclosed herein is a method comprising measuring expression levels of one or more genes listed in Table 10A or one or more miRNAs listed in Table 11A in a sample comprising tissue from a metastasis from a primary cancer tumor. These tables list genes and miRNAs whose expression is particularly valuable in classifying molecular subtypes of metastases. In some embodiments, expression of other genes and miRNAs are also measured. For example, any of the methods disclosed herein may involve measuring the expression of one or more genes listed in Tables 3A-C, which list genes that are differentially expressed in SNF1, SNF2, and SNF3 liver metastases from colorectal cancer primary tumors. Any of the methods disclosed herein may also include measuring expression of one or more miRNAs listed in Tables 4A-4C, which lists miRNAs that are differentially expressed in SNF1, SNF2, and SNF3 liver metastases from colorectal cancer primary tumors. Any of the methods disclosed herein may also include measuring expression of the genes listed in Table 7 (immune genes overexpressed in SNF2 metastases). In some embodiments, the methods disclosed herein also include determining whether one or more of the genes listed in Table 8 are mutated or whether one or more of the genomic alterations listed in Table 9 are present. In some embodiments, expression of both genes and miRNAs are measured as part of a method disclosed herein. The methods disclosed herein can be used specifically in the context of metastatic colorectal cancer. Thus, in some embodiments, the metastasis may be a liver metastasis, and the cancer may be colorectal cancer. The metastasis that is tested may also be in other parts of the body besides the liver, including the lung, peritoneum, brain, or bone. The methods disclosed herein can also be used in the context of other metastatic cancers including, for example, liver cancer, testicular cancer, biliary cancer, ovarian cancer, urinary tract cancer, pancreatic cancer, prostate cancer, esophageal cancer, gastric cancer, head and neck cancer, cervical cancer, lung cancer, neuroendocrine cancer, kidney cancer, breast cancer, and melanoma.

In some embodiments, the expression levels of at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, or 113 of the genes listed in Table 10A are measured, or any range derivable therein. In some embodiments, the expression levels of at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, or 113 of the genes listed in Table 10A are excluded from being measured, or any range derivable therein. In some embodiments, the expression levels of at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53 of the miRNAs listed in Table 11A are measured, or any range derivable therein. In some embodiments, the expression levels of at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53 of the miRNAs listed in Table 11A are excluded from being measured, or any range derivable therein.

It is contemplated that expression levels of any subset of the genes or miRNAs listed in Tables 3A-C, 4A-C, 10A, and 11A may be measured or may be excluded from being measured as part of a method disclosed herein. Certain subsets of these genes and miRNAs may be chosen for their greater usefulness in making classifications and differentiating between different types of metastases. A subset of genes or miRNAs that are to be examined as part of an assay to identify a sample metastasis as belonging to a particular molecular subtype may be identified by an analysis such as a nearest shrunken centroid analysis to identify subsets of genes and/or miRNAs, or a combination of genes and miRNAs, whose expression levels best characterize each subtype. Methods disclosed herein may include performing such an analysis to identify a set of genes and/or miRNAs that can provide for accurate and sensitive subtyping of individual metastases. In some embodiments, expression of a subset of at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 genes or miRNAs listed in Tables 3A-C and 4A-C, or any range derivable therein, are examined.

In some embodiments, the expression levels of one or more genes or one or more miRNAs are within a predetermined amount of the mean expression levels of the one or more genes or miRNAs, on a gene-by-gene and miRNA-by-miRNA basis, in metastases of a cohort of patients having an oligometastatic phenotype, of a cohort of patients who are likely to be healed without the administration of systemic cancer therapy, or of a cohort of patients having a mean ten-year overall survival expectation that is at least 60%. The mean levels may be determined by measuring the expression levels of genes in metastases of patients in the cohort and calculating a mean expression level for each gene. In some embodiments, the patients are patients having metastatic cancer or having metastatic colorectal cancer. Classification of a metastasis may be done by comparing the measured expression levels of genes and/or miRNAs to reference expression levels of the same genes and/or miRNAs. The reference expression levels may be identified as the mean expression levels in metastases of a cohort of patients having characteristics associated with a metastatic subtype, such as a cohort having a mean ten-year overall survival expectation that is at least 60%, or other characteristics of a molecular subtype, such as the characteristics of an SNF1, SNF2, or SNF3 subtype described herein. The reference expression levels of such cohorts, and of any patient cohorts described herein, may be established by measuring the expression levels in metastases of at least, at most, or exactly 50, 100, 150, 200, 250, 300, 350, 400, 450, 500, 600, 700, 800, 900, or 1000 subjects in the cohort, or any range derivable therein. In some embodiments, the cohort of patients comprises a representative sample of metastatic cancer patients, including metastatic colorectal cancer patients, having a certain characteristic, such as an oligometastatic phenotype, a relatively high likelihood of being successfully treated with immune checkpoint therapy, a mean ten-year survival expectation of at least 60%, or other characteristics of metastatic subtypes identified herein. If the expression levels of the genes and/or miRNAs measured in a sample metastasis are sufficiently close to the reference expression levels of a metastatic subtype, then the sample metastasis can be classified as being of that subtype. The degree of closeness in expression levels required to be classified as a match may be predetermined using a statistical analysis. In some embodiments, the predetermined amount of closeness is within one standard deviation of the mean expression level of the reference cohort. In some embodiments, the predetermined amount is within 0.1, 0.5, 1.0, 2.0, 3.0, 4.0, 5.0, 10, 15, or 20% of the reference expression level, or any range derivable therein. In some embodiments, a sample metastasis may be classified as belonging to a molecular subtype despite the expression levels of one or more genes or miRNAs deviating from a reference expression level by a substantial amount. For instance, if a substantial number of other gene or miRNA expression levels sufficiently match the reference expression, then the sample metastasis may be classified as belonging to the subtype. A computer-based classifier programmed to perform a statistical analysis may be used to determine whether expression levels of a sufficient number of genes and/or miRNAs in a sample metastasis are sufficiently close to the reference expression levels of a particular molecular subtype to classify the sample as belonging to that subtype.

It is contemplated that the methods described herein may involve a comparison between expression levels measured for a sample metastasis and reference expression levels that are indicative of metastatic subtypes or any of the characteristics of metastatic subtypes described herein. Thus, in some embodiments, the measured expression level for a gene or miRNA is lower than, higher than, close to, higher by a predetermined amount than, lower by a predetermined amount than, or within a predetermined amount of the expression level of the gene or miRNA in metastases from a cohort of metastatic cancer patients having any one of the following characteristics: (i) a mean ten-year overall survival expectation of at least 60%; (ii) a relatively high or low likelihood of experiencing metastatic recurrence after hepatic resection; (iii) a relatively high or low likelihood of being successfully treated without systemic cancer treatments; (iv) a relatively low likelihood of being successfully treated with local cancer treatments; (v) a relatively high likelihood of being successfully treated with immune checkpoint therapy; (vi) a mean ten-year overall survival expectation of less than 50%, 35%, or 20%; (vii) a relatively high degree of infiltration of immune cells; among other characteristics of the metastatic subtypes described herein. In some embodiments, wherein the expression levels of one or more genes listed in Table 10A or one or more miRNAs listed in Table 11A deviate by a predetermined amount from the mean expression levels of the one or more genes or the one or more miRNAs in metastases of a cohort of metastatic colorectal cancer patients having a mean ten-year overall survival expectation that is less than 50%. In some embodiments, the expression levels of one or more genes listed in Table 10B are higher by a predetermined amount than the mean expression level of the one or more genes in metastases of a cohort of metastatic colorectal cancer patients having a mean ten-year overall survival expectation that is less than 50%. In some embodiments, the measured expression levels of one or more genes listed in Table 10C are lower by a predetermined amount than the mean expression level of the one or more genes in metastases of a cohort of metastatic colorectal cancer patients having a mean ten-year overall survival expectation that is less than 50%. In some embodiments, the measured expression levels of one or more miRNAs listed in Table 11B are higher by a predetermined amount than the mean expression level of the one more more miRNAs in metastases of a cohort of metastatic colorectal cancer patients having a mean ten-year overall survival expectation that is less than 50%. In some embodiments, the measured expression levels of one or more miRNAs listed in Table 11C is lower by a predetermined amount than the mean expression level of the one or more miRNAs in metastases of a cohort of metastatic colorectal cancer patients having a mean ten-year overall survival expectation that is less than 50%. In any of the methods described herein, a cohort of patients may be a cohort of metastatic cancer patients, colorectal cancer patients, or metastatic colorectal cancer patients.

In some embodiments, the method further comprises calculating a Clinical Risk Score (“CRS”) for the patient, which is calculated using the following adverse clinical and pathological features: (1) disease-free interval between primary tumor diagnosis and development of metastasis <12 months, (2) number of liver metastases >1, (3) largest liver metastasis >5.0 cm, (4) lymph node-positive primary CRC, and (5) CEA>200 ng/mL. A patient with none of these features has a CRS of 0; a patient with one of these features has a CRS of 1; and so on up to a maximum CRS of 5.

In some embodiments, the method further comprises administering a cancer therapy to the patient. The cancer therapy may be chosen based on the gene or miRNA expression measurements, alone or in combination with the clinical risk score calculated for the patient. In some embodiments, the cancer therapy comprises a local cancer therapy. In some embodiments, the cancer therapy excludes a systemic cancer therapy. In some embodiments, the cancer therapy excludes a local therapy. In some embodiments, the cancer therapy comprises a local cancer therapy without the administration of a system cancer therapy. In some embodiments, the cancer therapy comprises an immunotherapy, which may be an immune checkpoint therapy. Any of these cancer therapies may also be excluded. Combinations of these therapies may also be administered. In some embodiments, the gene or miRNA expression measurement and analysis may indicate that one or more cancer therapies would be likely to be effective or ineffective. A particular advantage of methods disclosed herein is that they allow doctors for the first time to make a treatment decision based on the molecular subtype of a metastasis. The discoveries disclosed herein indicate that some metastatic subtypes, such as SNF2, for example, are more likely to respond to a local therapy such as resection, radiation therapy, and the like, without the need for a systemic cancer therapy, whereas it was previously thought that any metastatic cancer requires a systemic therapy. The discoveries disclosed herein also allow doctors to identify metastatic cancer for which a local therapy may not be helpful and/or for which systemic therapies, such as DNA damaging drugs, are appropriate.

Measuring the expression of genes and/or miRNAs may be done by a variety of methods. In some embodiments, the measurement comprises performing PCR using RNA obtained from a sample of metastatic tissue as a template. The method may include the use of sets of PCR primers that are complementary to sequences of genes or miRNAs listed in Tables 3A-C, 4A-C, 10A-C, or 11A-C, including any subsets thereof. Measuring expression may also comprise hybridizing nucleic acids to a microarray. The microarray may include nucleic acid sequences that correspond to or are complementary to sequences of genes or miRNAs listed in Tables 3A-C, 4A-C, 10A-C, or 11A-C, including any subsets thereof. Methods may also include the use of nucleic acid probes that correspond to or are complementary to sequences of genes or miRNAs listed in Tables 3A-C, 4A-C, 10A-C, or 11A-C. Any of the primers or probes used may be labeled or modified with fluorescent labels or other moieties that allow the primers or probes to be detected. In some embodiments, measuring expression comprises performing RNA sequencing.

Also disclosed is a method of treating metastatic cancer in a patient, the method comprising administering to the patient a local cancer therapy without administering systemic cancer therapy or administering to the patient an immunotherapy, wherein the patient has been determined to have expression levels of one or more genes listed in Table 10A or one or more miRNAs listed in Table 11B that are within a predetermined amount of the mean expression levels in metastases of a cohort of metastatic cancer patients having a mean overall ten-year survival expectation that is at least 60%. In some embodiments, the patient has been determined to have expression levels of at least, at most, or exactly 5, 10, 20, 30, 40, 50, 60, 70, 80, 90, 100, 110, or 113 genes listed in Table 10A, or any range derivable therein, and/or at least, at most, or exactly 5, 10, 20, 30, 40, 50, or 53 miRNAs listed in Table 11A, or any range derivable therein, that are within a predetermined amount of the mean expression level in metastases of a cohort of metastatic cancer patients having a mean overall ten-year survival expectation that is at least 60%. In some embodiments, the treatments are administered to a patient that has been determined to have expression levels of one or more genes and/or miRNAs that are indicative of an oligometastatic phenotype or of other characteristics of SNF2 metastases. In some embodiments, the patient has been determined to have expression levels of at least, at most, or exactly 5, 10, 20, 30, 40, 50, 60, 70, 80, 90, 100, 110, or 113 genes listed in Table 10A, or any range derivable therein, and/or at least, at most, or exactly 5, 10, 20, 30, 40, 50, or 53 miRNAs listed in Table 11A, or any range derivable therein, that are within a predetermined amount of the mean expression level of a cohort of metastatic cancer patients having a mean overall ten-year survival expectation that is at least 60%.

Also disclosed is a method of treating metastatic cancer in a patient, the method comprising administering to the patient a local cancer therapy without administering systemic cancer therapy, wherein the patient has been determined to have an mRNA and/or miRNA expression profile indicating an oligometastatic phenotype or a specific metastatic subtype that is likely to be successfully treated with local cancer therapy. In some embodiments, the mRNA expression profile is determined by determining the expression of one or more genes listed in Table 10A and the miRNA expression profile is determined by determining the expression of one or more genes listed in Table 11A. In some embodiments, the expression profile is determined by determining the expression levels of at least, at most, or exactly 5, 10, 20, 30, 40, 50, 60, 70, 80, 90, 100, 110, or 113 genes listed in Table 10A, or any range derivable therein, and/or at least, at most, or exactly 5, 10, 20, 30, 40, 50, or 53 miRNAs listed in Table 11A, or any range derivable therein. In some embodiments, the expression profile indicates a ten-year survival expectation of greater than 60% or less than 50, 35, or 20%, an increased likelihood of successful treatment with administration of local cancer therapies, an increased infiltration of immune cells, or other characteristics of any metastatic subtype as described herein.

Also disclosed is a method of treating cancer in a patient having a metastasis from a primary cancer tumor, the method comprising: administering to the patient an immune checkpoint therapy or administering to the patient a local cancer therapy without administering a systemic cancer therapy, wherein the patient has been identified based on the expression levels of one or more mRNA and/or miRNA species in the metastasis as belonging to a group of patients with one or more of the following characteristics: (a) a mean ten-year overall survival expectation of at least 60%; (b) a likelihood of experiencing metastatic recurrence after hepatic resection that is lower than the likelihood for patients outside of the group; and (c) a level of immune cell infiltration into the metastasis that is higher than the mean level for patients outside the group. In some embodiments, the one or more mRNA species comprise one or more transcripts of the genes listed in Table 10A. In some embodiments, the one or more miRNA species comprise one or more transcripts of the miRNAs listed in Table 11A. In some embodiments, the mRNA or miRNA species comprise at least, at most, or exactly 5, 10, 20, 30, 40, 50, 60, 70, 80, 90, 100, 110, or 113 genes listed in Table 10A, or any range derivable therein, and/or at least, at most, or exactly 5, 10, 20, 30, 40, 50, or 53 miRNAs listed in Table 11A, or any range derivable therein. In some embodiments, the metastasis is a liver metastasis and the cancer is colorectal cancer.

Also disclosed is a method of diagnosing a patient having a metastasis from a primary colorectal cancer tumor, the method comprising: (a) measuring the expression levels in the metastasis of one or more of the genes or of one or more miRNAs; (b) identifying the patient as having an oligometastatic phenotype, as being a responder to immune checkpoint cancer therapy, or as having a ten-year survival expectation of greater than 60% if the expression level of one or more of the genes or miRNAs is within a predetermined amount of a first reference expression level or deviates from a second reference expression level by a predetermined amount. In some embodiments, the first reference expression level represents the mean expression level in metastases of a cohort of metastatic cancer patients having an oligometastatic phenotype, being responders to immune checkpoint cancer therapy, and/or or having mean ten-year survival expectation of greater than 60%. In some embodiments, the second reference expression level represents the mean expression level in metastases of a cohort of metastatic cancer patients having a mean ten-year survival expectation of less than 50%. In some embodiments, the one or more genes and/or miRNAs comprise at least, at most, or exactly 5, 10, 20, 30, 40, 50, 60, 70, 80, 90, 100, 110, or 113 genes listed in Table 10A, or any range derivable therein, and/or at least, at most, or exactly 5, 10, 20, 30, 40, 50, or 53 miRNAs listed in Table 11A, or any range derivable therein.

Also disclosed is a method of diagnosing and treating a patient having a metastasis from a primary colorectal cancer tumor, the method comprising: (a) obtaining a tissue sample from the metastasis; (b) measuring the expression of one or more genes and/or miRNAs in the sample; (c) comparing the measured expression level of each gene or miRNA to a reference expression level for that gene or miRNA; (d) identifying the metastasis as an SNF1, SNF2, or SNF3-type metastasis based on the measured expression levels; and (e) administering to the patient an appropriate therapy based on the type of metastasis identified in step (d). In some embodiments, the one or more genes and/or miRNAs comprise at least, at most, or exactly 5, 10, 20, 30, 40, 50, 60, 70, 80, 90, 100, 110, or 113 genes listed in Table 10A, or any range derivable therein and/or at least, at most, or exactly 5, 10, 20, 30, 40, 50, or 53 miRNAs listed in Table 11A, or any range derivable therein. In some embodiments, the appropriate therapy for a patient with an SNF2-type metastasis comprises an immune checkpoint cancer therapy. In some embodiments, the appropriate therapy for a patient with an SNF2-type metastasis comprises a local cancer therapy unaccompanied by systemic cancer therapy. In some embodiments, the appropriate therapy for a patient with an SNF1 metastasis comprises a DNA-damaging cancer therapy. In some embodiments, the DNA-damaging cancer therapy comprises administering PARP inhibitors. In some embodiments, the appropriate therapy for a patient with an SNF1 or SNF3 metastasis comprises a systemic cancer therapy. In some embodiments, the appropriate therapy for a patient with an SNF1 or SNF3 metastasis excludes immune checkpoint cancer therapy.

Also disclosed is a method of providing a prognosis for a patient having metastatic colorectal cancer, the method comprising: (a) evaluating the expression of one or more genes and/or miRNAs in a tissue sample from a metastasis taken from the patient to identify the metastasis as an SNF1, SNF2, or SNF3-type metastasis; (b) determining the clinical risk score of the patient; (c) determining the ten-year survival expectation of the patient as follows: (i) identifying the patient as having a ten-year survival expectation of greater than 90% if the metastasis is type SNF1 or SNF2 and the clinical risk score is 0 or 1; (ii) identifying the patient as having a ten-year survival expectation of between 40 and 50% if the metastasis is type SNF2 and the clinical risk score is 2 or greater or if the metastasis is type SNF3 and the clinical risk score is 0 or 1; and (iii) identifying the patient as having a ten-year survival expectation of less than 24% if if the metastasis is type SNF1 or SNF3 and the clinical risk score is 2 or greater. In some embodiments, the genes and/or miRNAs comprise at least, at most, or exactly 5, 10, 20, 30, 40, 50, 60, 70, 80, 90, 100, 110, or 113 genes listed in Table 10A, or any range derivable therein, and/or at least 5, 10, 20, 30, 40, 50, or 53 miRNAs listed in Table 11A, or any range derivable therein.

Also disclosed is a method comprising evaluating the expression levels of multiple mRNA and/or miRNA species in a sample comprising tissue from a liver metastasis of a patient that has metastatic colorectal cancer to identify the patient as belonging to a first group of metastatic colorectal cancer patients or a second group of metastatic colorectal cancer patients, wherein: (a) the first group has one or more of the following characteristics: (i) a mean ten-year overall survival expectation of at least 60%; (ii) a mean ten-year overall survival expectation that is higher than that for patients outside of the first group; (iii) a likelihood of experiencing metastatic recurrence after hepatic resection that is lower than the likelihood for patients outside of the first group; (iv) a likelihood of being successfully treated without systemic cancer treatments that is higher than the likelihood for patients outside of the first group; and (v) a likelihood of being successfully treated with immune checkpoint therapy that is higher than the likelihood for patients outside of the first group; and (b) the second group has one or more of the following characteristics: (i) a mean ten-year overall survival expectation of less than 50%; (ii) a mean ten-year overall survival expectation that is lower than that for patients outside of the second group; (iii) a likelihood of experiencing metastatic recurrence after hepatic resection that is higher than for patients outside of the second group; (iv) a likelihood of being successfully treated without systemic cancer treatments that is lower than the likelihood for patients outside of the second group; (v) a likelihood of being successfully treated with immune checkpoint therapy that is lower than the likelihood for patients outside of the second group; and (vi) a likelihood of being successfully treated with DNA damaging cancer therapy that is higher than the likelihood for patients outside of the second group. In some embodiments, the mRNA species comprise transcripts of one or more genes listed in Table 10A. In some embodiments, the miRNA species comprise one or more of the miRNAs listed in Table 11A. In some embodiments, the patient is identified as belonging to the first group of patients if the expression of one or more genes listed in Table 10A is within a predetermined amount of a reference expression level of the one or more genes. In some embodiments, the patient is identified as belonging to the first group of patients if the expression of one or more miRNAs listed in Table 11A is within a predetermined amount of a reference expression level of the one or more miRNAs. In some embodiments, step (b) comprises using a classifier that has been trained to identify an RNA expression pattern associated with the first group of patients. In some embodiments, the classifier evaluates the expression levels of at least, at most, or exactly 5, 10, 20, 30, 40, 50, 60, 70, 80, 90, 100, 110, or 113 genes listed in Table 10A, or any range derivable therein. In some embodiments, the classifier evaluates the expression levels of at least, at most, or exactly 5, 10, 20, 30, 40, 50, or 53 of the miRNAs listed in Table 11A, or any range derivable therein. In some embodiments, the method further comprises administering an immune checkpoint therapy to a patient identified as belonging to the first group. In some embodiments, the method further comprises treating a patient identified as belonging to the first group with local treatment of liver metastases unaccompanied by systemic cancer treatment. In some embodiments, the method further comprises administering a DNA damaging cancer therapy to a patient identified as belonging to the second group of patients.

Also disclosed is a method of identifying a molecular subtype of metastatic cancer, the method comprising performing genome-wide expression profiling of a plurality of metastatic tissue samples to generate expression data of mRNA and miRNA in the tissue samples and analyzing the expression data using a similarity network fusion algorithm or other integrated molecular analysis technique that identifies similarities in both mRNA and miRNA expression data among samples to identify groups of samples having expression patterns that are similar to other samples in the group and that are dissimilar from samples outside the group. In some embodiments, the method further comprises identifying genes and miRNAs that are differentially expressed in a group of samples relative to either a mean expression level across all samples or a mean expression level of samples outside the group. In some embodiments, the method further comprises identifying a subset of the differentially expressed genes and/or miRNAs whose expression levels in a single sample can be used to accurately classify the sample as belonging to a particular molecular subtype or not belonging to a particular molecular subtype.

In any of the embodiments described herein, the patient may have already been diagnosed with cancer or already had tumor resection before any of the steps of methods described herein are performed.

Any step or aspect of an embodiment described herein may be implemented in the context of any other embodiment described herein

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-B show clinical outcomes following surgical resecton of limited liver metastases from colorectal cancer. Kaplan-Meier curves of overall survival by (A) clinical recurrence status (as determined by post-operative surveillance CT imaging and serum CEA measurements) or (B) Clinical Risk Scores (CRS) following hepatic resection of limited de novo CRCLM. Low CRS was defined as values less than two. P-values were determined using log-rank tests.

FIGS. 2A-E show the identification of intrinsic molecular subtypes of colorectal liver metastases. (A) Consensus Molecular Subtypes (CMS) of primary colorectal cancers obtained from the Colorectal Cancer Subtyping Consortium (CRCSC) or calculated in primary colorectal cancers of The Cancer Genome Atlas (TCGA). CMS subtypes were also determined in colorectal liver metastases from patients undergoing partial hepatectomy of resectable liver metastases (UC, NS, MSK1 and MSK2 cohorts) or biopsy of unresectable liver metastases (MSK3, Italian and French cohorts). Cohorts contain independent clinical and molecular datasets. (B) Consensus clustering based on Similarity Network Fusion (SNF) subtyping of colorectal liver metastases. (C) Kaplan-Meier curves of overall survival by SNF subtype. P-value was determined using a log-rank test. (D) Metastatic recurrence patterns by SNF subtype. Asterisks denote statistical significance based on Fisher's exact test for each SNF subtype versus the two other SNF subtypes. (E) Differentially expressed mRNAs (left) and miRNAs (right) between the three SNF subtypes (see Tables 3A-C and 4A-C)

FIGS. 3A-D show the molecular signatures of intrinsic subtypes of colorectal liver metastases. (A) Ensemble of Gene Set Enrichment Analyses (EGSEA) of significantly enriched ‘Hallmark’ and ‘Cellular Estimate’ gene signatures within each SNF subtype. Color intensity in EGSEA heatmaps is proportional to significance level (see Tables 5A-C and 6). SNF1=red, SNF2=green, SNF3=blue. (B) Functional categorization of differentially expressed immune genes overexpressed in SNF2. The SNF2 column indicates that many immune genes are overexpressed in SNF2, and the SNF1 and SNF3 columns indicate that many immune genes are suppressed in these subtypes. (C) OncoPrint plot of recurrent colorectal cancer mutations and copy number alterations by SNF subtype. (D) Frequencies of SNF-specific genomic alterations. Asterisks denote statistical significance based on Fisher's exact test comparing each SNF group to the two other SNF groups.

FIGS. 4A-C show the integration of intrinsic molecular subtypes and clinical risk stratification. (A) Kaplan-Meier curves of overall survival following initial hepatic resection of limited de novo CRCLM based on integrated risk classification of SNF subtype and Clinical Risk Scores (CRS). P-value was determined using a log-rank test. (B) Metastatic recurrence patterns for integrated risk groups. Asterisks denote statistical significance based on Fisher's exact test for each individual group versus the two additional groups. (C) Proposed classification of colorectal liver metastasis based on SNF subtypes

FIG. 5. Overview of study design.

FIG. 6. Overall survival by Consensus Molecular Subtypes (CMS) in patients with colorectal liver metastases. CMS subtypes were determined for 93 patients in our cohort from RNA Sequencing data using the methodology implemented in Sage-Bionetwork's CMS classifier R package (see Example 7 for materials and methods). Kaplan-Meier survival analysis of 10-year overall survival was performed for patients with CMS2, CMS4 and unclassified patterns. One patient with a CMS1 pattern was excluded from survival analysis. No. at risk denotes the number of patients at risk at each specified time point. P-value was determined using a log-rank test across groups.

FIGS. 7A-D. Consensus clustering analysis of the mRNA expression data for 95 patients with colorectal liver metastases. (A) Heatmaps of the consensus matrices for the predefined cluster numbers k (k=2, 3, 3, 4, 5, and 6); (B) Kaplan-Meier plot for 10-year overall survival of the patients stratified by their consensus cluster memberships. P-value was determined using a log-rank test across groups; (C) Consensus Cumulative Distribution Function (CDF) plot of the consensus matrix for each k, estimated by a histogram of 100 bins. The lower left portion of the CDF plot represents samples rarely clustered together, and the upper right portion represents those almost always clustered together, whereas the middle portion represents those with occasional co-assignments in different clustering runs; A flat middle segment, suggesting that very few sample pairs are ambiguous when k is correctly inferred, can be used to determine the optimal k of consensus clusters. (D) Proportion of ambiguous clustering (PAC) plot defined as the fraction of sample pairs with consensus index values falling in the intermediate sub-interval (0.1, 0.9). A low value of PAC indicates a flat middle segment in the CDF plot and is allowed to infer the optimal k (k=2).

FIGS. 8A-D. Consensus clustering analysis of the miRNA expression data for 116 patients with colorectal liver metastases. (A) Heatmaps of the consensus matrices for the predefined cluster numbers k (k=2, 3, 3, 4, 5, and 6); (B) Kaplan-Meier plot for 10-year overall survival of the patients stratified by their consensus cluster memberships. P-value was determined using a log-rank test across groups; (C) Consensus Cumulative Distribution Function (CDF) plot of the consensus matrix for each k, estimated by a histogram of 100 bins. The lower left portion of the CDF plot represents samples rarely clustered together, and the upper right portion represents those almost always clustered together, whereas the middle portion represents those with occasional co-assignments in different clustering runs; A flat middle segment suggesting that very few sample pairs are ambiguous when k is correctly inferred, can be used to determine the optimal k of consensus clusters. (D) Proportion of ambiguous clustering (PAC) plot defined as the fraction of sample pairs with consensus index values falling in the intermediate sub-interval (0.1, 0.9). A low value of PAC indicates a flat middle segment in the CDF plot and is allowed to infer the optimal k (k=2).

FIG. 9. Median Silhouette Index (SI) for the SNF clusters under 72 parameter settings. SI represents the separation distance between the resulting clusters under each parameter setting. The top 8 parameter settings with highest median SI (in red) were selected for further analysis, and the corresponding clustering results were used to determine the final SNF cluster memberships through majority voting.

FIGS. 10A-B. Associations of SNF subtypes and clinicopathological variables. Statistical significance was assessed using Fisher's exact tests for categorical variables and Student's t-test for continuous variables. Asterisks denote P-values ≤0.05 in the comparison of one SNF subtype versus the remaining subtypes.

FIG. 11. Non-random association of SNF network structure with overall survival in metastatic colorectal cancer patients. Shown is the density of the -log P-value for each simulation of the SNF cluster set with members closest to the consensus for illustrative purposes. The red line in the figure represents the empirical P-value for a particular parameter set (parameter settings: K=25, alpha=0.6, T=20). The table inset contains the key statistics for each of the top 8 SNF cluster parameterizations in order of decreasing median Silhouette Index; highlighted is a parameter setting which produced SNF clusters with memberships closest to the consensus SNF grouping differing only by two sample assignments.

FIG. 12. Distant metastasis-free survival by SNF subtype. SNF subtypes were determined for 93 patients in our cohort. Kaplan-Meier survival analysis of distant metastasis-free survival (event=first metastatic recurrence or death). No. at risk denotes number of patients at risk at each specified time point. P-value was determined using a log-rank test across groups.

FIG. 13. Primary CRC CMS subtype by SNF subtype. Shown is the distribution of primary colorectal cancer Consensus Molecular Subtypes (CMS) by SNF subtypes of colorectal liver metastases. CMS subtypes were determined for 93 patients in our cohort from RNA Sequencing data using the methodology implemented in Sage-Bionetwork's CMS classifier R package (see Example 7 for Methods and Materials). P-value denotes a Chi-Squared test across the three SNF groups.

FIGS. 14A-B. Perioperative chemotherapy regimens and associations with SNF subtype. (A) Types of perioperative chemotherapies received by patients which were included in the integrated SNF analysis. Specific details regarding chemotherapy regimens were available for 81 of 93 patients. (B) Association between type of chemotherapy received in perioperative setting and molecular subtype of metastasis derived from SNF analysis. P-value denotes a Chi-Squared test across the three SNF groups.

FIGS. 15A-C. Prediction Analysis of Microarrays (PAM)-based classifier to distinguish SNF subtypes. (A) Model evaluation on the test data set from our cohort samples. (B) The area under the ROC curve demonstrates the classification performance compared to a random classifier on the test data set (AUC=0.875 vs. AUC=0.50 for random classifier). (C) Mosaic plot showing the concordance between the predicted SNF cluster labels and the Clinical Risk Scores (CRS) in an independent data set of patients who underwent hepatic resection of limited colorectal liver metastases (Memorial Sloan-Kettering Cancer Center, n=96, ArrayExpress Identifier: E-MTAB-1951).

FIGS. 16A-D. Histologic analysis by SNF subtypes of liver metastasis. (A) Hematoxylin and eosin, (B) Trichome, (C) CD3, and (D) CD8 staining by SNF subtype. Shown are 10× magnification fields for three representative patients from each SNF subtype. Top row, SNF1. Middle row, SNF2. Bottom row, SNF3.

FIG. 17. Oncoprint plot of exomic mutations occurring in 59 patients with colorectal liver metastases. Genes mutated in ≥10% of samples are shown. Values to the left of the Oncoprint plot represent the percentage of samples that harbor a mutation (non-synonymous SNVs or indels) in a given gene. The horizontal bar plot indicates the number of mutations for each patient sample falling within these recurrently altered genes. The vertical bar plot to the right depicts the number of mutations seen in each gene across all 59 samples. ‘Splicing’ refers to mutations that affect a splice donor or acceptor site.

FIGS. 18A-E. Cytotoxic immune signature by SNF subtypes. (A) Distribution of cytotoxic immune gene scores' by SNF subtype. MSI-H and MSI-L, microsatellite instability-high and -low. MSS, microsatellite stable. N/A, missing data. (B) Mean (±S.E.M.) values of cytotoxic cell immune scores by SNF subtype. (C) Percentage of MSS patients within each SNF subtype. Differences in cytotoxic immune scores by somatic ARID2 (D) or SNF-specific mutations (E). Metastases classified as harboring SNF2-specific mutations included CDK12, NRAS, and EBF1 mutations, whereas SMAD3, NOTCH1, or PIK3C2B mutations characterized SNF1, 3-specific mutations. Data represent mean±S.E.M. values. Asterisks denote P-values ≤0.05.

FIG. 19. Overall survival by integration of SNF subtype and Clinical Risk Scores (CRS). High CRS denotes scores ≥2. Patient subgroups defined by SNF and CRS were classified into low-, intermediate-, and high-risk cohorts based on Kaplan-Meier analysis of overall survival rates. P-value was determined using a log-rank test across groups. No. at risk denotes number of patients at risk at each specified time point. Table inset denotes hazard ratios (95% confidence intervals) for Cox multivariate proportional hazard analysis of SNF and CRS (both as nominal variables). A multivariate interaction was assessed between SNF and CRS and removed from the final multivariate model due to non-significance. P-value was determined using likelihood ratio test.

FIGS. 20A-B. Metastatic recurrence patterns by integrated risk classification. Risk groups were determined for 87 patients in our cohort. (A) Kaplan-Meier survival analysis of distant metastasis-free survival (event=first metastatic recurrence or death). P-value was determined using a log-rank test across groups. No. at risk denotes number of patients at risk at each specified time point. (B) Association of molecular/clinical risk stratification groups with patterns of metastatic recurrence. Statistical significance was assessed using Fisher's exact tests between one risk group versus the two remaining groups. Asterisks denote P-values ≤0.05.

### DETAILED DESCRIPTION

Here, utilizing independent clinical cohorts of CRC patients who underwent resection of liver metastases, the inventors have identified integrated molecular patterns in liver metastases associated with long-term survival. The inventors' findings indicate a molecular basis for oligometastasis that is predictive of clinical outcome and complementary to established clinical risk factors associated with long-term survival following hepatic resection. Aspects of the current invention have important clinical implications in the selection of local therapy for those patients with potentially curable oligometastatic disease from those whose few metastases are a part of a large cascade of widespread disease. These concepts may be applicable to many histological types of cancer. Methods disclosed herein involve determining expression levels of genes and miRNAs in liver metastases to identify the molecular subtype of the metastasis. The subtype classification can be used to provide a prognosis and to guide treatment decisions. These and other aspects of the disclosed methods will be described in greater detail below.

### A. GENE AND MIRNA EXPRESSION LEVELS

Methods disclosed herein include measuring expression of genes and/or miRNAs. Measurement of expression can be done by a number of processes known in the art. The process of measuring expression may begin by extracting RNA from a metastasis tissue sample. Extracted mRNA and/or miRNA can be detected by hybridization (for example by means of Northern blot analysis or DNA or RNA arrays (microarrays) after converting mRNA into labeled cDNA) and/or amplification by means of a enzymatic chain reaction. Quantitative or semi-quantitative enzymatic amplification methods such as polymerase chain reaction (PCR) or quantitative real-time RT-PCR or semi-quantitative RT-PCR techniques can be used. Primer pairs may be designed for the purpose of superimposing an intron to distinguish cDNA amplification from the contamination from genomic DNA (gDNA). Additional primers or probes, which are preferably labeled, for example with fluorescence, which hybridize specifically in regions located between two exons, are optionally designed for the purpose of distinguishing cDNA amplification from the contamination from gDNA. If desired, said primers can be designed such that approximately the nucleotides comprised from the 5′ end to half the total length of the primer hybridize with one of the exons of interest, and approximately the nucleotides comprised from the 3′ end to half the total length of said primer hybridize with the other exon of interest. Suitable primers can be readily designed by a person skilled in the art. Other amplification methods include ligase chain reaction (LCR), transcription-mediated amplification (TMA), strand displacement amplification (SDA) and nucleic acid sequence based amplification (NASBA). Expression levels of mRNAs and/or miRNAs may also be measured by RNA sequencing methods known in the art.

To normalize the expression values of one gene among different samples, comparing the mRNA level of the gene of interest in the samples from the subject object of study with a control RNA level is possible. As it is used herein, a “control RNA” is an RNA of a gene for which the expression level does not differ among different metastatic subtypes, for example a gene that is constitutively expressed in all types of cells. A control RNA is preferably an mRNA derived from a housekeeping gene encoding a protein that is constitutively expressed and carrying out essential cell functions.

Methods disclosed herein may include comparing a measured expression level to a reference expression level. The term “reference expression level” refers to a value used as a reference for the values/data obtained from samples obtained from patients. The reference level can be an absolute value, a relative value, a value which has an upper and/or lower limit, a series of values, an average value, a median, a mean value, or a value expressed by reference to a control or reference value. A reference level can be based on the value obtained from an individual sample, such as, for example, a value obtained from a sample from the subject object of study but obtained at a previous point in time. The reference level can be based on a high number of samples, such as the levels obtained in a cohort of subjects having a particular characteristic. The reference level may be defined as the mean level of the patients in the cohort. For example, the reference expression level for a gene or miRNA can be based on the mean expression level of the gene or miRNA obtained from a number of patients who have SNF2 metastases. A reference level can be based on the expression levels of the markers to be compared obtained from samples from subjects who do not have a disease state or a particular phenotype. The person skilled in the art will see that the particular reference expression level can vary depending on the specific method to be performed.

Some embodiments include determining that a measured expression level is higher than, lower than, increased relative to, decreased relative to, equal to, or within a predetermined amount of a reference expression level. In some embodiments, a higher, lower, increased, or decreased expression level is at least 1, 1.5, 2, 2.5, 3, 3.5, 4, 4.5, 5, 5.5, 6, 7, 8, 9, 10, 12, 14, 16, 18, 20, 50, 100, 150, 200, 250, 500, or 1000 fold (or any derivable range therein) or at least 10, 20, 30, 40, 50, 60, 70, 80, 90, 100, 200, 300, 400, 500, 600, 700, 800, or 900% different than the reference level, or any derivable range therein. These values may represent a predetermined threshold level, and some embodiments include determining that the measured expression level is higher by a predetermined amount or lower by a predetermined amount than a reference level. In some embodiments, a level of expression may be qualified as “low” or “high,” which indicates the patient expresses a certain gene or miRNA at a level relative to a reference level or a level with a range of reference levels that are determined from multiple samples meeting particular criteria. The level or range of levels in multiple control samples is an example of this. In some embodiments, that certain level or a predetermined threshold value is at, below, or above 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percentile, or any range derivable therein. Moreover, a threshold level may be derived from a cohort of individuals meeting a particular criteria. The number in the cohort may be, be at least, or be at most 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 700, 800, 900, 1000, 1100, 1200, 1300, 1400, 1500, 1600, 1700, 1800, 1900, 2000 or more (or any range derivable therein). A measured expression level can be considered equal to a reference expression level if it is within a certain amount of the reference expression level, and such amount may be an amount that is predetermined. This can be the case, for example, when a classifier is used to identify the molecular subtype of a metastasis. The predetermined amount may be within 0.1, 0.2, 0.3, 0.4, 0.5, 0.6, 0.7, 0.8, 0.9, 1.0, 2, 3, 4, 5, 6, 7, 8, 9, 10, 11, 12, 13, 14, 15, 16, 17, 18, 19, 20, 21, 22, 23, 24, 25, 30, 35, 40, 45, or 50% of the reference level, or any range derivable therein.

For any comparison of gene or miRNA expression levels to a mean expression levels or a reference expression levels, the comparison is to be made on a gene-by-gene and miRNA-by-miRNA basis. For example, if the expression levels of gene A, gene B, and miRNA X in a patient's metastasis are measured, a comparison to mean expression levels in metastases of a cohort of patients would involve: comparing the expression level of gene A in the patient's metastasis with the mean expression level of gene A in metastases of the cohort of patients, comparing the expression level of gene B in the patient's metastasis with the mean expression level of gene B in metastases of the cohort of patients, and comparing the expression level of miRNA X in the patient's metastasis with the mean expression level of miRNA X in metastases of the cohort of patients. Comparisons that involve determining whether the expression level measured in a patient's metastasis is within a predetermined amount of a mean expression level or reference expression level are similarly done on a gene-by-gene and miRNA-by-miRNA basis, as applicable.

### B. IDENTIFYING MOLECULAR SUBTYPES OF METASTASES

Methods disclosed herein can be used to identify different molecular subtypes of metastatic cancer that correlate with different clinical outcomes and different sensitivities to particular treatment regimens. The subtypes can be identified using an integrated molecular analysis techniques. One such technique described in the Examples below is a similarity network fusion (SNF) algorithm, which incorporates parallel miRNA and mRNA expression networks in a number of patient samples. The SNF analysis established three subtypes of metastatic cancer based solely on expression data, but the subtypes exhibited heterogenous clinical outcomes. Other types of integrated approaches to identifying molecular subtypes can also be used. For example, the inventors analyzed the miRNA and mRNA expression data using consensus clustering of clusters and iClusterPlus and found that, similar to SNF, these approaches identified three distinct subtypes of metastases based on expression alone, and that the distinct subtypes showed statistically significant differences in clinical outcomes of patients. These data demonstrate that the intrinsic subtypes are independent of the type of integrated molecular analysis used to identify them. Furthermore, the discovery that metastatic cancers are heterogeneous and include distinct molecular subtypes enables skilled persons to identify metastatic subtypes of different types of metastatic cancers using integrated analyses of gene and miRNA expression, including liver cancer, testicular cancer, biliary cancer, ovarian cancer, urinary tract cancer, pancreatic cancer, prostate cancer, esophageal cancer, gastric cancer, head and neck cancer, cervical cancer, lung cancer, neuroendocrine cancer, kidney cancer, breast cancer, melanoma, and other cancers that can progress to metastatic cancer.

### C. CANCER TREATMENT

Methods disclosed herein may include administering a cancer therapy or determining a course of cancer treatment based on an identified metastatic subtype. Some embodiments include administering a local cancer treatment or determining that a local cancer treatment is appropriate. Local cancer treatments include those that target cancer tissue using a technique directed to a specific organ or limited area of the body. Local cancer treatments include surgery (i.e., resection), radiation therapy, cryotherapy, laser therapy, topical therapy, high intensity focused ultrasound, and photodynamic therapy. The local treatments may include stereotactic body radiotherapy (SBRT), stereotactic ablative body radiotherapy (SABR), stereotactic radiosurgery (SRS), radiofrequency ablation (RFA), percutaneous cryoablation therapy (PCT), and photodynamic therapy (PDT). The local therapies may be directed at the primary tumor and/or at one or more metastases.

Systemic cancer therapies are those that are distributed widely within the body, such as a variety of drug treatments, which may be delivered orally or intravenously. Examples of systemic therapies include chemotherapy, hormone therapy, immunotherapy, and targeted therapy (i.e., drugs that are distributed widely within the body, but have targeted effects on cancer cells). More specifically, chemotherapy includes administering drugs such as cyclophosphamide, paclitaxel, epirubicin, methotrexate, gemcitabine, albumin-bound paclitaxel, carboplatin, etoposide, doxorubicin, capecitabine, fluorouracil, vinorelbine, docetaxel, liposomal doxorubicin, eribulin, or irinotecan, including combinations thereof. Immunotherapy includes monoclonal antibodies, such as alemtuzumab, trastuzumab, ibritumomab tiuxetan, brentuximab vedotin, ado-trastuzumab emtansine, denileukin diftitox, and blinatumomab; immune checkpoint inhibitors, such as pembrolizumab, nivolumab, atezolizumab, avelumab, durvalumab, and ipilimumab; and cancer vaccines such as sipuleucel-T.

Identifying the molecular subtype of metastatic colorectal cancer can be used to determine an appropriate treatment regimen. In some embodiments, the appropriate treatment for SNF1 metastases include EGFR inhibitors, PARP inhibitors, PI3K inhibitors, NOTCH inhibitors, angiogensis inhibitors, DNA damaging agents, STING agonists, innate immune agonists, RNA vaccines, or combinations thereof. In some embodiments, the appropriate treatment for SNF2 metastases include PD-1/PD-L1 immunotherapies, other immunotherapies, beta-secretase inhibitors, lipid-lowering agents, and combinations thereof. In some embodiments, the appropriate treatment for SNF3 metastases include PDGF/PDGFR inhibitors, VEGF/VEGFR inhibitors, angiogenesis inhibitors, JAK1/JAK2 inhibitors, COX2 inhibitors, HDAC inhibitors, DNA demethylating agents, other epigenetic modifiers, and combinations thereof.

Methods disclosed herein can also include making treatment decisions based on an integrated risk group classification of a patient. This classification combines the molecular subtyping of the metastasis with a clinical risk score of the patient. Integration of SNF subtypes and CRS yielded three prognostic risk groups: (1) low-risk (22% of patients)—SNF1 and SNF2 subtypes with low CRS; (2) intermediate-risk (29% of patients)—SNF2 subtype with high CRS and SNF3 subtype with low CRS; (3) high-risk patients (49% of patients)—SNF1 and SNF3 subtypes with high CRS. A patient's integrated risk group indicates the likelihood of benefit from local metastasis-directed therapies such as surgical resection, stereotactic body radiotherapy (SBRT), stereotactic ablative body radiotherapy (SABR), stereotactic radiosurgery (SRS), radiofrequency ablation (RFA), percutaneous cryoablation therapy (PCT), and photodynamic therapy (PDT): low-risk patients have the highest likelihood of benefit from these therapies, high-risk patients have the lowest likelihood of benefit from these therapies, and intermediate-risk patients have an intermediate likelihood of benefit from these therapies.

Conventionally, it has been thought that metastatic cancer always requires a systemic therapy. However, the identification of molecular subtypes of metastatic cancer as described herein shows that some metastatic cancers are likely to respond favorably to local therapies and may not need an additional systemic therapy. Conversely, some metastatic cancers are not likely to respond to local therapy alone, or at all, and should therefore be treated with appropriate systemic therapies.

### D. EXAMPLES

### Example 1. Clinical Characteristics and Patient Outcomes

One hundred thirty-four patients with comprehensive clinical annotations underwent hepatic resection of limited CRCLM. The clinical characteristics of these patients are summarized in Table 1. The median patient age was 61 years (range, 29-85). Patients were diagnosed with primary adenocarcinoma of the colon (72%) or rectum (28%) and presented with either synchronous (47%) or metachronous (53%) liver metastasis. The initial number of liver metastases was one in 61%, two in 22% and three or more in 17% of patients. Liver metastases were limited to one hepatic lobe in 91% of patients and two hepatic lobes in 9% of patients. Our analysis focused on de novo liver metastases and excluded patients with extrahepatic disease or a history of previously resected metastasis. Patients received uniform treatment with 5-fluorouracil-based perioperative chemotherapy, curative intent management of primary colorectal tumors, and partial hepatectomy of all visible liver metastases (Table 1). Post-operatively all patients were surveilled with serial axial CT imaging and serum CEA levels.

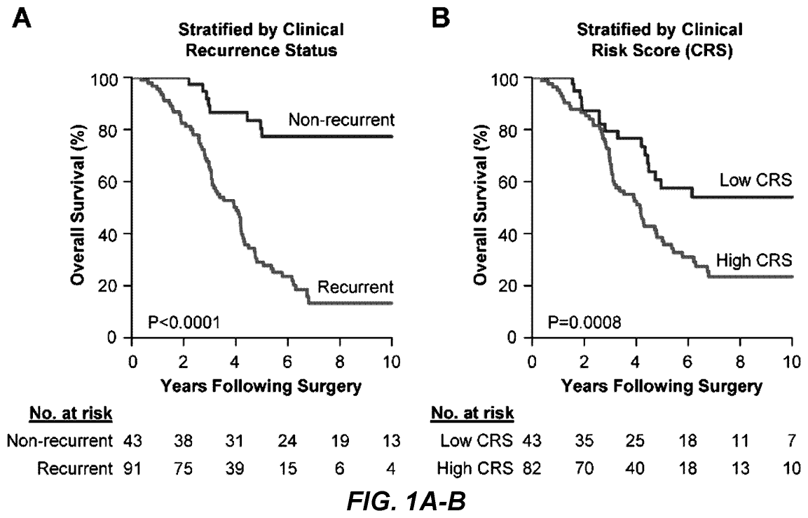

At a median follow-up of 49 months, 32% of patients had no evidence of metastatic recurrence. These patients had a 10-year OS of 77% whereas patients with clinically evident, recurrent metastases exhibited a 10-year OS of 13% (P<0.0001, log-rank test) (FIG. 1A). Clinical Risk Scores (CRS), a widely accepted prognostic tool for CRC patients undergoing liver metastasis resection^{9,12,13}, were calculated using the following adverse clinical and pathological features: (1) disease-free interval between primary tumor diagnosis and development of metastasis <12 months, (2) number of liver metastases >1, (3) largest liver metastasis >5.0 cm, (4) lymph node-positive primary CRC, and (5) CEA>200 ng/mL. 34% of patients exhibited a low CRS (less than two adverse features). As expected, OS was significantly greater for patients with low versus high CRS (two or more adverse features) (10-year: 62% vs. 22%, P=0.0008, log-rank test) (FIG. 1B). These outcomes were consistent with those previously reported in the literature⁹. In this context, the inventors investigated whether the intrinsic molecular features of CRCLM enhance the identification of patients with long-term survival after hepatic resection of limited metastases.

TABLE 1
Clinical and Pathological Characteristics of Colorectal
Cancer Patients with liver metastases selected for study

| | Clinical cohort | |
|---|---|---|
| Clinicopathological Variable | (n = 134) | |
| Age (median, range) | 61 | (29-85) |

| Sex | | |
|---|---|---|
| Male | | 57% |
| Female | | 43% |
| Primary tumor | | |
| Colon | | 72% |
| Rectum | | 28% |
| Metastatic presentation | | |
| Synchronous | | 47% |
| Metachronous | | 53% |
| Tumor size | | |
| <=5 | cm | 78% |
| >5 | cm | 22% |
| Primary lymph node status | | |
| Negative | | 36% |
| Positive | | 64% |
| Initial number of liver metastases | | |
| 1 | | 61% |
| 2 | | 22% |
| 3+ | | 17% |
| Disease-free interval from | | |
| primary tumor to metastasis | | |
| <12 | mo | 61% |
| >=12 | mo | 39% |
| CEA level | | |
| <200 | ng/mL | 95% |
| >=200 | ng/mL | 5% |

| Clinical Risk Scores (CRS) | | |
|---|---|---|
| <2 | 34% | |
| ≥2 | 66% | |
| Hepatic involvement | | |
| Unilobar | 91% | |
| Bilobar | 9% | |
| Extent of resection | | |
| Wedge/segmentectomy | 58% | |
| Lobectomy/ | 42% | |
| extended lobectomy | | |
| Resection margin | | |
| Negative | 85% | |
| Positive | 15% | |
| Peri-operative chemotherapy | 98% | |
| Follow-up (mo) (median, range) | 49 | (4 · 3-328) |

| | |
|---|---|
| Metastatic recurrence | 68% |
| Patterns of failure | |
| Liver only | 38% |
| Liver and lung | 34% |
| Other sites (e.g. peritoneum, | 28% |
| bone, adrenal, brain) | |
| Death event | 58% |

### Example 2. Consensus Molecular Subtypes of Primary Colorectal Cancer

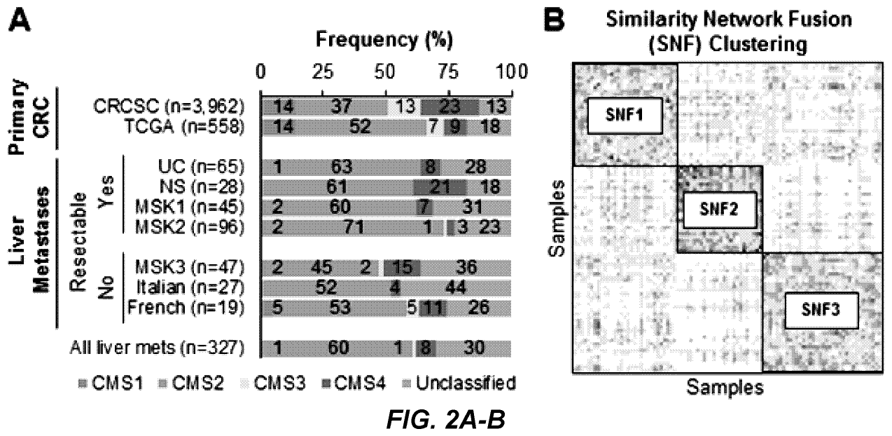

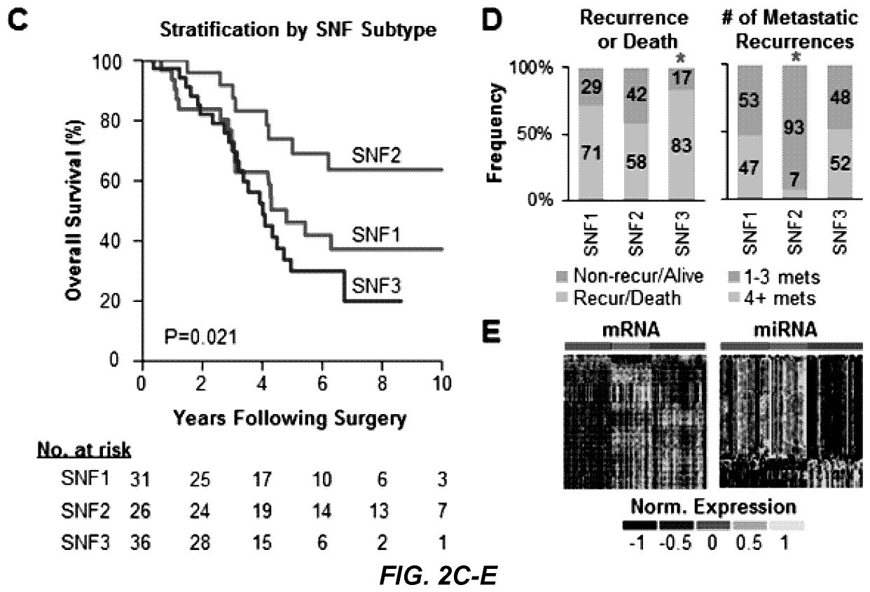

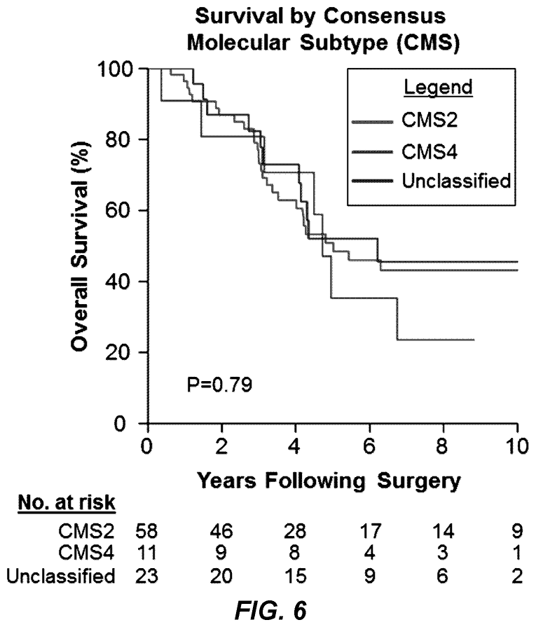

Gene expression analysis is an established approach for molecular subtyping of primary human cancers^{14,15}. The International Colorectal Cancer Subtyping Consortium (CRCSC) demonstrated the existence of four biologically and clinically distinct Consensus Molecular Subtypes (CMS) of CRC based on gene expression analysis of 3,962 primary tumors^{16,17}. However, it is unknown whether CMS subtypes also exist in CRCLM. First, the application of CMS classification to the analysis of RNA Sequencing data from 558 primary CRC tumors in The Cancer Genome Atlas (TCGA)¹⁸ was validated, which verified the expected frequencies of CMS subtypes (FIG. 2A). Then the single-sample CMS classifier was applied to the analysis of two independent CRCLM datasets derived from RNA Sequencing analysis of 93 patients in our study (Cohorts UC and NS in FIG. 2A), which demonstrated CMS2 and CMS4 patterns in 62% and 12% of liver metastases with a notable absence of CMS1 (1%) and CMS3 (0%) patterns (FIG. 2A). The inventors examined whether this result was related to selection bias for patients with limited, resectable metastatic disease or was generalizable to widely metastatic or unresectable CRCLM. In five independent datasets comprising an additional 234 CRCLMs derived from either hepatic resection (60% of samples) or biopsy due to unresectable or widely metastatic disease (40% of samples), similar frequencies of CMS2 and CMS4 patterns in 60% and 7% (vs. 37% and 23% of primary CRC) were observed. CMS1 and CMS3 subtypes comprised 2% and 1% of liver metastases (vs. 14% and 13% of primary CRC). In addition, 30% of CRCLM were unclassifiable based on CMS subtypes (FIG. 2A). While CMS classes were associated with distinct clinical outcomes in primary CRCs, no association between CMS subtypes and OS in patients with resected liver metastases was observed (FIG. 6).

### Example 3—Integrated Transcriptional Subtypes of Colorectal Liver Metastasis

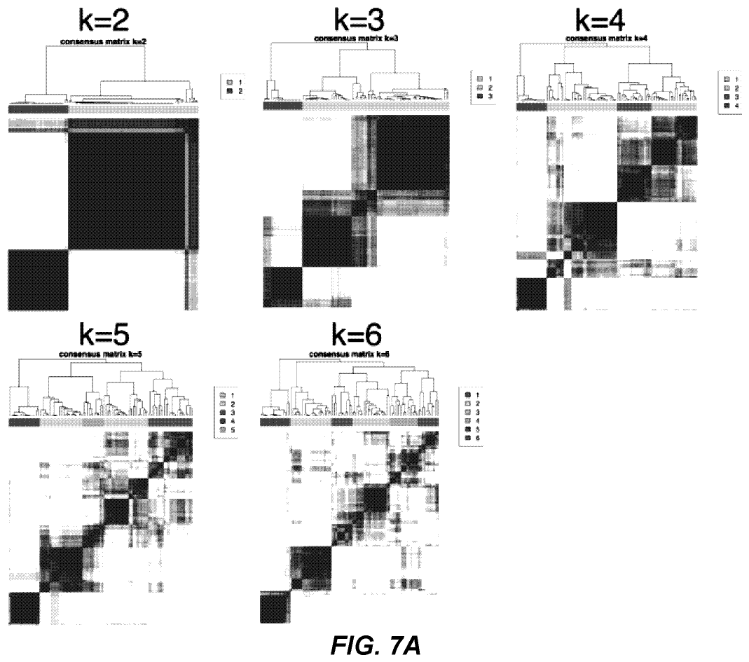

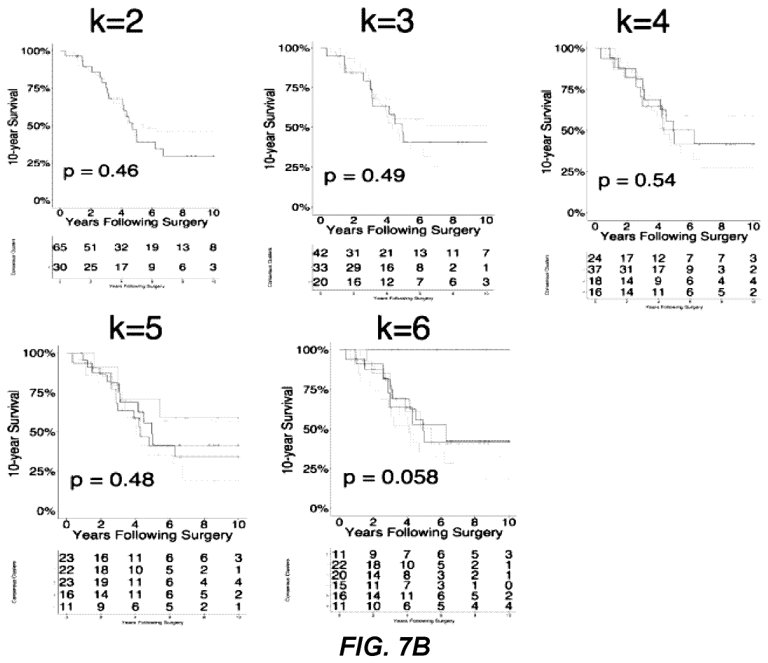

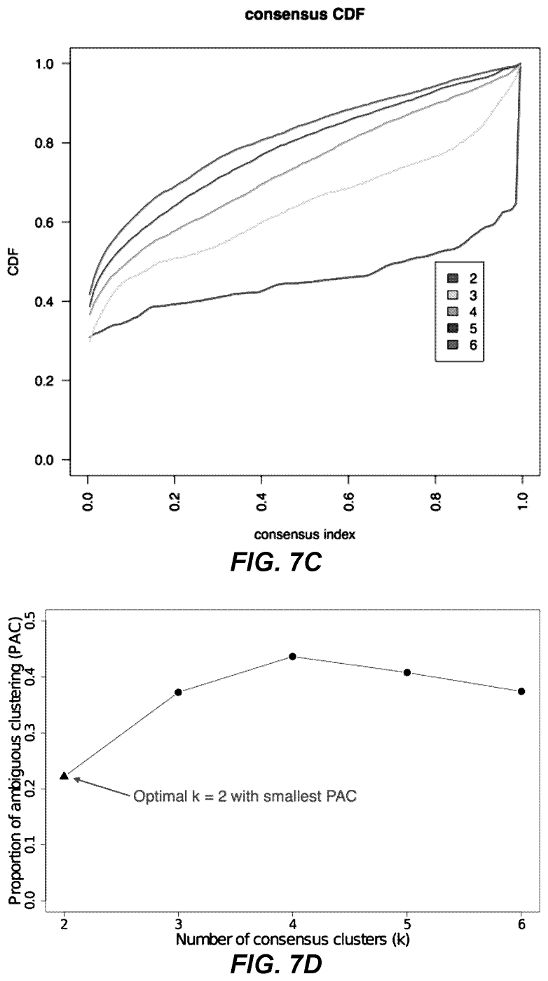

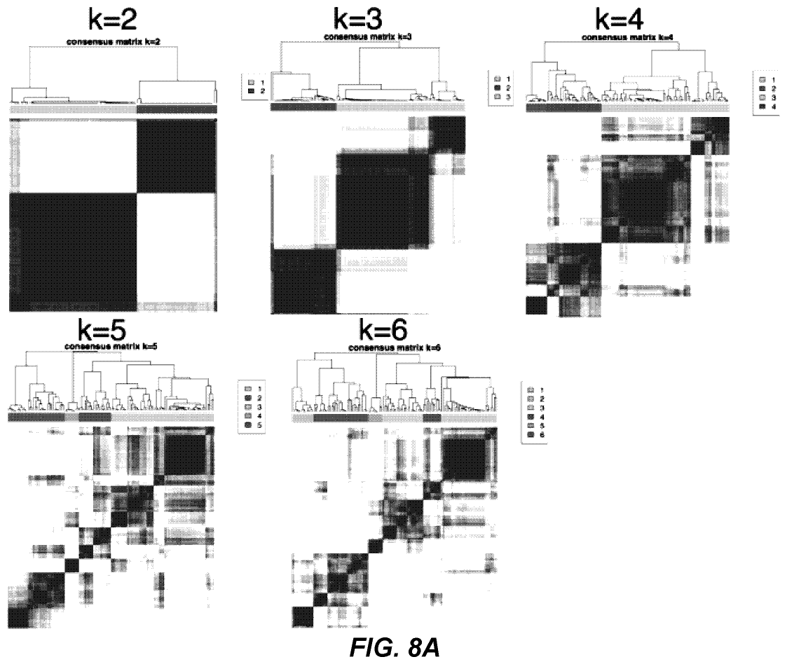

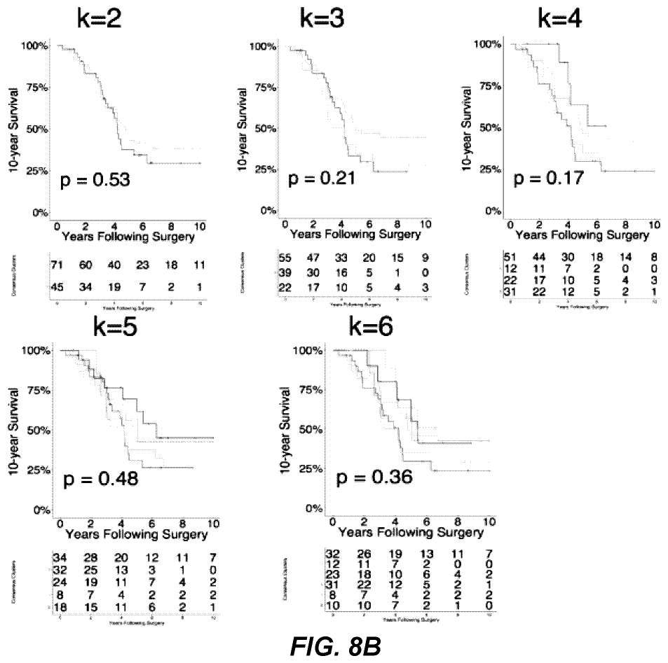

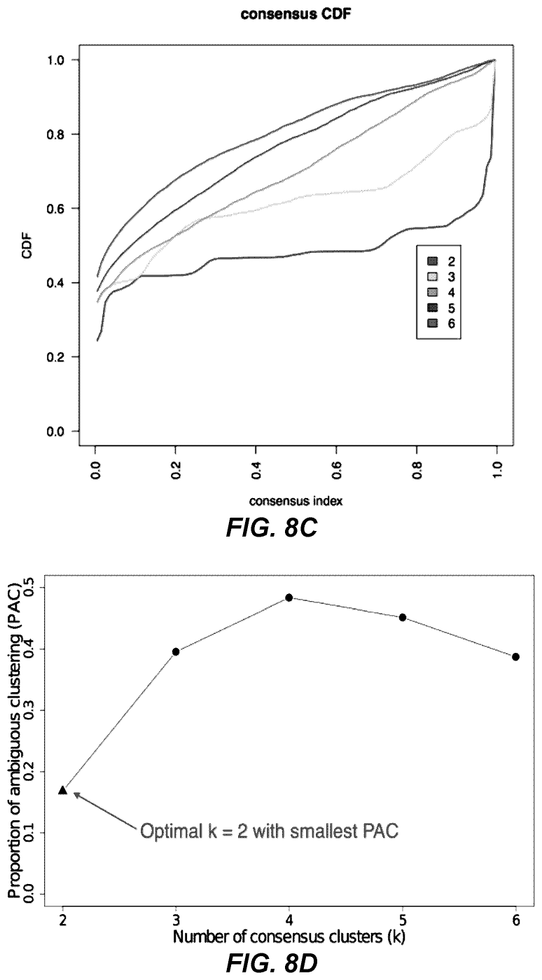

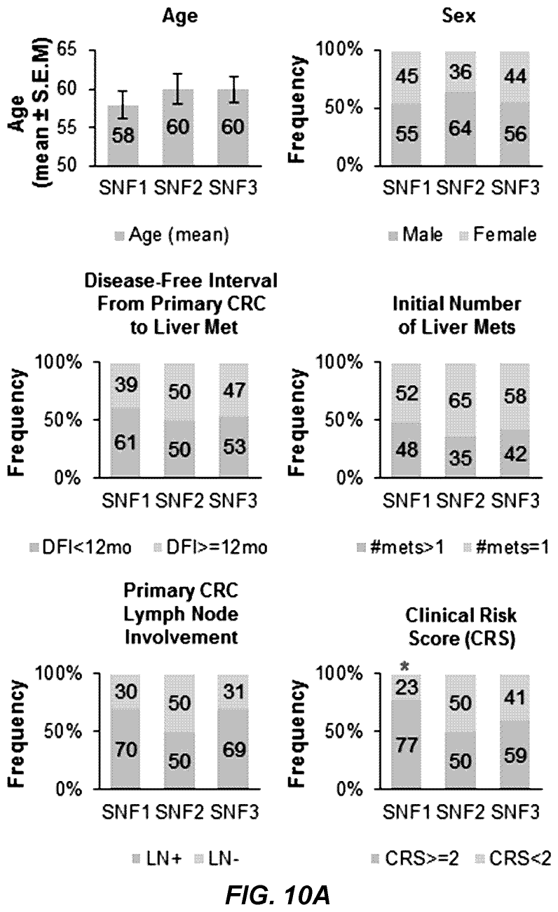

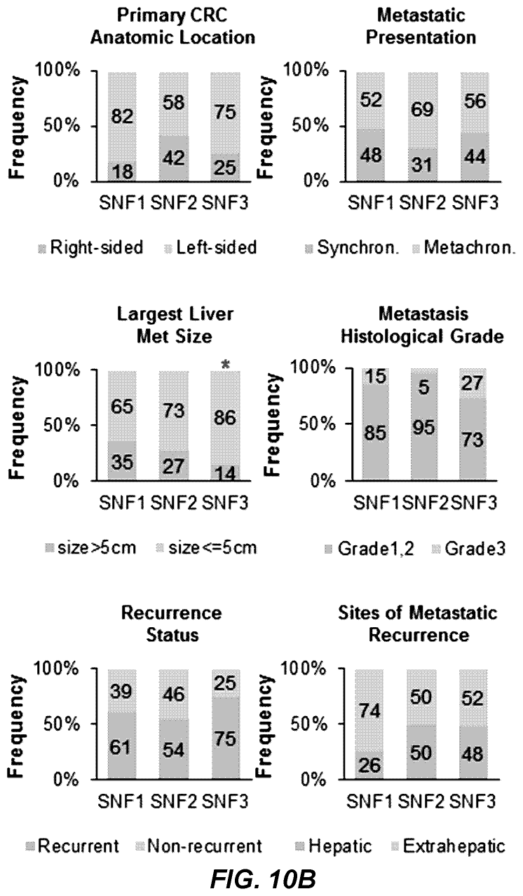

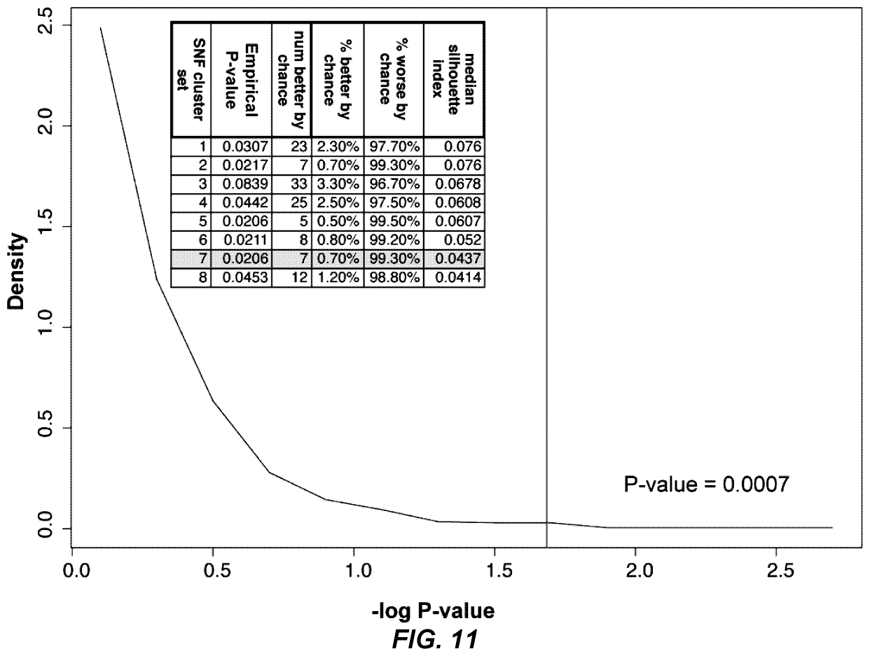

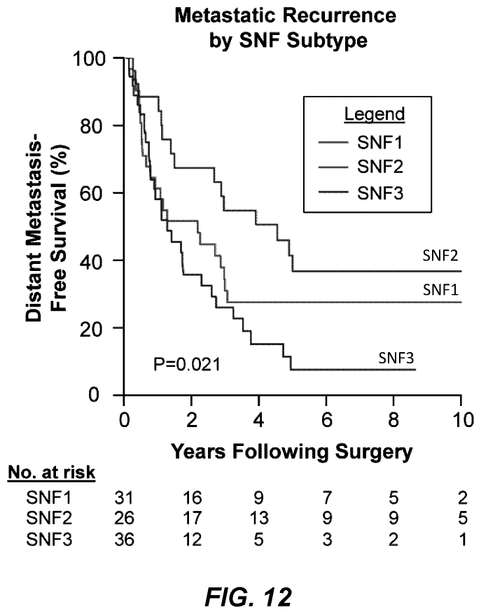

Transcriptomic analyses of individual mRNA or miRNA datasets were limited in the molecular subtyping of colorectal liver metastases (FIGS. 7 and 8). Based on previous work which demonstrated coordinated miRNA-mRNA transcriptional networks underlying metastatic phenotypes, as well as primary CRC subtypes¹⁹⁻²², an integrated expression analysis was performed to uncover novel intrinsic subtypes of CRCLM. The Similarity Network Fusion (SNF) algorithm was utilized to incorporate parallel miRNA and mRNA networks in 93 patient samples independently of clinical, pathological, or survival data. SNF is a computational method for integration of diverse types of data with superior performance in the identification of cancer subtypes when compared to single data and established integrative approaches²³. Three distinct molecular subtypes of CRCLM were observed, denoted SNF1 (33%), SNF2 (28%), and SNF3 (39%) (FIG. 2B). Despite the detection of SNF subtypes solely based on molecular features, the inventors found the SNF subtypes exhibited heterogeneous clinical outcomes with 10-year OS of 37%, 64%, and 20%, respectively (P=0.021, log-rank test) (FIG. 2C and FIG. 10). Using permutation analysis, they examined the importance of the SNF cluster structure on its association with OS. After 1,000 randomized mRNA-miRNA permutations, they found that the SNF clustering algorithm was unlikely to generate our empirical OS difference by chance (P=0.0007) (FIG. 11). Importantly, patients with SNF2 metastases experienced fewer metastatic recurrences or deaths after hepatic resection as compared to SNF1 or SNF3 metastases (FIG. 2D and FIG. 12), and metastatic recurrences of SNF2 metastases were significantly more likely to be limited in number, defined as 1-3 subsequent metastases, as compared to SNF1 or SNF3 metastases (FIG. 2D).

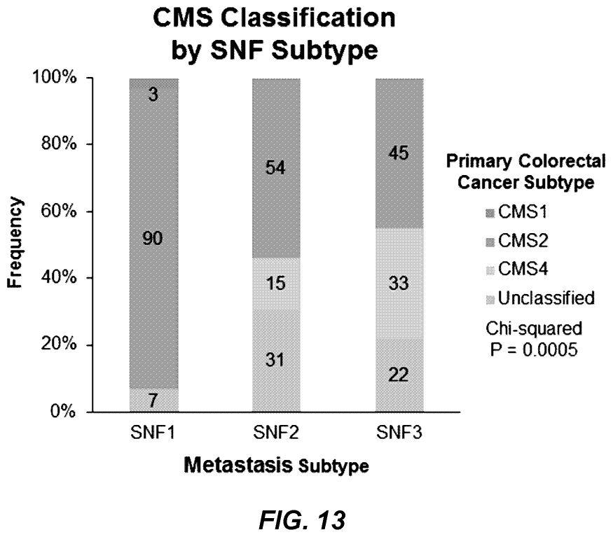

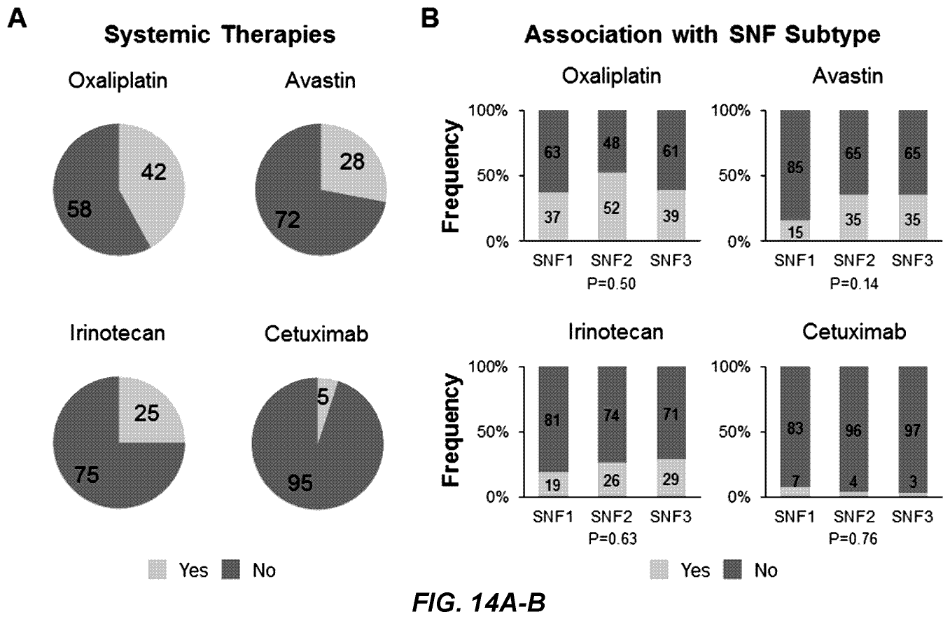

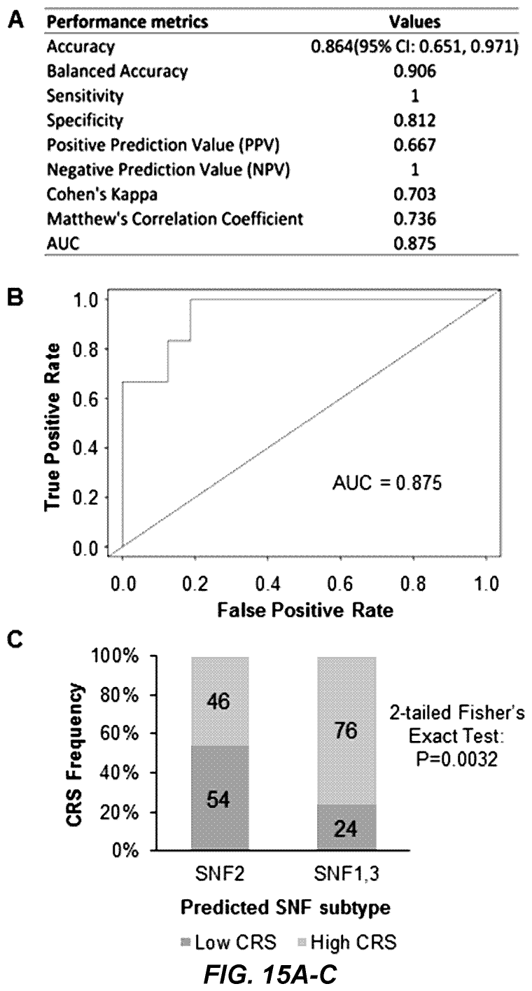

Each SNF subtype demonstrated distinct patterns of mRNA and miRNA expressions (FIG. 2E). SNF2 and SNF3 metastases displayed similar patterns of primary CRC CMS subtypes whereas, by contrast, SNF1 metastases almost exclusively exhibited the CMS2 pattern (FIG. 13). However, only 10%, 5.6%, and 16% of SNF1-, SNF2-, and SNF3-specific gene sets overlapped with the CMS classifier suggesting that SNF subtyping provided independent classification of CRC tumors. In addition, the type of perioperative chemotherapy had no effect on the molecular patterns (FIG. 14) or overall survival of resected CRCLM. Given that independent datasets of integrated molecular data do not exists for clinically annotated CRCLM, the inventors trained and validated an mRNA-based classifier to identify SNF2 patients. They found that the molecular classifier accurately identified SNF2 patients with 100% sensitivity and 81% specificity in their patient cohort (FIGS. 15A and 15B). In an independent dataset of CRCLM patients with similar clinical and pathological features treated with hepatic resection at the Memorial Sloan-Kettering Cancer Center (n=96), metastases classified as the SNF2 subtype were confirmed to demonstrate favorable clinical outcomes as compared to metastases with SNF1 or SNF3 patterns (FIG. 15C). These findings supported SNF subtypes and their associations with clinical outcomes in an independent dataset from a distinct institution.

### Example 4—Molecular Characterization of Intrinsic Liver Metastasis Subtypes

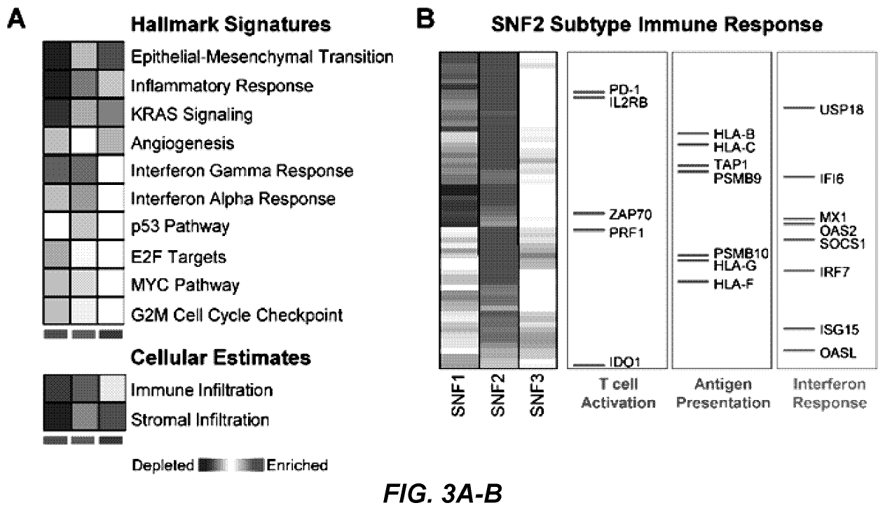

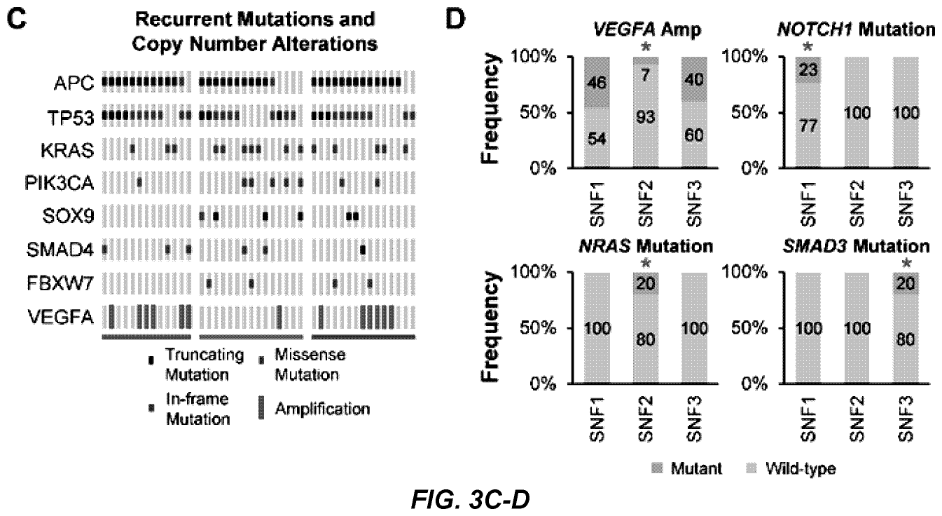

Ensemble of Gene Set Enrichment Analyses (EGSEA) provided substantial insight into the biological features of SNF subtypes (FIG. 3A). EGSEA quantifies the enrichment of biologically defined gene sets within a gene expression profile²⁴. SNF3 metastases were found to show enrichment for expression patterns associated with high stromal infiltration, epithelial-mesenchymal transition (EMT), extracellular matrix remodeling, angiogenesis, inflammatory response, and KRAS signaling (FIG. 3A). SNF2 metastases similarly exhibited enrichment for EMT and KRAS pathways; however, SNF2 metastases were distinguished by high immune infiltration, enrichment of interferon alpha and gamma signatures, and activation of p53 pathways. In concert with these findings, SNF2 metastases significantly overexpressed innate and adaptive immune genes, such as those which mediate T cell activation and crosstalk between antigen presenting cells and T cells, as compared to SNF1 and SNF3 metastases (FIG. 3B and Table 7). SNF1 metastases displayed both low stromal and low immune infiltration signatures but were markedly enriched for E2F/MYC signaling, including TERT (telomerase) overexpression, as well as abnormalities in DNA damage signaling and cell cycle checkpoints.

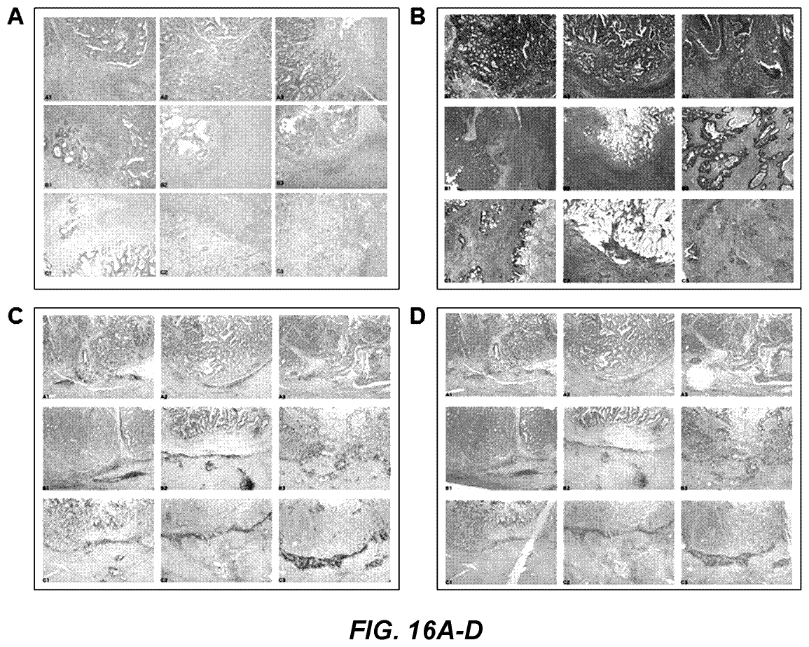

Importantly, CRCLM subtypes were also discernible at the histological level (FIG. 16). SNF2 metastases exhibited dense band-like peritumoral infiltration of CD3-positive and CD8-positive lymphocytes extending intratumorally, and trichrome staining demonstrated minimal fibrosis, whereas SNF3 metastases were distinguished by marked intratumoral and peritumoral fibrosis which harbored peritumorally restricted lymphocytic infiltrate. In contrast, SNF1 metastases revealed prominent nests of tumor cells with minimal CD3-positive or CD8-positive cells or fibrosis. These findings demonstrated unique molecular properties of SNF subtypes associate with differential patient outcomes after hepatic resection of limited CRCLM.

### Example 5—Mutational and Copy Number Landscapes

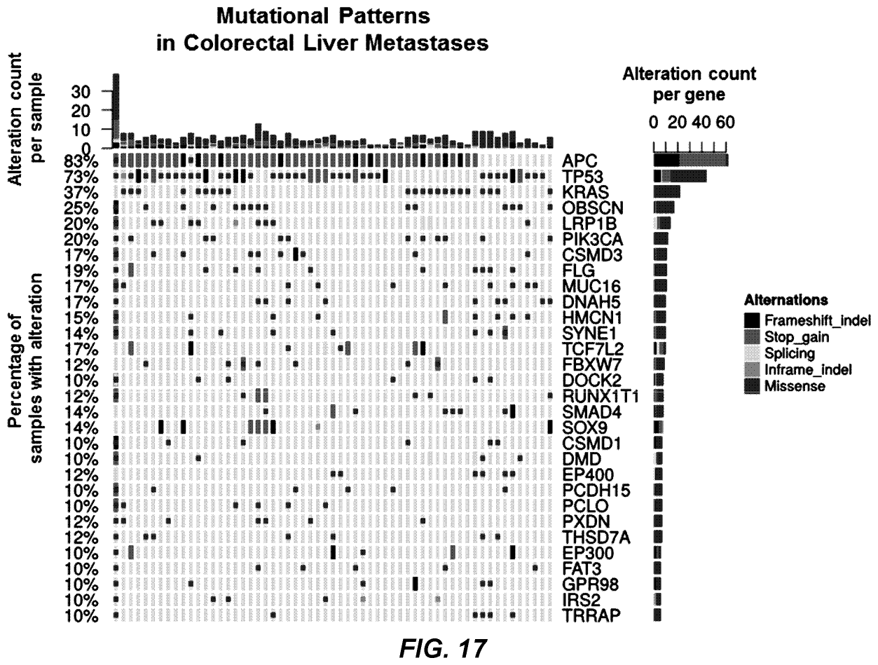

Fifty-nine liver metastases and matched normal liver specimens underwent next-generation genomic sequencing using OncoPlus, a clinically validated hybrid capture genomic sequencing platform comprising 1,212 commonly altered cancer genes for mutational and copy number analyses¹¹. Mutation Significance (MutSig) analysis confirmed enrichment in CRC driver gene mutations of APC, TP53, KRAS, PIK3CA, SOX9, SMAD4, and FBXW7 in 83%, 73%, 37%, 20%, 14%, 14% and 12% of liver metastases, respectively (FIG. 3C, FIG. 17, and Table 8). In addition, the inventors observed frequent gene-level copy number variations, including amplifications of VEGFA (FIG. 3C), MYC, and ERBB2 and deletion of MAP2K4, which were previously described for primary CRC¹⁸. They also found that mutational patterns reflected the anatomic location of the primary tumor origin of liver metastases. Eighty-three percent of liver metastases from right-sided colon cancers exhibited activating somatic mutations in KRAS, in contrast to 24% from left-sided cancers (P=0.0005, Fisher's exact test). Also, PIK3CA mutations occurred in 50% of metastases derived from right-sided primary tumors versus 8% from left-sided primary tumors (P=0.0038, Fisher's exact test).

These findings were extended by characterizing the mutational and copy number landscapes of CRCLM by SNF subtype. Unique somatic mutations in each SNF subtype were identified (FIG. 3D and Table 9). SNF3 metastases demonstrated exclusive somatic mutations in SMAD3, whereas NOTCH1 and PIK3C2B mutations occurred only in SNF1. By contrast, NRAS, CDK12, and EBF1 mutations were unique to SNF2 metastases (FIG. 3D). In addition, amplification of VEGFA was more prevalent in SNF1 and SNF3 metastases as compared to SNF2 metastases (FIG. 3D and Table 9). Notably, no significant differences were found in the frequency of KRAS or BRAF mutations across SNF subtypes. Taken together, these data support the notion that SNF subtypes of CRCLM harbor distinct genomic aberrations.

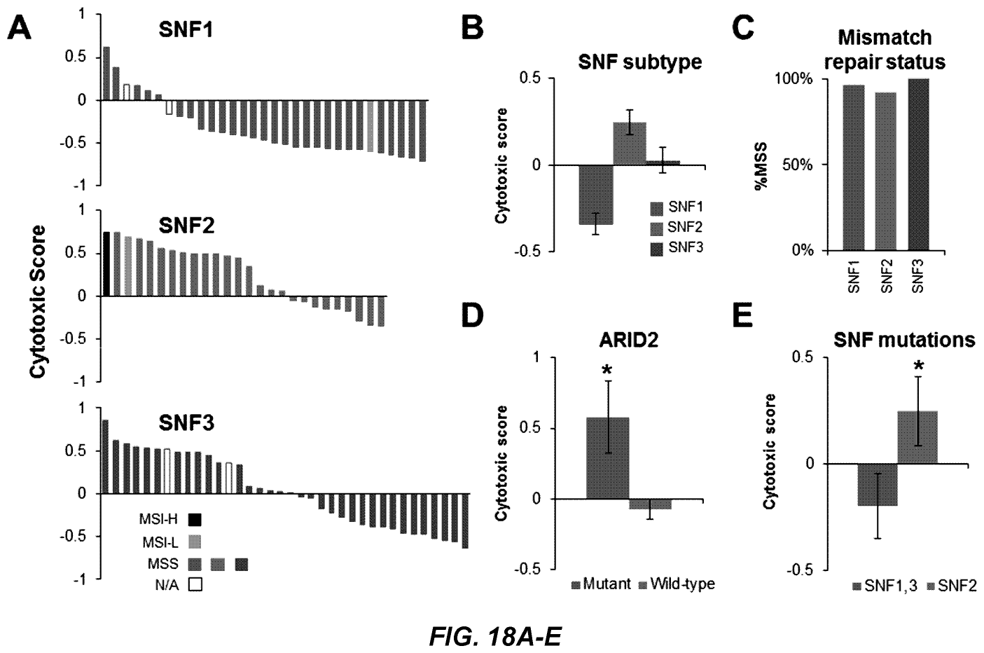

Furthermore, the inventors found that the median number of mutations per sample was not statistically different across SNF subtypes. Given that mismatch repair deficiency leading to microsatellite instability (MSI) contributes to tumor hypermutation in association with cytotoxic immune infiltration²⁵, the inventors investigated whether MSI explained the SNF2 subtype. They identified an MSI phenotype in 3.4% of patients, which is consistent with the incidence of MSI in metastatic colorectal cancer²⁶. However, only one SNF2 metastasis demonstrated an MSI-high phenotype, while two metastases—one from SNF1 and one from SNF2, exhibited an MSI-low phenotype. The SNF2 MSI-high and MSI-low metastases, but not SNF1 MSI-low metastasis, showed significant enrichment of cytotoxic cell signature expression (FIG. 18). Although mutational burden did not correspond to the SNF2 immune subtype, increased mutational burden associated with MSI in SNF2, including hypermutation (n=1,760 mutations) in the MSI-low metastasis related to POLE mutation. In addition, recent data suggest that specific mutations also predict cytotoxic immune responses²⁷. In this regard, ARID2 mutations are associated with increased cytolytic activity in multiple types of cancers. In the inventor's dataset, a significant elevation in cytotoxic immune responses in patients with somatic ARID2 mutations was found (FIG. 18). As well, metastases with SNF2-specific mutations in CDK12, NRAS, or EBF1, in contrast to metastases with SNF1- or SNF3-specific mutations in NOTCH1, PIK3C2B, or SMAD3, showed significant enrichment for cytotoxic immune responses (P=0034). Taken together, these findings identify novel mutations associated with immune responses and favorable clinical outcomes of CRCLM.

### Example 6—Integrated Risk Stratification

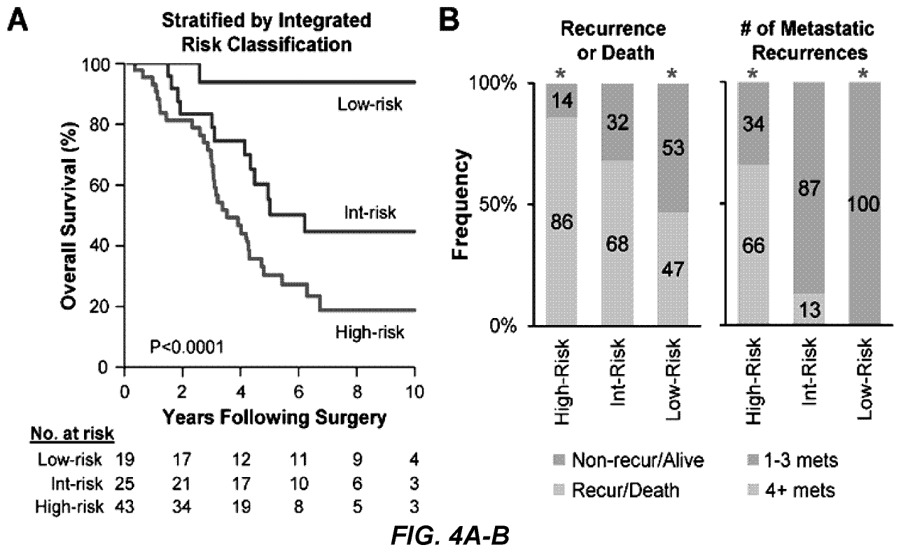

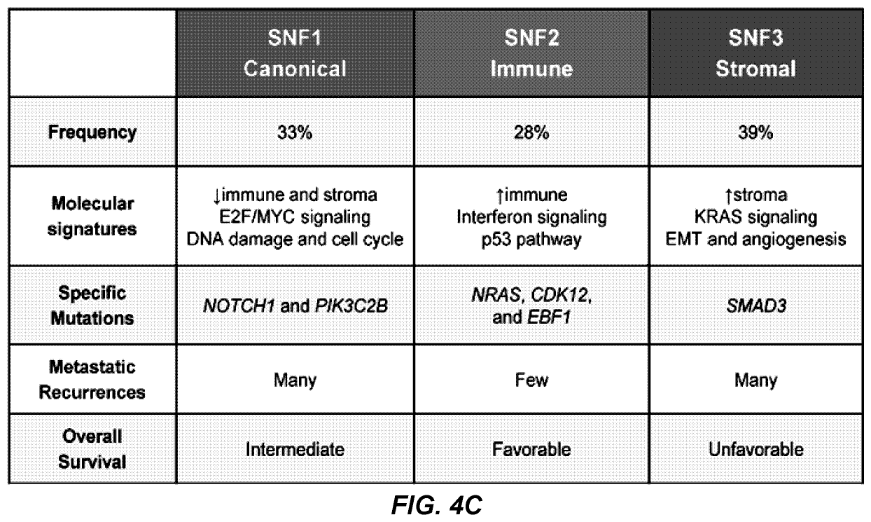

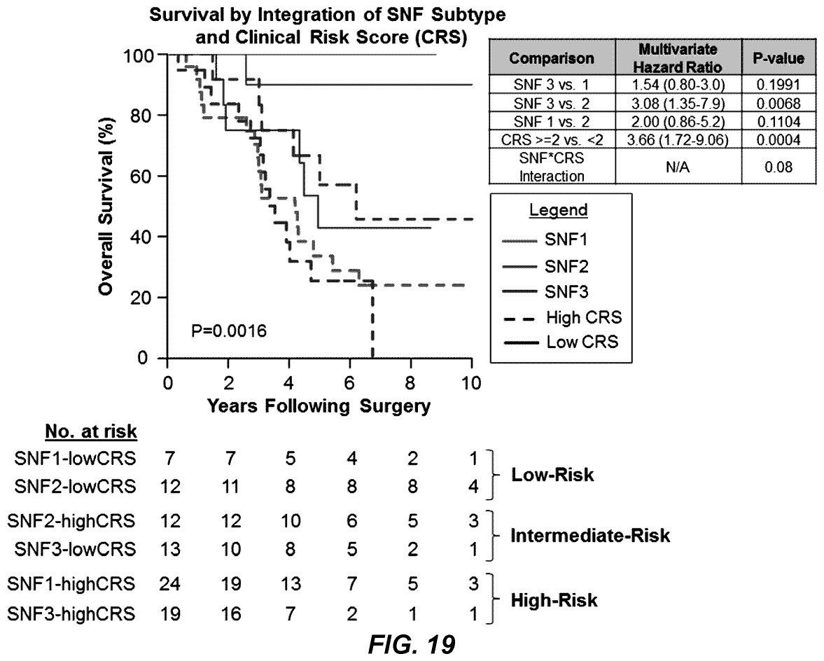

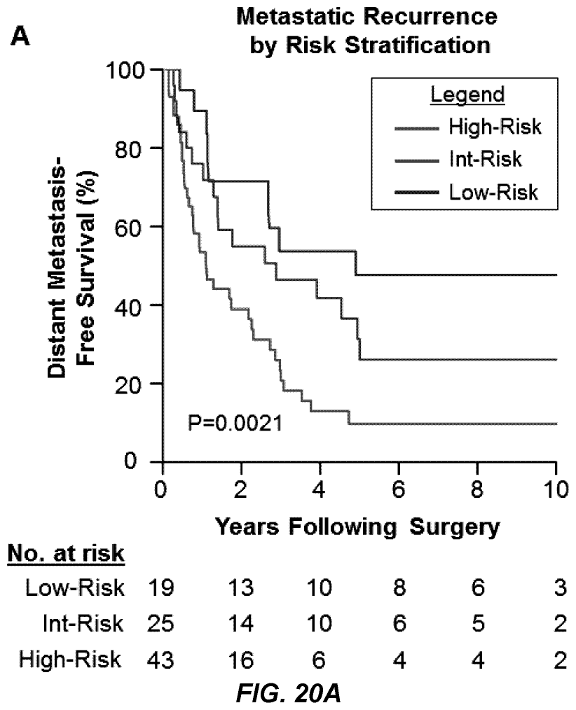

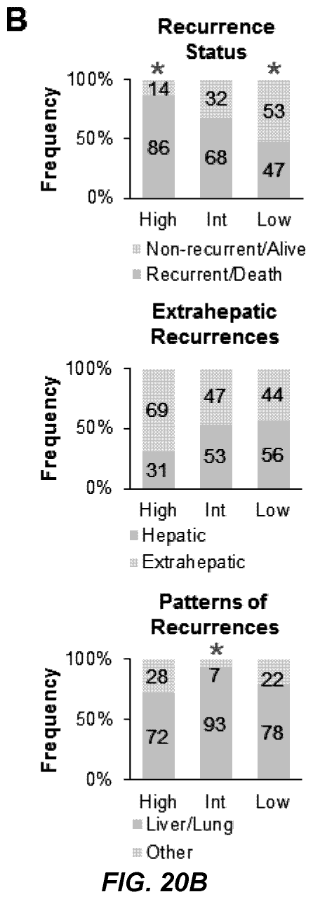

The inventors investigated whether SNF molecular subtyping could improve clinical risk stratification after hepatic resection of CRCLM by augmenting the prognostic effect of CRS. Multivariate Cox proportional hazard analysis indicated the prognostic impact of SNF subtypes was statistically independent of but complementary to CRS (FIG. 19). Integration of SNF subtypes and CRS yielded three prognostic risk groups: (1) low-risk (22% of patients)—SNF1 and SNF2 subtypes with low CRS; (2) intermediate-risk (29% of patients)—SNF2 subtype with high CRS and SNF3 subtype with low CRS; (3) high-risk patients (49% of patients)—SNF1 and SNF3 subtypes with high CRS (FIG. 4A and FIG. 19). Multivariate Cox proportional hazard analyses incorporating primary tumor anatomic site, type of perioperative chemotherapy, treatment year, or mutational data, including KRAS mutation, BRAF mutation, or MSI, did not independently contribute to prognostication in our cohort. Ten-year OS for low-, intermediate-, and high-risk groups were 94%, 45%, and 19%, respectively, at median follow-up times of 76, 54, and 40 months (FIG. 4A). Notably, while SNF1 metastases generally demonstrated unfavorable clinical outcomes, a subset of SNF1 metastases with low CRS (23%) achieved long-term survival, which correlated with a reduced frequency of extrahepatic metastatic recurrence in these patients in contrast to SNF1 metastases with high CRS (33% vs. 81%). While distant metastasis-free survival significantly differed across risk groups (median value—low-risk: 59 mo. vs. intermediate-risk: 35 mo. vs. high-risk: 13 mo; P=0.0021) (FIG. 20A), 47% of low-risk, 68% of intermediate-risk, and 86% of high-risk patients developed subsequent metastatic recurrence after hepatic resection (FIG. 20B). Importantly, metastatic recurrences were limited in number in 100% of low-risk patients in contrast to 87% of intermediate-risk and 34% of high-risk patients (FIG. 4B) (P<0.0001, Chi-square test across groups). These findings demonstrate SNF subtypes of CRCLM significantly improve clinical risk stratification for the identification of patients with favorable prognoses after hepatic resection of limited de novo CRCLM.

### Example 7—Methodology

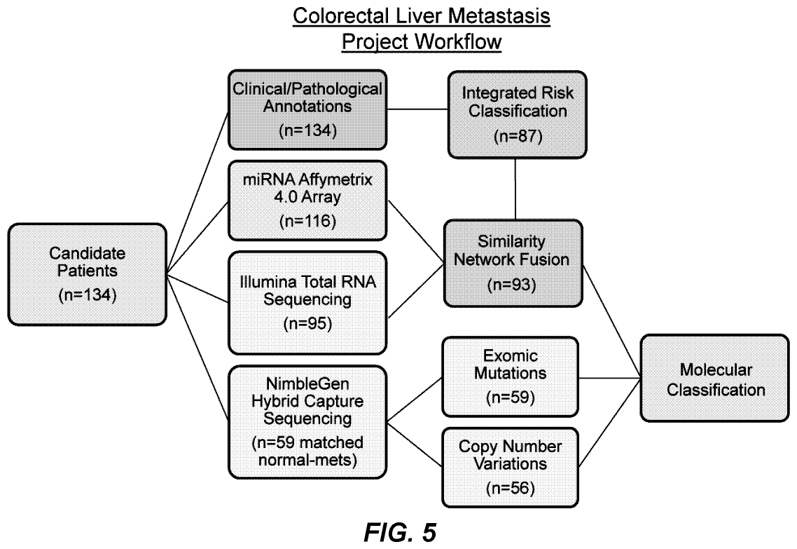

Patients: Samples from 134 adults with liver metastases from primary CRC of which 121 metastases from independent patients successfully underwent molecular analysis were analyzed (FIG. 5). The characteristics of these patients are described in Table 1, Table 2, and the Example 7. The inventors utilized a retrospective clinical cohort study design to identify patients who received uniform treatment for limited (defined as 1-5 lesions involving one or both hepatic lobes), resectable de novo CRC liver metastasis (CRCLM) at two collaborating institutions. Appropriate consent from Institutional Review Boards at each respective cancer center was obtained.

Analytic Platforms: microRNA (miRNA) profiling for 116 samples using Affymetrix miRNA 4.0 Arrays was performed as well as whole genome RNA sequencing for 95 samples using Illumina TruSeq Stranded Total RNA Sequencing. In addition, hybrid capture genomic sequencing of liver metastases and matched normal liver specimens from 59 patients using the OncoPlus panel was performed¹¹. All sequencing was conducted on Illumina HiSeq sequencers. Also performed was microsatellite instability (MSI) analysis on 89 samples using the Promega MSI 1.2 clinical assay according to FDA approved guidelines. Clinical data were frozen on Apr. 30, 2016 and molecular data were frozen on Jun. 26, 2016. Overall survival (OS), defined as the interval between hepatic resection and death from any cause or until censoring at the time the patient was last known to be alive, was chosen as the optimal primary endpoint. The complete list of datasets is provided in Table 2.

Statistical analysis: The statistical analysis included Fisher's exact tests for associations of categorical variables when there were two categories or Chi-square tests when there were three categories. Kaplan-Meier and Cox proportional hazard analyses were used to examine the associations of molecular features with clinical outcomes. Multiple testing corrections were performed using the Benjamini-Hochberg method. All reported P-values are two-sided. A complete description of the methods is in the methods described hereafter.

### Clinical Information

### 1. Patient Characteristics

A retrospective clinical cohort study was conducted on patients who underwent hepatic resection of histologically confirmed metastatic colorectal adenocarcinoma at the University of Chicago Medical Center (Chicago, Ill.) and NorthShore University Health System (Evanston, Ill.) between 1994 and 2012. During this time period, approximately 60-75 patients per year underwent hepatic resection of colorectal liver metastases at the two participating institutions. All available clinical, pathologic, radiologic, and outcome data were collected for patients using medical records. Patients with unresectable or extrahepatic disease at the time of metastatic diagnosis were excluded from this study. In total, 134 consecutive patients with metastatic colorectal cancer who underwent surgical resection of limited de novo liver metastases were selected for molecular analysis. Patients were uniformly treated with perioperative chemotherapy, definitive treatment of primary colorectal cancer, and partial hepatectomy for resection of liver metastases. Detailed cohort characteristics are provided in Table 1 and Table 2. This study was approved by the Institutional Review Boards at each respective institution. Dates of recurrence, death or last follow-up were extracted from medical records and Social Security Death databases. Clinical risk scores (CRS) were calculated as previously described¹.

### 2. Pathologic Examination

Formalin-fixed paraffin-embedded (FFPE) specimens were collected from archived pathologic tissue. FFPE specimens were catalogued and histologically reviewed by an expert pathologist (Dr. Nora Joseph) to ensure adequacy of the specimen and histologic quality control. Tissue blocks containing sufficient tumor tissue were subjected to 2 mm punch biopsies of both tumor and normal liver regions. For each surgical specimen, representative FFPE tissue blocks and corresponding H&E slides were analyzed to confirm the diagnosis of colorectal adenocarcinoma and identify regions containing high quantities of viable tumor cells, as well as independent regions containing normal liver parenchyma. Three cores from tumor and normal tissue regions were obtained. For each specimen, all three cores were combined to reduce intratumoral variability. This procedure was repeated for both tumor and normal biopsies for each patient.

Nucleic Acid Extraction

Punch biopsy specimens were deparaffinized and processed using the RecoverAll Total Nucleic Acid Isolation Kit (Ambion, TX) according to the manufacturer's instructions. Briefly, 200 μL of digestion buffer and 4 μL of protease were added to each sample and incubated overnight at 55° C. RNA and DNA were extracted following the RecoverAll protocol according to the manufacturer's recommendations. Nucleic acid quantification was performed using a NanoDrop 1000 Spectrophotometer and a Qubit® Fluorometer. Nucleic acid extracts were stored at −80° C. until further analysis.

RNA Sequencing

1. Library Construction: RNA integrity and quantity were evaluated using an Agilent 2100 Bioanalyzer (Agilent Technologies, CA). Reverse-stranded paired-end 75 base-pair sequencing libraries were constructed using Illumina Total RNA Stranded Kits. Ribosomal RNAs (rRNAs) were depleted by using the Ribo-Zero rRNA Removal Kit (Illumina). Libraries were sequenced on a HiSEQ2500 machine using standard reagents and protocols provided by Illumina. In total, 95 metastatic samples were successfully sequenced using this approach.

2. Read Alignment and Quantification: Unless otherwise specified, all data analyses were performed under the R programming and software environment for statistical computing and graphics version 3.3 (R Core Team, 2016). FastQ files for each sample were assessed for quality using the FastQC tool (version 0.11.2). Raw reads were aligned to the GRCh38 primary genome assembly using Spliced Transcripts Alignment to a Reference (STAR) aligner (version 2.4.2a) 1-pass algorithm². After sorting the bam files in lexicographical order with the sambamba program³, the inventors assigned the reads to exon features annotated in GENCODE (release 22) using the FeatureCounts tool from the subread package (version 1.4.6) and summarized the read counts by genes⁴. The post-alignment quality control was carried out with Picard tools (version 1.117) and RSeQC package (version 2.3.1). Specifically, the inventors examined the QC data regarding the alignment summary, gene body coverage, read distribution, and ribosomal RNA depletion rate.

3. Data Normalization: The inventors used functions in the R/Bioconductor package edgeR to extract the raw counts of the reads that were mapped to the protein-coding genes⁵. After removing the genes with zero read counts across all samples, the inventors calculated the normalization factors to scale the raw library sizes and the log 2-transformed count per million (log-CPM) for the expression level of each gene. The log-CPM values were corrected for batch effect (sequencing lane effect and institution) using removeBatchEffect function from the R/Bioconductor package limma⁶. 18,714 genes were retained for the subsequent analyses.

4. Detection of Differentially Expressed mRNAs: To identify differentially expressed mRNAs among samples grouped by Similarity Network Fusion (SNF—see Example 7, Similarity Network Fusion) clusters, the inventors first removed non/low-expressed genes in comparison groups by requiring read counts to be at least 1 across a minimum number of samples in one of the comparison groups, followed by trimmed mean of M-values (TMM) normalization using the calcNormFactors function in the edgeR package. Next, the inventors removed heteroscedascity from the count data using the voomWithQualityWeights function from the limma package with quantile normalization method enabled. The inventors then fit a linear model for each gene using the limma algorithm, adjusted for batch effect, and ranked the genes for differential expression using the empirical Bayes method with trend and robust options enabled. The differentially expressed genes were identified with the Benjamini-Hochberg procedure for multiple test adjustment and fold-change. The adjusted P-value threshold and fold-change threshold were set at 0.05 and 2.0, respectively (Tables 3A-C).

microRNA Expression Profiling

RNA integrity and quantity were evaluated using an Agilent 2100 Bioanalyzer (Agilent Technologies, CA). Total RNA (500 ng) was processed for biotin labeling according to the Affymetrix Flash Tag Biotin HSR RNA labeling guide (Affymetrix, CA). The biotin-labeled target was hybridized to Affymetrix miRNA 4.0 Array Chips for 16h at 48° C. and 60 rpm in an Affymetrix 640 hybridization oven. Arrays were washed and stained in an Affymetrix Fluidics Station 450 according to the Affymetrix GeneChip expression guide. The arrays were scanned using the Affymetrix GeneChip Scanner 3000 7G. CEL intensity files were generated using GCOS software. In total, 116 metastatic samples were successfully assayed using this approach.

1. Data Pre-Processing and Normalization: The methods used in this analysis are available as part of the R/Bioconductor packages affy, oligo, limma and sva^{6,7}. The raw Affymetrix GeneChip miRNA 4.0 Array CEL files were imported to R using the read.celfiles function from the oligo package. The inventors first performed robust normexp-by-control background correction using the nec function from the limma package with the robust option enabled⁸. They then normalized the log 2-transformed expression data using cyclic loess normalization with the array weight method. Finally, the inventors summarized the probes into probesets using the rma function from the affy package with the options normalize and background disabled. To remove batch effects caused by array processing dates and the patient cohorts, they applied the ComBat algorithm implemented in the sva package⁹. Two batch factors: (1) institution and (2) microarray scan date were considered. A single sample was run in batch 6 and combined with samples from batch 5. The inventors removed non/low-expressed probesets and retained the probesets representing 778 mature human miRNAs for the subsequent analyses.

2. Detection of Differentially Expressed miRNAs

The inventors applied the limma method to identify differentially expressed miRNAs among the samples grouped by SNF clusters. They first estimated the relative quality weights for each array using the arrayWeightsSimple function, and then fit a linear model for each probeset adjusted for batch effect, followed by ranking probesets for differential expression using empirical Bayes method. The differentially expressed miRNAs were identified with the Benjamini-Hochberg procedure for multiple test adjustment and fold-change. The adjusted P-value threshold and fold-change threshold were set at 0.05 and 2.0, respectively (Tables 4A-C).

Consensus Clustering of Expression Data

Unsupervised consensus clustering analysis was performed on independent mRNA and miRNA expression data sets using the R package ConsensusClusterPlus (version 1.38.0). The inventors selected the most informative mRNAs or miRNAs for clustering, which consisted of the top 25% most variable mRNAs or miRNAs, as measured by the median absolute deviation (MAD). Normalized expression data from previous procedures were first standardized using the data normalization function in the R package clusterSim (version 0.45-1). To run ConsensusClusterPlus, the inventors preset the options as a maximum evaluated cluster k=66, 80% samples per resampling, 1,000 resamplings, Euclidean distance, and k-means clustering algorithm. They chose complete and average linkage as the inner-linkage and final linkage, respectively. The optimal number of k clusters was inferred by inspecting the consensus cumulative distribution function (CDF) plot and the proportion of ambiguously clustered pairs (PAC) plot where the optimal k corresponds to the lowest PAC¹⁰ (k=2, FIGS. 7 and 8).

Consensus Molecular Subtyping (CMS) of Colorectal Liver Metastases.

Microarray expression data derived from 183 patients with colorectal liver metastasis were collected from ArrayExpress (study IDs: E-MTAB-1951, E-GEOD-62322, E-GEOD-41258, and E-GEOD-35834). Study E-MTAB-1951 contains 96 samples profiled on the Illumina HumanHT-12 v3.0 Expression BeadChip. E-GEOD-62322 and E-GEOD-41258 contain 19 and 47 samples that were profiled on Affymetrix HG-U133A Arrays, respectively. E-GEOD-35834 consists of 27 samples profiled on the Affymetrix Human Exon 1.0 ST Array. The inventors also used two sets of normalized RNA Sequencing data. One cohort includes 93 metastases from our cohort which were reanalyzed with RSEM to assess TPM abundances, while the other cohort contains 45 liver metastases that were obtained from the Memorial Sloan-Kettering Cancer Center and processed similar to previously described methods in Example 7 (RNA sequencing). For E-MTAB-1951, raw expression data was preprocessed with variance stabilizing transformation and quantile normalization using the lumi package (version 2.26.3). For the remaining microarray studies, CEL files were downloaded directly from ArrayExpress and processed with fRMA (version 1.28.0) for core annotation targets summarized by robust weighted average. Level 3 TCGA READ and COAD RNA Sequencing RSEM expression data was obtained from Sage-Bionetworks Synapse repository (syn: syn2320098, syn2320092, syn2320147, and syn2320079). TPM expression data corresponding to primary tumor samples were selected, offset by 1, and log 2 transformed. Multiple gene level mappings were resolved by singular value decomposition. Datasets from both tissues were merged, and a custom ComBat correction was performed to account for batch effects between HiSeq-RNASeqV2 and Illumina-GA platforms. All scripting and normalization methods are available for download via the CRC Subtyping Consortium's github including the merging protocol (https://github. com/S age-Bionetworks/crcsc/blob/dc58542555e281c1ccb55aeb73 d087e7d0bdf6bf/groups/G/dataQc/tcgaCrcRNAseq-merged.R) and miscellaneous normalization procedures (https://github.com/Sage-Bionetworks/crcsc/blob/dc58542555e281c1ccb55aeb73 d087e7d0bdf6bf/groups/G/dataQcaGnorm.R).

For both microarray and RNA Sequencing expression data, features were mapped to corresponding Entrez gene IDs using annotation sets provided by Ensembl GRCH38 and Bioconductor including hgu133a.db (version 3.2.3), huex10sttranscriptcluster.db (version 8.6.0), lumiHumanIDMapping (version 1.10.1), or org.Hs.eg.db (version 3.3.0). For multiple annotations mapping to a unique gene feature, either the median probeset value or the largest coefficient of variation across RNAseq samples was retained as an expression estimate for the corresponding gene feature. CMS classification was performed using the single sample procedure (SPP) (https://github.com/Sage-Bionetworks/CMSclassifier).

Similarity Network Fusion

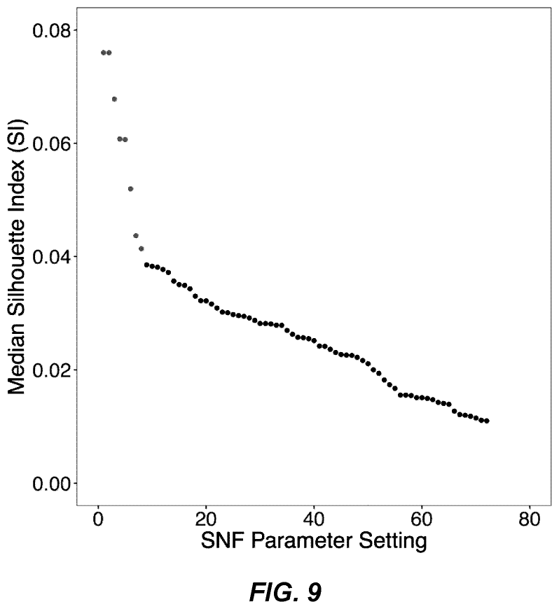

The matched normalized mRNA and miRNA expression data of 93 metastases were first separately standardized using the standardNormalization function from the R package SNFtool (version 2.2). The Euclidean distances between all pairs of samples in mRNA and miRNA data were calculated, respectively. An affinity matrix was computed using the function affinityMatrix with the number of nearest neighbors K and the variance for local model alpha. The inventors then performed similarity network fusion on affinity matrices of mRNA and miRNA with the number of iterations T, which was used in the subsequent spectral clustering step where samples were assigned to one of the SNF clusters. Three clusters were identified using default settings. In order to find other possible compositions of three clusters, the inventors tested 168 parameter combinations of K (10, 15, 20, 25, 30, 35, 40), alpha (0.3, 0.4, 0.5, 0.6, 0.7, 0.8), and T (20, 30, 40, 50). For each parameter setting, they applied the estimateNumberOfClustersGivenGraph function to estimate the possible number of clusters using two heuristic methods: (1) eigen gap and (2) rotation cost. The inventors retained the clustering results which comprised three clusters and calculated the median Silhouette index (SI) of each result. The top 8 clustering results that had the highest median SIs were selected (FIG. 9). A majority voting scheme was applied to determine the final cluster membership based on the top 8 clustering results. In the event of a tie, the inventors chose the membership defined by four clustering results with the largest median Sis.

Robustness of SNF Clustering on Overall Survival Previous work has shown that the SNF algorithm for clustering is statistically robust¹¹. The inventors examined whether the observed survival difference between SNF clusters could be reproduced by random chance. To this end, they performed permutation analyses. For each permutation, miRNA profiles were shuffled and randomly assigned to mRNA profiles. Subsequently, SNF clustering was performed de novo and each patient was assigned to one of three resulting groups. Differential overall survival across clusters was then assessed with a log-rank test. This process was repeated 1,000 times, and log-rank p-values were used to construct a null distribution. The inventors examined the number of instances when p-values from the null distribution were more extreme (i.e. smaller) than the empirical p-value (FIG. 11).

Ensemble of Gene Set Enrichment Analyses (EGSEA).

Raw gene feature counts were mapped to Entrez ID using the R/Bioconductor package org.Hs.eg.db v3.4.0⁵. Low/non-expressed genes with less than 1 CPM across the minimum number of samples in any SNF group were excluded from subsequent analysis using edgeR v3.16.5. Quality weighted, quantile, and log-normalized CPM were calculated using limma-voom v3.30.11. Gene set enrichment was performed using the R/Bioconductor package EGSEA v1.2.0¹² with planned contrasts of each SNF group against the average of the remaining groups. Independent EGSEA analyses were performed for gene lists provided by MSigDB v5.2¹³ (Tables 5A-C) and a custom gene list identifying numerous immunological, canonical, and metabolic pathways¹⁴ (Tables 6A-C). Intratumoral immunome profiling was performed as previously described¹⁵, and resulting gene lists were used to calculate SNF-level and single sample enrichment scores using EGSEA.

SNF Class Predictor

Data Preprocessing: To build a classifier to distinguish samples between SNF cluster 2 (C2) and SNF cluster 1 and 3 combined (C13), the normalized mRNA expression data of 93 patients was split into a training set, consisting of 20 SNF cluster 2 samples and 51 cluster 1 and 3 samples, and a test set, consisting of 6 cluster 2 samples and 16 cluster 1 and 3 samples. The class ratio remained unchanged during the partition. For the training set, the inventors first filtered genes with near zero-variance. They then identified highly correlated genes with a pair-wise absolute correlation coefficient greater than 0.7, and removed those with the largest mean absolute correlation. They further removed potential linear dependencies of the data using the findLinearCombos function from the R package Caret (version 6.0). They applied the preProcess function to center and scale the training and test data by mean and standard deviation, followed by rescaling data to −1 and 1.

Model Training and Testing: The inventors applied Prediction Analysis of Microarrays (PAMR, version 1.55)—a nearest shrunken centroid classification algorithm—on the training set¹⁶. A 10-fold cross-validation was performed to obtain the optimal threshold of 2.72 for the prediction, where the overall error rate was 0.056. The final classification model contains 113 genes (Tables 10A-C) and was evaluated using the held-out test data of 6 SNF cluster 2 samples and 16 SNF cluster 1 and 3 samples. Performance metrics such as accuracy, balanced accuracy, sensitivity, specificity, positive prediction value (PPV), negative prediction value (NPV), Cohen's Kappa, Matthew's correlation coefficient, and area under the curve (AUC) were calculated using the confusionMatrix function from the Caret package and an in-house script (FIG. 15A and FIG. 15B). A nearest shrunken centroid classification algorithm was also used to generate a classification model to identify SNF2 metastases based on miRNA expression. This classification model contains 53 miRNAs (Tables 11A-C).

Independent Validation of Classifier: The inventors downloaded the raw expression data of 96 patients from ArrayExpress (study ID: E-MTAB-1951). They prioritized the analysis of the E-MTAB-1951 samples as it is the only publicly available colorectal cancer liver metastasis dataset with available clinical annotations (i.e. Clinical Risk Scores (CRS)) to test for association with SNF membership grouping. The samples were profiled using the Illumina HumanHT-12 v3.0 Expression BeadChip. Using the R/Bioconductor package lumi (version 2.26.4)¹⁷, they transformed the expression data via variance-stabilizing transformation (VST) algorithm, followed by between-chip normalization with the robust spline normalization (RSN) algorithm. For multiple probes that mapped to the same Ensembl gene ID, they removed those with the smallest variance across samples. The inventors re-trained the PAM classifier on all 93 samples in our cohort using the 113 genes selected from the previous analysis and applied it to the normalized E-MTAB-1951 microarray data set. For genes that were missing in the microarray data, they replaced the expression values with −1 after scaling the data to −1 and 1. The concordance between the predicted SNF clustering memberships and the Clinical Risk Scores (CRS) from the E-MTAB-1951 samples was examined using contingency analysis (FIG. 15C).

Hybrid Capture Next Generation Sequencing

Targeted Capture Sequencing Panel: For each specimen, DNA from 1,212 exonic regions was captured using the UCM-OncoPlus panel based on the NimbleGen SeqCap EZ custom capture method as previously described¹⁸. In brief, this approach utilizes a tiered assay system in which highly clinically relevant genes (tier 1, n=316) are sequenced approximately 3-fold deeper than the remaining (tier 2) genes. Capture libraries were generated using the Illumina TruSeq platform. Libraries were multiplexed with 6 base-paired indexes up to 9 samples per lane and sequenced using Illumina HiSeq2000 and HiSeq2500 machines. FastQ files were generated using Illumina's BCL2FastQ1.8.4.

Sequencing Data Alignment: FastQ files were quality trimmed using cutadapt v1.9.1 (http://cutadapt.readthedocs.io/en/stable/guide.html) for Phred score quality on 3′ end Q>=30¹⁸ and a minimum length of 19 after trimming (bwa-mem recommended minimum read size). Remaining reads were aligned using the bwa-mem algorithm v0.7.8 (http://bio-bwa.sourceforge.net) against the hg19 reference. PCR duplicates were removed by Broad Institute Picard tools v1.128 MarkDuplicates (https://github.com/broadinstitute/picard). Bedtools v2.22.1 (http://bedtools.readthedocs.io/en/latest/) was used to ascertain coverage at tier 1 and tier 2 loci. Samples that did not have a mean 300× depth of coverage at tier 1 genes were excluded from subsequent analyses. In targeted-capture sequencing, oxidative damage can be pervasive and lead to false positive variant calls at sites with sequence context CCG being read as CAG¹⁹. Sample-level oxidative damage was calculated using Picard CollectOxoGMetrics. Sample with ArtQ¹⁹ scores less than 21 were removed. Overall, 59 unique metastasis-normal pairs were available for analysis.

Example alignment pipeline flow:
1. cutadapt -m 19 --quality-base=33 -q30 -a ZZZ-A ZZZ -o sample_filtered_r1.fastq.gz -p sample_filtered_r2.fastq.gz sample_r1.fastq.gz sample_r2.fastq.gz>>sample.cutadapt.log 2>> sample.cutadapt.log
2. bwa mem -t 8 -R “@RG\tID: sample flowcell id\tLB: sample\tSM: sample\tPL:illumina”-v 2 hg19.fa sample_filtered_r1.fastq.gz sample_filtered_r2.fastq.gz|samtools view -bT hg19.fa ->sample.bam)>sample.bwa.pe.log 2>&1
3. novosort -c 8 -m 30G --tmpdir novosort_tmp -o sample.srt.bam -i sample.bam>sample.novosort.log 2>&1
4. java -Xmx30g -jar picardjar MarkDuplicates CREATE INDEX=true TMP_DIR=picard_tmp REMOVE_DUPLICATES=true ASSUME_SORTED=true MAX_FILE_HANDLES_FOR_READ_ENDS_MAP=500 INPUT=sample. srt.bam OUTPUT=sample.rmdupsrt.bam METRICS_FILE=sample.rmdupsrt.metrics VALIDATION_STRINGENCY=LENIENT>sample.picard.rmdup.pe.log 2>&1
5. bedtools coverage -hist -abam sample.rmdup.srt.bam -b capture_panel_v3_t1.bed|grep all>sample.capture_t1.hist
6. bedtools coverage -hist -abam sample.rmdup.srt.bam -b capture_panel_v3_t2.bed|grep all>sample.capture_t2.hist
7. java -Xmx30g -jar picardjar CollectOxoGMetrics I=sample.rmdup.srt.bam O=sample.oxo_summary.txt R=hg19.fa INTERVALS=capture_panel_v3.intvl 2>sample.oxo.log;

Variant Calling and Filtering: Single nucleotide variants (SNVs) were called using MuTect v1.1.7 (http://archive.broadinstitute.org/cancer/cga/mutect). Insertions and deletions (indels) were called using scalpel-discovery 0.5.3 (http://scalpel.sourceforge.net/). Calls not annotated as “PASS” or “KEPT” were removed. For both SNVs and indels, only calls falling within genomic coordinates targeted by the capture panel were retained for subsequent analyses. Targeted capture libraries have been shown to be susceptible to oxidative damage. Even samples that do not have pervasive oxidative damage can have false positive calls attributable to this phenomenon¹⁹. All SNV calls were assigned a FoxoG score using metalfox (https://github.com/cpwardell/bin/blob/master/metalfox.py). Based on previously reported studies¹⁹, calls without a MuTect tumor_lod greater than −10+(100/3) * FoxoG were removed as they were likely a consequence of oxidative damage. All variants were annotated using snpeff v3.6c (http://snpeff.sourceforge.net/), hg19 reference. Only variants that exist within coding regions or disrupted splice sites were included in analyses. Calls with a variant allele frequency (VAF)<5%, position coverage<30, or an allele frequency>=0.01 in ExAC were removed. To further improve the quality of indel calls, two additional filters were implemented: (1) Dustmasker (https://www.ncbi.nlm.nih.gov/IEB/ToolBox/CPP_DOC/lxr/source/src/app/dustmasker/) was used to identify low complexity genomic regions, and indels falling within these regions were discarded; (2) A pseudo-panel of normal samples was constructed, such that across the matched normal samples, all putative indel calls that failed Scalpel filters due to ‘HighVafNormal’ or ‘HighAltCountNormal’ were aggregated. All indels that failed in two or more samples from unique patients were filtered. These methods helped to eliminate remaining noisy calls which passed previous filtering steps.

Example variant calling workflow:
1. java -Djava.io.tmpdir=./temp -Xmx2g -jar mutect-1.1.7.jar -T MuTect -R hg19 --intervals chr1.intvl --input file:normal normal bam tumor bam --max_alt_alleles_in_normal_count 1000 --max_alt_alleles_in_normal_qscore_sum 37 --max_alt_allele_in_normal_fraction 0.05 --out tumor_normal/tumor_normal.chr1.out -vcf tumor_normal/tumor_normal.chr1.vcf --enable_extended_output --strand_artifact_power_threshold 0 -log tumor_normal.mutect.chr1.log 2>>‘tumor_normal.mutect.chr1.log;
2. metalfox.py -f1 tumor_normal/tumor_normal.chr1.out.keep -f3 tumor.bam -m wgEncodeCrgMapabilityAlign100mer.bedGraph.gz>tumor_normal.foxog_scored_added.out
3. scalpel-discovery --somatic --logs --numprocs 8 --tumor tumor bam --normal normal bam --bed capture_panel_v3.bed --ref hg19.fa 2>>tumor_normal.indels.log
4. java -jar snpEff.jar eff -t hg19 (tumor_normal.out.keep.sift.vcf/tumor_normal.somatic_indel.PASS.sift.vcf) -v>(tumor_normal.out.keep.eff.vcf/tumor_normal.somatic_indel.PASS.eff.vcf) 2>>tumor_normal.snpeff.log

Mutation Significance (MutSig) Analysis: VCFs were annotated and converted to a MAF format using Oncotator²⁰. MAF files for all patients were merged and assessed for significant gene-centric mutation frequency using MutSigCV version 2 with default coverage and covariate tables provided by the Broad Institute²¹. Mutation Assessor²² and ClinVar²³ were used to predict the functional impact of protein-coding mutations.

Copy Number Variation Analysis: Copy number calling was carried out using CNVKit v0.7.12.dev0²⁴. All 59 matched-normal samples were used to calculate the pooled reference baseline using default parameters. Segmented log 2 ratios were used to call copy number gains and losses.

Identification of Prognostic Mutations: Multivariate Cox proportional hazard ratios were generated for each mutated gene feature as a binary factor across 59 liver metastasis-matched paired normal samples using the survival v2.40-1 R package. SNF subtype and Clinical Risk Score (CRS) were included as covariates in multivariate analyses. Ten-year overall survival was chosen as the primary endpoint of the analysis.

Microsatellite Instability (MSI) Analysis

H&E slides of normal and tumor specimens were reviewed by a molecular pathologist (Dr. Nora Joseph). Tumor sections with greater than 30% tumor percentage were used for DNA extraction by the Pinpoint Slide DNA Isolation System (Zymo Research). DNA was subsequently purified by using the Zymo-Spin I Column protocol. All samples were run on the Promega MSI 1.2 assay according to the FDA approved protocol and result interpretation. MSI testing was performed on 93 metastases with corresponding SNF subtypes of which 89 samples were successfully assayed. Four samples failed repeated testing.

Immunohistochemical Analysis

CRC liver metastases were preserved in formalin and embedded in paraffin. 5 μm tissue sections were created from paraffin blocks and mounted on glass slides. The slides were stained on Leica Bond RX Automatic Stainer using HTRC Bond Refine DAB protocol. After antigen retrieval treatment (epitope retrieval solution II, AR9640, Leica Biosystems) for 20 minutes, anti-human CD3 (DAKO, Cat#M7254, Clone: F7.2.38, mouse IgG) antibody (1:600) was applied on tissue sections for 25 minutes incubation. For CD8 staining, anti-human CD8 (DAKO, Cat#M7103, Clone: C8/144B, mouse IgG) antibody (1:400) was applied. The antigen-antibody binding was detected with Bond polymer refine detection (Leica Biosystems, DS9800). A coverslip was applied to the tissue sections. For Masson's trichrome staining, tissue sections were deparaffinized using heated Bouin's solution and then stained with Weigert's iron hematoxylin and Biebrich scarlet solutions. The tissue sections were then treated with phosphotungstic-phosphomolybdic acid and immediately stained with aniline blue solution. The tissue sections were rinsed and a coverslip was applied.

TABLE 2
Samples utilized for genome-wide analyses.

| | | | | | Next |
|---|---|---|---|---|---|
| Sample | | | | RNA | Gen |
| ID | Institution | Source | microRNA | Seq | Seq |
| NS1 | NorthShore Univ. | Liver Metastasis | x | x | x |
| NS10 | NorthShore Univ. | Liver Metastasis | x | x | |
| NS11 | NorthShore Univ. | Liver Metastasis | x | x | x |
| NS12 | NorthShore Univ. | Liver Metastasis | x | x | x |
| NS13 | NorthShore Univ. | Liver Metastasis | x | x | |
| NS14 | NorthShore Univ. | Liver Metastasis | x | x | |
| NS15 | NorthShore Univ. | Liver Metastasis | x | x | x |
| NS16 | NorthShore Univ. | Liver Metastasis | x | x | |
| NS17 | NorthShore Univ. | Liver Metastasis | x | x | |
| NS18 | NorthShore Univ. | Liver Metastasis | x | x | x |
| NS19 | NorthShore Univ. | Liver Metastasis | x | x | x |
| NS2 | NorthShore Univ. | Liver Metastasis | x | | |
| NS20 | NorthShore Univ. | Liver Metastasis | x | | x |
| NS21 | NorthShore Univ. | Liver Metastasis | x | x | |
| NS22 | NorthShore Univ. | Liver Metastasis | x | | x |
| NS23 | NorthShore Univ. | Liver Metastasis | x | | x |
| NS24 | NorthShore Univ. | Liver Metastasis | x | x | x |
| NS25 | NorthShore Univ. | Liver Metastasis | x | x | x |
| NS26 | NorthShore Univ. | Liver Metastasis | x | | x |
| NS27 | NorthShore Univ. | Liver Metastasis | x | x | |
| NS28 | NorthShore Univ. | Liver Metastasis | x | x | |
| NS29 | NorthShore Univ. | Liver Metastasis | x | | x |
| NS3 | NorthShore Univ. | Liver Metastasis | x | x | |
| NS30 | NorthShore Univ. | Liver Metastasis | x | x | x |
| NS31 | NorthShore Univ. | Liver Metastasis | x | x | x |
| NS32 | NorthShore Univ. | Liver Metastasis | x | x | |
| NS33 | NorthShore Univ. | Liver Metastasis | x | | |
| NS34 | NorthShore Univ. | Liver Metastasis | x | x | |
| NS35 | NorthShore Univ. | Liver Metastasis | x | x | x |
| NS4 | NorthShore Univ. | Liver Metastasis | x | x | x |
| NS5 | NorthShore Univ. | Liver Metastasis | x | x | |
| NS6 | NorthShore Univ. | Liver Metastasis | x | x | x |
| NS7 | NorthShore Univ. | Liver Metastasis | x | x | x |
| NS8 | NorthShore Univ. | Liver Metastasis | x | x | |
| NS9 | NorthShore Univ. | Liver Metastasis | x | x | x |
| UC1 | Univ. of Chicago | Liver Metastasis | x | x | |
| UC10 | Univ. of Chicago | Liver Metastasis | x | | |
| UC11 | Univ. of Chicago | Liver Metastasis | x | x | x |
| UC12 | Univ. of Chicago | Liver Metastasis | x | | |
| UC13 | Univ. of Chicago | Liver Metastasis | x | | |
| UC14 | Univ. of Chicago | Liver Metastasis | x | x | |
| UC15 | Univ. of Chicago | Liver Metastasis | x | x | |
| UC16 | Univ. of Chicago | Liver Metastasis | x | | |
| UC17 | Univ. of Chicago | Liver Metastasis | x | x | |
| UC18 | Univ. of Chicago | Liver Metastasis | x | x | |
| UC19 | Univ. of Chicago | Liver Metastasis | x | x | x |
| UC2 | Univ. of Chicago | Liver Metastasis | x | x | |
| UC20 | Univ. of Chicago | Liver Metastasis | x | x | |
| UC21 | Univ. of Chicago | Liver Metastasis | x | x | x |
| UC22 | Univ. of Chicago | Liver Metastasis | x | x | |
| UC23 | Univ. of Chicago | Liver Metastasis | | | x |
| UC24 | Univ. of Chicago | Liver Metastasis | x | x | |
| UC25 | Univ. of Chicago | Liver Metastasis | x | x | |
| UC26 | Univ. of Chicago | Liver Metastasis | x | | x |
| UC27 | Univ. of Chicago | Liver Metastasis | x | x | x |
| UC28 | Univ. of Chicago | Liver Metastasis | x | x | x |
| UC29 | Univ. of Chicago | Liver Metastasis | x | x | x |
| UC3 | Univ. of Chicago | Liver Metastasis | x | | |
| UC30 | Univ. of Chicago | Liver Metastasis | x | x | |
| UC31 | Univ. of Chicago | Liver Metastasis | x | x | x |
| UC32 | Univ. of Chicago | Liver Metastasis | x | x | |
| UC33 | Univ. of Chicago | Liver Metastasis | x | x | x |
| UC34 | Univ. of Chicago | Liver Metastasis | x | x | x |
| UC35 | Univ. of Chicago | Liver Metastasis | | | x |
| UC36 | Univ. of Chicago | Liver Metastasis | x | | x |
| UC37 | Univ. of Chicago | Liver Metastasis | x | x | x |
| UC38 | Univ. of Chicago | Liver Metastasis | x | | x |
| UC39 | Univ. of Chicago | Liver Metastasis | x | x | |
| UC4 | Univ. of Chicago | Liver Metastasis | x | x | |
| UC40 | Univ. of Chicago | Liver Metastasis | x | x | |
| UC41 | Univ. of Chicago | Liver Metastasis | x | x | x |
| UC42 | Univ. of Chicago | Liver Metastasis | x | x | |
| UC43 | Univ. of Chicago | Liver Metastasis | x | x | x |
| UC44 | Univ. of Chicago | Liver Metastasis | x | x | |
| UC45 | Univ. of Chicago | Liver Metastasis | x | x | x |
| UC46 | Univ. of Chicago | Liver Metastasis | x | x | |
| UC47 | Univ. of Chicago | Liver Metastasis | x | x | |
| UC48 | Univ. of Chicago | Liver Metastasis | x | | x |
| UC49 | Univ. of Chicago | Liver Metastasis | x | x | x |
| UC5 | Univ. of Chicago | Liver Metastasis | x | x | x |
| UC50 | Univ. of Chicago | Liver Metastasis | x | x | |
| UC51 | Univ. of Chicago | Liver Metastasis | x | x | x |
| UC52 | Univ. of Chicago | Liver Metastasis | x | x | |
| UC53 | Univ. of Chicago | Liver Metastasis | x | x | x |
| UC54 | Univ. of Chicago | Liver Metastasis | x | x | |
| UC55 | Univ. of Chicago | Liver Metastasis | x | x | x |
| UC56 | Univ. of Chicago | Liver Metastasis | | x | x |
| UC57 | Univ. of Chicago | Liver Metastasis | x | x | x |
| UC58 | Univ. of Chicago | Liver Metastasis | x | x | x |
| UC59 | Univ. of Chicago | Liver Metastasis | x | x | x |
| UC6 | Univ. of Chicago | Liver Metastasis | | x | |
| UC60 | Univ. of Chicago | Liver Metastasis | | | x |
| UC61 | Univ. of Chicago | Liver Metastasis | x | x | |
| UC62 | Univ. of Chicago | Liver Metastasis | x | x | x |
| UC63 | Univ. of Chicago | Liver Metastasis | x | | |
| UC64 | Univ. of Chicago | Liver Metastasis | x | | x |
| UC65 | Univ. of Chicago | Liver Metastasis | x | x | |
| UC66 | Univ. of Chicago | Liver Metastasis | x | x | |
| UC67 | Univ. of Chicago | Liver Metastasis | x | | |
| UC68 | Univ. of Chicago | Liver Metastasis | x | x | x |
| UC69 | Univ. of Chicago | Liver Metastasis | x | | |
| UC7 | Univ. of Chicago | Liver Metastasis | x | x | |
| UC70 | Univ. of Chicago | Liver Metastasis | x | x | x |
| UC71 | Univ. of Chicago | Liver Metastasis | x | x | |
| UC72 | Univ. of Chicago | Liver Metastasis | x | x | |
| UC73 | Univ. of Chicago | Liver Metastasis | x | x | x |
| UC74 | Univ. of Chicago | Liver Metastasis | x | x | x |
| UC75 | Univ. of Chicago | Liver Metastasis | x | x | |
| UC76 | Univ. of Chicago | Liver Metastasis | x | | x |
| UC77 | Univ. of Chicago | Liver Metastasis | x | | x |
| UC78 | Univ. of Chicago | Liver Metastasis | x | x | x |
| UC79 | Univ. of Chicago | Liver Metastasis | x | x | |
| UC8 | Univ. of Chicago | Liver Metastasis | x | x | |
| UC80 | Univ. of Chicago | Liver Metastasis | x | x | |
| UC81 | Univ. of Chicago | Liver Metastasis | x | x | |
| UC82 | Univ. of Chicago | Liver Metastasis | x | x | |
| UC83 | Univ. of Chicago | Liver Metastasis | x | x | |
| UC84 | Univ. of Chicago | Liver Metastasis | x | x | |
| UC85 | Univ. of Chicago | Liver Metastasis | x | x | |
| UC86 | Univ. of Chicago | Liver Metastasis | x | | |
| UC9 | Univ. of Chicago | Liver Metastasis | x | x | x |

Tables 3A-C. Differentially expressed genes across SNF clusters in 93 metastatic RNA Sequencing samples identified by the limma-voom method. (A) Differentially expressed genes (DEGs) between SNF1 versus SNF2 and 3. (B) DEGs between SNF2 versus SNF1 and 3. (C) DEGs between SNF3 versus SNF1 and 2. Log 2FC: estimate of the log 2 fold-change corresponding to the contrast. Adj.P.Val: Benjamini-Hochberg corrected P-value. Cutoff values for DEGs are log 2FC=±1 and adj.P.Val≤0.05.

TABLE 3A

| Ensembl ID | Gene Symbol | log2FC | adj P-Value |
|---|---|---|---|
| ENSG00000114113 | RBP2 | 2.402377682 | 0.000208982 |
| ENSG00000188176 | SMTNL2 | 2.397349614 | 0.000243497 |
| ENSG00000101958 | GLRA2 | 1.849125462 | 0.014880259 |
| ENSG00000165730 | STOX1 | 1.814566368 | 0.000376488 |
| ENSG00000124882 | EREG | 1.802200741 | 3.30361E−05 |
| ENSG00000068615 | REEP1 | 1.72856343 | 0.02439499 |
| ENSG00000149295 | DRD2 | 1.718068318 | 0.038733231 |
| ENSG00000188883 | KLRG2 | 1.686341802 | 0.015076777 |
| ENSG00000184368 | MAP7D2 | 1.670440677 | 0.042672544 |
| ENSG00000130234 | ACE2 | 1.558078536 | 0.011152632 |
| ENSG00000164076 | CAMKV | 1.507861424 | 0.036206093 |
| ENSG00000110195 | FOLR1 | 1.452851117 | 0.049653874 |
| ENSG00000168243 | GNG4 | 1.442401071 | 0.002155582 |
| ENSG00000135917 | SLC19A3 | 1.420649708 | 5.72027E−05 |
| ENSG00000186198 | SLC51B | 1.413076946 | 0.002412178 |
| ENSG00000064195 | DLX3 | 1.403694341 | 0.042259802 |
| ENSG00000109321 | AREG | 1.374721044 | 1.5288E−05 |
| ENSG00000151838 | CCDC175 | 1.356610589 | 0.00125377 |
| ENSG00000139292 | LGR5 | 1.330918379 | 0.001109408 |
| ENSG00000145569 | FAM105A | 1.275176254 | 9.64837E−08 |
| ENSG00000134215 | VAV3 | 1.269357856 | 3.00521E−05 |
| ENSG00000155850 | SLC26A2 | 1.261278624 | 0.008025503 |
| ENSG00000102924 | CBLN1 | 1.260630277 | 0.027551713 |
| ENSG00000108576 | SLC6A4 | 1.251556783 | 0.0342596 |
| ENSG00000275004 | ZNF280B | 1.24127452 | 5.1556E−05 |
| ENSG00000132821 | VSTM2L | 1.240352896 | 0.024453349 |
| ENSG00000130055 | GDPD2 | 1.212163873 | 0.016296313 |
| ENSG00000144354 | CDCA7 | 1.202383358 | 2.12792E−05 |
| ENSG00000159409 | CELF3 | 1.199684556 | 0.033506433 |
| ENSG00000155380 | SLC16A1 | 1.197394536 | 1.26489E−06 |
| ENSG00000186564 | FOXD2 | 1.194840276 | 0.001036938 |
| ENSG00000181690 | PLAG1 | 1.185059356 | 0.00538507 |
| ENSG00000196604 | POTEF | 1.169865687 | 0.039795376 |
| ENSG00000118557 | PMFBP1 | 1.159904285 | 0.0152575 |
| ENSG00000164649 | CDCA7L | 1.159525916 | 1.8842E−05 |
| ENSG00000116299 | KIAA1324 | 1.141930471 | 0.019276144 |
| ENSG00000117834 | SLC5A9 | 1.133319506 | 0.020497724 |
| ENSG00000154639 | CXADR | 1.115878739 | 8.00911E−08 |
| ENSG00000142606 | MMEL1 | 1.113779984 | 0.019290196 |
| ENSG00000021300 | PLEKHB1 | 1.109828939 | 0.003560571 |
| ENSG00000112742 | TTK | 1.107662416 | 1.46899E−05 |
| ENSG00000158555 | GDPD5 | 1.101681802 | 0.00095828 |
| ENSG00000088325 | TPX2 | 1.098363086 | 2.63438E−05 |
| ENSG00000196172 | ZNF681 | 1.098066536 | 0.001824751 |
| ENSG00000254726 | MEX3A | 1.087467248 | 0.000197599 |
| ENSG00000189431 | RASSF10 | 1.086839686 | 0.008703152 |
| ENSG00000164398 | ACSL6 | 1.084221924 | 0.0152218 |
| ENSG00000137251 | TINAG | 1.082801224 | 0.011697726 |
| ENSG00000132970 | WASF3 | 1.081994274 | 0.02734786 |
| ENSG00000131747 | TOP2A | 1.078065148 | 8.1531E−05 |
| ENSG00000000003 | TSPAN6 | 1.066413046 | 2.51158E−06 |
| ENSG00000188124 | OR2AG2 | 1.065088681 | 0.012084353 |
| ENSG00000198535 | C2CD4A | 1.062921578 | 0.016522229 |
| ENSG00000166866 | MYO1A | 1.041611068 | 0.001184125 |
| ENSG00000213160 | KLHL23 | 1.039218214 | 2.88262E−07 |
| ENSG00000161082 | CELF5 | 1.033491448 | 0.041100425 |
| ENSG00000164362 | TERT | 1.025951854 | 0.016841805 |
| ENSG00000184933 | OR6A2 | 1.02215216 | 0.008185591 |
| ENSG00000104537 | ANXA13 | 1.018421643 | 0.017649315 |
| ENSG00000104147 | OIP5 | 1.011329218 | 0.000574736 |
| ENSG00000136425 | CIB2 | 1.010255223 | 0.004913766 |
| ENSG00000106541 | AGR2 | 1.010190816 | 0.000511074 |
| ENSG00000126003 | PLAGL2 | 1.006386116 | 2.3823E−05 |
| ENSG00000136982 | DSCC1 | 1.004849438 | 0.000122738 |
| ENSG00000117069 | ST6GALNAC5 | −1.000782426 | 0.007780424 |
| ENSG00000102879 | CORO1A | −1.001054276 | 0.000318174 |
| ENSG00000214140 | PRCD | −1.001428963 | 0.002533169 |
| ENSG00000137868 | STRA6 | −1.001840186 | 0.02339985 |
| ENSG00000113657 | DPYSL3 | −1.002137358 | 0.000167553 |
| ENSG00000169902 | TPST1 | −1.002395305 | 8.10998E−05 |
| ENSG00000127951 | FGL2 | −1.002497793 | 0.000642762 |
| ENSG00000146859 | TMEM140 | −1.002747765 | 0.000108882 |
| ENSG00000172243 | CLEC7A | −1.003383024 | 0.000929502 |
| ENSG00000144824 | PHLDB2 | −1.004769482 | 0.00011297 |
| ENSG00000196209 | SIRPB2 | −1.005999695 | 6.78815E−05 |
| ENSG00000204389 | HSPA1A | −1.006401232 | 0.038503706 |
| ENSG00000071282 | LMCD1 | −1.007393734 | 1.14568E−05 |
| ENSG00000162461 | SLC25A34 | −1.007734946 | 0.000230625 |
| ENSG00000168779 | SHOX2 | −1.008751338 | 0.029715421 |
| ENSG00000213398 | LCAT | −1.008987592 | 0.000376827 |
| ENSG00000149591 | TAGLN | −1.009349405 | 8.33812E−05 |
| ENSG00000150656 | CNDP1 | −1.009526534 | 0.04648041 |
| ENSG00000173918 | C1QTNF1 | −1.009692419 | 2.47492E−06 |
| ENSG00000103544 | C16orf62 | −1.010080227 | 4.781E−05 |
| ENSG00000164220 | F2RL2 | −1.010267071 | 0.004009132 |
| ENSG00000005243 | COPZ2 | −1.010284615 | 3.00382E−05 |
| ENSG00000150681 | RGS18 | −1.011124365 | 0.004238641 |
| ENSG00000135046 | ANXA1 | −1.011681717 | 0.000329194 |
| ENSG00000184588 | PDE4B | −1.011804992 | 0.000489961 |
| ENSG00000004399 | PLXND1 | −1.012137845 | 1.33346E−06 |
| ENSG00000130270 | ATP8B3 | −1.0126323 | 0.001458946 |
| ENSG00000155269 | GPR78 | −1.013738113 | 0.014942782 |
| ENSG00000161835 | GRASP | −1.014017589 | 0.000287209 |
| ENSG00000078098 | FAP | −1.015132052 | 7.35271E−05 |
| ENSG00000007129 | CEACAM21 | −1.015538603 | 0.002250946 |
| ENSG00000162849 | KIF26B | −1.015776584 | 0.000624406 |
| ENSG00000120913 | PDLIM2 | −1.016116229 | 1.07962E−07 |
| ENSG00000129514 | FOXA1 | −1.017427806 | 0.020065896 |
| ENSG00000169129 | AFAP1L2 | −1.017642893 | 0.000693535 |
| ENSG00000133104 | SPG20 | −1.017758165 | 1.30418E−05 |
| ENSG00000184979 | USP18 | −1.017784816 | 0.003386386 |
| ENSG00000086288 | NME8 | −1.01848261 | 0.00069269 |
| ENSG00000174837 | EMR1 | −1.018771074 | 0.028479292 |
| ENSG00000170989 | S1PR1 | −1.019226184 | 0.000187363 |
| ENSG00000185652 | NTF3 | −1.01939436 | 0.00782992 |
| ENSG00000171700 | RGS19 | −1.021064945 | 1.02545E−05 |
| ENSG00000166292 | TMEM100 | −1.021075304 | 0.009254341 |
| ENSG00000135905 | DOCK10 | −1.022203289 | 0.00010271 |
| ENSG00000145244 | CORIN | −1.02236406 | 0.000378948 |
| ENSG00000113389 | NPR3 | −1.022421244 | 0.000778194 |
| ENSG00000183837 | PNMA3 | −1.022602374 | 0.028669553 |
| ENSG00000142227 | EMP3 | −1.022753035 | 3.02792E−06 |
| ENSG00000136689 | IL1RN | −1.022894131 | 0.004970645 |
| ENSG00000088538 | DOCK3 | −1.022986395 | 0.003476847 |
| ENSG00000104267 | CA2 | −1.023079238 | 0.003597149 |
| ENSG00000162946 | DISC1 | −1.023113136 | 3.83587E−05 |
| ENSG00000182885 | GPR97 | −1.023512349 | 0.003836755 |
| ENSG00000090339 | ICAM1 | −1.023933544 | 2.16095E−05 |
| ENSG00000174004 | NRROS | −1.025033931 | 0.001102818 |
| ENSG00000134326 | CMPK2 | −1.025302594 | 0.000825354 |
| ENSG00000178404 | KIAA1731NL | −1.026263787 | 1.63415E−05 |
| ENSG00000254535 | PABPC4L | −1.026264785 | 0.000606648 |
| ENSG00000127084 | FGD3 | −1.026564283 | 2.72501E−05 |
| ENSG00000260729 | RP11-106M3.2 | −1.026686491 | 0.003808553 |
| ENSG00000088756 | ARHGAP28 | −1.026798954 | 0.013969182 |
| ENSG00000174640 | SLCO2A1 | −1.027077345 | 0.004601407 |
| ENSG00000254521 | SIGLEC12 | −1.027098989 | 0.013153195 |
| ENSG00000150995 | ITPR1 | −1.027862169 | 3.14471E−07 |
| ENSG00000135094 | SDS | −1.028193464 | 0.013303757 |
| ENSG00000077092 | RARB | −1.028239627 | 1.24729E−05 |
| ENSG00000107518 | ATRNL1 | −1.028730709 | 0.002800469 |
| ENSG00000135842 | FAM129A | −1.028858655 | 6.49193E−06 |
| ENSG00000134242 | PTPN22 | −1.02976135 | 0.00024393 |
| ENSG00000135709 | KIAA0513 | −1.029785007 | 5.10681E−05 |
| ENSG00000171033 | PKIA | −1.029854887 | 0.047967857 |
| ENSG00000141497 | ZMYND15 | −1.030036851 | 2.90319E−05 |
| ENSG00000156113 | KCNMA1 | −1.031003391 | 0.002869868 |
| ENSG00000004776 | HSPB6 | −1.031066746 | 0.002274733 |
| ENSG00000143110 | C1orf162 | −1.0319018 | 4.781E−05 |
| ENSG00000185634 | SHC4 | −1.031905167 | 0.002917639 |
| ENSG00000132932 | ATP8A2 | −1.032621332 | 0.006321757 |
| ENSG00000160469 | BRSK1 | −1.03284979 | 0.000460022 |
| ENSG00000137094 | DNAJB5 | −1.033957151 | 6.18202E−06 |
| ENSG00000129116 | PALLD | −1.03409591 | 1.09763E−05 |
| ENSG00000204682 | CASC10 | −1.034497917 | 0.00605214 |
| ENSG00000197380 | DACT3 | −1.034720388 | 6.51406E−06 |
| ENSG00000138185 | ENTPD1 | −1.034814376 | 1.26735E−05 |
| ENSG00000164330 | EBF1 | −1.035602254 | 0.000146673 |
| ENSG00000036530 | CYP46A1 | −1.035945787 | 0.008262352 |
| ENSG00000151650 | VENTX | −1.035991037 | 0.031368411 |
| ENSG00000175262 | C1orf127 | −1.036174402 | 0.01204617 |
| ENSG00000269404 | SPIB | −1.036664975 | 0.013084021 |
| ENSG00000144837 | PLA1A | −1.037827634 | 0.01942901 |
| ENSG00000171951 | SCG2 | −1.038502613 | 0.03101343 |
| ENSG00000196923 | PDLIM7 | −1.03950379 | 2.10135E−05 |
| ENSG00000178498 | DTX3 | −1.039873107 | 7.03238E−05 |
| ENSG00000143387 | CTSK | −1.040149202 | 1.05242E−05 |
| ENSG00000160471 | COX6B2 | −1.040316079 | 0.002013588 |
| ENSG00000157654 | PALM2-AKAP2 | −1.040535078 | 0.00119669 |
| ENSG00000131015 | ULBP2 | −1.040894272 | 0.004465717 |
| ENSG00000143995 | MEIS1 | −1.040926559 | 3.30344E−05 |
| ENSG00000167549 | CORO6 | −1.041072554 | 0.004416835 |
| ENSG00000137507 | LRRC32 | −1.041274101 | 1.5046E−05 |
| ENSG00000167476 | JSRP1 | −1.042762963 | 0.00321037 |
| ENSG00000162687 | KCNT2 | −1.043380463 | 0.000359668 |
| ENSG00000167850 | CD300C | −1.043537764 | 0.000492358 |
| ENSG00000148516 | ZEB1 | −1.043553924 | 6.53044E−06 |
| ENSG00000119699 | TGFB3 | −1.043677454 | 5.10681E−05 |
| ENSG00000089692 | LAG3 | −1.043705335 | 0.000887602 |
| ENSG00000101307 | SIRPB1 | −1.043945303 | 0.001335425 |
| ENSG00000162426 | SLC45A1 | −1.043977512 | 0.000349024 |
| ENSG00000124813 | RUNX2 | −1.044316033 | 5.63003E−05 |
| ENSG00000166183 | ASPG | −1.044697411 | 0.046582938 |
| ENSG00000013619 | MAMLD1 | −1.04527249 | 3.14471E−07 |
| ENSG00000139572 | GPR84 | −1.045520338 | 0.001236327 |
| ENSG00000140519 | RHCG | −1.045539492 | 0.038860595 |
| ENSG00000049246 | PER3 | −1.045718783 | 5.42012E−05 |
| ENSG00000160321 | ZNF208 | −1.04580772 | 0.01357857 |
| ENSG00000140859 | KIFC3 | −1.045878857 | 5.19906E−05 |
| ENSG00000138639 | ARHGAP24 | −1.045932518 | 4.62262E−05 |
| ENSG00000266094 | RASSF5 | −1.04629921 | 4.08124E−05 |
| ENSG00000169418 | NPR1 | −1.047205485 | 0.00235917 |
| ENSG00000138944 | KIAA1644 | −1.047706437 | 0.001864123 |
| ENSG00000139899 | CBLN3 | −1.047818775 | 5.96005E−05 |
| ENSG00000067445 | TRO | −1.04859493 | 1.16543E−05 |
| ENSG00000169554 | ZEB2 | −1.049049731 | 1.27047E−05 |
| ENSG00000161298 | ZNF382 | −1.050061162 | 2.08379E−05 |
| ENSG00000057657 | PRDM1 | −1.050475131 | 2.76635E−05 |
| ENSG00000186354 | C9orf47 | −1.050665373 | 0.007978122 |
| ENSG00000105137 | SYDE1 | −1.050782007 | 2.58796E−06 |
| ENSG00000121068 | TBX2 | −1.051370833 | 8.64705E−05 |
| ENSG00000173320 | STOX2 | −1.052255578 | 0.002048736 |
| ENSG00000128918 | ALDH1A2 | −1.052278199 | 0.002941967 |
| ENSG00000196632 | WNK3 | −1.052391031 | 0.002434239 |
| ENSG00000143552 | NUP210L | −1.052581466 | 0.000741341 |
| ENSG00000185100 | ADSSL1 | −1.052778945 | 6.589E−05 |
| ENSG00000074966 | TXK | −1.053158781 | 0.000514017 |
| ENSG00000065413 | ANKRD44 | −1.053710376 | 1.313E−05 |
| ENSG00000104951 | IL4I1 | −1.053776753 | 0.004869228 |
| ENSG00000107738 | C10orf54 | −1.053866868 | 6.78455E−07 |
| ENSG00000082014 | SMARCD3 | −1.054298277 | 6.10459E−06 |
| ENSG00000244242 | IFITM10 | −1.05435803 | 3.12165E−05 |
| ENSG00000129244 | ATP1B2 | −1.054434713 | 0.000491418 |
| ENSG00000176049 | JAKMIP2 | −1.054842449 | 0.016921277 |
| ENSG00000112799 | LY86 | −1.055140672 | 0.006357425 |
| ENSG00000153064 | BANK1 | −1.055342773 | 0.021723394 |
| ENSG00000172322 | CLEC12A | −1.05774634 | 0.005306616 |
| ENSG00000074660 | SCARF1 | −1.05809983 | 2.82629E−07 |
| ENSG00000146054 | TRIM7 | −1.058384682 | 0.017215382 |
| ENSG00000171533 | MAP6 | −1.058716842 | 2.04347E−05 |
| ENSG00000187800 | PEAR1 | −1.05904284 | 3.9877E−05 |
| ENSG00000154734 | ADAMTS1 | −1.059294153 | 0.000810995 |
| ENSG00000113721 | PDGFRB | −1.059329869 | 4.81498E−05 |
| ENSG00000186469 | GNG2 | −1.059346757 | 3.46938E−05 |
| ENSG00000095370 | SH2D3C | −1.059764088 | 3.2622E−06 |
| ENSG00000185338 | SOCS1 | −1.060525597 | 0.000457649 |
| ENSG00000145284 | SCD5 | −1.060596058 | 0.000322701 |
| ENSG00000108691 | CCL2 | −1.0613407 | 0.000759205 |
| ENSG00000049249 | TNFRSF9 | −1.061690428 | 0.000508862 |
| ENSG00000118849 | RARRES1 | −1.062423869 | 0.000530439 |
| ENSG00000258405 | ZNF578 | −1.062551598 | 0.007548984 |
| ENSG00000010810 | FYN | −1.063074191 | 0.000265018 |
| ENSG00000166825 | ANPEP | −1.063700087 | 0.005121934 |
| ENSG00000204131 | NHSL2 | −1.063791035 | 0.000195959 |
| ENSG00000105472 | CLEC11A | −1.064427076 | 1.97624E−06 |
| ENSG00000148175 | STOM | −1.065098031 | 1.58442E−05 |
| ENSG00000118514 | ALDH8A1 | −1.065635125 | 0.036640022 |
| ENSG00000109944 | C11orf63 | −1.065705697 | 0.000368205 |
| ENSG00000205413 | SAMD9 | −1.066198494 | 7.82876E−05 |
| ENSG00000204642 | HLA-F | −1.066694092 | 0.001024249 |
| ENSG00000123342 | MMP19 | −1.066860848 | 0.002186035 |
| ENSG00000136383 | ALPK3 | −1.067183406 | 0.001228773 |
| ENSG00000132031 | MATN3 | −1.067240512 | 0.019227324 |
| ENSG00000186074 | CD300LF | −1.06734085 | 0.000615876 |
| ENSG00000105711 | SCN1B | −1.067554069 | 4.81558E−05 |
| ENSG00000178726 | THBD | −1.068320198 | 0.000108526 |
| ENSG00000153956 | CACNA2D1 | −1.068480709 | 8.11946E−06 |
| ENSG00000137573 | SULF1 | −1.069334753 | 0.001017246 |
| ENSG00000106605 | BLVRA | −1.069357577 | 0.000137105 |
| ENSG00000169085 | C8orf46 | −1.069479551 | 0.033990634 |
| ENSG00000132357 | CARD6 | −1.069897645 | 1.22507E−05 |
| ENSG00000150337 | FCGR1A | −1.070347526 | 0.005282353 |
| ENSG00000071967 | CYBRD1 | −1.070482987 | 0.000373622 |
| ENSG00000188921 | PTPLAD2 | −1.070615287 | 6.60266E−05 |
| ENSG00000178860 | MSC | −1.07066421 | 6.65599E−06 |
| ENSG00000145107 | TM4SF19 | −1.071086564 | 0.002429768 |
| ENSG00000121207 | LRAT | −1.071196068 | 0.007850145 |
| ENSG00000143819 | EPHX1 | −1.071596274 | 0.000873153 |
| ENSG00000165646 | SLC18A2 | −1.071809927 | 0.010164474 |
| ENSG00000131471 | AOC3 | −1.072052773 | 7.17896E−06 |
| ENSG00000035862 | TIMP2 | −1.072069427 | 1.48428E−05 |
| ENSG00000125510 | OPRL1 | −1.072672553 | 0.00056955 |
| ENSG00000080493 | SLC4A4 | −1.072857311 | 0.006470904 |
| ENSG00000113580 | NR3C1 | −1.073346897 | 1.05502E−05 |
| ENSG00000184613 | NELL2 | −1.074137359 | 0.010006497 |
| ENSG00000183186 | C2CD4C | −1.074179971 | 0.002277851 |
| ENSG00000206052 | DOK6 | −1.074258727 | 0.001001334 |
| ENSG00000137691 | C11orf70 | −1.074374696 | 0.002354684 |
| ENSG00000197816 | CCDC180 | −1.075377114 | 0.000562678 |
| ENSG00000123610 | TNFAIP6 | −1.075504166 | 0.001487586 |
| ENSG00000145431 | PDGFC | −1.07565821 | 2.33046E−05 |
| ENSG00000077420 | APBB1IP | −1.075833206 | 0.000148168 |
| ENSG00000011600 | TYROBP | −1.07691373 | 0.000149906 |
| ENSG00000136167 | LCP1 | −1.077069203 | 0.000284648 |
| ENSG00000166780 | C16orf45 | −1.078697479 | 1.14032E−06 |
| ENSG00000164440 | TXLNB | −1.078844273 | 0.00150505 |
| ENSG00000099864 | PALM | −1.079055262 | 7.4755E−05 |
| ENSG00000108798 | ABI3 | −1.079131192 | 1.00502E−05 |
| ENSG00000134571 | MYBPC3 | −1.079153579 | 0.00384447 |
| ENSG00000188171 | ZNF626 | −1.079313846 | 0.00050143 |
| ENSG00000121361 | KCNJ8 | −1.079416303 | 0.000658717 |
| ENSG00000155761 | SPAG17 | −1.079523013 | 0.019849521 |
| ENSG00000134460 | IL2RA | −1.07982295 | 0.00017803 |
| ENSG00000069667 | RORA | −1.080113903 | 0.000230625 |
| ENSG00000143119 | CD53 | −1.080681878 | 8.413E−05 |
| ENSG00000166963 | MAP1A | −1.08130821 | 2.38468E−06 |
| ENSG00000185386 | MAPK11 | −1.08154262 | 6.53044E−06 |
| ENSG00000073150 | PANX2 | −1.082028358 | 0.010100591 |
| ENSG00000153093 | ACOXL | −1.082303955 | 0.027831493 |
| ENSG00000027075 | PRKCH | −1.082894115 | 1.26489E−06 |
| ENSG00000149781 | FERMT3 | −1.083400399 | 1.39654E−06 |
| ENSG00000164088 | PPM1M | −1.083568678 | 4.71788E−07 |
| ENSG00000106689 | LHX2 | −1.083713658 | 0.005294238 |
| ENSG00000115956 | PLEK | −1.083751872 | 7.99128E−05 |
| ENSG00000101017 | CD40 | −1.085183047 | 0.000439681 |
| ENSG00000162722 | TRIM58 | −1.085234165 | 0.036793838 |
| ENSG00000128274 | A4GALT | −1.085847949 | 0.000424965 |
| ENSG00000101265 | RASSF2 | −1.086241441 | 4.54694E−06 |
| ENSG00000133800 | LYVE1 | −1.087559122 | 0.034749779 |
| ENSG00000162367 | TAL1 | −1.087642472 | 0.000404758 |
| ENSG00000127220 | ABHD8 | −1.088170824 | 0.000447151 |
| ENSG00000090376 | IRAK3 | −1.088340885 | 1.56288E−05 |
| ENSG00000081818 | PCDHB4 | −1.088478389 | 0.025502916 |
| ENSG00000117586 | TNFSF4 | −1.089585956 | 1.64751E−05 |
| ENSG00000115165 | CYTIP | −1.09091077 | 6.22813E−05 |
| ENSG00000137959 | IFI44L | −1.091015582 | 0.020972087 |
| ENSG00000117228 | GBP1 | −1.09132325 | 0.000309421 |
| ENSG00000172403 | SYNPO2 | −1.091326944 | 4.11799E−05 |
| ENSG00000020181 | GPR124 | −1.091663914 | 3.29587E−06 |
| ENSG00000151702 | FLI1 | −1.091781691 | 6.18202E−06 |
| ENSG00000139625 | MAP3K12 | −1.093430672 | 1.91479E−07 |
| ENSG00000164236 | ANKRD33B | −1.094590362 | 8.73266E−07 |
| ENSG00000186340 | THBS2 | −1.095496627 | 0.000173743 |
| ENSG00000271605 | MILR1 | −1.096240331 | 0.000158482 |
| ENSG00000115232 | ITGA4 | −1.09626015 | 3.16481E−05 |
| ENSG00000127129 | EDN2 | −1.096508376 | 0.019148295 |
| ENSG00000182013 | PNMAL1 | −1.096652021 | 0.003136705 |
| ENSG00000204262 | COL5A2 | −1.097604394 | 4.0212E−05 |
| ENSG00000131435 | PDLIM4 | −1.099355604 | 0.007330896 |
| ENSG00000183580 | FBXL7 | −1.09952859 | 1.11007E−05 |
| ENSG00000133401 | PDZD2 | −1.100262075 | 0.000872012 |
| ENSG00000171236 | LRG1 | −1.101288852 | 0.024666056 |
| ENSG00000140678 | ITGAX | −1.102029983 | 0.000175407 |
| ENSG00000165617 | DACT1 | −1.102233742 | 2.29257E−05 |
| ENSG00000132965 | ALOX5AP | −1.10283166 | 0.000205549 |
| ENSG00000232629 | HLA-DQB2 | −1.102911936 | 0.003997252 |
| ENSG00000162733 | DDR2 | −1.103417294 | 7.23692E−06 |
| ENSG00000169122 | FAM110B | −1.103515248 | 0.00012276 |
| ENSG00000155307 | SAMSN1 | −1.103960983 | 0.000360639 |
| ENSG00000185483 | ROR1 | −1.104126239 | 0.000103448 |
| ENSG00000053328 | METTL24 | −1.105532104 | 0.000503779 |
| ENSG00000182263 | FIGN | −1.106760156 | 0.001174942 |
| ENSG00000142694 | EVA1B | −1.106774972 | 1.3873E−05 |
| ENSG00000184986 | TMEM121 | −1.107016608 | 0.002028343 |
| ENSG00000182866 | LCK | −1.108219651 | 0.00105822 |
| ENSG00000156006 | NAT2 | −1.108306929 | 4.32466E−05 |
| ENSG00000171817 | ZNF540 | −1.108424962 | 0.000668849 |
| ENSG00000138615 | CILP | −1.108544385 | 0.00140159 |
| ENSG00000095321 | CRAT | −1.108933986 | 6.099E−06 |
| ENSG00000131018 | SYNE1 | −1.109202846 | 2.47631E−05 |
| ENSG00000178573 | MAF | −1.109395092 | 2.93627E−06 |
| ENSG00000087589 | CASS4 | −1.109552356 | 5.12081E−06 |
| ENSG00000133256 | PDE6B | −1.109657156 | 0.003190935 |
| ENSG00000138829 | FBN2 | −1.110204802 | 0.018183516 |
| ENSG00000173641 | HSPB7 | −1.110988949 | 0.000929502 |
| ENSG00000124496 | TRERF1 | −1.111200775 | 6.18202E−06 |
| ENSG00000150637 | CD226 | −1.111232082 | 0.000394449 |
| ENSG00000139737 | SLAIN1 | −1.112090463 | 0.006838191 |
| ENSG00000164692 | COL1A2 | −1.112403098 | 5.56759E−05 |
| ENSG00000135480 | KRT7 | −1.112877225 | 4.80699E−05 |
| ENSG00000117091 | CD48 | −1.113270186 | 0.000261918 |
| ENSG00000177294 | FBXO39 | −1.113293341 | 0.00072053 |
| ENSG00000149380 | P4HA3 | −1.114113044 | 1.67292E−05 |
| ENSG00000130487 | KLHDC7B | −1.114285337 | 0.000474403 |
| ENSG00000172348 | RCAN2 | −1.115210077 | 0.000246565 |
| ENSG00000120129 | DUSP1 | −1.115785394 | 0.000400056 |
| ENSG00000117016 | RIMS3 | −1.116823963 | 7.45298E−05 |
| ENSG00000177409 | SAMD9L | −1.117434192 | 0.000700465 |
| ENSG00000140030 | GPR65 | −1.118044245 | 9.40358E−06 |
| ENSG00000163239 | TDRD10 | −1.118422854 | 0.000907887 |
| ENSG00000198879 | SFMBT2 | −1.118938964 | 1.06695E−05 |
| ENSG00000147408 | CSGALNACT1 | −1.119739001 | 0.000192521 |
| ENSG00000182809 | CRIP2 | −1.120605935 | 2.38468E−06 |
| ENSG00000175003 | SLC22A1 | −1.121177319 | 0.031107642 |
| ENSG00000133985 | TTC9 | −1.121442899 | 9.18726E−05 |
| ENSG00000176438 | SYNE3 | −1.121646511 | 1.01098E−06 |
| ENSG00000092096 | SLC22A17 | −1.121778181 | 1.87861E−05 |
| ENSG00000137726 | FXYD6 | −1.122399792 | 0.000104671 |
| ENSG00000171051 | FPR1 | −1.122549291 | 0.000335715 |
| ENSG00000174945 | AMZ1 | −1.124101157 | 3.39034E−05 |
| ENSG00000146112 | PPP1R18 | −1.124331805 | 1.58453E−07 |
| ENSG00000184113 | CLDN5 | −1.1251397 | 0.000995088 |
| ENSG00000049768 | FOXP3 | −1.125765608 | 0.00019175 |
| ENSG00000134955 | SLC37A2 | −1.125880339 | 4.38625E−06 |
| ENSG00000164604 | GPR85 | −1.126590857 | 0.004144476 |
| ENSG00000135925 | WNT10A | −1.126950717 | 0.017351572 |
| ENSG00000181804 | SLC9A9 | −1.12790158 | 0.001278941 |
| ENSG00000265107 | GJA5 | −1.128189705 | 0.000131847 |
| ENSG00000225614 | ZNF469 | −1.128348931 | 1.58442E−05 |
| ENSG00000235568 | NFAM1 | −1.128563754 | 1.33212E−06 |
| ENSG00000138172 | CALHM2 | −1.12879888 | 6.63213E−07 |
| ENSG00000087116 | ADAMTS2 | −1.129190361 | 3.2622E−06 |
| ENSG00000093072 | CECR1 | −1.129217403 | 0.000164657 |
| ENSG00000182327 | GLTPD2 | −1.129995159 | 0.035168166 |
| ENSG00000160219 | GAB3 | −1.131016525 | 2.62136E−06 |
| ENSG00000130635 | COL5A1 | −1.131597309 | 3.29885E−06 |
| ENSG00000149633 | KIAA1755 | −1.131780689 | 4.09908E−06 |
| ENSG00000184058 | TBX1 | −1.131814913 | 0.00034455 |
| ENSG00000163219 | ARHGAP25 | −1.132316175 | 1.80964E−06 |
| ENSG00000084110 | HAL | −1.132621911 | 0.049765672 |
| ENSG00000203883 | SOX18 | −1.132872117 | 4.38273E−06 |
| ENSG00000139278 | GLIPR1 | −1.133166488 | 1.09064E−05 |
| ENSG00000168077 | SCARA3 | −1.133251266 | 0.00666479 |
| ENSG00000173535 | TNFRSF10C | −1.133514942 | 0.000447151 |
| ENSG00000043462 | LCP2 | −1.133719047 | 1.68559E−05 |
| ENSG00000240771 | ARHGEF25 | −1.134623068 | 1.98003E−06 |
| ENSG00000159674 | SPON2 | −1.135017344 | 3.86458E−07 |
| ENSG00000184349 | EFNA5 | −1.135927178 | 0.000210271 |
| ENSG00000145623 | OSMR | −1.13622243 | 1.44968E−05 |
| ENSG00000005249 | PRKAR2B | −1.136701435 | 0.000624934 |
| ENSG00000198417 | MT1F | −1.137534728 | 0.000779952 |
| ENSG00000125740 | FOSB | −1.138518114 | 0.01115871 |
| ENSG00000169604 | ANTXR1 | −1.138929461 | 0.000518846 |
| ENSG00000008513 | ST3GAL1 | −1.139346894 | 1.3891E−05 |
| ENSG00000153162 | BMP6 | −1.140070502 | 0.001057195 |
| ENSG00000117525 | F3 | −1.140080597 | 5.63035E−05 |
| ENSG00000024422 | EHD2 | −1.140832352 | 3.86458E−07 |
| ENSG00000101955 | SRPX | −1.140968253 | 0.00181741 |
| ENSG00000126759 | CFP | −1.141115715 | 0.000103448 |
| ENSG00000174130 | TLR6 | −1.141472276 | 1.61997E−05 |
| ENSG00000140937 | CDH11 | −1.141735103 | 4.91461E−05 |
| ENSG00000144229 | THSD7B | −1.141903892 | 0.005178232 |
| ENSG00000165633 | VSTM4 | −1.142100677 | 1.40047E−06 |
| ENSG00000160883 | HK3 | −1.142242025 | 0.000156299 |
| ENSG00000106366 | SERPINE1 | −1.142993544 | 0.00219023 |
| ENSG00000124491 | F13A1 | −1.144331261 | 0.003481187 |
| ENSG00000135363 | LMO2 | −1.145868483 | 0.000110163 |
| ENSG00000157017 | GHRL | −1.146256277 | 0.00045037 |
| ENSG00000185862 | EVI2B | −1.146362154 | 0.00015935 |
| ENSG00000188783 | PRELP | −1.146456575 | 0.000530734 |
| ENSG00000154721 | JAM2 | −1.146492986 | 5.99703E−05 |
| ENSG00000107186 | MPDZ | −1.14667538 | 1.80809E−05 |
| ENSG00000141968 | VAV1 | −1.147075794 | 4.49639E−05 |
| ENSG00000225968 | ELFN1 | −1.147216786 | 0.000330204 |
| ENSG00000147697 | GSDMC | −1.147240602 | 0.002444374 |
| ENSG00000175264 | CHST1 | −1.147664007 | 2.42428E−06 |
| ENSG00000214456 | PLIN5 | −1.147759689 | 0.034248162 |
| ENSG00000186827 | TNFRSF4 | −1.148544774 | 6.02213E−05 |
| ENSG00000136286 | MYO1G | −1.148977907 | 1.26977E−06 |
| ENSG00000163563 | MNDA | −1.149208708 | 3.43305E−05 |
| ENSG00000150471 | LPHN3 | −1.149573788 | 4.94402E−05 |
| ENSG00000136842 | TMOD1 | −1.150027249 | 0.012434916 |
| ENSG00000170962 | PDGFD | −1.15005092 | 0.004180529 |
| ENSG00000188596 | CFAP54 | −1.150400084 | 0.000191136 |
| ENSG00000169136 | ATF5 | −1.151263756 | 0.000496023 |
| ENSG00000172716 | SLFN11 | −1.151731402 | 2.06385E−06 |
| ENSG00000171049 | FPR2 | −1.151774052 | 0.009388316 |
| ENSG00000134321 | RSAD2 | −1.15268933 | 0.001401716 |
| ENSG00000115252 | PDE1A | −1.152693629 | 4.38625E−06 |
| ENSG00000169248 | CXCL11 | −1.152849475 | 0.038859034 |
| ENSG00000166927 | MS4A7 | −1.153085729 | 0.000208366 |
| ENSG00000158186 | MRAS | −1.153571411 | 1.93937E−06 |
| ENSG00000167680 | SEMA6B | −1.154940426 | 1.13595E−06 |
| ENSG00000010310 | GIPR | −1.155433522 | 1.33594E−05 |
| ENSG00000106123 | EPHB6 | −1.156630456 | 0.003812667 |
| ENSG00000170786 | SDR16C5 | −1.157339434 | 0.02161042 |
| ENSG00000118473 | SGIP1 | −1.158832566 | 6.66812E−06 |
| ENSG00000166432 | ZMAT1 | −1.159213368 | 3.16481E−05 |
| ENSG00000176014 | TUBB6 | −1.159261511 | 6.51487E−07 |
| ENSG00000177374 | HIC1 | −1.159337792 | 4.89951E−07 |
| ENSG00000143502 | SUSD4 | −1.159827297 | 0.009295065 |
| ENSG00000120337 | TNFSF18 | −1.160871924 | 0.000855507 |
| ENSG00000119138 | KLF9 | −1.162445981 | 3.66474E−05 |
| ENSG00000124334 | IL9R | −1.162572201 | 0.002278566 |
| ENSG00000147573 | TRIM55 | −1.162600898 | 0.041543957 |
| ENSG00000186994 | KANK3 | −1.16339897 | 2.20908E−05 |
| ENSG00000109113 | RAB34 | −1.164082237 | 2.07859E−06 |
| ENSG00000182168 | UNC5C | −1.16408554 | 3.10231E−06 |
| ENSG00000196569 | LAMA2 | −1.164637929 | 0.000695574 |
| ENSG00000131831 | RAI2 | −1.164930933 | 0.000153154 |
| ENSG00000110446 | SLC15A3 | −1.165008055 | 9.45503E−06 |
| ENSG00000166923 | GREMI | −1.165067579 | 0.008425316 |
| ENSG00000119865 | CNRIP1 | −1.1653631 | 2.93382E−06 |
| ENSG00000204442 | FAM155A | −1.165398098 | 0.039253389 |
| ENSG00000122986 | HVCN1 | −1.165640049 | 4.05188E−05 |
| ENSG00000167895 | TMC8 | −1.166270436 | 0.000274175 |
| ENSG00000189056 | RELN | −1.1663162 | 0.008258713 |
| ENSG00000255587 | RAB44 | −1.166543722 | 0.000531022 |
| ENSG00000125538 | IL1B | −1.166866232 | 0.002501545 |
| ENSG00000115590 | IL1R2 | −1.166984229 | 0.000426636 |
| ENSG00000135063 | FAM189A2 | −1.167428531 | 0.007331374 |
| ENSG00000107099 | DOCK8 | −1.167694574 | 4.61847E−06 |
| ENSG00000137841 | PLCB2 | −1.168442317 | 1.94372E−05 |
| ENSG00000232810 | TNF | −1.16848809 | 0.001389679 |
| ENSG00000242574 | HLA-DMB | −1.168729004 | 3.57708E−05 |
| ENSG00000130052 | STARD8 | −1.168935307 | 1.7421E−05 |
| ENSG00000122641 | INHBA | −1.169397155 | 0.000183548 |
| ENSG00000181007 | ZFP82 | −1.169814254 | 0.000464191 |
| ENSG00000169026 | MFSD7 | −1.170266056 | 1.20046E−05 |
| ENSG00000078596 | ITM2A | −1.170384262 | 0.00189795 |
| ENSG00000165521 | EML5 | −1.170468916 | 0.001052901 |
| ENSG00000163359 | COL6A3 | −1.170669016 | 2.95551E−05 |
| ENSG00000167077 | MEI1 | −1.171680933 | 7.46667E−06 |
| ENSG00000160791 | CCR5 | −1.171845258 | 3.16236E−05 |
| ENSG00000173698 | GPR64 | −1.172686597 | 0.043142038 |
| ENSG00000115935 | WIPF1 | −1.173078636 | 8.06061E−06 |
| ENSG00000182853 | VMO1 | −1.173308348 | 0.004895509 |
| ENSG00000150048 | CLEC1A | -1.174214173 | 1.31665E−05 |
| ENSG00000106823 | ECM2 | −1.174646561 | 9.44593E−06 |
| ENSG00000146122 | DAAM2 | −1.174740771 | 6.25084E−06 |
| ENSG00000105366 | SIGLEC8 | −1.176430354 | 0.007019332 |
| ENSG00000185499 | MUC1 | −1.176632863 | 0.027474941 |
| ENSG00000159189 | C1QC | −1.176676781 | 8.78221E−05 |
| ENSG00000171488 | LRRC8C | −1.177362834 | 4.73932E−07 |
| ENSG00000182175 | RGMA | −1.177716915 | 5.42012E−05 |
| ENSG00000182871 | COL18A1 | −1.178386322 | 1.58362E−05 |
| ENSG00000135898 | GPR55 | −1.1786331 | 0.04168832 |
| ENSG00000127472 | PLA2G5 | −1.179871732 | 0.001987752 |
| ENSG00000244607 | CCDC13 | -1.179985727 | 0.00065737 |
| ENSG00000164197 | RNF180 | -1.180272508 | 6.62915E−06 |
| ENSG00000179909 | ZNF154 | −1.180353169 | 6.86309E−07 |
| ENSG00000173546 | CSPG4 | −1.18060396 | 0.000105814 |
| ENSG00000154822 | PLCL2 | −1.180646062 | 4.8614E−06 |
| ENSG00000130775 | THEMIS2 | −1.181185522 | 5.4965E−06 |
| ENSG00000134532 | SOX5 | −1.181650351 | 0.000533483 |
| ENSG00000100336 | APOL4 | −1.182157909 | 0.000220807 |
| ENSG00000084636 | COL16A1 | −1.183034006 | 1.08776E−05 |
| ENSG00000010327 | STAB1 | −1.183814881 | 4.6063E−06 |
| ENSG00000139187 | KLRG1 | −1.18393578 | 0.000242333 |
| ENSG00000164741 | DLC1 | −1.184152784 | 1.34948E−06 |
| ENSG00000231672 | DIRC3 | −1.185149757 | 0.000243148 |
| ENSG00000065325 | GLP2R | −1.185304374 | 0.00219023 |
| ENSG00000198223 | CSF2RA | −1.185323954 | 8.52127E−06 |
| ENSG00000140511 | HAPLN3 | −1.185925451 | 2.10135E−05 |
| ENSG00000198756 | COLGALT2 | −1.186158932 | 0.005416943 |
| ENSG00000005381 | MPO | −1.186385139 | 0.005928183 |
| ENSG00000154319 | FAM167A | −1.186397209 | 0.000388261 |
| ENSG00000178297 | TMPRSS9 | −1.186723965 | 0.01349752 |
| ENSG00000104447 | TRPS1 | −1.187451768 | 2.52627E−06 |
| ENSG00000166035 | LIPC | −1.187870772 | 0.029387324 |
| ENSG00000189350 | FAM179A | −1.18806183 | 3.32172E−05 |
| ENSG00000115380 | EFEMP1 | −1.188320205 | 6.88103E−05 |
| ENSG00000167613 | LAIR1 | −1.188563461 | 7.82772E−06 |
| ENSG00000139970 | RTN1 | −1.189584441 | 1.07408E−06 |
| ENSG00000182050 | MGAT4C | −1.189786392 | 0.017909786 |
| ENSG00000123338 | NCKAP1L | −1.18998499 | 4.27658E−06 |
| ENSG00000075340 | ADD2 | −1.190068477 | 0.009190393 |
| ENSG00000000938 | FGR | −1.190229233 | 1.94053E−05 |
| ENSG00000180871 | CXCR2 | −1.191139331 | 0.000491418 |
| ENSG00000174600 | CMKLR1 | −1.191782971 | 8.38988E−05 |
| ENSG00000099260 | PALMD | −1.191848962 | 0.000119091 |
| ENSG00000221866 | PLXNA4 | −1.192245818 | 6.84456E−05 |
| ENSG00000142748 | FCN3 | −1.192261941 | 0.042493263 |
| ENSG00000132530 | XAF1 | −1.19264312 | 0.00024758 |
| ENSG00000108821 | COL1A1 | −1.192686522 | 2.06351E−05 |
| ENSG00000158163 | DZIP1L | −1.192924992 | 9.62415E−07 |
| ENSG00000173917 | HOXB2 | −1.195630744 | 1.35023E−06 |
| ENSG00000198300 | PEG3 | −1.196089492 | 6.73493E−05 |
| ENSG00000137857 | DUOX1 | −1.196165246 | 0.006588702 |
| ENSG00000118596 | SLC16A7 | −1.196287822 | 3.89164E−05 |
| ENSG00000005513 | SOX8 | −1.196891539 | 0.023936211 |
| ENSG00000111962 | UST | −1.197117891 | 0.001217187 |
| ENSG00000109738 | GLRB | −1.197213302 | 0.002769453 |
| ENSG00000175287 | PHYHD1 | −1.199148936 | 0.000601286 |
| ENSG00000133574 | GIMAP4 | −1.199331097 | 0.000108277 |
| ENSG00000118523 | CTGF | −1.199512142 | 5.89074E−05 |
| ENSG00000101298 | SNPH | −1.199878151 | 2.06707E−06 |
| ENSG00000137558 | PI15 | −1.200980713 | 0.001198322 |
| ENSG00000162654 | GBP4 | −1.201334905 | 0.000644827 |
| ENSG00000159761 | C1orf86 | −1.201521832 | 0.00031607 |
| ENSG00000105383 | CD33 | −1.201802317 | 6.589E−05 |
| ENSG00000067798 | NAV3 | −1.201989516 | 0.000172006 |
| ENSG00000129675 | ARHGEF6 | −1.202118353 | 1.77842E−06 |
| ENSG00000173258 | ZNF483 | −1.202983572 | 0.005882316 |
| ENSG00000244694 | PTCHD4 | −1.204273641 | 0.012782289 |
| ENSG00000100321 | SYNGR1 | −1.204841818 | 0.000255014 |
| ENSG00000171115 | GIMAP8 | −1.204860222 | 3.88115E−05 |
| ENSG00000003989 | SLC7A2 | −1.204966223 | 0.006561721 |
| ENSG00000198108 | CHSY3 | −1.20499247 | 2.43913E−06 |
| ENSG00000131187 | F12 | −1.205066496 | 0.01116517 |
| ENSG00000172260 | NEGR1 | −1.205715615 | 0.004931555 |
| ENSG00000241644 | INMT | −1.20587723 | 0.000143646 |
| ENSG00000177103 | DSCAML1 | −1.206484225 | 0.007907869 |
| ENSG00000250120 | PCDHA10 | -1.206740837 | 0.045672655 |
| ENSG00000110076 | NRXN2 | −1.207544301 | 6.05614E−05 |
| ENSG00000184557 | SOCS3 | −1.208085693 | 9.71093E−05 |
| ENSG00000099860 | GADD45B | −1.208300182 | 0.000141517 |
| ENSG00000120875 | DUSP4 | −1.208891098 | 0.000179051 |
| ENSG00000188064 | WNT7B | −1.209142013 | 0.010610886 |
| ENSG00000134516 | DOCK2 | −1.209168062 | 1.49442E−05 |
| ENSG00000072952 | MRVI1 | −1.209447861 | 4.7121E−06 |
| ENSG00000181291 | TMEM132E | −1.209659251 | 0.00218064 |
| ENSG00000165124 | SVEP1 | −1.20983018 | 3.0356E−05 |
| ENSG00000155970 | MICU3 | −1.209865134 | 8.55803E−06 |
| ENSG00000146374 | RSPO3 | −1.209873799 | 0.001275565 |
| ENSG00000111728 | ST8SIA1 | −1.21101385 | 0.004710055 |
| ENSG00000120833 | SOCS2 | −1.211505742 | 1.64174E−05 |
| ENSG00000178175 | ZNF366 | −1.211564579 | 2.31768E−05 |
| ENSG00000185737 | NRG3 | −1.212342654 | 0.001327903 |
| ENSG00000152527 | PLEKHH2 | −1.213090507 | 5.57419E−05 |
| ENSG00000134874 | DZIP1 | −1.21356678 | 7.57031E−08 |
| ENSG00000144642 | RBMS3 | −1.213652734 | 3.86952E−06 |
| ENSG00000170956 | CEACAM3 | −1.21382941 | 0.004531583 |
| ENSG00000113749 | HRH2 | −1.213893715 | 0.00057991 |
| ENSG00000062524 | LTK | −1.213934508 | 0.007342167 |
| ENSG00000130755 | GMFG | −1.214020953 | 4.1255E−06 |
| ENSG00000109956 | B3GAT1 | −1.214052199 | 0.008399257 |
| ENSG00000122254 | HS3ST2 | −1.215727907 | 0.006137718 |
| ENSG00000136999 | NOV | −1.216119385 | 2.26194E−05 |
| ENSG00000008118 | CAMK1G | −1.216465351 | 0.000323103 |
| ENSG00000077782 | FGFR1 | −1.216659536 | 7.61758E−05 |
| ENSG00000105329 | TGFB1 | −1.216730912 | 7.548E−07 |
| ENSG00000125430 | HS3ST3B1 | −1.217189858 | 0.000204523 |
| ENSG00000184271 | POU6F1 | −1.217315751 | 3.16218E−07 |
| ENSG00000107611 | CUBN | −1.217602366 | 9.03133E−06 |
| ENSG00000103313 | MEFV | −1.217686693 | 0.000901215 |
| ENSG00000104368 | PLAT | −1.219462117 | 1.60205E−05 |
| ENSG00000187037 | GPR141 | −1.219708612 | 0.00034455 |
| ENSG00000224389 | C4B | −1.219744569 | 0.03778999 |
| ENSG00000204252 | HLA-DOA | −1.219840235 | 2.9222E−05 |
| ENSG00000168309 | FAM107A | −1.220022435 | 0.00054014 |
| ENSG00000100344 | PNPLA3 | −1.220399677 | 0.044051237 |
| ENSG00000130558 | OLFM1 | −1.220810425 | 0.004399836 |
| ENSG00000198046 | ZNF667 | −1.220830046 | 5.28912E−06 |
| ENSG00000108823 | SGCA | −1.221078587 | 0.000298154 |
| ENSG00000158517 | NCF1 | −1.221294717 | 0.000831881 |
| ENSG00000239998 | LILRA2 | −1.22159887 | 3.7788E−05 |
| ENSG00000123095 | BHLHE41 | −1.222564294 | 5.59088E−06 |
| ENSG00000152049 | KCNE4 | −1.222624045 | 4.08965E−06 |
| ENSG00000135272 | MDFIC | −1.223105728 | 2.00784E−06 |
| ENSG00000143369 | ECM1 | −1.223201413 | 1.56575E−05 |
| ENSG00000010610 | CD4 | −1.223221024 | 1.22209E−06 |
| ENSG00000141622 | RNF165 | −1.223643902 | 0.008455567 |
| ENSG00000102383 | ZDHHC15 | −1.224320897 | 0.006470904 |
| ENSG00000163565 | IFI16 | −1.224602115 | 4.92172E−06 |
| ENSG00000186818 | LILRB4 | −1.22555358 | 1.80011E−05 |
| ENSG00000157765 | SLC34A2 | −1.22590591 | 0.012532003 |
| ENSG00000118004 | COLEC11 | −1.226439558 | 0.001119969 |
| ENSG00000077264 | PAK3 | −1.226648361 | 0.004295558 |
| ENSG00000139926 | FRMD6 | −1.226682397 | 8.31715E−07 |
| ENSG00000198796 | ALPK2 | −1.227302741 | 0.000130756 |
| ENSG00000173210 | ABLIM3 | −1.227546546 | 0.001906516 |
| ENSG00000158246 | FAM46B | −1.227774062 | 0.001327891 |
| ENSG00000135835 | KIAA1614 | −1.227859531 | 1.09105E−05 |
| ENSG00000120658 | ENOX1 | −1.22871094 | 0.000120292 |
| ENSG00000145362 | ANK2 | −1.229270391 | 0.000539649 |
| ENSG00000157514 | TSC22D3 | −1.22944806 | 2.10135E−05 |
| ENSG00000129038 | LOXL1 | −1.229597535 | 9.30958E−08 |
| ENSG00000010295 | IFFO1 | −1.230998441 | 1.04551E−07 |
| ENSG00000196218 | RYR1 | −1.231926697 | 0.001466195 |
| ENSG00000169495 | HTRA4 | −1.232533396 | 0.000269144 |
| ENSG00000183098 | GPC6 | −1.23264938 | 3.44828E−06 |
| ENSG00000019102 | VSIG2 | −1.232939228 | 0.00487217 |
| ENSG00000172578 | KLHL6 | −1.23385044 | 5.71004E−05 |
| ENSG00000173421 | CCDC36 | −1.233872763 | 5.08468E−06 |
| ENSG00000185215 | TNFAIP2 | −1.233905637 | 1.92084E−05 |
| ENSG00000237541 | HLA-DQA2 | −1.234364566 | 0.017383524 |
| ENSG00000067840 | PDZD4 | −1.234984539 | 8.09621E−05 |
| ENSG00000175879 | HOXD8 | −1.235002643 | 0.047873132 |
| ENSG00000156299 | TIAM1 | −1.235032713 | 2.84269E−06 |
| ENSG00000175489 | LRRC25 | −1.23504695 | 1.50406E−05 |
| ENSG00000125551 | PLGLB2 | −1.235824933 | 0.020069572 |
| ENSG00000187116 | LILRA5 | −1.236606775 | 0.001666785 |
| ENSG00000162711 | NLRP3 | −1.237364079 | 2.18735E−06 |
| ENSG00000081237 | PTPRC | −1.237467457 | 2.97105E−05 |
| ENSG00000008516 | MMP25 | −1.237731453 | 1.0864E−05 |
| ENSG00000079215 | SLC1A3 | −1.23840385 | 0.000415863 |
| ENSG00000108387 | SEPT4 | −1.238949239 | 6.53044E−06 |
| ENSG00000179954 | SSC5D | −1.239358618 | 5.02948E−06 |
| ENSG00000161929 | SCIMP | −1.240027423 | 0.00011331 |
| ENSG00000115556 | PLCD4 | −1.240338243 | 0.000411355 |
| ENSG00000204287 | HLA-DRA | −1.240436516 | 0.000138259 |
| ENSG00000239697 | TNFSF12 | −1.24050702 | 5.97701E−08 |
| ENSG00000182492 | BGN | −1.240775055 | 1.62675E−05 |
| ENSG00000166106 | ADAMTS15 | −1.240999726 | 0.010601951 |
| ENSG00000100342 | APOL1 | −1.241062252 | 6.18162E−05 |
| ENSG00000111077 | TNS2 | −1.241479647 | 2.21115E−07 |
| ENSG00000104899 | AMH | −1.241538324 | 0.001198322 |
| ENSG00000173372 | C1QA | −1.241766344 | 0.000504732 |
| ENSG00000204381 | LAYN | −1.242542509 | 5.05882E−06 |
| ENSG00000163600 | ICOS | −1.242894599 | 0.001098585 |
| ENSG00000169282 | KCNAB1 | −1.243743285 | 0.001110994 |
| ENSG00000167693 | NXN | −1.243837222 | 6.1219E−06 |
| ENSG00000133063 | CHIT1 | −1.243932804 | 0.016066024 |
| ENSG00000069431 | ABCC9 | −1.244417217 | 2.20898E−05 |
| ENSG00000163145 | C1QTNF7 | −1.245214393 | 0.000835544 |
| ENSG00000136859 | ANGPTL2 | −1.245224033 | 4.92172E−06 |
| ENSG00000189129 | PLAC9 | −1.24534814 | 0.000221819 |
| ENSG00000114654 | EFCC1 | −1.245378439 | 5.40913E−05 |
| ENSG00000166405 | RIC3 | −1.245781251 | 0.004240274 |
| ENSG00000117154 | IGSF21 | −1.245898827 | 0.003965059 |
| ENSG00000184828 | ZBTB7C | −1.246309911 | 0.000607743 |
| ENSG00000115461 | IGFBP5 | −1.246924626 | 0.000204529 |
| ENSG00000019582 | CD74 | −1.247020794 | 3.22201E−05 |
| ENSG00000162520 | SYNC | −1.247398028 | 8.37346E−06 |
| ENSG00000140090 | SLC24A4 | −1.247409502 | 1.63415E−05 |
| ENSG00000120949 | TNFRSF8 | −1.247938845 | 0.000415863 |
| ENSG00000196126 | HLA-DRB1 | −1.248165776 | 5.49421E−05 |
| ENSG00000112852 | PCDHB2 | −1.24855528 | 0.002868391 |
| ENSG00000123096 | SSPN | −1.248840998 | 3.34177E−07 |
| ENSG00000088882 | CPXM1 | −1.249480083 | 1.16543E−05 |
| ENSG00000173369 | C1QB | −1.250210526 | 0.000370687 |
| ENSG00000157927 | RADIL | −1.250353421 | 4.99087E−05 |
| ENSG00000134138 | MEIS2 | −1.250579671 | 4.12937E−06 |
| ENSG00000176971 | FIBIN | −1.250818033 | 7.09634E−06 |
| ENSG00000143867 | OSR1 | −1.250847091 | 0.002687218 |
| ENSG00000196990 | FAM163B | −1.251514865 | 0.005702874 |
| ENSG00000012779 | ALOX5 | −1.251558885 | 9.25031E−06 |
| ENSG00000277632 | CCL3 | −1.251852795 | 0.001088763 |
| ENSG00000259030 | FPGT-TNNI3K | −1.252099756 | 7.79554E−05 |
| ENSG00000121904 | CSMD2 | −1.252642618 | 5.72693E−05 |
| ENSG00000168995 | SIGLEC7 | −1.253049397 | 0.000211989 |
| ENSG00000042781 | USH2A | −1.254282147 | 0.012941087 |
| ENSG00000012124 | CD22 | −1.25449932 | 6.46719E−05 |
| ENSG00000106952 | TNFSF8 | −1.255518784 | 0.000342742 |
| ENSG00000198832 | SELM | −1.256701248 | 1.32513E−06 |
| ENSG00000103196 | CRISPLD2 | −1.256801651 | 3.81028E−05 |
| ENSG00000182511 | FES | −1.257683426 | 4.16362E−06 |
| ENSG00000161681 | SHANK1 | −1.258360578 | 6.70875E−06 |
| ENSG00000169896 | ITGAM | −1.258921389 | 4.44127E−06 |
| ENSG00000101336 | HCK | −1.258973841 | 2.82361E−06 |
| ENSG00000131080 | EDA2R | −1.259179161 | 0.000177705 |
| ENSG00000138685 | FGF2 | −1.259243723 | 0.000405516 |
| ENSG00000130592 | LSP1 | −1.259748483 | 1.66007E−06 |
| ENSG00000186642 | PDE2A | −1.260719144 | 0.000143239 |
| ENSG00000158714 | SLAMF8 | −1.260731734 | 2.10135E−05 |
| ENSG00000171408 | PDE7B | −1.26081495 | 0.000733498 |
| ENSG00000139194 | RBP5 | −1.262108874 | 0.01202083 |
| ENSG00000175040 | CHST2 | −1.26369885 | 2.11475E−08 |
| ENSG00000000971 | CFH | −1.26436294 | 0.0018355 |
| ENSG00000128284 | APOL3 | −1.265295833 | 5.82398E−06 |
| ENSG00000101134 | DOK5 | −1.266010005 | 1.35482E−06 |
| ENSG00000163453 | IGFBP7 | −1.267085411 | 4.16105E−06 |
| ENSG00000073282 | TP63 | −1.267574404 | 0.000793904 |
| ENSG00000143382 | ADAMTSL4 | −1.268399388 | 6.51103E−06 |
| ENSG00000061455 | PRDM6 | −1.268515966 | 1.40843E−06 |
| ENSG00000170153 | RNF150 | −1.26878095 | 8.00283E−05 |
| ENSG00000156127 | BATF | −1.268835188 | 4.98746E−05 |
| ENSG00000131849 | ZNF132 | −1.268912419 | 1.44968E−05 |
| ENSG00000050165 | DKK3 | −1.269658216 | 3.16481E−05 |
| ENSG00000128573 | FOXP2 | −1.270199233 | 0.002865004 |
| ENSG00000153822 | KCNJ16 | −1.270927062 | 0.009894448 |
| ENSG00000171310 | CHST11 | −1.271667281 | 7.67678E−07 |
| ENSG00000129946 | SHC2 | −1.27191939 | 1.20582E−05 |
| ENSG00000110077 | MS4A6A | −1.272018639 | 8.02783E−05 |
| ENSG00000110887 | DAO | −1.272940567 | 0.038705581 |
| ENSG00000135604 | STX11 | −1.27362525 | 2.34861E−05 |
| ENSG00000143127 | ITGA10 | −1.27434303 | 1.63616E−05 |
| ENSG00000131378 | RFTN1 | −1.275036452 | 1.03636E−07 |
| ENSG00000005471 | ABCB4 | −1.27504517 | 0.021354689 |
| ENSG00000088827 | SIGLEC1 | −1.275248866 | 0.001161319 |
| ENSG00000133048 | CHI3L1 | −1.275930342 | 0.015476928 |
| ENSG00000101082 | SLA2 | −1.276914495 | 5.66399E−05 |
| ENSG00000133561 | GIMAP6 | −1.277080684 | 1.27047E−05 |
| ENSG00000167601 | AXL | −1.277150996 | 6.21921E−07 |
| ENSG00000151726 | ACSL1 | −1.277649272 | 0.000179175 |
| ENSG00000048052 | HDAC9 | −1.27780238 | 7.38287E−06 |
| ENSG00000172915 | NBEA | −1.279100707 | 8.58737E−05 |
| ENSG00000106571 | GLI3 | −1.279119677 | 6.37669E−06 |
| ENSG00000142347 | MYO1F | −1.280247206 | 2.33821E−07 |
| ENSG00000154263 | ABCA10 | −1.280275968 | 7.79115E−06 |
| ENSG00000105639 | JAK3 | −1.280516459 | 2.37869E−06 |
| ENSG00000060718 | COL11A1 | −1.280755681 | 0.000473859 |
| ENSG00000184347 | SLIT3 | −1.280859693 | 1.87622E−05 |
| ENSG00000163154 | TNFAIP8L2 | −1.281119032 | 6.24608E−06 |
| ENSG00000231389 | HLA-DPA1 | −1.281151326 | 3.98236E−05 |
| ENSG00000180353 | HCLS1 | −1.281208272 | 9.14566E−07 |
| ENSG00000169403 | PTAFR | −1.281319195 | 1.21654E−06 |
| ENSG00000145075 | CCDC39 | −1.281457227 | 0.004113882 |
| ENSG00000007372 | PAX6 | −1.281666878 | 0.011434259 |
| ENSG00000123685 | BATF3 | −1.281698919 | 8.73266E−07 |
| ENSG00000103449 | SALL1 | −1.281808222 | 0.048202618 |
| ENSG00000122223 | CD244 | −1.281817741 | 0.001575787 |
| ENSG00000151067 | CACNA1C | −1.283399954 | 1.82887E−06 |
| ENSG00000160326 | SLC2A6 | −1.283525121 | 4.61249E−06 |
| ENSG00000169442 | CD52 | −1.283852449 | 3.13843E−05 |
| ENSG00000143869 | GDF7 | −1.283939364 | 0.002159459 |
| ENSG00000188906 | LRRK2 | −1.284364899 | 4.34378E−05 |
| ENSG00000073712 | FERMT2 | −1.285060092 | 1.26489E−06 |
| ENSG00000164691 | TAGAP | −1.285333766 | 2.05026E−05 |
| ENSG00000023902 | PLEKHO1 | −1.285698986 | 8.75035E−08 |
| ENSG00000173198 | CYSLTR1 | −1.286982597 | 0.000173899 |
| ENSG00000107249 | GLIS3 | −1.287570918 | 1.97989E−06 |
| ENSG00000155926 | SLA | −1.287799377 | 1.57852E−05 |
| ENSG00000173068 | BNC2 | −1.288356134 | 7.97644E−06 |
| ENSG00000182636 | NDN | −1.289091341 | 2.2782E−05 |
| ENSG00000214063 | TSPAN4 | −1.289501439 | 1.55933E−06 |
| ENSG00000145147 | SLIT2 | −1.290042287 | 1.65513E−06 |
| ENSG00000118308 | LRMP | −1.290771912 | 0.000145328 |
| ENSG00000146674 | IGFBP3 | −1.291303554 | 1.84669E−05 |
| ENSG00000274618 | HIST1H4F | −1.292844217 | 0.000531384 |
| ENSG00000165457 | FOLR2 | −1.294061611 | 0.008304119 |
| ENSG00000121297 | TSHZ3 | −1.294305445 | 6.01962E−08 |
| ENSG00000244486 | SCARF2 | −1.294334674 | 1.4429E−07 |
| ENSG00000099958 | DERL3 | −1.294783854 | 0.000580699 |
| ENSG00000169992 | NLGN2 | −1.295120775 | 6.53704E−07 |
| ENSG00000138378 | STAT4 | −1.29543975 | 1.19766E−05 |
| ENSG00000171502 | COL24A1 | −1.295886151 | 9.38941E−05 |
| ENSG00000053524 | MCF2L2 | −1.296416705 | 4.61249E−06 |
| ENSG00000165246 | NLGN4Y | −1.296638324 | 0.031538531 |
| ENSG00000188517 | COL25A1 | −1.297150023 | 0.004307607 |
| ENSG00000168542 | COL3A1 | −1.297227553 | 7.74067E−06 |
| ENSG00000130222 | GADD45G | −1.297476279 | 0.000261918 |
| ENSG00000160255 | ITGB2 | −1.297511297 | 3.95752E−06 |
| ENSG00000154654 | NCAM2 | −1.297833226 | 0.000573734 |
| ENSG00000065320 | NTN1 | −1.299325227 | 0.011148482 |
| ENSG00000177575 | CD163 | −1.299893086 | 0.000390015 |
| ENSG00000117245 | KIF17 | −1.300534329 | 0.000200591 |
| ENSG00000276231 | PIK3R6 | −1.300549794 | 1.91479E−07 |
| ENSG00000160801 | PTH1R | −1.302062097 | 0.003466999 |
| ENSG00000086730 | LAT2 | −1.302433939 | 2.31493E−07 |
| ENSG00000197471 | SPN | −1.302532526 | 1.74498E−06 |
| ENSG00000140009 | ESR2 | −1.30293787 | 0.005107637 |
| ENSG00000242732 | RGAG4 | −1.303090886 | 5.61793E−06 |
| ENSG00000126264 | HCST | −1.30343371 | 0.00015882 |
| ENSG00000198400 | NTRK1 | −1.303453983 | 0.002234139 |
| ENSG00000074706 | IPCEF1 | −1.303676007 | 0.000105006 |
| ENSG00000164483 | SAMD3 | −1.304642308 | 3.69683E−05 |
| ENSG00000154330 | PGM5 | −1.304850142 | 0.01764485 |
| ENSG00000179542 | SLITRK4 | −1.305385651 | 0.00073584 |
| ENSG00000138722 | MMRN1 | −1.305607805 | 0.019467695 |
| ENSG00000172575 | RASGRP1 | −1.305665733 | 6.38526E−06 |
| ENSG00000120820 | GLT8D2 | −1.306404154 | 3.59864E−06 |
| ENSG00000025708 | TYMP | −1.306791046 | 6.88891E−06 |
| ENSG00000135914 | HTR2B | −1.306928718 | 0.000742508 |
| ENSG00000018869 | ZNF582 | −1.307031585 | 7.55275E−05 |
| ENSG00000182578 | CSF1R | −1.308063603 | 7.89301E−07 |
| ENSG00000128652 | HOXD3 | −1.308428032 | 0.042593636 |
| ENSG00000041515 | MYO16 | −1.309271715 | 0.004238641 |
| ENSG00000115468 | EFHD1 | −1.3097124 | 3.0664E−06 |
| ENSG00000177706 | FAM20C | −1.310293141 | 3.86458E−07 |
| ENSG00000142494 | SLC47A1 | −1.310742871 | 0.000442139 |
| ENSG00000204936 | CD177 | −1.311062655 | 0.00137739 |
| ENSG00000066336 | SPI1 | −1.311466097 | 5.07593E−07 |
| ENSG00000144648 | ACKR2 | −1.311541006 | 7.51745E−05 |
| ENSG00000125910 | S1PR4 | −1.311857708 | 0.000280538 |
| ENSG00000167711 | SERPINF2 | −1.312573286 | 0.027314297 |
| ENSG00000110876 | SELPLG | −1.312992457 | 1.09675E−06 |
| ENSG00000181631 | P2RY13 | −1.313224407 | 8.50524E−05 |
| ENSG00000160408 | ST6GALNAC6 | −1.313444839 | 1.89078E−09 |
| ENSG00000172935 | MRGPRF | −1.314382734 | 1.97624E−06 |
| ENSG00000171246 | NPTX1 | −1.31515225 | 0.00010151 |
| ENSG00000090534 | THPO | −1.315369162 | 0.003521046 |
| ENSG00000091106 | NLRC4 | −1.315628669 | 3.0764E−05 |
| ENSG00000166448 | TMEM130 | −1.316288618 | 0.001135103 |
| ENSG00000141505 | ASGR1 | −1.316625086 | 0.044561092 |
| ENSG00000149294 | NCAM1 | −1.317114455 | 0.000253712 |
| ENSG00000125508 | SRMS | −1.317996432 | 0.024352628 |
| ENSG00000173200 | PARP15 | −1.31871982 | 5.34552E−05 |
| ENSG00000112303 | VNN2 | −1.318751662 | 0.000188548 |
| ENSG00000111860 | CEP85L | −1.319376477 | 2.62136E−06 |
| ENSG00000104903 | LYL1 | −1.319477213 | 2.23061E−07 |
| ENSG00000132837 | DMGDH | −1.320758807 | 0.020816064 |
| ENSG00000171649 | ZIK1 | −1.320964286 | 3.70539E−06 |
| ENSG00000206190 | ATP10A | −1.321006994 | 4.38137E−07 |
| ENSG00000257242 | C120rf79 | −1.321305486 | 0.000907887 |
| ENSG00000046889 | PREX2 | −1.322313956 | 3.43678E−07 |
| ENSG00000198502 | HLA-DRB5 | −1.322986017 | 0.000541791 |
| ENSG00000152217 | SETBP1 | −1.323499748 | 3.31931E−06 |
| ENSG00000170390 | DCLK2 | −1.324259917 | 3.23652E−07 |
| ENSG00000179144 | GIMAP7 | −1.324311921 | 5.65388E−05 |
| ENSG00000136574 | GATA4 | −1.325975478 | 0.002098347 |
| ENSG00000133687 | TMTC1 | −1.32598008 | 1.33594E−05 |
| ENSG00000175899 | A2M | −1.32658222 | 8.69517E−05 |
| ENSG00000169515 | CCDC8 | −1.327236999 | 1.33243E−05 |
| ENSG00000152137 | HSPB8 | −1.327754381 | 1.69986E−06 |
| ENSG00000122679 | RAMP3 | −1.328515607 | 2.83652E−05 |
| ENSG00000107485 | GATA3 | −1.328554159 | 0.000155867 |
| ENSG00000260314 | MRC1 | −1.328988195 | 0.000176206 |
| ENSG00000183801 | OLFML1 | −1.329281354 | 7.5462E−06 |
| ENSG00000206432 | TMEM200C | −1.329441229 | 1.62807E−05 |
| ENSG00000111863 | ADTRP | −1.329916534 | 0.000158492 |
| ENSG00000269948 | RP11-248J23.6 | −1.330372451 | 1.78916E−06 |
| ENSG00000079102 | RUNX1T1 | −1.330528167 | 1.26528E−06 |
| ENSG00000135502 | SLC26A10 | −1.331173412 | 3.67599E−05 |
| ENSG00000181444 | ZNF467 | −1.33136971 | 7.70788E−06 |
| ENSG00000089041 | P2RX7 | −1.331483229 | 3.42517E−07 |
| ENSG00000167083 | GNGT2 | −1.331616205 | 5.65388E−05 |
| ENSG00000111186 | WNT5B | −1.332259291 | 0.00800442 |
| ENSG00000135439 | AGAP2 | −1.333217564 | 8.29471E−06 |
| ENSG00000091844 | RGS17 | −1.333570377 | 0.00046005 |
| ENSG00000083814 | ZNF671 | −1.333785964 | 1.76507E−05 |
| ENSG00000185046 | ANKS1B | −1.334046718 | 0.003477125 |
| ENSG00000170955 | PRKCDBP | −1.334151626 | 7.548E−07 |
| ENSG00000239713 | APOBEC3G | −1.334850049 | 4.0212E−05 |
| ENSG00000144152 | FBLN7 | −1.335334356 | 4.4053E−07 |
| ENSG00000125144 | MT1G | −1.335609178 | 0.016611049 |
| ENSG00000119535 | CSF3R | −1.336845559 | 6.53044E−06 |
| ENSG00000142512 | SIGLEC10 | −1.337447232 | 1.20046E−05 |
| ENSG00000112619 | PRPH2 | −1.33866708 | 2.87088E−06 |
| ENSG00000088881 | EBF4 | −1.339348505 | 5.38866E−07 |
| ENSG00000163884 | KLF15 | −1.339432665 | 0.014437879 |
| ENSG00000144619 | CNTN4 | −1.339647048 | 2.60728E−06 |
| ENSG00000109625 | CPZ | −1.343177158 | 0.002327224 |
| ENSG00000168994 | PXDC1 | −1.344912997 | 4.38273E−06 |
| ENSG00000136630 | HLX | −1.345021844 | 2.11475E−08 |
| ENSG00000164935 | DCSTAMP | −1.345718397 | 0.00099329 |
| ENSG00000139329 | LUM | −1.346025378 | 2.08379E−05 |
| ENSG00000178343 | SHISA3 | −1.346216604 | 3.25415E−05 |
| ENSG00000154175 | ABI3BP | −1.347094852 | 2.44453E−05 |
| ENSG00000111341 | MGP | −1.347287081 | 0.000167446 |
| ENSG00000073146 | MOV10L1 | −1.347343511 | 2.87088E−06 |
| ENSG00000163751 | CPA3 | −1.347495163 | 0.000471133 |
| ENSG00000092607 | TBX15 | −1.347502849 | 0.009330017 |
| ENSG00000156218 | ADAMTSL3 | −1.347800829 | 0.00065811 |
| ENSG00000166501 | PRKCB | −1.347966306 | 4.88776E−06 |
| ENSG00000106809 | OGN | −1.348866721 | 0.04030366 |
| ENSG00000049089 | COL9A2 | −1.349039206 | 0.000477145 |
| ENSG00000166831 | RBPMS2 | −1.349104821 | 5.79938E−06 |
| ENSG00000104972 | LILRB1 | −1.349256104 | 3.17927E−06 |
| ENSG00000071575 | TRIB2 | −1.349463531 | 8.48499E−08 |
| ENSG00000204851 | PNMAL2 | −1.349510321 | 2.16739E−05 |
| ENSG00000186517 | ARHGAP30 | −1.34999955 | 3.42653E−08 |
| ENSG00000131355 | EMR3 | −1.351019854 | 0.000162246 |
| ENSG00000151322 | NPAS3 | −1.351096064 | 0.002700003 |
| ENSG00000204577 | LILRB3 | −1.351489569 | 1.06562E−05 |
| ENSG00000187688 | TRPV2 | −1.351689403 | 1.03618E−07 |
| ENSG00000120217 | CD274 | −1.352018951 | 0.000186348 |
| ENSG00000130303 | BST2 | −1.352200439 | 3.55771E−05 |
| ENSG00000187955 | COL14A1 | −1.352408958 | 1.70562E−05 |
| ENSG00000198795 | ZNF521 | −1.352652582 | 2.66038E−06 |
| ENSG00000136305 | CIDEB | −1.352905676 | 1.41945E−05 |
| ENSG00000185518 | SV2B | −1.352927982 | 0.001480163 |
| ENSG00000034053 | APBA2 | −1.353722727 | 5.87163E−05 |
| ENSG00000158008 | EXTL1 | −1.35435216 | 0.000116194 |
| ENSG00000198753 | PLXNB3 | −1.356290561 | 0.00942131 |
| ENSG00000075884 | ARHGAP15 | −1.357759474 | 4.15343E−07 |
| ENSG00000232653 | GOLGA8N | −1.358202793 | 6.87665E−06 |
| ENSG00000105825 | TFPI2 | −1.359265276 | 0.007582781 |
| ENSG00000143028 | SYPL2 | −1.360179735 | 4.64967E−05 |
| ENSG00000137203 | TFAP2A | −1.360200468 | 0.007854124 |
| ENSG00000179921 | GPBAR1 | −1.360454991 | 7.36894E−06 |
| ENSG00000151789 | ZNF385D | −1.361677353 | 0.000281211 |
| ENSG00000139174 | PRICKLE1 | −1.362285328 | 9.96917E−07 |
| ENSG00000137727 | ARHGAP20 | −1.363402347 | 0.000268589 |
| ENSG00000111087 | GLI1 | −1.363950949 | 6.93396E−07 |
| ENSG00000204482 | LST1 | −1.36411559 | 7.66626E−06 |
| ENSG00000185736 | ADARB2 | −1.364256843 | 0.008092941 |
| ENSG00000203710 | CR1 | −1.364350738 | 9.04798E−05 |
| ENSG00000107984 | DKK1 | −1.364586457 | 0.011140226 |
| ENSG00000186310 | NAP1L3 | −1.365077229 | 3.65785E−05 |
| ENSG00000196090 | PTPRT | −1.365704215 | 0.002284951 |
| ENSG00000131042 | LILRB2 | −1.367205569 | 1.96556E−05 |
| ENSG00000007174 | DNAH9 | −1.367394384 | 0.002900476 |
| ENSG00000079263 | SP140 | −1.36785107 | 1.3134E−05 |
| ENSG00000119681 | LTBP2 | −1.368127362 | 6.10597E−06 |
| ENSG00000141052 | MYOCD | −1.36854398 | 3.04178E−05 |
| ENSG00000151948 | GLT1D1 | −1.369184391 | 0.00319049 |
| ENSG00000147576 | ADHFE1 | −1.369743451 | 0.000415856 |
| ENSG00000147100 | SLC16A2 | −1.369747445 | 2.96438E−05 |
| ENSG00000124212 | PTGIS | −1.369922229 | 2.13007E−05 |
| ENSG00000130203 | APOE | −1.369948171 | 0.000177552 |
| ENSG00000111058 | ACSS3 | −1.370093223 | 0.000199953 |
| ENSG00000167483 | FAM129C | −1.370153299 | 0.003804528 |
| ENSG00000130584 | ZBTB46 | −1.37104526 | 9.733E−08 |
| ENSG00000109339 | MAPK10 | −1.37213797 | 3.86458E−07 |
| ENSG00000184838 | PRR16 | −1.372651407 | 8.57424E−07 |
| ENSG00000183813 | CCR4 | −1.373417547 | 2.99849E−05 |
| ENSG00000174123 | TLR10 | −1.373495023 | 0.000719912 |
| ENSG00000148346 | LCN2 | −1.377542448 | 0.011442001 |
| ENSG00000111181 | SLC6A12 | −1.378635316 | 0.000474755 |
| ENSG00000112320 | SOBP | −1.378774929 | 2.34766E−05 |
| ENSG00000184371 | CSF1 | −1.379520207 | 7.07184E−08 |
| ENSG00000170500 | LONRF2 | −1.379602914 | 0.020055703 |
| ENSG00000100842 | EFS | −1.37976028 | 4.72689E−07 |
| ENSG00000120457 | KCNJ5 | −1.380906549 | 3.40174E−05 |
| ENSG00000163629 | PTPN13 | −1.381887352 | 5.61759E−05 |
| ENSG00000159261 | CLDN14 | −1.382221288 | 0.002563638 |
| ENSG00000125730 | C3 | −1.382364557 | 0.019656541 |
| ENSG00000136732 | GYPC | −1.382700631 | 6.76163E−07 |
| ENSG00000069535 | MAOB | −1.382763003 | 0.000708729 |
| ENSG00000125869 | LAMP5 | −1.382937902 | 3.0356E−05 |
| ENSG00000181847 | TIGIT | −1.383017285 | 0.000127192 |
| ENSG00000055955 | ITIH4 | −1.384463334 | 0.011239072 |
| ENSG00000158786 | PLA2G2F | −1.38452622 | 0.002641231 |
| ENSG00000171303 | KCNK3 | −1.385123011 | 0.001069729 |
| ENSG00000163431 | LMOD1 | −1.386020175 | 4.26452E−07 |
| ENSG00000173208 | ABCD2 | −1.386459547 | 0.001643421 |
| ENSG00000126882 | FAM78A | −1.387007814 | 1.32024E−07 |
| ENSG00000275302 | CCL4 | −1.387251557 | 4.6769E−05 |
| ENSG00000184922 | FMNL1 | −1.387597199 | 1.07962E−07 |
| ENSG00000163599 | CTLA4 | −1.387607292 | 0.00268844 |
| ENSG00000082175 | PGR | −1.389867437 | 3.57778E−07 |
| ENSG00000223865 | HLA-DPB1 | −1.391952069 | 7.01003E−06 |
| ENSG00000019991 | HGF | −1.39215834 | 4.72444E−05 |
| ENSG00000006638 | TBXA2R | −1.392218154 | 1.24432E−06 |
| ENSG00000149090 | PAMR1 | −1.392251281 | 8.90555E−06 |
| ENSG00000105419 | MEIS3 | −1.394935236 | 1.50003E−08 |
| ENSG00000170458 | CD14 | −1.395154873 | 3.42103E−05 |
| ENSG00000105426 | PTPRS | −1.396136464 | 7.90124E−07 |
| ENSG00000099769 | IGFALS | −1.39841302 | 0.014093131 |
| ENSG00000196724 | ZNF418 | −1.398478329 | 2.47697E−06 |
| ENSG00000164344 | KLKB1 | −1.399527697 | 0.008139283 |
| ENSG00000205403 | CFI | −1.400491941 | 0.000225745 |
| ENSG00000198670 | LPA | −1.400790691 | 0.037679104 |
| ENSG00000015285 | WAS | −1.40095576 | 1.28669E−06 |
| ENSG00000171451 | DSEL | −1.401184119 | 7.25095E−07 |
| ENSG00000211448 | DIO2 | −1.402743681 | 0.00099329 |
| ENSG00000157168 | NRG1 | −1.403108401 | 0.017067172 |
| ENSG00000131203 | IDO1 | −1.403413305 | 0.023073967 |
| ENSG00000117560 | FASLG | −1.40413241 | 0.001541034 |
| ENSG00000180509 | KCNE1 | −1.404776361 | 0.000350888 |
| ENSG00000196557 | CACNA1H | -1.406476726 | 4.86586E−07 |
| ENSG00000139344 | AMDHD1 | −1.406623034 | 0.031688173 |
| ENSG00000143297 | FCRL5 | −1.40681718 | 0.007995403 |
| ENSG00000182162 | P2RY8 | −1.408331696 | 2.37136E−06 |
| ENSG00000204103 | MAFB | −1.409430633 | 7.71039E−08 |
| ENSG00000171476 | HOPX | −1.410188178 | 2.62136E−06 |
| ENSG00000106991 | ENG | −1.410800156 | 4.1422E−08 |
| ENSG00000180155 | LYNX1 | −1.410808547 | 0.000131545 |
| ENSG00000244482 | LILRA6 | −1.410838552 | 4.93995E−05 |
| ENSG00000113209 | PCDHB5 | −1.410935916 | 0.000570709 |
| ENSG00000188641 | DPYD | −1.41099067 | 1.26489E−06 |
| ENSG00000162576 | MXRA8 | −1.411211889 | 2.92985E−06 |
| ENSG00000187824 | TMEM220 | −1.412792849 | 0.000151776 |
| ENSG00000159212 | CLIC6 | −1.413294685 | 0.002501545 |
| ENSG00000183484 | GPR132 | -1.413513868 | 4.12937E−06 |
| ENSG00000091986 | CCDC80 | −1.413746341 | 1.5288E−05 |
| ENSG00000125735 | TNFSFI4 | -1.414431004 | 0.001728779 |
| ENSG00000163520 | FBLN2 | −1.415408525 | 1.56411E−07 |
| ENSG00000174807 | CD248 | −1.415713111 | 5.97701E−08 |
| ENSG00000100055 | CYTH4 | −1.416880465 | 1.20416E−07 |
| ENSG00000188536 | HBA2 | −1.417836366 | 0.000364417 |
| ENSG00000104894 | CD37 | −1.418932735 | 2.94372E−06 |
| ENSG00000142156 | COL6A1 | −1.419394887 | 3.57778E−07 |
| ENSG00000107736 | CDH23 | −1.419845685 | 2.48367E−06 |
| ENSG00000177807 | KCNJ10 | −1.420156049 | 0.003561478 |
| ENSG00000168546 | GFRA2 | −1.420256336 | 0.007027914 |
| ENSG00000007237 | GAS7 | −1.420426969 | 7.11386E−08 |
| ENSG00000176928 | GCNT4 | −1.421597699 | 1.20461E−06 |
| ENSG00000115594 | IL1R1 | −1.421732637 | 6.55067E−07 |
| ENSG00000153253 | SCN3A | −1.422622063 | 0.004076984 |
| ENSG00000177875 | CCDC184 | -1.422806829 | 6.80303E−06 |
| ENSG00000102452 | NALCN | −1.423964742 | 9.44593E−06 |
| ENSG00000165507 | C10orf10 | −1.424175818 | 2.67921E−06 |
| ENSG00000112139 | MDGA1 | −1.424370102 | 1.1552E−05 |
| ENSG00000122367 | LDB3 | −1.425971299 | 0.000635937 |
| ENSG00000133246 | PRAM1 | −1.427538999 | 1.13171E−07 |
| ENSG00000106624 | AEBP1 | −1.427665793 | 2.90319E−05 |
| ENSG00000196735 | HLA-DQA1 | −1.427729423 | 0.000109363 |
| ENSG00000180096 | SEPT1 | −1.428104072 | 3.14232E−06 |
| ENSG00000188305 | C19orf35 | −1.429967092 | 9.02335E−05 |
| ENSG00000179593 | ALOX15B | -1.43061843 | 0.001063826 |
| ENSG00000113594 | LIFR | −1.430655583 | 0.002521619 |
| ENSG00000127124 | HIVEP3 | −1.432113664 | 5.9378E−07 |
| ENSG00000198771 | RCSD1 | −1.433589871 | 3.75075E−08 |
| ENSG00000162676 | GFI1 | −1.434153621 | 1.93745E−05 |
| ENSG00000142303 | ADAMTS10 | -1.434246952 | 3.42653E−08 |
| ENSG00000143816 | WNT9A | −1.436038799 | 8.14408E−06 |
| ENSG00000204161 | C10orf128 | −1.436084505 | 2.10218E−06 |
| ENSG00000091592 | NLRP1 | −1.436094337 | 8.17503E−07 |
| ENSG00000188015 | S100A3 | −1.436569384 | 0.000896234 |
| ENSG00000136490 | LIMD2 | −1.436957681 | 4.79665E−07 |
| ENSG00000093134 | VNN3 | −1.439618817 | 0.003014081 |
| ENSG00000128815 | WDFY4 | −1.440484646 | 3.34177E−07 |
| ENSG00000115226 | FNDC4 | −1.441984776 | 7.15723E−05 |
| ENSG00000104415 | WISP1 | −1.442348917 | 1.42448E−06 |
| ENSG00000153404 | PLEKHG4B | −1.442485316 | 0.003493783 |
| ENSG00000130208 | APOC1 | −1.443020777 | 0.003780691 |
| ENSG00000182195 | LDOC1 | −1.445187497 | 1.69986E−06 |
| ENSG00000178562 | CD28 | −1.446037961 | 7.08224E−05 |
| ENSG00000162551 | ALPL | −1.446724779 | 0.002006821 |
| ENSG00000177989 | ODF3B | −1.44701482 | 3.16493E−07 |
| ENSG00000138080 | EMILIN1 | −1.449660676 | 3.86458E−07 |
| ENSG00000169946 | ZFPM2 | −1.45014781 | 2.26549E−07 |
| ENSG00000227507 | LTB | −1.452433452 | 0.000139002 |
| ENSG00000166676 | TVP23A | −1.452869264 | 1.12963E−05 |
| ENSG00000011028 | MRC2 | −1.452971353 | 5.97701E−08 |
| ENSG00000100385 | IL2RB | −1.453413248 | 2.49212E−06 |
| ENSG00000007933 | FMO3 | −1.453747592 | 0.000546202 |
| ENSG00000110665 | C11orf21 | −1.453900259 | 0.001654857 |
| ENSG00000078295 | ADCY2 | −1.454015822 | 0.00117231 |
| ENSG00000129450 | SIGLEC9 | −1.454866574 | 1.19976E−07 |
| ENSG00000186891 | TNFRSF18 | −1.455068405 | 8.02058E−05 |
| ENSG00000185070 | FLRT2 | −1.455496456 | 3.37022E−07 |
| ENSG00000144596 | GRIP2 | −1.455568908 | 5.08238E−05 |
| ENSG00000263961 | C1orf186 | −1.456739139 | 7.53646E−05 |
| ENSG00000138315 | OIT3 | −1.456783399 | 0.01233807 |
| ENSG00000107562 | CXCL12 | −1.45719748 | 0.000157736 |
| ENSG00000184408 | KCND2 | −1.458804262 | 1.05224E−05 |
| ENSG00000009694 | TENM1 | −1.459308234 | 0.010433276 |
| ENSG00000163606 | CD200R1 | −1.459667858 | 0.000147918 |
| ENSG00000250510 | GPR162 | −1.46050917 | 4.28987E−06 |
| ENSG00000123843 | C4BPB | −1.461529734 | 0.000179871 |
| ENSG00000168874 | ATOH8 | −1.461785258 | 0.01863606 |
| ENSG00000182771 | GRID1 | −1.462063879 | 0.000131466 |
| ENSG00000143851 | PTPN7 | −1.462663503 | 7.79115E−06 |
| ENSG00000070190 | DAPP1 | −1.463152553 | 2.05078E−05 |
| ENSG00000105851 | PIK3CG | −1.463748694 | 3.82695E−08 |
| ENSG00000160013 | PTGIR | −1.463858823 | 7.38276E−07 |
| ENSG00000142173 | COL6A2 | −1.464505224 | 6.80791E−07 |
| ENSG00000105609 | LILRB5 | −1.464802202 | 0.004137589 |
| ENSG00000176293 | ZNF135 | −1.464846766 | 3.89986E−06 |
| ENSG00000109794 | FAM149A | −1.465397328 | 2.06106E−05 |
| ENSG00000137672 | TRPC6 | −1.465736331 | 1.57852E−05 |
| ENSG00000158270 | COLEC12 | −1.46579545 | 0.000307779 |
| ENSG00000143515 | ATP8B2 | −1.465958032 | 2.11475E−08 |
| ENSG00000188153 | COL4A5 | −1.466509884 | 1.37202E−05 |
| ENSG00000141506 | PIK3R5 | −1.46752066 | 3.57627E−07 |
| ENSG00000122122 | SASH3 | −1.468149185 | 1.14871E−07 |
| ENSG00000141469 | SLC14A1 | −1.469695897 | 0.013631147 |
| ENSG00000234465 | PINLYP | −1.470357295 | 9.14566E−07 |
| ENSG00000116194 | ANGPTL1 | −1.471124132 | 0.000718491 |
| ENSG00000111913 | FAM65B | −1.471340117 | 2.21816E−06 |
| ENSG00000122877 | EGR2 | −1.473373856 | 8.6066E−07 |
| ENSG00000096996 | IL12RB1 | −1.476435937 | 1.56632E−06 |
| ENSG00000132329 | RAMP1 | −1.477056239 | 3.65341E−06 |
| ENSG00000146555 | SDK1 | −1.477506838 | 5.90996E−05 |
| ENSG00000132274 | TRIM22 | −1.47813492 | 1.61701E−06 |
| ENSG00000116132 | PRRX1 | −1.478614381 | 5.83943E−05 |
| ENSG00000185739 | SRL | −1.47907594 | 0.000232338 |
| ENSG00000165072 | MAMDC2 | −1.479375871 | 0.002441418 |
| ENSG00000100024 | UPB1 | −1.479500636 | 0.01373858 |
| ENSG00000169862 | CTNND2 | −1.479738121 | 0.001140847 |
| ENSG00000112182 | BACH2 | −1.480035707 | 1.9412E−07 |
| ENSG00000132321 | IQCA1 | −1.480413044 | 4.15713E−05 |
| ENSG00000180644 | PRF1 | −1.481514029 | 0.000691017 |
| ENSG00000144712 | CAND2 | −1.48205596 | 0.000115084 |
| ENSG00000198963 | RORB | −1.483142457 | 0.001261372 |
| ENSG00000171777 | RASGRP4 | −1.484499328 | 3.86458E−07 |
| ENSG00000166546 | BEAN1 | −1.486354863 | 0.00021393 |
| ENSG00000160654 | CD3G | −1.48798618 | 1.05447E−05 |
| ENSG00000188820 | FAM26F | −1.48936868 | 0.000221408 |
| ENSG00000168421 | RHOH | −1.490154237 | 3.62887E−06 |
| ENSG00000146938 | NLGN4X | −1.491016537 | 8.90555E−06 |
| ENSG00000196405 | EVL | −1.492261635 | 7.33986E−08 |
| ENSG00000116690 | PRG4 | −1.492579766 | 0.011600499 |
| ENSG00000213203 | GIMAP1 | −1.494333415 | 4.7121E−06 |
| ENSG00000049540 | ELN | −1.494537514 | 4.9683E−06 |
| ENSG00000140287 | HDC | −1.495638222 | 4.0389E−05 |
| ENSG00000181790 | BAI1 | −1.49727332 | 0.000174264 |
| ENSG00000168875 | SOX14 | −1.498521925 | 0.015329029 |
| ENSG00000166741 | NNMT | −1.499407723 | 9.12715E−05 |
| ENSG00000168140 | VASN | −1.500069865 | 2.3508E−08 |
| ENSG00000124256 | ZBP1 | −1.501827474 | 8.46724E−05 |
| ENSG00000136244 | IL6 | −1.502133365 | 0.00018496 |
| ENSG00000211445 | GPX3 | −1.502476421 | 7.44696E−05 |
| ENSG00000111796 | KLRB1 | −1.503537324 | 0.001140171 |
| ENSG00000111452 | GPR133 | −1.504413354 | 7.32874E−07 |
| ENSG00000130224 | LRCH2 | −1.505032102 | 7.99716E−05 |
| ENSG00000072163 | LIMS2 | −1.505234356 | 1.86227E−08 |
| ENSG00000173762 | CD7 | −1.507000954 | 0.000639191 |
| ENSG00000106327 | TFR2 | −1.507799563 | 0.015023864 |
| ENSG00000101445 | PPP1R16B | −1.508082577 | 9.93664E−07 |
| ENSG00000183549 | ACSM5 | −1.508248292 | 0.011197472 |
| ENSG00000154277 | UCHL1 | −1.5087134 | 1.69988E−05 |
| ENSG00000105369 | CD79A | −1.509321396 | 0.000240457 |
| ENSG00000174348 | PODN | −1.509428102 | 5.35127E−07 |
| ENSG00000134201 | GSTM5 | −1.509980041 | 2.05026E−05 |
| ENSG00000140274 | DUOXA2 | −1.510700006 | 0.007182098 |
| ENSG00000197943 | PLCG2 | −1.510731018 | 2.11475E−08 |
| ENSG00000091656 | ZFHX4 | −1.510945405 | 7.68702E−06 |
| ENSG00000104974 | LILRA1 | −1.512817854 | 2.4424E−05 |
| ENSG00000013725 | CD6 | −1.513688335 | 3.65773E−06 |
| ENSG00000176533 | GNG7 | −1.514775354 | 1.50099E−06 |
| ENSG00000162692 | VCAM1 | −1.515374623 | 2.18735E−06 |
| ENSG00000054938 | CHRDL2 | −1.51790575 | 0.005020885 |
| ENSG00000187260 | WDR86 | −1.517998511 | 3.2638E−06 |
| ENSG00000138696 | BMPR1B | −1.519879182 | 4.77818E−05 |
| ENSG00000100368 | CSF2RB | −1.520882383 | 7.71039E−08 |
| ENSG00000161405 | IKZF3 | −1.524231413 | 1.40958E−05 |
| ENSG00000165125 | TRPV6 | −1.524418345 | 0.006282434 |
| ENSG00000188404 | SELL | −1.524526012 | 1.67292E−05 |
| ENSG00000131634 | TMEM204 | −1.527418737 | 7.67289E−09 |
| ENSG00000115604 | IL18R1 | −1.527507371 | 2.06106E−05 |
| ENSG00000184226 | PCDH9 | −1.527583929 | 4.10717E−07 |
| ENSG00000162739 | SLAMF6 | −1.528926599 | 5.63686E−05 |
| ENSG00000074047 | GLI2 | −1.529208704 | 5.88447E−09 |
| ENSG00000104332 | SFRP1 | −1.529634224 | 8.34178E−06 |
| ENSG00000275395 | FCGBP | −1.529686158 | 0.002214567 |
| ENSG00000072694 | FCGR2B | −1.531458196 | 7.92606E−06 |
| ENSG00000153563 | CD8A | −1.531510557 | 1.70823E−05 |
| ENSG00000235098 | ANKRD65 | −1.532017749 | 9.38941E−05 |
| ENSG00000138964 | PARVG | −1.533014221 | 1.03618E−07 |
| ENSG00000171444 | MCC | −1.533092846 | 1.65611E−06 |
| ENSG00000044524 | EPHA3 | −1.534789634 | 1.26977E−06 |
| ENSG00000110324 | IL10RA | −1.535684867 | 1.19976E−07 |
| ENSG00000149527 | PLCH2 | −1.540087018 | 5.02948E−06 |
| ENSG00000134853 | PDGFRA | −1.541650809 | 2.93382E−06 |
| ENSG00000115602 | IL1RL1 | −1.543852151 | 0.000336124 |
| ENSG00000072840 | EVC | −1.546053845 | 2.11475E−08 |
| ENSG00000188385 | JAKMIP3 | −1.548233697 | 1.65301E−06 |
| ENSG00000017427 | IGF1 | −1.548256962 | 0.000769952 |
| ENSG00000167261 | DPEP2 | −1.548790353 | 9.47397E−08 |
| ENSG00000139800 | ZIC5 | −1.549320247 | 0.007504945 |
| ENSG00000163071 | SPATA18 | −1.550345627 | 1.95046E−06 |
| ENSG00000020633 | RUNX3 | −1.55042504 | 1.67438E−08 |
| ENSG00000244731 | C4A | −1.550464023 | 0.005818325 |
| ENSG00000122691 | TWIST1 | −1.551224176 | 0.00039783 |
| ENSG00000138061 | CYP1B1 | −1.551931643 | 2.4673E−06 |
| ENSG00000094755 | GABRP | −1.552008805 | 0.007818468 |
| ENSG00000064300 | NGFR | −1.553953529 | 6.5295E−05 |
| ENSG00000172817 | CYP7B1 | −1.554195748 | 1.26489E−06 |
| ENSG00000005102 | MEOX1 | −1.554438791 | 0.000130756 |
| ENSG00000179348 | GATA2 | −1.556681027 | 4.6063E−06 |
| ENSG00000171608 | PIK3CD | −1.558634197 | 3.7402E−08 |
| ENSG00000129993 | CBFA2T3 | −1.55949201 | 1.78916E−06 |
| ENSG00000122188 | LAX1 | −1.562512852 | 0.000209843 |
| ENSG00000136155 | SCEL | −1.563969201 | 0.008172459 |
| ENSG00000156222 | SLC28A1 | −1.565856008 | 0.021093744 |
| ENSG00000179583 | CIITA | −1.56914011 | 2.47492E−06 |
| ENSG00000148053 | NTRK2 | −1.569472794 | 0.001412759 |
| ENSG00000179344 | HLA-DQB1 | −1.57087044 | 4.99227E−05 |
| ENSG00000108950 | FAM20A | −1.571607534 | 4.3045E−05 |
| ENSG00000183287 | CCBE1 | −1.571882164 | 2.15791E−05 |
| ENSG00000115607 | IL18RAP | −1.57397144 | 0.000340421 |
| ENSG00000149131 | SERPING1 | −1.574477195 | 6.28766E−05 |
| ENSG00000018625 | ATP1A2 | −1.574584571 | 0.000677893 |
| ENSG00000160282 | FTCD | −1.576568217 | 0.045728705 |
| ENSG00000115523 | GNLY | −1.577209668 | 0.000358699 |
| ENSG00000130812 | ANGPTL6 | −1.577413446 | 0.000176276 |
| ENSG00000100628 | ASB2 | −1.578587567 | 3.93982E−05 |
| ENSG00000126603 | GL1S2 | −1.578644116 | 5.47145E−08 |
| ENSG00000162894 | FAIM3 | −1.578890821 | 1.25594E−05 |
| ENSG00000203805 | PPAPDC1A | −1.579129572 | 7.66724E−05 |
| ENSG00000123360 | PDE1B | −1.580223798 | 1.27701E−07 |
| ENSG00000050730 | TNIP3 | −1.580726231 | 0.000100104 |
| ENSG00000243696 | RPS-966M1.6 | −1.580785901 | 0.002853316 |
| ENSG00000141431 | ASXL3 | −1.581098614 | 3.19022E−05 |
| ENSG00000253304 | TMEM200B | −1.583600932 | 3.7402E−08 |
| ENSG00000115648 | MLPH | −1.584496312 | 8.46724E−05 |
| ENSG00000182463 | TSHZ2 | −1.585228768 | 1.51929E−07 |
| ENSG00000145708 | CRHBP | −1.586345108 | 0.02136514 |
| ENSG00000139547 | RDH16 | −1.587228603 | 0.005684036 |
| ENSG00000117215 | PLA2G2D | −1.587484636 | 0.014025318 |
| ENSG00000101463 | SYNDIG1 | −1.58750882 | 0.0001669 |
| ENSG00000187193 | MT1X | −1.587516155 | 0.002355558 |
| ENSG00000127528 | KLF2 | −1.588749817 | 3.42653E−08 |
| ENSG00000007908 | SELE | −1.591517313 | 0.003609117 |
| ENSG00000187479 | C11orf96 | −1.59175367 | 5.33407E−06 |
| ENSG00000110448 | CD5 | −1.593521292 | 2.70401E−05 |
| ENSG00000101825 | MXRA5 | −1.593583615 | 3.09227E−05 |
| ENSG00000177098 | SCN4B | −1.594088271 | 5.71046E−05 |
| ENSG00000144681 | STAC | −1.594603568 | 0.000191125 |
| ENSG00000138311 | ZNF365 | −1.595470841 | 1.67571E−05 |
| ENSG00000077942 | FBLN1 | −1.596319762 | 0.002377355 |
| ENSG00000169083 | AR | −1.597168407 | 0.00095126 |
| ENSG00000146192 | FGD2 | −1.597387366 | 1.67438E−08 |
| ENSG00000078589 | P2RY10 | −1.597696437 | 0.000116257 |
| ENSG00000179388 | EGR3 | −1.597852607 | 3.28506E−06 |
| ENSG00000174944 | P2RY14 | −1.60050109 | 3.46148E−05 |
| ENSG00000005844 | ITGAL | −1.601350362 | 4.54336E−07 |
| ENSG00000172215 | CXCR6 | −1.602020984 | 6.66974E−05 |
| ENSG00000104435 | STMN2 | −1.602700919 | 0.000840704 |
| ENSG00000186479 | RGS7BP | −1.603707527 | 0.000105798 |
| ENSG00000153976 | HS3ST3A1 | −1.604351934 | 8.32817E−06 |
| ENSG00000150051 | MKX | −1.605162917 | 3.39392E−05 |
| ENSG00000106483 | SFRP4 | −1.605254889 | 2.10734E−05 |
| ENSG00000105374 | NKG7 | −1.60525897 | 0.000512273 |
| ENSG00000002746 | HECW1 | −1.605297814 | 1.39095E−05 |
| ENSG00000149970 | CNKSR2 | −1.607381055 | 1.80011E−05 |
| ENSG00000157103 | SLC6A1 | −1.607433083 | 1.47296E−05 |
| ENSG00000142871 | CYR61 | −1.607445176 | 1.33133E−05 |
| ENSG00000196329 | GIMAP5 | −1.608196974 | 4.38102E−05 |
| ENSG00000161544 | CYGB | −1.608829885 | 2.00477E−07 |
| ENSG00000132386 | SERPINF1 | −1.609846768 | 5.04228E−06 |
| ENSG00000073754 | CD5L | −1.610830571 | 0.031773904 |
| ENSG00000080007 | DDX43 | −1.611173367 | 0.001214143 |
| ENSG00000162804 | SNED1 | −1.611865848 | 5.42959E−08 |
| ENSG00000145864 | GABRB2 | −1.614793569 | 0.036956319 |
| ENSG00000104059 | FAM189A1 | −1.616655842 | 0.048118333 |
| ENSG00000158473 | CD1D | −1.616742846 | 1.46245E−06 |
| ENSG00000197859 | ADAMTSL2 | −1.619083779 | 5.69019E−07 |
| ENSG00000154451 | GBP5 | −1.623010684 | 8.48054E−05 |
| ENSG00000196358 | NTNG2 | −1.623642423 | 1.54908E−07 |
| ENSG00000110848 | CD69 | −1.624764075 | 0.000777544 |
| ENSG00000159650 | UROC1 | −1.625472907 | 0.006451973 |
| ENSG00000185905 | C16orf54 | −1.626526294 | 7.89301E−07 |
| ENSG00000102962 | CCL22 | −1.626752417 | 0.000659023 |
| ENSG00000168334 | XIRP1 | −1.627111341 | 0.000254513 |
| ENSG00000145649 | GZMA | −1.628084686 | 0.000807992 |
| ENSG00000167779 | IGFBP6 | −1.628820423 | 8.42388E−06 |
| ENSG00000132704 | FCRL2 | −1.628822089 | 0.000176206 |
| ENSG00000196159 | FAT4 | −1.629098699 | 2.11744E−07 |
| ENSG00000198542 | ITGBL1 | −1.631791452 | 9.61473E−07 |
| ENSG00000175857 | GAPT | −1.631827958 | 3.37022E−07 |
| ENSG00000124191 | TOX2 | −1.632292529 | 1.82257E−07 |
| ENSG00000185274 | WBSCR17 | −1.632407931 | 5.51436E−06 |
| ENSG00000167755 | KLK6 | −1.632699949 | 0.029537038 |
| ENSG00000168490 | PHYHIP | −1.634254375 | 3.86458E−07 |
| ENSG00000197888 | UGT2B17 | −1.635503209 | 0.017174225 |
| ENSG00000169031 | COL4A3 | −1.636189826 | 0.000137029 |
| ENSG00000154258 | ABCA9 | −1.6368406 | 4.61249E−06 |
| ENSG00000170801 | HTRA3 | −1.637259495 | 8.38017E−07 |
| ENSG00000132840 | BHMT2 | −1.638443812 | 0.011133062 |
| ENSG00000182326 | C1S | −1.639428931 | 9.3156E−06 |
| ENSG00000198821 | CD247 | −1.643666082 | 7.50175E−06 |
| ENSG00000087258 | GNAO1 | −1.644143425 | 4.92172E−06 |
| ENSG00000168477 | TNXB | −1.645553842 | 4.79665E−07 |
| ENSG00000185811 | IKZF1 | −1.647004425 | 1.20416E−07 |
| ENSG00000169252 | ADRB2 | −1.650121928 | 4.73559E−06 |
| ENSG00000140092 | FBLN5 | −1.651961926 | 4.92172E−06 |
| ENSG00000183918 | SH2D1A | −1.652848808 | 0.000221726 |
| ENSG00000135298 | BAI3 | −1.653296552 | 1.45136E−05 |
| ENSG00000182183 | FAM159A | −1.655531231 | 8.90555E−06 |
| ENSG00000175189 | INHBC | −1.655769735 | 0.013044116 |
| ENSG00000141338 | ABCA8 | −1.656965425 | 0.001659299 |
| ENSG00000182902 | SLC25A18 | −1.65954025 | 0.039343824 |
| ENSG00000219438 | FAM19A5 | −1.660585957 | 2.11475E−08 |
| ENSG00000117594 | HSD11B1 | −1.661539712 | 0.000131636 |
| ENSG00000107742 | SPOCK2 | −1.661835729 | 9.56676E−07 |
| ENSG00000167757 | KLK11 | −1.662496263 | 0.001152839 |
| ENSG00000101204 | CHRNA4 | −1.663842628 | 0.01603096 |
| ENSG00000122224 | LY9 | −1.664420811 | 6.38526E−06 |
| ENSG00000134193 | REG4 | −1.664764625 | 0.00455041 |
| ENSG00000094963 | FMO2 | −1.666056541 | 2.47492E−06 |
| ENSG00000123329 | ARHGAP9 | −1.666384228 | 3.7402E−08 |
| ENSG00000109943 | CRTAM | −1.66655714 | 1.14568E−05 |
| ENSG00000177990 | DPY19L2 | −1.668198172 | 0.000278385 |
| ENSG00000159231 | CBR3 | −1.66959019 | 6.18202E−06 |
| ENSG00000163687 | DNASE1L3 | −1.669605674 | 0.007120277 |
| ENSG00000160856 | FCRL3 | −1.671885886 | 0.001019428 |
| ENSG00000104814 | MAP4K1 | −1.671903082 | 7.37367E−07 |
| ENSG00000185669 | SNAI3 | −1.671932594 | 2.30806E−05 |
| ENSG00000181143 | MUC16 | −1.674323132 | 0.018339127 |
| ENSG00000149256 | TENM4 | −1.675077475 | 3.08739E−07 |
| ENSG00000149534 | MS4A2 | −1.67656614 | 3.74637E−06 |
| ENSG00000116824 | CD2 | −1.676720886 | 6.86303E−05 |
| ENSG00000166428 | PLD4 | −1.677504294 | 6.62967E−07 |
| ENSG00000124785 | NRN1 | −1.679063148 | 0.030891954 |
| ENSG00000106333 | PCOLCE | −1.681114376 | 3.86029E−10 |
| ENSG00000163564 | PYHIN1 | −1.682661941 | 0.000669298 |
| ENSG00000198846 | TOX | −1.683762457 | 0.00012471 |
| ENSG00000172236 | TPSAB1 | −1.685002747 | 3.24427E−05 |
| ENSG00000100652 | SLC10A1 | −1.687961533 | 0.043142038 |
| ENSG00000130005 | GAMT | −1.688620091 | 0.002831199 |
| ENSG00000118526 | TCF21 | −1.689694726 | 1.82257E−07 |
| ENSG00000185479 | KRT6B | −1.689922723 | 0.024466871 |
| ENSG00000144218 | AFF3 | −1.694414456 | 2.44028E−06 |
| ENSG00000187048 | CYP4A11 | −1.695406305 | 0.035374251 |
| ENSG00000172497 | ACOT12 | −1.696164082 | 0.031654747 |
| ENSG00000116106 | EPHA4 | −1.698555283 | 0.000147918 |
| ENSG00000089012 | SIRPG | −1.700868238 | 0.000153154 |
| ENSG00000125148 | MT2A | −1.701211079 | 5.66399E−05 |
| ENSG00000013297 | CLDN11 | −1.702569289 | 7.30473E−08 |
| ENSG00000117090 | SLAMF1 | −1.703205322 | 3.42517E−07 |
| ENSG00000091831 | ESR1 | −1.705792155 | 2.62136E−06 |
| ENSG00000157551 | KCNJ15 | −1.706433055 | 0.00011331 |
| ENSG00000099994 | SUSD2 | −1.70688574 | 1.67438E−08 |
| ENSG00000129009 | ISLR | −1.70768258 | 8.30629E−08 |
| ENSG00000042062 | FAM65C | −1.709721194 | 0.000409868 |
| ENSG00000271503 | CCL5 | −1.711093117 | 1.25536E−06 |
| ENSG00000186265 | BTLA | −1.711192354 | 7.89159E−05 |
| ENSG00000009790 | TRAF3IP3 | −1.712431415 | 9.733E−08 |
| ENSG00000140279 | DUOX2 | −1.718269856 | 0.001839583 |
| ENSG00000153283 | CD96 | −1.722446739 | 4.2153E−05 |
| ENSG00000145936 | KCNMB1 | −1.722680759 | 2.54675E−08 |
| ENSG00000131196 | NFATC1 | −1.725169239 | 2.11475E−08 |
| ENSG00000117643 | MAN1Cl | −1.726462069 | 2.11475E−08 |
| ENSG00000085265 | FCN1 | −1.72667995 | 3.16967E−06 |
| ENSG00000154262 | ABCA6 | −1.726777063 | 2.38468E−06 |
| ENSG00000170476 | MZB1 | −1.726853788 | 0.000127618 |
| ENSG00000174307 | PHLDA3 | −1.72817549 | 3.14471E−07 |
| ENSG00000139220 | PPFIA2 | −1.729023899 | 1.92084E−05 |
| ENSG00000109758 | HGFAC | −1.732231032 | 0.012227415 |
| ENSG00000112175 | BMP5 | −1.732616549 | 0.000255946 |
| ENSG00000106034 | CPED1 | −1.734018794 | 4.54336E−07 |
| ENSG00000100351 | GRAP2 | −1.736449607 | 3.92415E−08 |
| ENSG00000105664 | COMP | −1.736599018 | 7.85645E−05 |
| ENSG00000120907 | ADRA1A | −1.738244194 | 0.045728705 |
| ENSG00000263264 | CTB-133G6.1 | −1.740327674 | 0.000141667 |
| ENSG00000167286 | CD3D | −1.740381311 | 5.30868E−05 |
| ENSG00000204866 | IGFL2 | −1.742775941 | 1.4293E−06 |
| ENSG00000103522 | IL21R | −1.74310075 | 1.16962E−06 |
| ENSG00000105352 | CEACAM4 | −1.744678061 | 2.38468E−06 |
| ENSG00000172349 | IL16 | −1.745060183 | 1.86227E−08 |
| ENSG00000126231 | PROZ | −1.747955437 | 0.016371477 |
| ENSG00000197057 | DTHD1 | −1.749866928 | 0.000139515 |
| ENSG00000139193 | CD27 | −1.751846546 | 2.10734E−05 |
| ENSG00000025423 | HSD17B6 | −1.755989093 | 0.008558788 |
| ENSG00000039537 | C6 | −1.757524211 | 0.04246576 |
| ENSG00000170373 | CST1 | −1.757993922 | 0.036301707 |
| ENSG00000163568 | AIM2 | −1.758409452 | 9.88776E−05 |
| ENSG00000109684 | CLNK | −1.760001006 | 8.55803E−06 |
| ENSG00000099998 | GGT5 | −1.760531218 | 1.67438E−08 |
| ENSG00000081052 | COL4A4 | −1.763469313 | 1.56991E−07 |
| ENSG00000109906 | ZBTB16 | −1.763666181 | 0.002274733 |
| ENSG00000164107 | HAND2 | −1.765677171 | 1.4631E−07 |
| ENSG00000173040 | EVC2 | −1.76745187 | 1.89078E−09 |
| ENSG00000147443 | DOK2 | −1.772561202 | 6.51487E−07 |
| ENSG00000169715 | MT1E | −1.775885047 | 0.000152182 |
| ENSG00000196188 | CTSE | −1.776727632 | 0.002499377 |
| ENSG00000152969 | JAKMIP1 | −1.778936736 | 9.41953E−07 |
| ENSG00000168229 | PTGDR | −1.779594433 | 0.000508899 |
| ENSG00000108381 | ASPA | −1.784171677 | 6.28688E−05 |
| ENSG00000135426 | TESPA1 | −1.784541597 | 3.75146E−06 |
| ENSG00000064205 | WISP2 | −1.785179649 | 7.5572E−05 |
| ENSG00000160593 | AMICA1 | −1.788120659 | 3.34865E−08 |
| ENSG00000105509 | HAS1 | −1.789625772 | 3.86669E−05 |
| ENSG00000050555 | LAMC3 | −1.791831208 | 8.02783E−05 |
| ENSG00000106538 | RARRES2 | −1.792914945 | 7.89301E−07 |
| ENSG00000218336 | TENM3 | −1.793825315 | 0.000330204 |
| ENSG00000132465 | IGJ | −1.794909121 | 0.000268589 |
| ENSG00000144649 | FAM198A | −1.795310856 | 5.71046E−05 |
| ENSG00000110777 | POU2AF1 | −1.796428571 | 3.12173E−05 |
| ENSG00000131459 | GFPT2 | −1.796743187 | 7.30473E−08 |
| ENSG00000140368 | PSTPIP1 | −1.797172537 | 5.40421E−08 |
| ENSG00000166250 | CLMP | −1.799025045 | 4.64967E−05 |
| ENSG00000167178 | ISLR2 | −1.80040134 | 2.28172E−07 |
| ENSG00000174175 | SELP | −1.800654289 | 6.65599E−06 |
| ENSG00000156886 | ITGAD | −1.805375882 | 1.36379E−05 |
| ENSG00000134539 | KLRD1 | −1.813285483 | 1.95046E−06 |
| ENSG00000159403 | C1R | −1.813324895 | 2.07859E−06 |
| ENSG00000151224 | MAT1A | −1.816081848 | 0.043016212 |
| ENSG00000110436 | SLC1A2 | −1.81621643 | 0.007372737 |
| ENSG00000151892 | GFRA1 | −1.820928128 | 0.000809181 |
| ENSG00000172346 | CSDC2 | −1.824103968 | 2.58144E−05 |
| ENSG00000162365 | CYP4A22 | −1.825563247 | 0.019467695 |
| ENSG00000090539 | CHRD | −1.826733716 | 6.099E−06 |
| ENSG00000180447 | GAS1 | −1.826917582 | 1.46245E−06 |
| ENSG00000105122 | RASAL3 | −1.828017072 | 2.38902E−08 |
| ENSG00000105989 | WNT2 | −1.836139896 | 3.64866E−08 |
| ENSG00000205045 | SLFN12L | −1.836470932 | 2.32958E−08 |
| ENSG00000162493 | PDPN | −1.836830676 | 7.23692E−06 |
| ENSG00000163492 | CCDC141 | −1.83690351 | 1.52249E−05 |
| ENSG00000171385 | KCND3 | −1.837334691 | 1.63415E−05 |
| ENSG00000087245 | MMP2 | −1.839379635 | 2.027E−07 |
| ENSG00000189233 | NUGGC | −1.839733753 | 0.00180492 |
| ENSG00000152495 | CAMK4 | −1.839956771 | 6.36692E−08 |
| ENSG00000169851 | PCDH7 | −1.842391319 | 5.66683E−07 |
| ENSG00000077984 | CST7 | −1.843553715 | 4.84108E−05 |
| ENSG00000126353 | CCR7 | −1.845908873 | 0.000121606 |
| ENSG00000091513 | TF | −1.847238949 | 0.042339945 |
| ENSG00000272636 | DOC2B | −1.84776899 | 2.67007E−05 |
| ENSG00000145423 | SFRP2 | −1.851133938 | 0.007875051 |
| ENSG00000029534 | ANK1 | −1.852965994 | 7.07184E−08 |
| ENSG00000152784 | PRDM8 | −1.856334997 | 8.2985E−08 |
| ENSG00000127954 | STEAP4 | −1.8615322 | 1.13189E−05 |
| ENSG00000011465 | DCN | −1.862962082 | 9.9073E−08 |
| ENSG00000144857 | BOC | −1.865221827 | 5.92715E−08 |
| ENSG00000122025 | FLT3 | −1.86617214 | 1.64174E−05 |
| ENSG00000214402 | LCNL1 | −1.869824232 | 0.000138259 |
| ENSG00000100346 | CACNA1I | −1.869836037 | 0.000559201 |
| ENSG00000156475 | PPP2R2B | −1.870638406 | 7.91103E−07 |
| ENSG00000113263 | ITK | −1.876696984 | 3.00113E−06 |
| ENSG00000163395 | IGFN1 | −1.880251791 | 0.000205549 |
| ENSG00000189320 | FAM180A | −1.881797326 | 1.69401E−06 |
| ENSG00000152583 | SPARCL1 | −1.881878215 | 1.9548E−07 |
| ENSG00000140285 | FGF7 | −1.884272369 | 0.000344444 |
| ENSG00000205795 | CYS1 | −1.884441345 | 1.17091E−06 |
| ENSG00000205784 | ARRDC5 | −1.899477613 | 1.05902E−06 |
| ENSG00000172482 | AGXT | −1.900227176 | 0.023398913 |
| ENSG00000060709 | RIMBP2 | −1.901046353 | 0.00191795 |
| ENSG00000175463 | TBC1D10C | −1.90407368 | 4.79665E−07 |
| ENSG00000250799 | PRODH2 | −1.910127685 | 0.007837018 |
| ENSG00000153292 | GPR110 | −1.911516006 | 0.000392148 |
| ENSG00000162552 | WNT4 | −1.912081822 | 6.55218E−08 |
| ENSG00000182836 | PLCXD3 | −1.919350408 | 2.67007E−05 |
| ENSG00000183778 | B3GALT5 | −1.921511887 | 5.4965E−06 |
| ENSG00000125384 | PTGER2 | −1.926307882 | 7.11386E−08 |
| ENSG00000198851 | CD3E | −1.926834494 | 1.9412E−07 |
| ENSG00000205364 | MT1M | −1.93278971 | 0.001076927 |
| ENSG00000068831 | RASGRP2 | −1.934148078 | 9.47397E−08 |
| ENSG00000149451 | ADAM33 | −1.939863642 | 1.52496E−05 |
| ENSG00000173705 | SUSD5 | −1.941554039 | 1.68078E−05 |
| ENSG00000172673 | THEMIS | −1.942331165 | 5.9626E−07 |
| ENSG00000168685 | IL7R | −1.944567611 | 1.80121E−05 |
| ENSG00000113088 | GZMK | −1.95209464 | 5.62555E−05 |
| ENSG00000161031 | PGLYRP2 | −1.957638292 | 0.027182082 |
| ENSG00000180828 | BHLHE22 | −1.961742811 | 2.93627E−06 |
| ENSG00000167984 | NLRC3 | −1.968699444 | 2.3508E−08 |
| ENSG00000079931 | MOXD1 | −1.970809581 | 2.3508E−08 |
| ENSG00000073861 | TBX21 | −1.972710767 | 3.64223E−05 |
| ENSG00000072818 | ACAP1 | −1.973493751 | 3.64866E−08 |
| ENSG00000163508 | EOMES | −1.975206409 | 2.18735E−06 |
| ENSG00000071991 | CDH19 | −1.976525894 | 6.92606E−06 |
| ENSG00000275063 | AC233755.1 | −1.980211896 | 0.001328112 |
| ENSG00000183160 | TMEM119 | −1.98856722 | 3.9501E−06 |
| ENSG00000197253 | TPSB2 | −1.989161318 | 1.67571E−05 |
| ENSG00000135144 | DTX1 | −1.989482455 | 4.0212E−05 |
| ENSG00000137265 | IRF4 | −1.991133162 | 3.58567E−07 |
| ENSG00000121410 | A1BG | −1.994194759 | 2.39205E−05 |
| ENSG00000244734 | HBB | −1.996693638 | 1.82887E−06 |
| ENSG00000134538 | SLCO1B1 | −1.997247617 | 0.024258756 |
| ENSG00000140505 | CYP1A2 | −1.998480963 | 0.007359436 |
| ENSG00000145934 | TENM2 | −2.005163875 | 2.88302E−05 |
| ENSG00000115085 | ZAP70 | −2.008362263 | 3.43678E−07 |
| ENSG00000188389 | PDCD1 | −2.013304666 | 1.0855E−05 |
| ENSG00000110900 | TSPAN11 | −2.013727616 | 2.99849E−05 |
| ENSG00000144908 | ALDH1L1 | −2.02257893 | 0.007027914 |
| ENSG00000136011 | STAB2 | −2.023675331 | 0.001918481 |
| ENSG00000160185 | UBASH3A | −2.024512063 | 1.6877E−06 |
| ENSG00000166482 | MFAP4 | −2.032069985 | 3.36232E−06 |
| ENSG00000123453 | SARDH | −2.036085745 | 1.17091E−06 |
| ENSG00000047617 | ANO2 | −2.042553124 | 4.10994E−07 |
| ENSG00000171812 | COL8A2 | −2.043031965 | 1.89078E−09 |
| ENSG00000198650 | TAT | −2.049538974 | 0.039892923 |
| ENSG00000122787 | AKR1D1 | −2.050473152 | 0.002259074 |
| ENSG00000177272 | KCNA3 | −2.059440975 | 2.64354E−08 |
| ENSG00000140107 | SLC25A47 | −2.074759218 | 0.016748536 |
| ENSG00000155657 | TTN | −2.077272513 | 1.73385E−07 |
| ENSG00000172061 | LRRC15 | −2.088920797 | 3.29885E−06 |
| ENSG00000215644 | GCGR | −2.094491352 | 0.00455041 |
| ENSG00000147138 | GPR174 | −2.098204569 | 2.87088E−06 |
| ENSG00000117601 | SERPINC1 | −2.101960398 | 0.046802435 |
| ENSG00000169035 | KLK7 | −2.119202838 | 0.001130698 |
| ENSG00000101188 | NTSR1 | −2.136271515 | 0.001365223 |
| ENSG00000184956 | MUC6 | −2.142966072 | 0.007582781 |
| ENSG00000138135 | CH25H | −2.143580695 | 0.000289829 |
| ENSG00000105697 | HAMP | −2.147268816 | 0.000790785 |
| ENSG00000129596 | CDO1 | −2.152713049 | 0.004311464 |
| ENSG00000003137 | CYP26B1 | −2.154130063 | 1.65513E−06 |
| ENSG00000132514 | CLEC10A | −2.164002441 | 6.18202E−06 |
| ENSG00000145692 | BHMT | −2.172393141 | 0.022336562 |
| ENSG00000121807 | CCR2 | −2.17920285 | 3.94158E−07 |
| ENSG00000174946 | GPR171 | −2.180670676 | 8.19986E−05 |
| ENSG00000163519 | TRAT1 | −2.183040612 | 2.15639E−05 |
| ENSG00000154529 | CNTNAP3B | −2.19207145 | 3.03667E−05 |
| ENSG00000173578 | XCR1 | −2.197511235 | 1.28433E−05 |
| ENSG00000143196 | DPT | −2.200975281 | 4.61599E−06 |
| ENSG00000119714 | GPR68 | −2.200996178 | 1.67438E−08 |
| ENSG00000254709 | IGLL5 | −2.201894109 | 5.90052E−06 |
| ENSG00000123500 | COL10A1 | −2.203898449 | 1.4631E−07 |
| ENSG00000131386 | GALNT15 | −2.21041109 | 2.11475E−08 |
| ENSG00000110799 | VWF | −2.217632153 | 5.88447E−09 |
| ENSG00000140835 | CHST4 | −2.248832453 | 0.000211595 |
| ENSG00000110243 | APOA5 | −2.249011197 | 0.021109571 |
| ENSG00000187634 | SAMD11 | −2.268098478 | 1.86227E−08 |
| ENSG00000110245 | APOC3 | −2.279400193 | 0.044167829 |
| ENSG00000118137 | APOA1 | −2.292998066 | 0.029453145 |
| ENSG00000206384 | COL6A6 | −2.337054387 | 8.75035E−08 |
| ENSG00000137077 | CCL21 | −2.339382377 | 1.8088E−05 |
| ENSG00000166148 | AVPR1A | −2.347452419 | 0.001434051 |
| ENSG00000130649 | CYP2E1 | −2.365713616 | 0.001806626 |
| ENSG00000255974 | CYP2A6 | −2.366850771 | 0.029623804 |
| ENSG00000124203 | ZNF831 | −2.384341125 | 7.11386E−08 |
| ENSG00000129451 | KLK10 | −2.423477225 | 0.00019224 |
| ENSG00000196616 | ADH1B | −2.436635188 | 0.005517265 |
| ENSG00000140538 | NTRK3 | −2.441826477 | 5.0103E−08 |
| ENSG00000156738 | MS4A1 | −2.503377804 | 8.38017E−07 |
| ENSG00000136573 | BLK | −2.521099289 | 3.35863E−06 |
| ENSG00000136546 | SCN7A | −2.534193956 | 1.35959E−05 |
| ENSG00000107317 | PTGDS | −2.567982401 | 3.42653E−08 |
| ENSG00000172543 | CTSW | −2.601624003 | 2.62703E−08 |
| ENSG00000112936 | C7 | −2.734427236 | 6.99257E−06 |
| ENSG00000172724 | CCL19 | −3.10276147 | 1.78025E−06 |

TABLE 3B

| Ensembl ID | Gene Symbol | log2FC | adj P-Value |
|---|---|---|---|
| ENSG00000118137 | APOA1 | 4.03342127 | 0.00020128 |
| ENSG00000110245 | APOC3 | 3.817494407 | 0.000895708 |
| ENSG00000172482 | AGXT | 3.649556969 | 3.19938E−05 |
| ENSG00000110243 | APOA5 | 3.555420548 | 0.000365209 |
| ENSG00000101204 | CHRNA4 | 3.521926862 | 2.38615E−06 |
| ENSG00000255974 | CYP2A6 | 3.400280295 | 0.002039671 |
| ENSG00000137204 | SLC22A7 | 3.344034575 | 0.000526078 |
| ENSG00000055957 | ITIH1 | 3.318666831 | 0.000632477 |
| ENSG00000215644 | GCGR | 3.313633037 | 2.0805E−05 |
| ENSG00000140107 | SLC25A47 | 3.214504508 | 0.000308981 |
| ENSG00000145192 | AHSG | 3.201706797 | 0.002890618 |
| ENSG00000140505 | CYP1A2 | 3.164111782 | 5.71982E−05 |
| ENSG00000163631 | ALB | 3.157004897 | 0.002452464 |
| ENSG00000141485 | SLC13A5 | 3.153757229 | 0.000102754 |
| ENSG00000117601 | SERPINC1 | 3.13709961 | 0.003306975 |
| ENSG00000122194 | PLG | 3.12327998 | 0.004005088 |
| ENSG00000161031 | PGLYRP2 | 3.097960789 | 0.000606349 |
| ENSG00000158104 | HPD | 3.095102273 | 0.001768852 |
| ENSG00000228278 | ORM2 | 3.091738751 | 0.001237175 |
| ENSG00000173432 | SAA1 | 3.091244007 | 0.001422055 |
| ENSG00000156222 | SLC28A1 | 3.074992314 | 1.67555E−05 |
| ENSG00000109758 | HGFAC | 3.045511033 | 2.85412E−05 |
| ENSG00000120907 | ADRA1A | 3.037827378 | 0.000607279 |
| ENSG00000257017 | HP | 3.037527154 | 0.01404727 |
| ENSG00000198650 | TAT | 3.008538872 | 0.002902688 |
| ENSG00000130649 | CYP2E1 | 2.981681371 | 0.000162521 |
| ENSG00000105707 | HPN | 2.981679795 | 6.19929E−05 |
| ENSG00000172724 | CCL19 | 2.963589155 | 5.23278E−06 |
| ENSG00000145692 | BHMT | 2.940080806 | 0.002326865 |
| ENSG00000159650 | UROC1 | 2.931689347 | 4.46195E−06 |
| ENSG00000105398 | SULT2A1 | 2.895943776 | 0.001125162 |
| ENSG00000161944 | ASGR2 | 2.886375795 | 0.000244032 |
| ENSG00000073754 | CD5L | 2.884937538 | 0.000185574 |
| ENSG00000175189 | INHBC | 2.879896728 | 3.97202E−05 |
| ENSG00000157131 | C8A | 2.871228568 | 0.006196416 |
| ENSG00000147647 | DPYS | 2.861699945 | 0.000605481 |
| ENSG00000151224 | MAT1A | 2.853444253 | 0.001735388 |
| ENSG00000118271 | TTR | 2.852019202 | 0.007471748 |
| ENSG00000122787 | AKR1D1 | 2.851571077 | 4.68552E−05 |
| ENSG00000079557 | AFM | 2.847947396 | 0.004612123 |
| ENSG00000009724 | MASP2 | 2.840850223 | 0.000860058 |
| ENSG00000126231 | PROZ | 2.815787191 | 0.000192046 |
| ENSG00000109072 | VTN | 2.805293077 | 0.001565125 |
| ENSG00000178772 | CPN2 | 2.801411839 | 0.001099451 |
| ENSG00000250799 | PRODH2 | 2.80028653 | 0.000173312 |
| ENSG00000187048 | CYP4A11 | 2.796840372 | 0.000719964 |
| ENSG00000162365 | CYP4A22 | 2.782937306 | 0.000532431 |
| ENSG00000160282 | FTCD | 2.765798702 | 0.000559263 |
| ENSG00000131187 | F12 | 2.755837253 | 1.92409E−07 |
| ENSG00000158874 | APOA2 | 2.755273623 | 0.013072978 |
| ENSG00000100652 | SLC10A1 | 2.749245822 | 0.001183352 |
| ENSG00000196600 | SLC22A25 | 2.73014871 | 0.000628883 |
| ENSG00000066813 | ACSM2B | 2.726292317 | 0.001969372 |
| ENSG00000105697 | HAMP | 2.712303827 | 4.53987E−05 |
| ENSG00000113905 | HRG | 2.70641239 | 0.015993876 |
| ENSG00000106927 | AMBP | 2.704162113 | 0.006237735 |
| ENSG00000180210 | F2 | 2.699176393 | 0.002605332 |
| ENSG00000171560 | FGA | 2.693980117 | 0.00255617 |
| ENSG00000139269 | INHBE | 2.688059199 | 0.000230634 |
| ENSG00000131482 | G6PC | 2.684880323 | 0.003603728 |
| ENSG00000166148 | AVPR1A | 2.670331491 | 0.000408681 |
| ENSG00000187758 | ADH1A | 2.666188722 | 0.008182502 |
| ENSG00000182902 | SLC25A18 | 2.649043378 | 0.001183394 |
| ENSG00000180432 | CYP8B1 | 2.643870861 | 0.004220946 |
| ENSG00000138207 | RBP4 | 2.63884479 | 0.002229992 |
| ENSG00000101323 | HAO1 | 2.63869104 | 0.001750212 |
| ENSG00000244731 | C4A | 2.638540175 | 9.85035E−06 |
| ENSG00000162267 | ITIH3 | 2.616213914 | 0.001394604 |
| ENSG00000156738 | MS4A1 | 2.614889948 | 4.72612E−07 |
| ENSG00000091513 | TF | 2.599465774 | 0.004936377 |
| ENSG00000186910 | SERPINA11 | 2.586296044 | 0.005890983 |
| ENSG00000181790 | BAI1 | 2.568319627 | 1.87301E−08 |
| ENSG00000168509 | HFE2 | 2.565607791 | 0.001891022 |
| ENSG00000189233 | NUGGC | 2.542269974 | 3.59335E−05 |
| ENSG00000141505 | ASGR1 | 2.54217327 | 0.000163093 |
| ENSG00000106327 | TFR2 | 2.541081148 | 7.88371E−05 |
| ENSG00000167711 | SERPINF2 | 2.536203015 | 4.71562E−05 |
| ENSG00000167588 | GPD1 | 2.516540158 | 2.10921E−06 |
| ENSG00000134389 | CFHR5 | 2.51553596 | 0.003266532 |
| ENSG00000163687 | DNASE1L3 | 2.51349642 | 9.92817E−05 |
| ENSG00000099769 | IGFALS | 2.511431301 | 2.84079E−05 |
| ENSG00000060566 | CREB3L3 | 2.51092543 | 0.00037388 |
| ENSG00000183747 | ACSM2A | 2.499331636 | 0.001401275 |
| ENSG00000138109 | CYP2C9 | 2.496237434 | 0.002477368 |
| ENSG00000167676 | PLIN4 | 2.484747629 | 0.000100961 |
| ENSG00000163581 | SLC2A2 | 2.474354607 | 0.016215986 |
| ENSG00000125144 | MT1G | 2.467085048 | 2.71347E−05 |
| ENSG00000142748 | FCN3 | 2.464592475 | 5.11234E−05 |
| ENSG00000172497 | ACOT12 | 2.460074753 | 0.002117129 |
| ENSG00000244414 | CFHR1 | 2.455634101 | 0.006590488 |
| ENSG00000021852 | C8B | 2.452331363 | 0.009125228 |
| ENSG00000184956 | MUC6 | 2.448967667 | 0.002600485 |
| ENSG00000166183 | ASPG | 2.441975552 | 1.07579E−05 |
| ENSG00000134339 | SAA2 | 2.436739409 | 0.002929755 |
| ENSG00000118520 | ARG1 | 2.431413131 | 0.004864395 |
| ENSG00000214456 | PLIN5 | 2.411934168 | 2.15966E−05 |
| ENSG00000146678 | IGFBP1 | 2.400626989 | 0.005979301 |
| ENSG00000088926 | F11 | 2.392702358 | 0.008411284 |
| ENSG00000084674 | APOB | 2.380551386 | 0.011620388 |
| ENSG00000139547 | RDH16 | 2.378004222 | 7.0753E−05 |
| ENSG00000157087 | ATP2B2 | 2.368883131 | 0.002117311 |
| ENSG00000113889 | KNG1 | 2.367626386 | 0.018695719 |
| ENSG00000130173 | C190rf80 | 2.364577006 | 0.004209745 |
| ENSG00000171236 | LRG1 | 2.36171303 | 6.87212E−06 |
| ENSG00000129988 | LBP | 2.359732338 | 0.005841357 |
| ENSG00000025423 | HSD17B6 | 2.359462566 | 0.000634618 |
| ENSG00000113600 | C9 | 2.330342283 | 0.027453219 |
| ENSG00000182327 | GLTPD2 | 2.326087705 | 3.50261E−05 |
| ENSG00000091583 | APOH | 2.324805638 | 0.047565572 |
| ENSG00000198099 | ADH4 | 2.314677646 | 0.017707948 |
| ENSG00000130005 | GAMT | 2.313115142 | 8.2458E−05 |
| ENSG00000145850 | TIMD4 | 2.311992794 | 0.00014789 |
| ENSG00000039537 | C6 | 2.311294572 | 0.008045726 |
| ENSG00000116785 | CFHR3 | 2.2878727 | 0.008480607 |
| ENSG00000162551 | ALPL | 2.280386871 | 5.46105E−06 |
| ENSG00000143627 | PKLR | 2.272664786 | 0.000602546 |
| ENSG00000261701 | HPR | 2.272424321 | 0.01420778 |
| ENSG00000229314 | ORM1 | 2.26704992 | 0.025128422 |
| ENSG00000084734 | GCKR | 2.266094526 | 0.000587665 |
| ENSG00000143257 | NR1I3 | 2.264813532 | 0.003087031 |
| ENSG00000175336 | APOF | 2.264376716 | 0.008749459 |
| ENSG00000198670 | LPA | 2.241502788 | 0.001099601 |
| ENSG00000277893 | SRD5A2 | 2.24024151 | 0.000766218 |
| ENSG00000144908 | ALDH1L1 | 2.239002548 | 0.003371363 |
| ENSG00000187045 | TMPRSS6 | 2.232259824 | 0.000121488 |
| ENSG00000107317 | PTGDS | 2.22816392 | 7.1707E−07 |
| ENSG00000101981 | F9 | 2.227038159 | 0.02450203 |
| ENSG00000124203 | ZNF831 | 2.21137554 | 4.2107E−07 |
| ENSG00000110887 | DAO | 2.210982589 | 0.000458996 |
| ENSG00000174990 | CA5A | 2.206853287 | 0.000184293 |
| ENSG00000136872 | ALDOB | 2.20497856 | 0.005457894 |
| ENSG00000145626 | UGT3A1 | 2.204846842 | 0.004959351 |
| ENSG00000110169 | HPX | 2.198953128 | 0.002946105 |
| ENSG00000160868 | CYP3A4 | 2.188304321 | 0.025404392 |
| ENSG00000103449 | SALL1 | 2.187835144 | 0.000884892 |
| ENSG00000125730 | C3 | 2.184242808 | 0.000424251 |
| ENSG00000197444 | OGDHL | 2.183148343 | 0.000652796 |
| ENSG00000090539 | CHRD | 2.177775965 | 3.46838E−07 |
| ENSG00000145321 | GC | 2.17078114 | 0.043649274 |
| ENSG00000172543 | CTSW | 2.166094783 | 1.22945E−06 |
| ENSG00000129596 | CDO1 | 2.157586834 | 0.004892539 |
| ENSG00000138315 | OIT3 | 2.154636981 | 0.000348535 |
| ENSG00000121410 | A1BG | 2.154024269 | 9.12813E−06 |
| ENSG00000136011 | STAB2 | 2.153895927 | 0.001170482 |
| ENSG00000112337 | SLC17A2 | 2.151273679 | 0.002490273 |
| ENSG00000143921 | ABCG8 | 2.140849758 | 0.006281255 |
| ENSG00000120054 | CPN1 | 2.140412287 | 0.008337442 |
| ENSG00000163884 | KLF15 | 2.136804934 | 0.000176568 |
| ENSG00000196616 | ADH1B | 2.120909383 | 0.017166589 |
| ENSG00000136573 | BLK | 2.120259343 | 7.79626E−05 |
| ENSG00000188257 | PLA2G2A | 2.11905233 | 0.01547861 |
| ENSG00000105374 | NKG7 | 2.109664434 | 1.43089E−05 |
| ENSG00000161896 | IP6K3 | 2.106714009 | 0.001809755 |
| ENSG00000073734 | ABCB11 | 2.09464907 | 0.006671003 |
| ENSG00000145708 | CRHBP | 2.080523576 | 0.003098848 |
| ENSG00000169136 | ATF5 | 2.074975467 | 3.34382E−08 |
| ENSG00000170956 | CEACAM3 | 2.071071455 | 6.53981E−06 |
| ENSG00000162896 | PIGR | 2.067595291 | 0.037879176 |
| ENSG00000138115 | CYP2C8 | 2.062714257 | 0.030384223 |
| ENSG00000177238 | TRIM72 | 2.06137635 | 0.000541229 |
| ENSG00000100024 | UPB1 | 2.056548897 | 0.000887421 |
| ENSG00000132840 | BHMT2 | 2.056293226 | 0.001897798 |
| ENSG00000100344 | PNPLA3 | 2.055811223 | 0.000831964 |
| ENSG00000111249 | CUX2 | 2.052733446 | 0.000470832 |
| ENSG00000120885 | CLU | 2.051071157 | 0.000430434 |
| ENSG00000123453 | SARDH | 2.043937302 | 1.45867E−06 |
| ENSG00000171564 | FGB | 2.038597238 | 0.044898114 |
| ENSG00000168229 | PTGDR | 2.037806451 | 0.000134693 |
| ENSG00000151948 | GLT1D1 | 2.036591898 | 3.40188E−05 |
| ENSG00000263264 | CTB-133G6.1 | 2.032222551 | 1.84836E−05 |
| ENSG00000183549 | ACSM5 | 2.031757331 | 0.000901791 |
| ENSG00000162897 | FCAMR | 2.02756664 | 4.90232E−06 |
| ENSG00000188389 | PDCD1 | 2.022802834 | 1.33473E−05 |
| ENSG00000171798 | KNDC1 | 2.01574676 | 0.004354954 |
| ENSG00000151892 | GFRA1 | 2.011732065 | 0.000368787 |
| ENSG00000149527 | PLCH2 | 2.008865677 | 4.66585E−08 |
| ENSG00000176919 | C8G | 2.007593899 | 0.000165191 |
| ENSG00000104760 | FGL1 | 2.007114479 | 0.035150116 |
| ENSG00000113492 | AGXT2 | 2.005682188 | 0.008337442 |
| ENSG00000115718 | PROC | 2.005098773 | 0.000808643 |
| ENSG00000072818 | ACAP1 | 2.003565112 | 2.83645E−08 |
| ENSG00000165471 | MBL2 | 1.99513021 | 0.020127504 |
| ENSG00000090534 | THPO | 1.994330177 | 2.76651E−05 |
| ENSG00000146147 | MLIP | 1.991597761 | 0.003784608 |
| ENSG00000099937 | SERPIND1 | 1.98233912 | 0.0305223 |
| ENSG00000243696 | RPS-966M1.6 | 1.978888481 | 0.000292106 |
| ENSG00000132703 | APCS | 1.976538082 | 0.0252356 |
| ENSG00000224389 | C4B | 1.975395043 | 0.000960287 |
| ENSG00000132514 | CLEC10A | 1.964789409 | 4.44362E−05 |
| ENSG00000021826 | CPS1 | 1.963326101 | 0.028428885 |
| ENSG00000135144 | DTX1 | 1.958723636 | 6.69335E−05 |
| ENSG00000005421 | PON1 | 1.957557669 | 0.007436136 |
| ENSG00000130222 | GADD45G | 1.953456897 | 4.3641E−07 |
| ENSG00000136574 | GATA4 | 1.9515903 | 2.0805E−05 |
| ENSG00000275152 | CCL16 | 1.94269905 | 0.00406139 |
| ENSG00000132693 | CRP | 1.940142174 | 0.026905704 |
| ENSG00000122679 | RAMP3 | 1.939738533 | 3.46324E−08 |
| ENSG00000138798 | EGF | 1.938228743 | 0.011819496 |
| ENSG00000159261 | CLDN14 | 1.928305056 | 6.01076E−05 |
| ENSG00000178297 | TMPRSS9 | 1.927239153 | 0.000121913 |
| ENSG00000100346 | CACNA1I | 1.919037239 | 0.000515575 |
| ENSG00000073861 | TBX21 | 1.918366563 | 7.86285E−05 |
| ENSG00000124721 | DNAH8 | 1.913703166 | 0.003148436 |
| ENSG00000138356 | AOX1 | 1.9118762 | 0.015369331 |
| ENSG00000187193 | MT1X | 1.910199557 | 0.000396357 |
| ENSG00000140835 | CHST4 | 1.910062582 | 0.001748491 |
| ENSG00000105122 | RASAL3 | 1.897616857 | 1.18923E−08 |
| ENSG00000196990 | FAM163B | 1.896368518 | 6.23831E−05 |
| ENSG00000138075 | ABCG5 | 1.8908586 | 0.008784112 |
| ENSG00000131910 | NR0B2 | 1.882096536 | 6.71783E−05 |
| ENSG00000103313 | MEFV | 1.879687195 | 1.87175E−06 |
| ENSG00000093134 | VNN3 | 1.876205783 | 0.00020497 |
| ENSG00000125735 | TNFSF14 | 1.875354108 | 7.64183E−05 |
| ENSG00000129451 | KLK10 | 1.873471736 | 0.003739833 |
| ENSG00000003137 | CYP26B1 | 1.870052122 | 2.68514E−05 |
| ENSG00000111713 | GYS2 | 1.869332676 | 0.022629452 |
| ENSG00000101188 | NTSR1 | 1.868718916 | 0.00664869 |
| ENSG00000177989 | ODF3B | 1.866218494 | 2.01349E−09 |
| ENSG00000110799 | VWF | 1.865960751 | 1.98697E−07 |
| ENSG00000111181 | SLC6Al2 | 1.863396594 | 7.39367E−06 |
| ENSG00000187634 | SAMD11 | 1.863359824 | 9.81754E−07 |
| ENSG00000123561 | SERPINA7 | 1.863050591 | 0.011816054 |
| ENSG00000154529 | CNTNAP3B | 1.862816347 | 0.000420871 |
| ENSG00000116882 | HAO2 | 1.861459243 | 0.010122264 |
| ENSG00000170099 | SERPINA6 | 1.861176824 | 0.017215754 |
| ENSG00000173762 | CD7 | 1.859863899 | 4.91527E−05 |
| ENSG00000175463 | TBC1D10C | 1.857724152 | 9.81643E−07 |
| ENSG00000275395 | FCGBP | 1.856369357 | 0.000393254 |
| ENSG00000115085 | ZAP70 | 1.852736123 | 2.24704E−06 |
| ENSG00000064205 | WISP2 | 1.848100926 | 8.29329E−05 |
| ENSG00000109181 | UGT2B10 | 1.84802304 | 0.007673297 |
| ENSG00000130203 | APOE | 1.847688106 | 2.41717E−06 |
| ENSG00000104814 | MAP4K1 | 1.845552821 | 1.35732E−07 |
| ENSG00000147573 | TRIM55 | 1.844837192 | 0.001479829 |
| ENSG00000168874 | ATOH8 | 1.842839027 | 0.003377482 |
| ENSG00000115523 | GNLY | 1.838744124 | 7.3608E−05 |
| ENSG00000167483 | FAM129C | 1.838608685 | 0.000170872 |
| ENSG00000153086 | ACMSD | 1.836524586 | 0.008566197 |
| ENSG00000100197 | CYP2D6 | 1.829953247 | 3.18759E−05 |
| ENSG00000152969 | JAKMIP1 | 1.825972754 | 9.78322E−07 |
| ENSG00000231852 | CYP21A2 | 1.823917763 | 0.000903462 |
| ENSG00000140279 | DUOX2 | 1.823491986 | 0.001415904 |
| ENSG00000100665 | SERPINA4 | 1.82299406 | 0.016959498 |
| ENSG00000042062 | FAM65C | 1.822157789 | 0.000235797 |
| ENSG00000234906 | APOC2 | 1.821542303 | 0.000209567 |
| ENSG00000214402 | LCNL1 | 1.820340091 | 0.000265435 |
| ENSG00000205358 | MT1H | 1.810540297 | 0.006501419 |
| ENSG00000062524 | LTK | 1.80931079 | 0.000121085 |
| ENSG00000169715 | MT1E | 1.808512373 | 0.000180499 |
| ENSG00000136457 | CHAD | 1.804802571 | 0.001625712 |
| ENSG00000126353 | CCR7 | 1.803144527 | 0.000218428 |
| ENSG00000205364 | MT1M | 1.801744376 | 0.002967506 |
| ENSG00000139194 | RBP5 | 1.801713544 | 0.000565551 |
| ENSG00000182156 | ENPP7 | 1.801390039 | 0.000822806 |
| ENSG00000126759 | CFP | 1.800923596 | 4.41807E−08 |
| ENSG00000167476 | JSRP1 | 1.79911915 | 2.44145E−06 |
| ENSG00000083807 | SLC27A5 | 1.798153138 | 2.76661E−05 |
| ENSG00000145934 | TENM2 | 1.796457258 | 0.000206303 |
| ENSG00000110665 | C11orf21 | 1.794329286 | 0.000202608 |
| ENSG00000188282 | RUFY4 | 1.79268797 | 0.000135845 |
| ENSG00000188488 | SERPINA5 | 1.792614048 | 0.009784986 |
| ENSG00000243955 | GSTA1 | 1.792056651 | 0.039652316 |
| ENSG00000129993 | CBFA2T3 | 1.791485388 | 1.4732E−07 |
| ENSG00000186891 | TNFRSF18 | 1.789424486 | 3.96031E−06 |
| ENSG00000085265 | FCN1 | 1.786385951 | 3.02782E−06 |
| ENSG00000084110 | HAL | 1.786196272 | 0.002407662 |
| ENSG00000183971 | NPW | 1.784741632 | 5.98994E−05 |
| ENSG00000180875 | GREM2 | 1.783092041 | 0.004913974 |
| ENSG00000171385 | KCND3 | 1.783042994 | 3.94309E−05 |
| ENSG00000105352 | CEACAM4 | 1.777033354 | 2.74807E−06 |
| ENSG00000156234 | CXCL13 | 1.773676728 | 0.006035938 |
| ENSG00000158517 | NCF1 | 1.77360529 | 5.40951E−06 |
| ENSG00000008516 | MMP25 | 1.770426818 | 1.41769E−08 |
| ENSG00000073150 | PANX2 | 1.767576887 | 6.59553E−05 |
| ENSG00000068831 | RASGRP2 | 1.764610407 | 8.61631E−07 |
| ENSG00000173531 | MST1 | 1.762894187 | 5.3942E−06 |
| ENSG00000167984 | NLRC3 | 1.762572294 | 2.60598E−07 |
| ENSG00000065320 | NTN1 | 1.759391095 | 0.000799961 |
| ENSG00000244734 | HBB | 1.757053542 | 3.25222E−05 |
| ENSG00000124713 | GNMT | 1.755868292 | 2.97612E−05 |
| ENSG00000162460 | TMEM82 | 1.754068694 | 0.002354755 |
| ENSG00000175003 | SLC22A1 | 1.752026696 | 0.000997327 |
| ENSG00000183837 | PNMA3 | 1.749215247 | 0.000301841 |
| ENSG00000197249 | SERPINA1 | 1.748262514 | 0.004605234 |
| ENSG00000123838 | C4BPA | 1.746288822 | 0.028529213 |
| ENSG00000153404 | PLEKHG4B | 1.745974706 | 0.000654856 |
| ENSG00000157168 | NRG1 | 1.74589881 | 0.003843138 |
| ENSG00000089692 | LAG3 | 1.74502125 | 4.73742E−07 |
| ENSG00000105369 | CD79A | 1.741335371 | 5.69744E−05 |
| ENSG00000180155 | LYNX1 | 1.738389995 | 8.02263E−06 |
| ENSG00000104899 | AMH | 1.738354537 | 2.66697E−05 |
| ENSG00000170476 | MZB1 | 1.734569723 | 0.000194664 |
| ENSG00000128011 | LRFN1 | 1.732331141 | 6.28875E−05 |
| ENSG00000125148 | MT2A | 1.730950364 | 7.13542E−05 |
| ENSG00000275063 | AC233755.1 | 1.729322212 | 0.006172039 |
| ENSG00000055955 | ITIH4 | 1.728177973 | 0.001930577 |
| ENSG00000106538 | RARRES2 | 1.726013818 | 2.76745E−06 |
| ENSG00000156096 | UGT2B4 | 1.725495377 | 0.043548679 |
| ENSG00000146054 | TRIM7 | 1.72495903 | 0.000205372 |
| ENSG00000160856 | FCRL3 | 1.722631911 | 0.000936308 |
| ENSG00000005471 | ABCB4 | 1.719080718 | 0.002526075 |
| ENSG00000172955 | ADH6 | 1.716421501 | 0.020567262 |
| ENSG00000167874 | TMEM88 | 1.715010922 | 0.000109369 |
| ENSG00000054938 | CHRDL2 | 1.713564832 | 0.001977651 |
| ENSG00000088827 | SIGLEC1 | 1.711830538 | 3.12674E−05 |
| ENSG00000227507 | LTB | 1.708765213 | 1.53852E−05 |
| ENSG00000204632 | HLA-G | 1.70456412 | 0.000110853 |
| ENSG00000131203 | IDO1 | 1.703930695 | 0.006901392 |
| ENSG00000137077 | CCL21 | 1.701411468 | 0.001644306 |
| ENSG00000254521 | SIGLEC12 | 1.698716313 | 9.94122E−05 |
| ENSG00000163959 | SLC51A | 1.698224278 | 0.00247345 |
| ENSG00000177272 | KCNA3 | 1.697480235 | 1.94598E−06 |
| ENSG00000139800 | ZIC5 | 1.689500353 | 0.005955401 |
| ENSG00000104059 | FAM189A1 | 1.685372264 | 0.041153881 |
| ENSG00000159423 | ALDH4A1 | 1.685164073 | 1.09581E−05 |
| ENSG00000120949 | TNFRSF8 | 1.67495086 | 7.49844E−06 |
| ENSG00000117215 | PLA2G2D | 1.674749325 | 0.010529388 |
| ENSG00000248144 | ADH1C | 1.669403836 | 0.04953767 |
| ENSG00000180767 | CHST13 | 1.66820775 | 0.004391163 |
| ENSG00000182183 | FAM159A | 1.666525444 | 1.41849E−05 |
| ENSG00000181444 | ZNF467 | 1.666197793 | 1.66452E−07 |
| ENSG00000140961 | OSGIN1 | 1.665643214 | 9.24674E−08 |
| ENSG00000187608 | ISG15 | 1.662520176 | 0.001746152 |
| ENSG00000110436 | SLC1A2 | 1.659988783 | 0.016249596 |
| ENSG00000206384 | COL6A6 | 1.653792903 | 0.000117908 |
| ENSG00000165125 | TRPV6 | 1.652612132 | 0.00368298 |
| ENSG00000225968 | ELFN1 | 1.651208878 | 1.47141E−06 |
| ENSG00000254788 | CKLF-CMTM1 | 1.64958526 | 9.08287E−07 |
| ENSG00000057593 | F7 | 1.647953549 | 0.024882783 |
| ENSG00000168477 | TNXB | 1.646578783 | 7.81703E−07 |
| ENSG00000138823 | MTTP | 1.6461343 | 0.016189652 |
| ENSG00000189410 | SH2D5 | 1.643968913 | 0.002705987 |
| ENSG00000166823 | MESP1 | 1.642477892 | 0.000445012 |
| ENSG00000185338 | SOCS1 | 1.639791845 | 6.65962E−07 |
| ENSG00000140368 | PSTPIP1 | 1.637320915 | 4.81596E−07 |
| ENSG00000015520 | NPC1L1 | 1.635491238 | 0.001045583 |
| ENSG00000167772 | ANGPTL4 | 1.634398094 | 1.98855E−05 |
| ENSG00000131386 | GALNT15 | 1.633087575 | 9.47614E−06 |
| ENSG00000166106 | ADAMTS15 | 1.632711035 | 0.00114866 |
| ENSG00000109906 | ZBTB16 | 1.629223454 | 0.0061776 |
| ENSG00000116690 | PRG4 | 1.628432381 | 0.007704889 |
| ENSG00000134571 | MYBPC3 | 1.628364503 | 3.18019E−05 |
| ENSG00000132704 | FCRL2 | 1.627384887 | 0.000283186 |
| ENSG00000173578 | XCR1 | 1.626175318 | 0.001112116 |
| ENSG00000147443 | DOK2 | 1.621123096 | 4.19841E−06 |
| ENSG00000161653 | NAGS | 1.62093036 | 6.88702E−07 |
| ENSG00000168546 | GFRA2 | 1.618891903 | 0.002653066 |
| ENSG00000123843 | C4BPB | 1.615265955 | 6.72693E−05 |
| ENSG00000185499 | MUC1 | 1.614882918 | 0.003146832 |
| ENSG00000089012 | SIRPG | 1.614629752 | 0.000410911 |
| ENSG00000180644 | PRF1 | 1.60984838 | 0.000353497 |
| ENSG00000130487 | KLHDC7B | 1.609401829 | 2.95717E−06 |
| ENSG00000130812 | ANGPTL6 | 1.609247334 | 0.000203163 |
| ENSG00000271503 | CCL5 | 1.608717196 | 6.72864E−06 |
| ENSG00000174326 | SLC16A11 | 1.603389799 | 0.000400232 |
| EN5G00000164850 | GPER1 | 1.602617044 | 0.000628996 |
| ENSG00000198417 | MT1F | 1.602377217 | 8.1754E−06 |
| EN5G00000280789 | PAGR1 | 1.602104346 | 3.03492E−06 |
| EN5G00000129946 | SHC2 | 1.600778342 | 2.53183E−07 |
| ENSG00000141574 | SECTM1 | 1.598597388 | 1.16026E−05 |
| ENSG00000140274 | DUOXA2 | 1.597244432 | 0.006036338 |
| ENSG00000124256 | ZBP1 | 1.593895754 | 5.12655E−05 |
| ENSG00000176720 | BOK | 1.59208335 | 1.61964E−08 |
| ENSG00000180096 | SEPT1 | 1.590954812 | 6.12664E−07 |
| ENSG00000171234 | UGT2B7 | 1.588703972 | 0.004784474 |
| ENSG00000133246 | PRAM1 | 1.588436941 | 1.65774E−08 |
| ENSG00000007908 | SELE | 1.587950519 | 0.004511819 |
| ENSG00000096996 | IL12RB1 | 1.585877225 | 5.33991E−07 |
| ENSG00000136490 | LIMD2 | 1.585202182 | 9.87742E−08 |
| ENSG00000166035 | LIPC | 1.585189629 | 0.004337306 |
| ENSG00000115226 | FNDC4 | 1.584730002 | 2.62044E−05 |
| ENSG00000163508 | EOMES | 1.584296602 | 0.000129922 |
| ENSG00000198610 | AKR1C4 | 1.582907202 | 0.0223585 |
| ENSG00000167680 | SEMA6B | 1.581726298 | 2.56029E−09 |
| ENSG00000118804 | FAM47E−STBD1 | 1.581707821 | 6.61276E−07 |
| ENSG00000092607 | TBX15 | 1.581137159 | 0.002725107 |
| ENSG00000188536 | HBA2 | 1.580952966 | 0.000145343 |
| ENSG00000258472 | RP11-192H23.4 | 1.580107392 | 4.78837E−08 |
| ENSG00000203883 | SOX18 | 1.579196193 | 9.0363E−09 |
| ENSG00000105609 | LILRB5 | 1.579144678 | 0.002356928 |
| ENSG00000099377 | HSD3B7 | 1.578351001 | 2.01349E−09 |
| ENSG00000187260 | WDR86 | 1.578246045 | 2.72846E−06 |
| ENSG00000139193 | CD27 | 1.576787928 | 0.000167422 |
| ENSG00000108950 | FAM20A | 1.569323478 | 5.89966E−05 |
| ENSG00000142512 | SIGLEC10 | 1.558451603 | 1.16876E−06 |
| ENSG00000186994 | KANK3 | 1.558328138 | 1.61808E−07 |
| ENSG00000134363 | FST | 1.558053549 | 0.012573902 |
| ENSG00000179348 | GATA2 | 1.556181299 | 6.31175E−06 |
| ENSG00000064201 | TSPAN32 | 1.556158659 | 0.004222363 |
| ENSG00000186642 | PDE2A | 1.555302924 | 8.08213E−06 |
| ENSG00000143196 | DPT | 1.55438042 | 0.001172053 |
| ENSG00000104972 | LILRB1 | 1.552571458 | 3.1956E−07 |
| ENSG00000107147 | KCNT1 | 1.550415416 | 0.023504834 |
| ENSG00000184979 | USP18 | 1.548316322 | 2.28753E−05 |
| ENSG00000122223 | CD244 | 1.545519949 | 0.00024182 |
| ENSG00000158445 | KCNB1 | 1.544809945 | 0.013214337 |
| ENSG00000160185 | UBASH3A | 1.53615082 | 0.000194244 |
| ENSG00000102879 | CORO1A | 1.53399304 | 4.94145E−07 |
| ENSG00000185739 | SRL | 1.532845494 | 0.000197044 |
| ENSG00000204642 | HLA-F | 1.532253799 | 9.84429E−06 |
| ENSG00000180871 | CXCR2 | 1.531703143 | 2.47874E−05 |
| ENSG00000169083 | AR | 1.531236164 | 0.002033423 |
| ENSG00000187116 | LILRA5 | 1.531201843 | 0.000166288 |
| ENSG00000146192 | FGD2 | 1.530806368 | 3.709E−08 |
| ENSG00000025708 | TYMP | 1.526764551 | 6.36576E−07 |
| ENSG00000170458 | CD14 | 1.523186839 | 1.22091E−05 |
| ENSG00000182853 | VMO1 | 1.520279287 | 0.000413367 |
| ENSG00000145217 | SLC26A1 | 1.519696327 | 5.34812E−05 |
| ENSG00000162676 | GFI1 | 1.519455566 | 9.85814E−06 |
| ENSG00000130270 | ATP8B3 | 1.51752289 | 8.04517E−06 |
| ENSG00000204099 | NEU4 | 1.514399269 | 0.000284209 |
| ENSG00000166825 | ANPEP | 1.514176695 | 0.000156096 |
| ENSG00000104974 | LILRA1 | 1.513547989 | 3.66484E−05 |
| ENSG00000135094 | SDS | 1.512678166 | 0.000420758 |
| ENSG00000159761 | C16orf86 | 1.511460887 | 1.41334E−05 |
| ENSG00000197558 | SSPO | 1.51104646 | 4.27128E−05 |
| ENSG00000124191 | TOX2 | 1.510051746 | 1.65239E−06 |
| ENSG00000009694 | TENM1 | 1.50553148 | 0.010618274 |
| ENSG00000272636 | DOC2B | 1.501787011 | 0.000706486 |
| ENSG00000160883 | HK3 | 1.501399726 | 2.90159E−06 |
| ENSG00000136305 | CIDEB | 1.501381456 | 4.48948E−06 |
| ENSG00000113088 | GZMK | 1.500758813 | 0.002084863 |
| ENSG00000167992 | VWCE | 1.49937759 | 0.003394657 |
| ENSG00000103522 | IL21R | 1.497217502 | 2.3103E−05 |
| ENSG00000130208 | APOC1 | 1.496848549 | 0.003538667 |
| ENSG00000180509 | KCNE1 | 1.493422865 | 0.000223845 |
| ENSG00000185669 | SNAI3 | 1.490036928 | 0.000170417 |
| ENSG00000107159 | CA9 | 1.48921772 | 0.012780816 |
| ENSG00000243649 | CFB | 1.48806145 | 0.000145343 |
| ENSG00000188305 | C19orf35 | 1.487580548 | 7.42914E−05 |
| ENSG00000160801 | PTH1R | 1.487389486 | 0.001178722 |
| ENSG00000196090 | PTPRT | 1.486605225 | 0.001355525 |
| ENSG00000188517 | COL25A1 | 1.485873647 | 0.001531804 |
| ENSG00000254709 | IGLL5 | 1.485808498 | 0.001990999 |
| ENSG00000134962 | KLB | 1.48180961 | 0.005137395 |
| ENSG00000159307 | SCUBE1 | 1.481616838 | 0.000256796 |
| ENSG00000107742 | SPOCK2 | 1.480342214 | 1.13584E−05 |
| ENSG00000162747 | FCGR3B | 1.479926439 | 0.000394616 |
| ENSG00000184557 | SOCS3 | 1.479596519 | 6.52137E−06 |
| ENSG00000173599 | PC | 1.479467656 | 1.49065E−05 |
| ENSG00000244474 | UGT1A4 | 1.479087428 | 0.032746482 |
| ENSG00000187889 | C1orf168 | 1.47363403 | 0.008517697 |
| ENSG00000105509 | HAS1 | 1.473448956 | 0.000911515 |
| ENSG00000182885 | GPR97 | 1.472956397 | 7.99165E−05 |
| ENSG00000150656 | CNDP1 | 1.472736717 | 0.004443654 |
| ENSG00000160932 | LY6E | 1.470833109 | 3.50003E−06 |
| ENSG00000115457 | IGFBP2 | 1.468493667 | 0.002859981 |
| ENSG00000158473 | CD1D | 1.465852202 | 1.49296E−05 |
| ENSG00000127220 | ABHD8 | 1.463150875 | 7.6543E−06 |
| ENSG00000074660 | SCARF1 | 1.463099519 | 6.50364E−10 |
| ENSG00000162572 | SCNN1D | 1.461799829 | 3.78361E−06 |
| ENSG00000156127 | BATF | 1.460660543 | 1.01302E−05 |
| ENSG00000135447 | PPP1R1A | 1.459770857 | 0.042126222 |
| ENSG00000105246 | EBI3 | 1.458884775 | 0.000671819 |
| ENSG00000131042 | LILRB2 | 1.458348537 | 1.08064E−05 |
| ENSG00000182472 | CAPN12 | 1.45730071 | 0.0001162 |
| ENSG00000106123 | EPHB6 | 1.456289268 | 0.00039008 |
| ENSG00000169862 | CTNND2 | 1.455089876 | 0.001836041 |
| ENSG00000196092 | PAX5 | 1.454324621 | 0.049558989 |
| ENSG00000119535 | CSF3R | 1.453178275 | 2.59486E−06 |
| ENSG00000172889 | EGFL7 | 1.451660278 | 6.27202E−09 |
| ENSG00000100628 | ASB2 | 1.447979187 | 0.000189938 |
| ENSG00000047617 | ANO2 | 1.447971918 | 0.000188114 |
| ENSG00000188290 | HES4 | 1.44527957 | 7.46094E−06 |
| ENSG00000108798 | ABI3 | 1.445012041 | 5.35764E−08 |
| ENSG00000167701 | GPT | 1.444221162 | 1.88403E−05 |
| ENSG00000158571 | PFKFB1 | 1.443803865 | 0.001480445 |
| ENSG00000188820 | FAM26F | 1.443616952 | 0.000454748 |
| ENSG00000136546 | SCN7A | 1.441838627 | 0.013750712 |
| ENSG00000182511 | FES | 1.441405484 | 4.76048E−07 |
| ENSG00000135744 | AGT | 1.440999485 | 0.010848761 |
| ENSG00000157551 | KCNJ15 | 1.439531321 | 0.001364319 |
| ENSG00000204682 | CASC10 | 1.439011293 | 0.000253335 |
| ENSG00000005844 | ITGAL | 1.438845566 | 4.87749E−06 |
| ENSG00000087258 | GNAO1 | 1.438412397 | 6.97306E−05 |
| ENSG00000043039 | BARX2 | 1.43741433 | 0.005788686 |
| ENSG00000182162 | P2RY8 | 1.437004206 | 2.87771E−06 |
| ENSG00000029534 | ANK1 | 1.436837917 | 1.12422E−05 |
| ENSG00000144891 | AGTR1 | 1.436715397 | 0.020393409 |
| ENSG00000101605 | MYOM1 | 1.435106967 | 0.000223748 |
| ENSG00000189350 | FAM179A | 1.434943722 | 2.53473E−06 |
| ENSG00000100385 | IL2RB | 1.434556638 | 4.96027E−06 |
| ENSG00000166816 | LDHD | 1.433738263 | 0.000243597 |
| ENSG00000142156 | COL6A1 | 1.433101784 | 4.66917E−07 |
| ENSG00000010327 | STAB1 | 1.431917332 | 2.04579E−07 |
| ENSG00000036473 | OTC | 1.430512453 | 0.045203191 |
| ENSG00000154451 | GBPS | 1.430340878 | 0.000634328 |
| ENSG00000132938 | MTUS2 | 1.425975562 | 0.011604099 |
| ENSG00000007933 | FMO3 | 1.423946685 | 0.001203078 |
| ENSG00000149131 | SERPING1 | 1.422621932 | 0.000396357 |
| ENSG00000172828 | CES3 | 1.419384028 | 0.000655981 |
| ENSG00000171608 | PIK3CD | 1.41905466 | 3.53588E−07 |
| ENSG00000064300 | NGFR | 1.417992298 | 0.000350911 |
| ENSG00000118514 | ALDH8A1 | 1.417317924 | 0.006378936 |
| ENSG00000167286 | CD3D | 1.416497792 | 0.001019335 |
| ENSG00000135519 | KCNH3 | 1.415735452 | 0.00145232 |
| ENSG00000169856 | ONECUT1 | 1.412136864 | 0.010666397 |
| ENSG00000160870 | CYP3A7 | 1.408712629 | 0.024446748 |
| ENSG00000050730 | TNIP3 | 1.406955332 | 0.000669154 |
| ENSG00000140538 | NTRK3 | 1.40651603 | 0.000794662 |
| ENSG00000171759 | PAH | 1.404407736 | 0.025034659 |
| ENSG00000130303 | BST2 | 1.403284722 | 3.13548E−05 |
| ENSG00000124253 | PCK1 | 1.402736368 | 0.028467751 |
| ENSG00000135439 | AGAP2 | 1.399151069 | 6.57005E−06 |
| ENSG00000137265 | IRF4 | 1.398129085 | 0.000204594 |
| ENSG00000139144 | PIK3C2G | 1.395418582 | 0.039355522 |
| ENSG00000106333 | PCOLCE | 1.395255366 | 2.75062E−08 |
| ENSG00000163239 | TDRD10 | 1.395113098 | 8.1805E−05 |
| ENSG00000140853 | NLRC5 | 1.394013874 | 1.3912E−05 |
| ENSG00000159753 | RLTPR | 1.392255618 | 0.001169798 |
| ENSG00000175489 | LRRC25 | 1.391156103 | 2.68833E−06 |
| ENSG00000135114 | OASL | 1.390001261 | 0.00059017 |
| ENSG00000185215 | TNFAIP2 | 1.389383822 | 4.1407E−06 |
| ENSG00000139344 | AMDHD1 | 1.387981179 | 0.03684003 |
| ENSG00000153093 | ACOXL | 1.387844601 | 0.005899064 |
| ENSG00000198821 | CD247 | 1.387820922 | 0.000154722 |
| ENSG00000169085 | C8orf46 | 1.387524713 | 0.006875701 |
| ENSG00000100368 | CSF2RB | 1.384962825 | 7.8841E−07 |
| ENSG00000167895 | TMC8 | 1.384767221 | 3.27719E−05 |
| ENSG00000155657 | TTN | 1.383979127 | 0.000246024 |
| ENSG00000141622 | RNF165 | 1.383144496 | 0.003791615 |
| ENSG00000162552 | WNT4 | 1.378701388 | 4.52634E−05 |
| ENSG00000185905 | C16orf54 | 1.37848352 | 2.21048E−05 |
| ENSG00000176383 | B3GNT4 | 1.378040428 | 0.000189887 |
| ENSG00000110777 | POU2AF1 | 1.376583458 | 0.001575907 |
| ENSG00000169248 | CXCL11 | 1.376451509 | 0.016253559 |
| ENSG00000183615 | FAM167B | 1.374963365 | 6.58579E−06 |
| ENSG00000123329 | ARHGAP9 | 1.374840469 | 2.56953E−06 |
| ENSG00000205436 | EXOC3L4 | 1.374064892 | 2.26388E−08 |
| ENSG00000143869 | GDF7 | 1.373721574 | 0.001414276 |
| ENSG00000042781 | USH2A | 1.373645934 | 0.007969349 |
| ENSG00000166428 | PLD4 | 1.373505959 | 3.55186E−05 |
| ENSG00000020633 | RUNX3 | 1.371645797 | 1.88926E−07 |
| ENSG00000174175 | SELP | 1.370020088 | 0.000587098 |
| ENSG00000148803 | FUOM | 1.367938366 | 2.11789E−06 |
| ENSG00000126709 | IFI6 | 1.366363786 | 0.000985708 |
| ENSG00000177807 | KCNJ10 | 1.366234564 | 0.006225239 |
| ENSG00000269404 | SPIB | 1.364097082 | 0.00156549 |
| ENSG00000160323 | ADAMTS13 | 1.363633887 | 9.78244E−06 |
| ENSG00000169710 | FASN | 1.361278264 | 4.1289E−08 |
| ENSG00000125910 | S1PR4 | 1.360852503 | 0.00025185 |
| ENSG00000110719 | TCIRG1 | 1.358038633 | 4.78837E−08 |
| ENSG00000134539 | KLRD1 | 1.355600234 | 0.000299136 |
| ENSG00000127954 | STEAP4 | 1.354701727 | 0.001339098 |
| ENSG00000104903 | LYL1 | 1.353724395 | 1.84304E−07 |
| ENSG00000162894 | FAIM3 | 1.352538626 | 0.000188982 |
| ENSG00000169418 | NPR1 | 1.352033962 | 0.00015855 |
| ENSG00000158786 | PLA2G2F | 1.351553394 | 0.005457894 |
| EN5G00000077984 | CST7 | 1.350919972 | 0.003159923 |
| ENSG00000137877 | SPTBN5 | 1.350588945 | 0.00110595 |
| EN5G00000159189 | C1QC | 1.3498868 | 1.58024E−05 |
| ENSG00000127528 | KLF2 | 1.349131569 | 1.46824E−06 |
| EN5G00000015285 | WAS | 1.348736349 | 3.7502E−06 |
| EN5G00000169750 | RAC3 | 1.347394699 | 1.40004E−05 |
| ENSG00000117643 | MAN1C1 | 1.346757216 | 3.86305E−06 |
| ENSG00000159403 | C1R | 1.346255133 | 0.000342542 |
| ENSG00000160326 | SLC2A6 | 1.344283779 | 2.73676E−06 |
| ENSG00000123360 | PDE1B | 1.343772761 | 4.91224E−06 |
| ENSG00000112936 | C7 | 1.343573236 | 0.024446748 |
| ENSG00000135063 | FAM189A2 | 1.342142585 | 0.002592105 |
| ENSG00000116176 | TPSG1 | 1.340310765 | 0.005059233 |
| ENSG00000189056 | RELN | 1.339749907 | 0.00342347 |
| ENSG00000101445 | PPP1R16B | 1.338140508 | 1.243E−05 |
| ENSG00000169903 | TM4SF4 | 1.33728372 | 0.023002889 |
| ENSG00000154133 | ROBO4 | 1.335671373 | 4.17897E−07 |
| ENSG00000130489 | SCO2 | 1.331302036 | 1.01974E−07 |
| ENSG00000171049 | FPR2 | 1.3312927 | 0.003415446 |
| ENSG00000198753 | PLXNB3 | 1.330747625 | 0.012771388 |
| EN5G00000204577 | LILRB3 | 1.330242639 | 2.17679E−05 |
| ENSG00000179044 | EXOC3L1 | 1.329930893 | 1.02312E−06 |
| ENSG00000105227 | PRX | 1.326965637 | 1.21468E−06 |
| ENSG00000163395 | IGFN1 | 1.326541078 | 0.010632208 |
| ENSG00000186827 | TNFRSF4 | 1.322769209 | 1.07579E−05 |
| ENSG00000157734 | SNX22 | 1.32193201 | 0.00142706 |
| ENSG00000144648 | ACKR2 | 1.321631463 | 9.88382E−05 |
| EN5G00000089820 | ARHGAP4 | 1.32092602 | 7.86819E−06 |
| ENSG00000175262 | C1orf127 | 1.320722032 | 0.001786508 |
| ENSG00000152784 | PRDM8 | 1.31985205 | 7.7156E−05 |
| ENSG00000198598 | MMP17 | 1.318597563 | 0.036850845 |
| ENSG00000172673 | THEMIS | 1.318181784 | 0.000493136 |
| ENSG00000013725 | CD6 | 1.31619461 | 5.66554E−05 |
| ENSG00000241635 | UGT1A1 | 1.31614142 | 0.028644359 |
| ENSG00000143028 | SYPL2 | 1.315335386 | 0.000128952 |
| ENSG00000122490 | PQLC1 | 1.3140364 | 1.54159E−09 |
| ENSG00000137825 | ITPKA | 1.313445676 | 0.000866035 |
| ENSG00000167508 | MVD | 1.312584378 | 2.75893E−08 |
| EN5G00000006638 | TBXA2R | 1.311844461 | 5.62888E−06 |
| ENSG00000166897 | ELFN2 | 1.31073156 | 0.031142854 |
| ENSG00000132386 | SERPINF1 | 1.310719711 | 0.000210727 |
| ENSG00000186907 | RTN4RL2 | 1.310690555 | 3.01527E−05 |
| ENSG00000135929 | CYP27A1 | 1.309960107 | 4.05725E−06 |
| ENSG00000188157 | AGRN | 1.309823267 | 3.79E−08 |
| ENSG00000198851 | CD3E | 1.309233136 | 0.000205617 |
| ENSG00000204444 | APOM | 1.308606774 | 0.008652124 |
| ENSG00000099958 | DERL3 | 1.307484028 | 0.000674201 |
| ENSG00000171130 | ATP6V0E2 | 1.303641323 | 3.47728E−05 |
| ENSG00000122122 | SASH3 | 1.303562183 | 1.8073E−06 |
| ENSG00000122971 | ACADS | 1.303201091 | 7.64985E−10 |
| ENSG00000158717 | RNF166 | 1.301732784 | 1.94568E−09 |
| ENSG00000095321 | CRAT | 1.299698193 | 4.79614E−07 |
| ENSG00000168995 | SIGLEC7 | 1.299681 | 0.000168534 |
| ENSG00000180447 | GAS1 | 1.299034643 | 0.000512172 |
| ENSG00000092929 | UNC13D | 1.298770733 | 0.000302272 |
| ENSG00000169252 | ADRB2 | 1.296270277 | 0.000337699 |
| ENSG00000196218 | RYR1 | 1.294363789 | 0.001141674 |
| ENSG00000182871 | COL18A1 | 1.294145625 | 5.30035E−06 |
| ENSG00000081479 | LRP2 | 1.292962974 | 0.014450832 |
| ENSG00000141338 | ABCA8 | 1.292590541 | 0.016007005 |
| ENSG00000179403 | VWA1 | 1.291929758 | 1.09581E−05 |
| ENSG00000124334 | IL9R | 1.291031644 | 0.001017975 |
| ENSG00000161405 | IKZF3 | 1.290585177 | 0.000264059 |
| ENSG00000126264 | HCST | 1.290384181 | 0.000289026 |
| ENSG00000075399 | VPS9D1 | 1.289396862 | 8.96158E−09 |
| ENSG00000076662 | ICAM3 | 1.289393988 | 1.75721E−08 |
| ENSG00000170214 | ADRA1B | 1.288566885 | 0.004623661 |
| ENSG00000173372 | C1QA | 1.288030022 | 0.000419427 |
| ENSG00000185507 | IRF7 | 1.287857093 | 8.25769E−07 |
| ENSG00000197253 | TPSB2 | 1.287847104 | 0.004754687 |
| ENSG00000169220 | RGS14 | 1.286980501 | 1.59818E−06 |
| ENSG00000119714 | GPR68 | 1.286839432 | 0.000234779 |
| ENSG00000089558 | KCNH4 | 1.286186482 | 0.003005539 |
| ENSG00000105639 | JAK3 | 1.28579567 | 3.54358E−06 |
| ENSG00000148346 | LCN2 | 1.285775626 | 0.022139553 |
| ENSG00000070985 | TRPM5 | 1.284113725 | 0.006536381 |
| ENSG00000274286 | ADRA2B | 1.283875269 | 0.006677597 |
| ENSG00000126882 | FAM78A | 1.283087195 | 1.02284E−06 |
| ENSG00000185340 | GAS2L1 | 1.282914186 | 5.31352E−09 |
| ENSG00000205078 | SYCE1L | 1.28266468 | 0.000114747 |
| ENSG00000125551 | PLGLB2 | 1.282193221 | 0.017770393 |
| ENSG00000021461 | CYP3A43 | 1.282118341 | 0.024675843 |
| ENSG00000122224 | LY9 | 1.279929863 | 0.000453 |
| ENSG00000159231 | CBR3 | 1.278960636 | 0.000476315 |
| ENSG00000196700 | ZNF512B | 1.278867404 | 4.6102E−06 |
| ENSG00000135898 | GPR55 | 1.27861113 | 0.029853596 |
| ENSG00000138030 | KHK | 1.278576173 | 3.99403E−05 |
| ENSG00000145649 | GZMA | 1.276984142 | 0.009737375 |
| ENSG00000185386 | MAPK11 | 1.276319561 | 5.04464E−07 |
| ENSG00000169738 | DCXR | 1.2753609 | 1.5413E−06 |
| ENSG00000142173 | COL6A2 | 1.275131727 | 1.28826E−05 |
| ENSG00000173581 | CCDC106 | 1.272140387 | 5.49353E−06 |
| ENSG00000185347 | C14orf80 | 1.270216159 | 3.19395E−08 |
| ENSG00000196169 | KIF19 | 1.270058837 | 0.010790431 |
| ENSG00000178685 | PARP10 | 1.269021594 | 2.26146E−09 |
| ENSG00000121807 | CCR2 | 1.268453693 | 0.001794151 |
| ENSG00000163492 | CCDC141 | 1.268330074 | 0.002739587 |
| ENSG00000100079 | LGALS2 | 1.268198947 | 0.029705602 |
| ENSG00000185924 | RTN4RL1 | 1.267257666 | 0.04679419 |
| ENSG00000116661 | FBXO2 | 1.266954006 | 0.013783534 |
| ENSG00000198959 | TGM2 | 1.266576781 | 1.43366E−05 |
| ENSG00000012124 | CD22 | 1.266360544 | 8.00988E−05 |
| ENSG00000141506 | PIK3R5 | 1.266229852 | 8.88741E−06 |
| ENSG00000105538 | RASIP1 | 1.266170986 | 4.78102E−05 |
| ENSG00000122025 | FLT3 | 1.26489447 | 0.003175215 |
| ENSG00000103254 | FAM173A | 1.264595232 | 5.26363E−09 |
| ENSG00000156886 | ITGAD | 1.2610692 | 0.002119374 |
| ENSG00000177706 | FAM20C | 1.260244979 | 1.25398E−06 |
| ENSG00000188064 | WNT7B | 1.260152118 | 0.009699119 |
| ENSG00000132622 | HSPA12B | 1.259050734 | 1.99475E−06 |
| ENSG00000144331 | ZNF385B | 1.258917589 | 0.038403342 |
| ENSG00000168140 | VASN | 1.256361222 | 1.40715E−06 |
| ENSG00000173227 | SYT12 | 1.255402275 | 0.000952545 |
| ENSG00000128283 | CDC42EP1 | 1.254646849 | 1.19128E−08 |
| ENSG00000144649 | FAM198A | 1.253770609 | 0.005101862 |
| ENSG00000108242 | CYP2C18 | 1.252384579 | 0.049445495 |
| ENSG00000243414 | TICAM2 | 1.251576637 | 3.39174E−05 |
| ENSG00000183186 | C2CD4C | 1.250886399 | 0.000615976 |
| ENSG00000153563 | CD8A | 1.250678303 | 0.000488152 |
| ENSG00000049089 | COL9A2 | 1.250591387 | 0.001579205 |
| ENSG00000157927 | RADIL | 1.248223524 | 0.000100417 |
| ENSG00000133048 | CHI3L1 | 1.245811801 | 0.021325178 |
| ENSG00000161618 | ALDH16A1 | 1.244370735 | 8.4127E−09 |
| ENSG00000149781 | FERMT3 | 1.243405838 | 1.43792E−07 |
| ENSG00000123689 | G0S2 | 1.242808848 | 0.00273989 |
| ENSG00000250510 | GPR162 | 1.240962742 | 8.85735E−05 |
| ENSG00000087237 | CETP | 1.240660267 | 0.011419735 |
| ENSG00000009950 | MLXIPL | 1.237482445 | 0.001141373 |
| ENSG00000165886 | UBTD1 | 1.236166956 | 3.20006E−07 |
| ENSG00000214063 | TSPAN4 | 1.235756555 | 4.7405E−06 |
| ENSG00000143382 | ADAMTSL4 | 1.235690029 | 1.66736E−05 |
| ENSG00000196358 | NTNG2 | 1.234298082 | 3.33722E−05 |
| ENSG00000179761 | PIPOX | 1.232024601 | 0.007340453 |
| ENSG00000184922 | FMNL1 | 1.230287144 | 1.9695E−06 |
| ENSG00000172602 | RND1 | 1.228522457 | 0.001807763 |
| ENSG00000115255 | REEP6 | 1.228306019 | 0.000846713 |
| ENSG00000213145 | CRIP1 | 1.226925682 | 0.000207155 |
| ENSG00000137078 | SIT1 | 1.22673322 | 0.004366955 |
| ENSG00000232810 | TNF | 1.226621359 | 0.001125236 |
| ENSG00000108551 | RASD1 | 1.225543767 | 0.011690412 |
| ENSG00000244242 | IFITM10 | 1.224034083 | 3.98716E−06 |
| ENSG00000198691 | ABCA4 | 1.223798301 | 0.009398914 |
| ENSG00000163874 | ZC3H12A | 1.223246451 | 2.28949E−07 |
| ENSG00000143878 | RHOB | 1.221639417 | 1.68701E−05 |
| ENSG00000184113 | CLDN5 | 1.218254387 | 0.000542657 |
| ENSG00000104894 | CD37 | 1.217712684 | 5.74712E−05 |
| ENSG00000128016 | ZFP36 | 1.216595371 | 5.72502E−06 |
| ENSG00000197471 | SPN | 1.215716502 | 9.0198E−06 |
| ENSG00000166289 | PLEKHF1 | 1.213445619 | 9.3501E−05 |
| ENSG00000111913 | FAM65B | 1.213112434 | 8.25317E−05 |
| ENSG00000107736 | CDH23 | 1.21124224 | 5.45205E−05 |
| ENSG00000141497 | ZMYND15 | 1.210263071 | 2.68191E−06 |
| ENSG00000102032 | RENBP | 1.209315238 | 2.08456E−05 |
| ENSG00000110448 | CD5 | 1.208394398 | 0.001390494 |
| ENSG00000235568 | NFAM1 | 1.208350739 | 5.73402E−07 |
| ENSG00000205795 | CYS1 | 1.208277485 | 0.001457831 |
| ENSG00000137857 | DUOX1 | 1.208233759 | 0.007487157 |
| ENSG00000132329 | RAMP1 | 1.207596418 | 0.000151863 |
| ENSG00000213203 | GIMAP1 | 1.206650397 | 0.000188673 |
| ENSG00000159335 | PTMS | 1.203464099 | 1.03003E−07 |
| ENSG00000081052 | COL4A4 | 1.202960631 | 0.000201211 |
| ENSG00000168994 | PXDC1 | 1.202678576 | 4.72077E−05 |
| ENSG00000171777 | RASGRP4 | 1.202265459 | 2.81665E−05 |
| ENSG00000160654 | CD3G | 1.20196118 | 0.000365209 |
| ENSG00000080573 | COL5A3 | 1.200805737 | 6.07373E−06 |
| ENSG00000091831 | ESR1 | 1.200583376 | 0.000774744 |
| ENSG00000171303 | KCNK3 | 1.20014995 | 0.005928289 |
| ENSG00000185274 | WBSCR17 | 1.19994384 | 0.000845441 |
| ENSG00000175287 | PHYHD1 | 1.198776424 | 0.000837289 |
| ENSG00000066336 | SPI1 | 1.198381682 | 4.26718E−06 |
| ENSG00000132837 | DMGDH | 1.19707948 | 0.041104751 |
| ENSG00000163519 | TRAT1 | 1.196156055 | 0.018935271 |
| ENSG00000171700 | RGS19 | 1.196010764 | 9.53999E−07 |
| ENSG00000187583 | PLEKHN1 | 1.195422572 | 1.02071E−05 |
| ENSG00000179583 | CIITA | 1.195282259 | 0.000271761 |
| ENSG00000197019 | SERTAD1 | 1.193431276 | 1.29925E−07 |
| ENSG00000144596 | GRIP2 | 1.192904741 | 0.000998238 |
| ENSG00000151651 | ADAM8 | 1.191465884 | 0.000214876 |
| ENSG00000167549 | CORO6 | 1.191456288 | 0.001594523 |
| ENSG00000139899 | CBLN3 | 1.190828966 | 1.18604E−05 |
| ENSG00000130052 | STARD8 | 1.190480825 | 1.85432E−05 |
| ENSG00000168993 | CPLX1 | 1.190376407 | 0.002199554 |
| ENSG00000125089 | SH3TC1 | 1.189769624 | 1.16481E−06 |
| ENSG00000181035 | SLC25A42 | 1.189715251 | 2.93113E−07 |
| ENSG00000095370 | SH2D3C | 1.189470049 | 6.05052E−07 |
| ENSG00000165716 | FAM69B | 1.188838195 | 0.012226404 |
| ENSG00000164344 | KLKB1 | 1.188755753 | 0.029216703 |
| ENSG00000140511 | HAPLN3 | 1.188670216 | 3.4617E−05 |
| ENSG00000204301 | NOTCH4 | 1.186538696 | 2.33235E−08 |
| ENSG00000126246 | IGFLR1 | 1.186443876 | 0.001222 |
| ENSG00000176533 | GNG7 | 1.185998076 | 0.000123399 |
| ENSG00000100342 | APOL1 | 1.185024196 | 0.000193231 |
| ENSG00000100918 | REC8 | 1.184690164 | 3.76756E−08 |
| ENSG00000127129 | EDN2 | 1.184507462 | 0.014275303 |
| ENSG00000235098 | ANKRD65 | 1.184396682 | 0.003096456 |
| ENSG00000114923 | SLC4A3 | 1.183439405 | 0.000723447 |
| ENSG00000091592 | NLRP1 | 1.182959918 | 3.84042E−05 |
| ENSG00000205560 | CPT1B | 1.182632796 | 1.32115E−05 |
| ENSG00000146094 | DOK3 | 1.181661839 | 3.28488E−07 |
| ENSG00000114378 | HYAL1 | 1.181384394 | 0.000113442 |
| ENSG00000130201 | EXOC3L2 | 1.181023012 | 8.10527E−06 |
| ENSG00000066735 | KIF26A | 1.180397791 | 0.019796758 |
| ENSG00000177294 | FBXO39 | 1.179991347 | 0.000556418 |
| ENSG00000172236 | TPSAB1 | 1.178949031 | 0.00366878 |
| ENSG00000184985 | SORCS2 | 1.178037587 | 0.001641863 |
| ENSG00000175920 | DOK7 | 1.176041109 | 0.011281996 |
| ENSG00000100299 | ARSA | 1.17508065 | 1.94568E−09 |
| ENSG00000225614 | ZNF469 | 1.175078359 | 1.37323E−05 |
| ENSG00000173267 | SNCG | 1.174670869 | 0.003930678 |
| ENSG00000170955 | PRKCDBP | 1.174293625 | 1.1794E−05 |
| ENSG00000158050 | DUSP2 | 1.173650189 | 0.002134271 |
| ENSG00000102962 | CCL22 | 1.173459271 | 0.015542899 |
| ENSG00000205784 | ARRDC5 | 1.172873645 | 0.001814455 |
| ENSG00000134463 | ECHDC3 | 1.172738848 | 0.03883662 |
| ENSG00000138131 | LOXL4 | 1.172728047 | 0.004815049 |
| ENSG00000115590 | IL1R2 | 1.172239851 | 0.000617956 |
| ENSG00000173210 | ABLIM3 | 1.172167801 | 0.003996414 |
| ENSG00000099860 | GADD45B | 1.17198885 | 0.000336154 |
| ENSG00000138623 | SEMA7A | 1.171977056 | 0.001270896 |
| ENSG00000165644 | COMTD1 | 1.17157671 | 3.14594E−06 |
| ENSG00000138964 | PARVG | 1.170941194 | 2.2748E−05 |
| ENSG00000261221 | ZNF865 | 1.17089339 | 1.17477E−10 |
| ENSG00000129450 | SIGLEC9 | 1.17087874 | 1.09789E−05 |
| ENSG00000173369 | C1QB | 1.170411294 | 0.001035201 |
| ENSG00000269190 | FBXO17 | 1.170232238 | 0.022139891 |
| ENSG00000130307 | USHBP1 | 1.170049949 | 0.000144128 |
| ENSG00000205403 | CFI | 1.169992923 | 0.00248748 |
| ENSG00000188897 | CTD-3088G3.8 | 1.169677435 | 2.88291E−05 |
| ENSG00000179862 | CITED4 | 1.169301175 | 0.000543753 |
| ENSG00000161243 | FBXO27 | 1.168148306 | 0.024452406 |
| ENSG00000186765 | FSCN2 | 1.168084426 | 0.00024425 |
| ENSG00000179388 | EGR3 | 1.166963889 | 0.000671962 |
| ENSG00000186265 | BTLA | 1.166027379 | 0.007645981 |
| ENSG00000198844 | ARHGEF15 | 1.166006227 | 1.4018E−05 |
| ENSG00000181625 | SLX1B | 1.165410826 | 0.018464653 |
| ENSG00000140859 | KIFC3 | 1.165172611 | 1.50409E−05 |
| ENSG00000099994 | SUSD2 | 1.165085326 | 2.44696E−05 |
| ENSG00000130758 | MAP3K10 | 1.165018455 | 8.81112E−09 |
| ENSG00000130775 | THEMIS2 | 1.164140071 | 1.1141E−05 |
| ENSG00000019169 | MARCO | 1.163899272 | 0.03788321 |
| ENSG00000174562 | KLK15 | 1.163514709 | 0.03074354 |
| ENSG00000107562 | CXCL12 | 1.163277714 | 0.003116543 |
| ENSG00000276231 | PIK3R6 | 1.16274259 | 2.5923E−06 |
| ENSG00000100055 | CYTH4 | 1.162660663 | 8.31237E−06 |
| ENSG00000081041 | CXCL2 | 1.162017607 | 0.001710475 |
| ENSG00000188385 | JAKMIP3 | 1.161023941 | 0.00025117 |
| ENSG00000100726 | TELO2 | 1.16019289 | 1.77681E−08 |
| ENSG00000127084 | FGD3 | 1.160181671 | 5.821E−06 |
| ENSG00000170442 | KRT86 | 1.160047609 | 0.010902873 |
| ENSG00000146215 | CRIP3 | 1.159728565 | 0.012036054 |
| ENSG00000175040 | CHST2 | 1.159387841 | 1.74927E−07 |
| ENSG00000113263 | ITK | 1.157962814 | 0.002994329 |
| ENSG00000066056 | TIE1 | 1.157121327 | 2.57919E−06 |
| ENSG00000064932 | SBNO2 | 1.154842996 | 2.01349E−09 |
| ENSG00000142303 | ADAMTS10 | 1.154016736 | 3.50003E−06 |
| ENSG00000121270 | ABCC11 | 1.153425253 | 0.007349047 |
| ENSG00000142347 | MYO1F | 1.153301943 | 2.80959E−06 |
| ENSG00000211445 | GPX3 | 1.152979864 | 0.002642485 |
| ENSG00000139626 | ITGB7 | 1.152816513 | 2.59729E−05 |
| ENSG00000150051 | MKX | 1.152675665 | 0.003591418 |
| ENSG00000106605 | BLVRA | 1.152291726 | 7.38312E−05 |
| ENSG00000117984 | CTSD | 1.150406941 | 7.23012E−07 |
| ENSG00000262814 | MRPL12 | 1.14980132 | 1.01717E−06 |
| ENSG00000037280 | FLT4 | 1.149616206 | 1.91092E−05 |
| ENSG00000049768 | FOXP3 | 1.148572275 | 0.000210255 |
| ENSG00000119673 | ACOT2 | 1.148562993 | 1.96284E−05 |
| ENSG00000244607 | CCDC13 | 1.148402517 | 0.001083259 |
| ENSG00000174004 | NRROS | 1.146957235 | 0.000398563 |
| ENSG00000242114 | MTFP1 | 1.146834792 | 0.000127226 |
| ENSG00000234745 | HLA-B | 1.14449985 | 0.000192326 |
| ENSG00000160712 | IL6R | 1.144394841 | 6.18274E−05 |
| ENSG00000155666 | KDM8 | 1.14393085 | 7.1391E−06 |
| ENSG00000274897 | PANO1 | 1.143671055 | 0.006883127 |
| ENSG00000144712 | CAND2 | 1.143301833 | 0.003189252 |
| ENSG00000072163 | LIMS2 | 1.14253052 | 5.08751E−06 |
| ENSG00000180089 | TMEM86B | 1.142254851 | 3.58273E−05 |
| ENSG00000136244 | IL6 | 1.142155804 | 0.005306614 |
| ENSG00000140564 | FURIN | 1.14099648 | 4.8433E−08 |
| ENSG00000180448 | HMHA1 | 1.140877238 | 3.16186E−07 |
| ENSG00000041515 | MYO16 | 1.137936033 | 0.014846776 |
| ENSG00000108821 | COL1A1 | 1.137906751 | 6.96901E−05 |
| ENSG00000167617 | CDC42EP5 | 1.137640568 | 2.06907E−05 |
| ENSG00000110900 | TSPAN11 | 1.137384026 | 0.01727461 |
| ENSG00000153976 | HS3ST3A1 | 1.136861692 | 0.00145564 |
| ENSG00000159958 | TNFRSF13C | 1.135763754 | 0.000505142 |
| ENSG00000136383 | ALPK3 | 1.134521046 | 0.000911625 |
| ENSG00000124507 | PACSIN1 | 1.133485154 | 0.01802573 |
| ENSG00000131355 | EMR3 | 1.133123347 | 0.002138295 |
| ENSG00000182866 | LCK | 1.132590929 | 0.001221493 |
| ENSG00000137841 | PLCB2 | 1.131199585 | 4.80977E−05 |
| ENSG00000166741 | NNMT | 1.130139929 | 0.003612422 |
| ENSG00000244617 | ASPRV1 | 1.129943712 | 8.47997E−05 |
| ENSG00000141753 | IGFBP4 | 1.12987189 | 2.06992E−07 |
| ENSG00000131584 | ACAP3 | 1.128585412 | 5.13604E−08 |
| ENSG00000186517 | ARHGAP30 | 1.128385549 | 1.78438E−06 |
| ENSG00000107281 | NPDC1 | 1.128109941 | 0.000153557 |
| ENSG00000224051 | CPTP | 1.127327512 | 7.12605E−08 |
| ENSG00000137225 | CAPN11 | 1.127116709 | 0.000800018 |
| ENSG00000198355 | PIM3 | 1.126715724 | 2.06238E−08 |
| ENSG00000065054 | SLC9A3R2 | 1.126549161 | 4.41674E−07 |
| ENSG00000128815 | WDFY4 | 1.126187069 | 3.84042E−05 |
| ENSG00000160446 | ZDHHC12 | 1.125407504 | 2.69962E−08 |
| ENSG00000166250 | CLMP | 1.125265155 | 0.012732841 |
| ENSG00000064687 | ABCA7 | 1.124560595 | 2.12037E−06 |
| ENSG00000177301 | KCNA2 | 1.124375927 | 0.030590556 |
| ENSG00000089847 | ANKRD24 | 1.123147631 | 0.00010158 |
| ENSG00000124780 | KCNK17 | 1.123054975 | 0.039607548 |
| ENSG00000183484 | GPR132 | 1.122788398 | 0.000233572 |
| ENSG00000177098 | SCN4B | 1.121852752 | 0.004936442 |
| ENSG00000167646 | DNAAF3 | 1.121818701 | 0.01541232 |
| ENSG00000179344 | HLA-DQB1 | 1.121194027 | 0.003860617 |
| ENSG00000175756 | AURKAIP1 | 1.120692217 | 5.56991E−08 |
| ENSG00000128594 | LRRC4 | 1.120596728 | 0.031922975 |
| ENSG00000153902 | LGI4 | 1.120555731 | 0.01945264 |
| ENSG00000169026 | MFSD7 | 1.120383414 | 4.02105E−05 |
| ENSG00000196329 | GIMAP5 | 1.119693858 | 0.004400199 |
| ENSG00000147082 | CCNB3 | 1.119606628 | 0.002188382 |
| ENSG00000204936 | CD177 | 1.119224649 | 0.008447061 |
| ENSG00000164082 | GRM2 | 1.118910518 | 0.000505892 |
| ENSG00000174946 | GPR171 | 1.118335829 | 0.04527799 |
| ENSG00000100258 | LMF2 | 1.116907105 | 1.61252E−09 |
| ENSG00000138316 | ADAMTS14 | 1.116832224 | 0.000353376 |
| ENSG00000177103 | DSCAML1 | 1.115647195 | 0.018105046 |
| ENSG00000179094 | PER1 | 1.115552017 | 1.65195E−05 |
| ENSG00000171631 | P2RY6 | 1.115532374 | 0.001882189 |
| ENSG00000140678 | ITGAX | 1.115131768 | 0.000229531 |
| ENSG00000108515 | ENO3 | 1.114503016 | 1.00695E−05 |
| ENSG00000186350 | RXRA | 1.114500407 | 3.56261E−09 |
| ENSG00000172349 | IL16 | 1.114433597 | 7.70443E−05 |
| ENSG00000001630 | CYP51A1 | 1.113923372 | 3.37844E−05 |
| ENSG00000166819 | PLIN1 | 1.11373741 | 0.000662335 |
| ENSG00000213445 | SIPA1 | 1.113237843 | 3.13066E−08 |
| ENSG00000130592 | LSP1 | 1.112880732 | 2.25894E−05 |
| ENSG00000197483 | ZNF628 | 1.112760768 | 6.95363E−09 |
| ENSG00000232434 | C9orf172 | 1.112181438 | 1.66071E−05 |
| ENSG00000140691 | ARMC5 | 1.111440744 | 6.03792E−08 |
| ENSG00000184163 | FAM132A | 1.110471489 | 0.000926624 |
| ENSG00000126603 | GLIS2 | 1.110337998 | 5.2843E−05 |
| ENSG00000115648 | MLPH | 1.108800611 | 0.006797003 |
| ENSG00000100399 | CHADL | 1.10872322 | 0.000236686 |
| ENSG00000143546 | S100A8 | 1.108121715 | 0.018184067 |
| ENSG00000132361 | CLUH | 1.107468616 | 1.70231E−07 |
| ENSG00000167705 | RILP | 1.106984107 | 1.16038E−05 |
| ENSG00000177943 | MAMDC4 | 1.105766892 | 4.92773E−05 |
| EN5G00000204389 | HSPA1A | 1.105573406 | 0.026955025 |
| ENSG00000164406 | LEAP2 | 1.105554855 | 0.00483751 |
| EN5G00000090339 | ICAM1 | 1.105299336 | 1.01699E−05 |
| ENSG00000107738 | C10orf54 | 1.104818641 | 4.21805E−07 |
| EN5G00000075340 | ADD2 | 1.104459077 | 0.019321465 |
| EN5G00000205502 | C2CD4B | 1.103521648 | 0.006132399 |
| ENSG00000142494 | SLC47A1 | 1.10185006 | 0.003457899 |
| ENSG00000167604 | NFKBID | 1.101651665 | 3.19923E−07 |
| ENSG00000110876 | SELPLG | 1.101255733 | 3.92388E−05 |
| ENSG00000138080 | EMILIN1 | 1.100491411 | 7.37093E−05 |
| EN5G00000239697 | TNFSF12 | 1.100488267 | 1.02589E−06 |
| EN5G00000099864 | PALM | 1.099080013 | 9.65948E−05 |
| ENSG00000183287 | CCBE1 | 1.098938707 | 0.003307171 |
| ENSG00000117016 | RIMS3 | 1.098925724 | 0.000159024 |
| ENSG00000129911 | KLF16 | 1.098914019 | 1.87301E−08 |
| ENSG00000148832 | PAOX | 1.098751829 | 2.04579E−07 |
| ENSG00000159069 | FBXW5 | 1.098608594 | 1.24453E−08 |
| ENSG00000115556 | PLCD4 | 1.098338046 | 0.002321536 |
| ENSG00000126461 | SCAF1 | 1.097461767 | 7.64985E−10 |
| ENSG00000108405 | P2RX1 | 1.095392688 | 0.000560723 |
| ENSG00000187796 | CARD9 | 1.095357432 | 0.00020497 |
| EN5G00000099365 | STX1B | 1.095014517 | 7.83139E−05 |
| ENSG00000111863 | ADTRP | 1.094270076 | 0.002274409 |
| ENSG00000080007 | DDX43 | 1.094255237 | 0.033692994 |
| ENSG00000168389 | MFSD2A | 1.093807606 | 0.000368653 |
| ENSG00000188833 | ENTPD8 | 1.092008372 | 0.004385372 |
| ENSG00000105329 | TGFB1 | 1.091719304 | 8.49811E−06 |
| ENSG00000178175 | ZNF366 | 1.090809513 | 0.000176135 |
| ENSG00000173264 | GPR137 | 1.090306677 | 1.61252E−09 |
| ENSG00000164362 | TERT | 1.090135651 | 0.012315101 |
| ENSG00000168394 | TAP1 | 1.088779671 | 9.37046E−05 |
| ENSG00000185522 | LMNTD2 | 1.088505352 | 3.75729E−07 |
| ENSG00000134326 | CMPK2 | 1.088235158 | 0.000574024 |
| ENSG00000171766 | GATM | 1.08705008 | 0.007418665 |
| EN5G00000083454 | P2RX5 | 1.08688171 | 0.005272454 |
| ENSG00000125384 | PTGER2 | 1.086778309 | 0.001099626 |
| ENSG00000186810 | CXCR3 | 1.086635323 | 2.41732E−05 |
| ENSG00000166592 | RRAD | 1.086211138 | 0.005249801 |
| EN5G00000087076 | HSD17B14 | 1.086016867 | 0.0006444 |
| ENSG00000170638 | TRABD | 1.085976717 | 1.1093E−08 |
| ENSG00000172346 | CSDC2 | 1.085401102 | 0.013051358 |
| ENSG00000100365 | NCF4 | 1.083411322 | 0.000882812 |
| ENSG00000167261 | DPEP2 | 1.082913732 | 8.40121E−05 |
| ENSG00000132530 | XAF1 | 1.082746903 | 0.001133135 |
| ENSG00000244482 | LILRA6 | 1.081344765 | 0.001907085 |
| ENSG00000173208 | ABCD2 | 1.080547139 | 0.016361716 |
| ENSG00000127948 | POR | 1.080517393 | 1.98599E−06 |
| ENSG00000168421 | RHOH | 1.080458885 | 0.000631768 |
| ENSG00000205045 | SLFN12L | 1.079701747 | 0.000329739 |
| ENSG00000185100 | ADSSL1 | 1.079301793 | 7.89069E−05 |
| ENSG00000184371 | CSF1 | 1.078895296 | 1.15396E−05 |
| ENSG00000079432 | CIC | 1.078272473 | 9.56751E−10 |
| ENSG00000135636 | DYSF | 1.078190227 | 2.43404E−05 |
| ENSG00000169129 | AFAP1L2 | 1.077772049 | 0.000590772 |
| ENSG00000149451 | ADAM33 | 1.077718443 | 0.01593276 |
| ENSG00000071655 | MBD3 | 1.076724274 | 5.2406E−08 |
| ENSG00000143851 | PTPN7 | 1.076655864 | 0.00112032 |
| ENSG00000160447 | PKN3 | 1.076244771 | 0.000102006 |
| ENSG00000100351 | GRAP2 | 1.075498285 | 0.000247936 |
| ENSG00000100060 | MFNG | 1.075446319 | 3.10163E−05 |
| ENSG00000146112 | PPP1R18 | 1.075439687 | 6.37535E−07 |
| ENSG00000144857 | BOC | 1.075195822 | 0.000855281 |
| ENSG00000164742 | ADCY1 | 1.07511608 | 0.009076307 |
| ENSG00000166405 | RIC3 | 1.075107073 | 0.015776542 |
| ENSG00000008517 | IL32 | 1.075098165 | 2.3075E−06 |
| ENSG00000069399 | BCL3 | 1.07418887 | 1.97112E−07 |
| ENSG00000007314 | SCN4A | 1.074153208 | 0.030157553 |
| ENSG00000007264 | MATK | 1.073659668 | 0.003255642 |
| ENSG00000177542 | SLC25A22 | 1.073637351 | 1.28739E−07 |
| ENSG00000114654 | EFCC1 | 1.073143748 | 0.000577098 |
| ENSG00000160471 | COX6B2 | 1.072662335 | 0.002420663 |
| ENSG00000132016 | C19orf57 | 1.069884421 | 0.001051846 |
| ENSG00000008513 | ST3GAL1 | 1.069062286 | 6.28731E−05 |
| ENSG00000258429 | PDF | 1.068805467 | 1.60914E−05 |
| ENSG00000131196 | NFATC1 | 1.068662284 | 0.000153644 |
| ENSG00000168490 | PHYHIP | 1.067989173 | 0.000655414 |
| ENSG00000132321 | IQCA1 | 1.067262986 | 0.003653666 |
| ENSG00000142694 | EVA1B | 1.066699002 | 3.79044E−05 |
| ENSG00000179593 | ALOX15B | 1.066673812 | 0.017470576 |
| ENSG00000069535 | MAOB | 1.066281127 | 0.01103073 |
| ENSG00000021488 | SLC7A9 | 1.065153217 | 0.040473085 |
| ENSG00000104856 | RELB | 1.064842196 | 6.80014E−08 |
| ENSG00000143502 | SUSD4 | 1.064151902 | 0.020512908 |
| ENSG00000197766 | CFD | 1.063499521 | 0.006661899 |
| ENSG00000141873 | SLC39A3 | 1.06286932 | 1.61252E−09 |
| ENSG00000144655 | CSRNP1 | 1.062852718 | 3.78207E−06 |
| ENSG00000179431 | FJX1 | 1.062736663 | 0.001746152 |
| ENSG00000010295 | IFFO1 | 1.06198314 | 3.08202E−06 |
| ENSG00000154146 | NRGN | 1.061895795 | 0.000514682 |
| ENSG00000255587 | RAB44 | 1.061875177 | 0.002263706 |
| ENSG00000136689 | 1L1RN | 1.061614732 | 0.004473602 |
| ENSG00000212123 | PRR22 | 1.060148319 | 0.000329992 |
| ENSG00000184986 | TMEM121 | 1.060127995 | 0.004337306 |
| ENSG00000156218 | ADAMTSL3 | 1.059442455 | 0.009010825 |
| ENSG00000104518 | GSDMD | 1.058899122 | 1.68397E−08 |
| ENSG00000127586 | CHTF18 | 1.05847033 | 1.92166E−06 |
| ENSG00000169885 | CALML6 | 1.058443891 | 0.006025347 |
| ENSG00000185432 | METTL7A | 1.058210929 | 0.000931459 |
| ENSG00000197859 | ADAMTSL2 | 1.058191896 | 0.000680205 |
| ENSG00000173535 | TNFRSF10C | 1.057654248 | 0.001415259 |
| ENSG00000205744 | DENND1C | 1.057627265 | 2.07749E−05 |
| ENSG00000178226 | PRSS36 | 1.057624918 | 0.00016216 |
| ENSG00000104951 | IL4I1 | 1.057323492 | 0.005793889 |
| ENSG00000160255 | ITGB2 | 1.056839191 | 0.000161016 |
| ENSG00000198719 | DLL1 | 1.05599471 | 0.001184828 |
| ENSG00000133800 | LYVE1 | 1.055850159 | 0.043808287 |
| ENSG00000100292 | HMOX1 | 1.05514357 | 0.000370937 |
| ENSG00000228300 | C19orf24 | 1.054694366 | 3.91459E−08 |
| ENSG00000250506 | CDK3 | 1.054690478 | 8.09788E−05 |
| ENSG00000213638 | ADAT3 | 1.054231629 | 1.26392E−05 |
| ENSG00000143819 | EPHX1 | 1.053600025 | 0.001432138 |
| ENSG00000157873 | TNFRSF14 | 1.053508443 | 2.32105E−07 |
| ENSG00000075618 | FSCN1 | 1.053500204 | 0.000139237 |
| ENSG00000239389 | PCDHA13 | 1.053422679 | 0.021095719 |
| ENSG00000166831 | RBPMS2 | 1.053327917 | 0.000473809 |
| ENSG00000154102 | C16orf74 | 1.052542155 | 0.021325178 |
| ENSG00000183918 | SH2D1A | 1.050176267 | 0.022002936 |
| ENSG00000204103 | MAFB | 1.050165961 | 2.79729E−05 |
| ENSG00000142185 | TRPM2 | 1.050001696 | 4.60613E−06 |
| ENSG00000187513 | GJA4 | 1.049591295 | 0.000260326 |
| ENSG00000171223 | JUNB | 1.049275198 | 4.53987E−05 |
| ENSG00000162881 | OXER1 | 1.048671881 | 0.002008756 |
| ENSG00000117318 | ID3 | 1.048359003 | 0.000365298 |
| ENSG00000167964 | RAB26 | 1.048012744 | 0.008594494 |
| ENSG00000090975 | PITPNM2 | 1.04785399 | 1.25695E−08 |
| ENSG00000088882 | CPXM1 | 1.047614103 | 0.000275308 |
| ENSG00000092295 | TGM1 | 1.047489427 | 0.000318786 |
| ENSG00000073169 | SELO | 1.047267301 | 1.04001E−08 |
| ENSG00000198756 | COLGALT2 | 1.046923157 | 0.017938451 |
| ENSG00000196557 | CACNA1H | 1.046895388 | 0.000112454 |
| ENSG00000204839 | MROH6 | 1.046814689 | 0.00223642 |
| ENSG00000142627 | EPHA2 | 1.046491068 | 0.000121085 |
| ENSG00000129968 | ABHD17A | 1.046154536 | 2.67234E−09 |
| ENSG00000141968 | VAV1 | 1.046065514 | 0.000227518 |
| ENSG00000109956 | B3GAT1 | 1.045800497 | 0.026258692 |
| ENSG00000134824 | FADS2 | 1.04551583 | 0.013619109 |
| ENSG00000112195 | TREML2 | 1.045199242 | 0.027528123 |
| ENSG00000117090 | SLAMF1 | 1.045027998 | 0.00104207 |
| ENSG00000129467 | ADCY4 | 1.044890545 | 1.88926E−07 |
| ENSG00000110324 | IL10RA | 1.043561468 | 0.000159814 |
| ENSG00000171703 | TCEA2 | 1.042710176 | 7.3573E−05 |
| ENSG00000257594 | GALNT4 | 1.042103173 | 0.002255574 |
| ENSG00000275074 | NUDT18 | 1.041739758 | 1.09773E−07 |
| ENSG00000132613 | MTSS1L | 1.041172779 | 2.38161E−05 |
| ENSG00000177000 | MTHFR | 1.040644704 | 1.62671E−09 |
| ENSG00000173918 | C1QTNF1 | 1.040605797 | 2.45491E−06 |
| ENSG00000059915 | PSD | 1.040036189 | 0.006261123 |
| ENSG00000005381 | MPO | 1.039726111 | 0.018016543 |
| ENSG00000188130 | MAPK12 | 1.039036831 | 0.002218465 |
| ENSG00000139865 | TTC6 | 1.038538765 | 0.042982628 |
| ENSG00000117594 | HSD11B1 | 1.038346244 | 0.01897543 |
| ENSG00000111186 | WNT5B | 1.038143085 | 0.045872871 |
| ENSG00000140479 | PCSK6 | 1.038010488 | 0.000125867 |
| ENSG00000159674 | SPON2 | 1.037337589 | 3.36554E−06 |
| ENSG00000115607 | IL18RAP | 1.036524919 | 0.020819185 |
| ENSG00000269313 | MAGIX | 1.035664732 | 0.000237527 |
| ENSG00000171115 | GIMAP8 | 1.034684315 | 0.00043775 |
| ENSG00000167083 | GNGT2 | 1.03468063 | 0.001995109 |
| ENSG00000040487 | PQLC2 | 1.034389549 | 2.48735E−09 |
| ENSG00000219200 | RNASEK | 1.033863267 | 1.99409E−06 |
| ENSG00000007171 | NOS2 | 1.033693076 | 0.029503201 |
| ENSG00000143845 | ETNK2 | 1.033666496 | 0.014297837 |
| ENSG00000169169 | CPT1C | 1.033096164 | 0.000280624 |
| ENSG00000100003 | SEC14L2 | 1.032643853 | 8.05301E−06 |
| ENSG00000133561 | GIMAP6 | 1.032162261 | 0.000410499 |
| ENSG00000130748 | TMEM160 | 1.031899717 | 3.04521E−05 |
| ENSG00000176428 | VPS37D | 1.031638204 | 0.006069576 |
| ENSG00000067840 | PDZD4 | 1.031635532 | 0.001182899 |
| ENSG00000260729 | RP11-106M3.2 | 1.031523699 | 0.004422184 |
| ENSG00000138615 | CILP | 1.031441487 | 0.004050808 |
| ENSG00000136286 | MYO1G | 1.031431358 | 1.32038E−05 |
| ENSG00000101306 | MYLK2 | 1.03111681 | 0.015931012 |
| ENSG00000130813 | C19orf66 | 1.03098173 | 2.3625E−07 |
| ENSG00000157881 | PANK4 | 1.030716284 | 1.55066E−09 |
| ENSG00000197943 | PLCG2 | 1.03053882 | 3.32973E−05 |
| ENSG00000088881 | EBF4 | 1.030300777 | 7.54433E−05 |
| ENSG00000169403 | PTAFR | 1.029756668 | 7.56013E−05 |
| ENSG00000173156 | RHOD | 1.029683172 | 0.00046796 |
| ENSG00000168685 | IL7R | 1.0288166 | 0.021919768 |
| ENSG00000164877 | MICALL2 | 1.028563364 | 1.53624E−06 |
| ENSG00000198400 | NTRK1 | 1.028519589 | 0.017969678 |
| ENSG00000278272 | HIST1H3C | 1.02809485 | 0.04527799 |
| ENSG00000135426 | TESPA1 | 1.027798615 | 0.006769597 |
| ENSG00000185112 | FAM43A | 1.02773188 | 0.000706383 |
| ENSG00000133069 | TMCC2 | 1.027431033 | 0.000504625 |
| ENSG00000152669 | CCNO | 1.02741933 | 0.003849632 |
| ENSG00000150281 | CTF1 | 1.024938478 | 0.005641207 |
| ENSG00000180340 | FZD2 | 1.024674756 | 0.000793735 |
| ENSG00000181847 | TIGIT | 1.024205903 | 0.005273732 |
| ENSG00000167543 | TP53I13 | 1.023958089 | 1.27231E−08 |
| ENSG00000171119 | NRTN | 1.023021234 | 0.02325124 |
| ENSG00000100242 | SUN2 | 1.022893722 | 2.01349E−09 |
| ENSG00000147813 | NAPRT | 1.022430177 | 6.39556E−06 |
| ENSG00000117407 | ARTN | 1.022353399 | 0.0008376 |
| ENSG00000105426 | PTPRS | 1.021824017 | 0.000212169 |
| ENSG00000114270 | COL7A1 | 1.021598787 | 0.001215547 |
| ENSG00000153822 | KCNJ16 | 1.021459687 | 0.044482696 |
| ENSG00000077150 | NFKB2 | 1.021430482 | 4.49101E−07 |
| ENSG00000107404 | DVL1 | 1.021045107 | 1.01962E−07 |
| ENSG00000205220 | PSMB10 | 1.021040341 | 3.62571E−05 |
| ENSG00000125657 | TNFSF9 | 1.019603439 | 0.031914053 |
| ENSG00000078808 | SDF4 | 1.019227571 | 2.01349E−09 |
| ENSG00000154277 | UCHL1 | 1.018802658 | 0.003540154 |
| ENSG00000162490 | DRAXIN | 1.018565781 | 0.005194579 |
| ENSG00000157103 | SLC6A1 | 1.017456846 | 0.006172039 |
| ENSG00000167700 | MFSD3 | 1.017275872 | 4.18874E−06 |
| ENSG00000174307 | PHLDA3 | 1.017000493 | 0.001408229 |
| ENSG00000142733 | MAP3K6 | 1.016883678 | 8.16815E−06 |
| ENSG00000139737 | SLAIN1 | 1.016814932 | 0.015892184 |
| ENSG00000213398 | LCAT | 1.01669424 | 0.000482415 |
| ENSG00000107872 | FBXL15 | 1.016569441 | 1.29236E−06 |
| EN5G00000154783 | FGD5 | 1.016439449 | 0.000753317 |
| ENSG00000130881 | LRP3 | 1.016427576 | 0.000308748 |
| ENSG00000183307 | CECR6 | 1.016246615 | 0.004475632 |
| ENSG00000188566 | NDOR1 | 1.015176081 | 1.74927E−07 |
| ENSG00000040608 | RTN4R | 1.013297884 | 0.005004347 |
| ENSG00000278817 | DGCR6 | 1.013286788 | 0.00290828 |
| ENSG00000128918 | ALDH1A2 | 1.013137666 | 0.005867698 |
| ENSG00000164867 | NOS3 | 1.013029428 | 1.6008E−06 |
| ENSG00000172382 | PRSS27 | 1.012692081 | 0.005160783 |
| ENSG00000172771 | EFCAB12 | 1.011647683 | 0.000719841 |
| ENSG00000175592 | FOSL1 | 1.011353509 | 0.001165444 |
| ENSG00000250588 | IQCJ-SCHIP1 | 1.010670959 | 0.002552664 |
| ENSG00000158246 | FAM46B | 1.010098634 | 0.009784986 |
| ENSG00000000938 | FGR | 1.009675657 | 0.000350001 |
| ENSG00000111077 | TNS2 | 1.009082599 | 1.60914E−05 |
| ENSG00000091262 | ABCC6 | 1.008007735 | 0.001454326 |
| ENSG00000179921 | GPBAR1 | 1.007582784 | 0.000895708 |
| ENSG00000187556 | NANOS3 | 1.007482149 | 0.005960624 |
| ENSG00000163599 | CTLA4 | 1.007446501 | 0.034491458 |
| ENSG00000173269 | MMRN2 | 1.007103915 | 7.63617E−06 |
| ENSG00000099957 | P2RX6 | 1.005774931 | 0.014788148 |
| ENSG00000075702 | WDR62 | 1.005394304 | 3.82812E−05 |
| ENSG00000140254 | DUOXA1 | 1.005197626 | 0.030157553 |
| ENSG00000160408 | ST6GALNAC6 | 1.004182254 | 6.00075E−07 |
| ENSG00000162591 | MEGF6 | 1.003455165 | 0.000102697 |
| ENSG00000099998 | GGT5 | 1.002930452 | 0.000349259 |
| ENSG00000124126 | PREX1 | 1.002854047 | 9.05886E−06 |
| ENSG00000132879 | FBXO44 | 1.00159982 | 4.6691E−05 |
| ENSG00000179023 | KLHDC7A | 1.000944037 | 0.044341993 |
| ENSG00000153233 | PTPRR | -1.001554855 | 0.033837303 |
| ENSG00000076053 | RBM7 | -1.001759951 | 6.95363E−09 |
| ENSG00000272602 | ZNF595 | -1.002136961 | 0.000561463 |
| ENSG00000120256 | LRP11 | -1.002266863 | 1.70427E−06 |
| ENSG00000137831 | UACA | -1.003056551 | 6.66332E−08 |
| ENSG00000104412 | EMC2 | -1.003528223 | 2.00163E−07 |
| ENSG00000085719 | CPNE3 | -1.004877956 | 1.29006E−06 |
| ENSG00000168152 | THAP9 | -1.004879803 | 8.60975E−07 |
| ENSG00000170759 | KIF5B | -1.007465609 | 1.99409E−06 |
| ENSG00000101928 | MOSPD1 | -1.008717348 | 5.821E−06 |
| ENSG00000144895 | EIF2A | -1.009295057 | 6.56118E−09 |
| ENSG00000162980 | ARL5A | -1.009810828 | 3.56622E−09 |
| ENSG00000170647 | TMEM133 | -1.010755081 | 1.40464E−06 |
| ENSG00000176731 | C8orf59 | -1.011266379 | 2.72451E−07 |
| ENSG00000173467 | AGR3 | -1.012732957 | 0.038558978 |
| ENSG00000113494 | PRLR | -1.01466158 | 0.000333796 |
| ENSG00000167232 | ZNF91 | -1.016817351 | 7.07164E−08 |
| ENSG00000128609 | NDUFA5 | -1.019382369 | 1.27231E−08 |
| ENSG00000158290 | CUL4B | -1.019448197 | 2.48735E−09 |
| ENSG00000031691 | CENPQ | -1.019776978 | 0.000122089 |
| ENSG00000169446 | MMGT1 | -1.020403146 | 6.37063E−09 |
| ENSG00000121957 | GPSM2 | -1.020454851 | 0.000162521 |
| ENSG00000127995 | CASD1 | -1.020493061 | 1.01688E−06 |
| ENSG00000149050 | ZNF214 | -1.021570453 | 2.23608E−05 |
| ENSG00000110723 | EXPH5 | -1.022006186 | 4.71212E−05 |
| ENSG00000272398 | CD24 | -1.024145293 | 0.002898564 |
| ENSG00000281593 | GS1-114I9.3 | -1.0242454 | 0.000750007 |
| ENSG00000205133 | TRIQK | -1.024705878 | 1.81165E−07 |
| ENSG00000001561 | ENPP4 | -1.025669868 | 3.20006E−07 |
| ENSG00000151846 | PABPC3 | -1.025857769 | 1.83273E−05 |
| ENSG00000241186 | TDGF1 | -1.025932935 | 0.003572067 |
| ENSG00000170231 | FABP6 | -1.028359134 | 0.027437455 |
| ENSG00000172292 | CERS6 | -1.030789935 | 2.82194E−08 |
| ENSG00000085365 | SCAMP1 | -1.033529302 | 1.94568E−09 |
| ENSG00000135338 | LCA5 | -1.035563463 | 2.38152E−05 |
| ENSG00000176563 | CNTDI | -1.036449608 | 0.030900597 |
| ENSG00000147654 | EBAG9 | -1.037428459 | 6.87837E−08 |
| ENSG00000149212 | SESN3 | -1.037498892 | 0.000439262 |
| ENSG00000114248 | LRRC31 | -1.038660117 | 0.002789137 |
| ENSG00000164934 | DCAF13 | -1.039870112 | 1.5195E−07 |
| ENSG00000175548 | ALG10B | -1.043186279 | 0.000200663 |
| ENSG00000142875 | PRKACB | -1.043355361 | 0.00044685 |
| ENSG00000183742 | MACC1 | -1.04566096 | 1.18699E−05 |
| ENSG00000155850 | SLC26A2 | -1.047315134 | 0.034353852 |
| ENSG00000165028 | NIPSNAP3B | -1.049187677 | 0.00033694 |
| ENSG00000156162 | DPY19L4 | -1.050491072 | 2.90403E−07 |
| ENSG00000014123 | UFL1 | -1.052174033 | 2.79903E−10 |
| ENSG00000112742 | TTK | -1.052232254 | 6.83677E−05 |
| ENSG00000137501 | SYTL2 | -1.055045818 | 2.08215E−05 |
| ENSG00000163806 | SPDYA | -1.055660565 | 5.33991E−07 |
| ENSG00000162620 | LRRIQ3 | -1.058277612 | 0.004212656 |
| ENSG00000151239 | TWF1 | -1.062799425 | 1.61252E−09 |
| ENSG00000164414 | SLC35A1 | -1.063652786 | 3.77487E−07 |
| ENSG00000009844 | VTA1 | -1.068127232 | 1.52318E−09 |
| ENSG00000175445 | LPL | -1.068881665 | 0.003051402 |
| ENSG00000104413 | ESRP1 | -1.069139697 | 3.22421E−06 |
| ENSG00000180938 | ZNF572 | -1.071548068 | 4.8019E−05 |
| ENSG00000137942 | FNBP1L | -1.073865136 | 6.31494E−09 |
| ENSG00000154153 | FAM134B | -1.079187306 | 0.000616268 |
| ENSG00000164754 | RAD21 | -1.080437352 | 3.40709E−07 |
| ENSG00000101166 | SLMO2 | -1.083564414 | 1.20742E−07 |
| ENSG00000180354 | MTURN | -1.083961787 | 4.25839E−07 |
| ENSG00000187325 | TAF9B | -1.084956129 | 1.20282E−05 |
| ENSG00000166323 | C11orf65 | -1.090229659 | 0.000165651 |
| ENSG00000101132 | PFDN4 | -1.09076519 | 3.62677E−08 |
| ENSG00000198478 | SH3BGRL2 | -1.092538935 | 4.66585E−08 |
| ENSG00000241127 | YAE1D1 | -1.092681775 | 2.60598E−07 |
| ENSG00000144362 | PHOSPHO2 | -1.099868358 | 1.20111E−07 |
| ENSG00000023287 | RB1CC1 | -1.101297092 | 1.34567E−08 |
| ENSG00000106819 | ASPN | -1.107141062 | 0.000229665 |
| ENSG00000106460 | TMEM106B | -1.109324776 | 9.87742E−08 |
| ENSG00000164176 | EDIL3 | -1.110129582 | 0.000472742 |
| ENSG00000204899 | MZT1 | -1.113296799 | 6.92512E−07 |
| ENSG00000137251 | TINAG | -1.114764129 | 0.014297837 |
| ENSG00000281760 | AL138751.1 | -1.118352932 | 0.00123223 |
| ENSG00000112796 | ENPP5 | -1.118903111 | 6.8546E−05 |
| ENSG00000152558 | TMEM123 | -1.124055055 | 1.45358E−08 |
| ENSG00000184305 | CCSER1 | -1.129691637 | 0.002193699 |
| ENSG00000255552 | LY6G6E | -1.133450266 | 0.017549745 |
| ENSG00000078114 | NEBL | -1.134644322 | 4.2107E−07 |
| ENSG00000114120 | SLC25A36 | -1.151854169 | 1.56096E−09 |
| ENSG00000150783 | TEX12 | -1.153278512 | 0.003400066 |
| ENSG00000280433 | CH507-9B2.9 | -1.157622705 | 2.1793E−07 |
| ENSG00000183889 | AC138969.4 | -1.159897393 | 0.006281546 |
| ENSG00000087253 | LPCAT2 | -1.167126908 | 1.09414E−05 |
| ENSG00000213096 | ZNF254 | -1.169045539 | 1.02593E−08 |
| ENSG00000203943 | SAMD13 | -1.169118931 | 5.54874E−06 |
| ENSG00000124613 | ZNF391 | -1.169851393 | 2.08133E−06 |
| ENSG00000139292 | LGR5 | -1.17614207 | 0.005411767 |
| ENSG00000152580 | IGSF10 | -1.190907242 | 0.017317289 |
| ENSG00000143882 | ATP6V1C2 | -1.19445769 | 0.02964975 |
| ENSG00000104408 | EIF3E | -1.195527466 | 1.08404E−07 |
| ENSG00000000003 | TSPAN6 | -1.204923145 | 4.77464E−07 |
| ENSG00000173890 | GPR160 | -1.20590218 | 0.000168715 |
| ENSG00000134215 | VAV3 | -1.207076873 | 0.000113993 |
| ENSG00000124205 | EDN3 | -1.212678763 | 0.032419727 |
| ENSG00000250641 | XXbac-BPG32J3.19 | -1.216169136 | 0.021509264 |
| ENSG00000213160 | KLHL23 | -1.219625769 | 2.1531E−08 |
| ENSG00000267508 | ZNF285 | -1.221435784 | 0.040665124 |
| ENSG00000124882 | EREG | -1.221469332 | 0.005457894 |
| ENSG00000168300 | PCMTD1 | -1.221506242 | 3.29417E−07 |
| ENSG00000113389 | NPR3 | -1.224080887 | 0.000211447 |
| ENSG00000104427 | ZC2HC1A | -1.230963234 | 1.43368E−06 |
| ENSG00000165730 | STOX1 | -1.267526619 | 0.015565296 |
| ENSG00000253506 | NACA2 | -1.273329502 | 8.79573E−09 |
| ENSG00000168772 | CXXC4 | -1.275875518 | 0.002203819 |
| ENSG00000154639 | CXADR | -1.285684188 | 6.95363E−09 |
| ENSG00000087495 | PHACTR3 | -1.289857846 | 0.005176516 |
| ENSG00000124429 | POF1B | -1.303781347 | 2.38912E−07 |
| ENSG00000196172 | ZNF681 | -1.305686053 | 0.000412086 |
| ENSG00000259823 | LYPD8 | -1.308508498 | 0.013208003 |
| ENSG00000072133 | RPS6KA6 | -1.316470671 | 0.000276829 |
| ENSG00000178662 | CSRNP3 | -1.33898624 | 0.006283148 |
| ENSG00000180785 | OR51E1 | -1.349625675 | 0.013011723 |
| ENSG00000184434 | LRRC19 | -1.367459821 | 0.000306926 |
| ENSG00000279342 | AP000866.1 | -1.386529623 | 1.96401E−08 |
| ENSG00000204361 | NXPE2 | -1.43610326 | 0.003272598 |
| ENSG00000145569 | FAM105A | -1.447185798 | 1.27231E−08 |
| ENSG00000164398 | ACSL6 | -1.499101213 | 0.001665712 |
| ENSG00000146250 | PRSS35 | -1.520224914 | 0.000313552 |
| ENSG00000164946 | FREM1 | -1.564863684 | 0.013976956 |
| ENSG00000137968 | SLC44A5 | -1.598358074 | 0.000528403 |
| ENSG00000095110 | NXPE1 | -1.615714581 | 0.030390621 |
| ENSG00000101958 | GLRA2 | -1.616805688 | 0.041539661 |
| ENSG00000151838 | CCDC175 | -1.737974925 | 0.000126766 |
| ENSG00000181690 | PLAG1 | -1.751394808 | 0.000131268 |
| ENSG00000007306 | CEACAM7 | -2.114674283 | 0.001548556 |

TABLE 3C

| Ensembl ID | Gene Symbol | log2FC | adj P-Value |
|---|---|---|---|
| ENSG00000123500 | COL10A1 | 2.753077645 | 1.94504E−10 |
| ENSG00000135914 | HTR2B | 2.299775445 | 2.03486E−08 |
| ENSG00000105664 | COMP | 2.250725891 | 2.79135E−07 |
| ENSG00000113389 | NPR3 | 2.24650213 | 9.31564E−11 |
| ENSG00000111341 | MGP | 2.141302084 | 1.52321E−08 |
| ENSG00000198542 | ITGBL1 | 2.019042034 | 3.41856E−09 |
| ENSG00000139329 | LUM | 2.0150524 | 3.41856E−09 |
| ENSG00000011465 | DCN | 1.933536371 | 1.46255E−08 |
| ENSG00000106483 | SFRP4 | 1.9282142 | 2.31144E−07 |
| ENSG00000044524 | EPHA3 | 1.926985658 | 3.41856E−09 |
| ENSG00000104435 | STMN2 | 1.923594455 | 2.66078E−05 |
| ENSG00000163145 | C1QTNF7 | 1.922263003 | 3.25587E−07 |
| ENSG00000171476 | HOPX | 1.919610126 | 1.64571E−09 |
| ENSG00000170373 | CST1 | 1.916865824 | 0.01433618 |
| ENSG00000206052 | DOK6 | 1.871623631 | 4.35587E−08 |
| ENSG00000169604 | ANTXR1 | 1.848403126 | 6.74299E−08 |
| ENSG00000198523 | PLN | 1.843972102 | 2.56068E−06 |
| ENSG00000170962 | PDGFD | 1.828019725 | 3.4197E−06 |
| ENSG00000102452 | NALCN | 1.810294149 | 2.9295E−08 |
| ENSG00000166448 | TMEM130 | 1.809017594 | 4.29921E−06 |
| ENSG00000164176 | EDIL3 | 1.798363909 | 1.08047E−08 |
| ENSG00000164220 | F2RL2 | 1.795758993 | 4.16792E−07 |
| ENSG00000163629 | PTPN13 | 1.782990663 | 1.87323E−07 |
| ENSG00000123096 | SSPN | 1.780091089 | 1.0293E−11 |
| ENSG00000132031 | MATN3 | 1.723529561 | 7.0153E−05 |
| ENSG00000140937 | CDH11 | 1.716527677 | 9.72444E−09 |
| ENSG00000178662 | CSRNP3 | 1.712333533 | 5.91801E−05 |
| ENSG00000179542 | SLITRK4 | 1.706883782 | 5.3193E−06 |
| ENSG00000141469 | SLC14A1 | 1.701274635 | 0.002158153 |
| ENSG00000166923 | GREM1 | 1.697378284 | 6.07649E−05 |
| ENSG00000109738 | GLRB | 1.688798567 | 1.28131E−05 |
| ENSG00000122641 | INHBA | 1.681574152 | 1.34487E−07 |
| ENSG00000144810 | COL8A1 | 1.660577626 | 4.3985E−08 |
| ENSG00000050165 | DKK3 | 1.656991196 | 7.88927E−08 |
| ENSG00000180785 | OR51E1 | 1.656338792 | 0.000523247 |
| ENSG00000078098 | FAP | 1.648343594 | 3.41856E−09 |
| ENSG00000134853 | PDGFRA | 1.64591078 | 2.42506E−07 |
| ENSG00000106819 | ASPN | 1.624699824 | 3.2959E−08 |
| ENSG00000130224 | LRCH2 | 1.621049308 | 7.70087E−06 |
| ENSG00000060709 | RIMBP2 | 1.610582302 | 0.00442324 |
| ENSG00000168334 | XIRP1 | 1.607165827 | 0.00010874 |
| ENSG00000134193 | REG4 | 1.60569186 | 0.003181932 |
| ENSG00000259030 | FPGT-TNNI3K | 1.605104369 | 3.3962E−07 |
| ENSG00000139220 | PPFIA2 | 1.60435623 | 2.03684E−05 |
| ENSG00000116132 | PRRX1 | 1.598455218 | 5.05329E−06 |
| ENSG00000105989 | WNT2 | 1.589894336 | 1.5746E−07 |
| ENSG00000188783 | PRELP | 1.579932373 | 1.3397E−06 |
| ENSG00000189320 | FAM180A | 1.579704065 | 9.56288E−06 |
| ENSG00000101230 | ISM1 | 1.57969389 | 1.82586E−06 |
| ENSG00000123094 | RASSF8 | 1.568362816 | 1.05386E−07 |
| ENSG00000101825 | MXRA5 | 1.55718306 | 1.54809E−05 |
| ENSG00000170369 | CST2 | 1.556271823 | 1.34657E−06 |
| ENSG00000171016 | PYGO1 | 1.550732543 | 2.41385E−07 |
| ENSG00000171246 | NPTX1 | 1.54930529 | 2.16518E−06 |
| ENSG00000164197 | RNF180 | 1.548429976 | 1.01886E−08 |
| ENSG00000038427 | VCAN | 1.547202163 | 8.35279E−08 |
| ENSG00000152583 | SPARCL1 | 1.545464546 | 2.05733E−06 |
| ENSG00000055732 | MCOLN3 | 1.544706438 | 0.002283337 |
| ENSG00000165617 | DACT1 | 1.540192459 | 1.3355E−08 |
| ENSG00000141052 | MYOCD | 1.533839495 | 1.27097E−06 |
| ENSG00000189184 | PCDH18 | 1.524966521 | 7.52638E−08 |
| ENSG00000079931 | MOXD1 | 1.52206901 | 8.62559E−07 |
| ENSG00000138696 | BMPR1B | 1.520562773 | 1.46147E−05 |
| ENSG00000164932 | CTHRC1 | 1.517331528 | 1.98345E−08 |
| ENSG00000109339 | MAPK10 | 1.514795308 | 1.3355E−08 |
| ENSG00000111799 | COL12A1 | 1.514488875 | 9.54671E−06 |
| ENSG00000170624 | SGCD | 1.513532236 | 1.34398E−07 |
| ENSG00000182168 | UNC5C | 1.512500422 | 5.05212E−09 |
| ENSG00000203805 | PPAPDC1A | 1.50990084 | 5.05862E−05 |
| ENSG00000120337 | TNFSF18 | 1.505492041 | 7.53394E−06 |
| ENSG00000145687 | SSBP2 | 1.498267108 | 1.64571E−09 |
| ENSG00000079102 | RUNX1T1 | 1.498049751 | 3.31914E−08 |
| ENSG00000157680 | DGKI | 1.492753415 | 6.09951E−07 |
| ENSG00000071967 | CYBRD1 | 1.49130056 | 6.66385E−07 |
| ENSG00000086991 | NOX4 | 1.487601307 | 2.38406E−08 |
| ENSG00000182013 | PNMAL1 | 1.4704151 | 3.47752E−05 |
| ENSG00000166928 | MS4A14 | 1.460689948 | 1.28986E−06 |
| ENSG00000158270 | COLEC12 | 1.456479264 | 0.000123409 |
| ENSG00000113578 | FGF1 | 1.455640093 | 1.08615E−06 |
| ENSG00000147027 | TMEM47 | 1.454243074 | 1.55362E−07 |
| ENSG00000137573 | SULF1 | 1.450407418 | 5.42184E−06 |
| ENSG00000061455 | PRDM6 | 1.449956993 | 2.72945E−08 |
| ENSG00000138685 | FGF2 | 1.449712268 | 1.90869E−05 |
| ENSG00000219438 | FAM19A5 | 1.447573037 | 6.97472E−08 |
| ENSG00000113361 | CDH6 | 1.447278152 | 9.72444E−09 |
| ENSG00000206538 | VGLL3 | 1.446662197 | 1.24067E−08 |
| ENSG00000269948 | RP11−248J23.6 | 1.443017485 | 9.79357E−08 |
| ENSG00000184838 | PRR16 | 1.439275355 | 8.78211E−08 |
| ENSG00000175445 | LPL | 1.431925332 | 7.82242E−06 |
| ENSG00000123095 | BHLHE41 | 1.431917846 | 7.90581E−08 |
| ENSG00000120594 | PLXDC2 | 1.429475002 | 1.56156E−07 |
| ENSG00000132465 | IGJ | 1.42583631 | 0.001616392 |
| ENSG00000006468 | ETV1 | 1.423844057 | 9.72444E−09 |
| ENSG00000163751 | CPA3 | 1.42075873 | 8.80096E−05 |
| ENSG00000082175 | PGR | 1.420382884 | 6.15718E−08 |
| ENSG00000153956 | CACNA2D1 | 1.419787753 | 1.01886E−08 |
| ENSG00000273173 | SNURF | 1.418489439 | 0.000689692 |
| ENSG00000171951 | SCG2 | 1.417775686 | 0.001557801 |
| ENSG00000253250 | C8orf88 | 1.417096006 | 2.90069E−05 |
| ENSG00000138061 | CYP1B1 | 1.408888534 | 3.92298E−06 |
| ENSG00000186310 | NAP1L3 | 1.406286559 | 6.70314E−06 |
| ENSG00000144642 | RBMS3 | 1.406201717 | 6.71096E−08 |
| ENSG00000117020 | AKT3 | 1.404517394 | 5.08642E−08 |
| ENSG00000118523 | CTGF | 1.400129682 | 1.57263E−06 |
| ENSG00000112936 | C7 | 1.390854001 | 0.009810176 |
| ENSG00000172061 | LRRC15 | 1.389505599 | 0.000484441 |
| ENSG00000128045 | RASL11B | 1.385779054 | 0.000177713 |
| ENSG00000164949 | GEM | 1.385443872 | 7.49878E−07 |
| ENSG00000162998 | FRZB | 1.384895497 | 1.93262E−05 |
| ENSG00000186479 | RGS7BP | 1.380174985 | 0.000292203 |
| ENSG00000120820 | GLT8D2 | 1.378308382 | 3.58732E−07 |
| ENSG00000144619 | CNTN4 | 1.377872505 | 4.0634E−07 |
| ENSG00000104368 | PLAT | 1.369843778 | 6.09508E−07 |
| ENSG00000170153 | RNF150 | 1.362822582 | 8.14213E−06 |
| ENSG00000180730 | SHISA2 | 1.362386308 | 0.000271359 |
| ENSG00000184226 | PCDH9 | 1.361369498 | 8.92336E−07 |
| ENSG00000136155 | SCEL | 1.358497995 | 0.013519504 |
| ENSG00000112837 | TBX18 | 1.357167308 | 0.015764677 |
| ENSG00000173705 | SUSD5 | 1.352563993 | 0.000866381 |
| ENSG00000145431 | PDGFC | 1.352483377 | 1.10659E−07 |
| ENSG00000091986 | CCDC80 | 1.352248452 | 1.08521E−05 |
| ENSG00000125869 | LAMP5 | 1.348587285 | 1.38474E−05 |
| ENSG00000181104 | F2R | 1.34574369 | 1.14245E−07 |
| ENSG00000171817 | ZNF540 | 1.34309348 | 1.67177E−05 |
| ENSG00000174099 | MSRB3 | 1.340803477 | 3.69366E−08 |
| ENSG00000244694 | PTCHD4 | 1.339435089 | 0.003004708 |
| ENSG00000182050 | MGAT4C | 1.338163591 | 0.004097168 |
| ENSG00000145244 | CORIN | 1.336481727 | 1.98313E−06 |
| ENSG00000162493 | PDPN | 1.334674174 | 0.000278822 |
| ENSG00000172348 | RCAN2 | 1.334220756 | 5.21317E−06 |
| ENSG00000106809 | OGN | 1.334183583 | 0.027938697 |
| ENSG00000126860 | EVI2A | 1.334181518 | 3.75687E−05 |
| ENSG00000117069 | ST6GALNAC5 | 1.333984274 | 0.000168565 |
| ENSG00000122786 | CALD1 | 1.329513327 | 7.07147E−08 |
| ENSG00000140285 | FGF7 | 1.327620123 | 0.005858699 |
| ENSG00000186354 | C9orf47 | 1.327193509 | 0.000363669 |
| ENSG00000143248 | RGS5 | 1.326377353 | 3.48966E−05 |
| ENSG00000162733 | DDR2 | 1.325854104 | 6.97472E−08 |
| ENSG00000118473 | SGIP1 | 1.32523897 | 1.53101E−07 |
| ENSG00000123610 | TNFAIP6 | 1.318294514 | 4.38333E−05 |
| ENSG00000114948 | ADAM23 | 1.31458896 | 0.00040247 |
| ENSG00000118922 | KLF12 | 1.313963927 | 2.93943E−05 |
| ENSG00000152527 | PLEKHH2 | 1.307669268 | 5.05054E−06 |
| ENSG00000166292 | TMEM100 | 1.307432826 | 0.000384496 |
| ENSG00000142871 | CYR61 | 1.305804906 | 0.000113848 |
| ENSG00000115380 | EFEMP1 | 1.305757305 | 4.8433E−06 |
| ENSG00000172403 | SYNPO2 | 1.302382009 | 6.44659E−07 |
| ENSG00000183801 | OLFML1 | 1.301498794 | 3.03294E−06 |
| ENSG00000182667 | NTM | 1.299038157 | 1.37123E−07 |
| ENSG00000092969 | TGFB2 | 1.298440651 | 8.81306E−07 |
| ENSG00000102383 | ZDHHC15 | 1.296671398 | 0.001991458 |
| ENSG00000164935 | DCSTAMP | 1.293345365 | 0.000673846 |
| ENSG00000148516 | ZEB1 | 1.290822813 | 3.49298E−08 |
| ENSG00000172476 | RAB40A | 1.290170038 | 0.000141934 |
| ENSG00000280188 | AC005477.1 | 1.289835364 | 0.009237267 |
| ENSG00000121898 | CPXM2 | 1.28965125 | 0.000314622 |
| ENSG00000168461 | RAB31 | 1.289572671 | 6.8273E−07 |
| ENSG00000111339 | ART4 | 1.289492512 | 0.000176902 |
| ENSG00000168329 | CX3CR1 | 1.286832974 | 8.8475E−05 |
| ENSG00000186340 | THBS2 | 1.286597108 | 5.35484E−06 |
| ENSG00000184564 | SLITRK6 | 1.285783204 | 0.03427456 |
| ENSG00000007306 | CEACAM7 | 1.284722771 | 0.01660678 |
| ENSG00000143341 | HMCN1 | 1.282445088 | 6.52761E−07 |
| ENSG00000109684 | CLNK | 1.281226278 | 0.000311256 |
| ENSG00000149380 | P4HA3 | 1.280152222 | 3.99847E−07 |
| ENSG00000183160 | TMEM119 | 1.27777833 | 0.000796722 |
| ENSG00000173068 | BNC2 | 1.275532583 | 2.65783E−06 |
| ENSG00000091844 | RGS17 | 1.271354855 | 0.000336905 |
| ENSG00000094963 | FMO2 | 1.270693765 | 6.25198E−05 |
| ENSG00000071991 | CDH19 | 1.270654613 | 0.001176266 |
| ENSG00000171659 | GPR34 | 1.269314632 | 7.95073E−05 |
| ENSG00000243244 | STON1 | 1.269130245 | 2.70879E−08 |
| ENSG00000169851 | PCDH7 | 1.268726148 | 8.34772E−05 |
| ENSG00000019991 | HGF | 1.268406656 | 6.34326E−05 |
| ENSG00000122691 | TWIST1 | 1.268232991 | 0.001671374 |
| ENSG00000144681 | STAC | 1.267386375 | 0.00128752 |
| ENSG00000162614 | NEXN | 1.26725904 | 7.42402E−07 |
| ENSG00000125355 | TMEM255A | 1.265417711 | 0.000590037 |
| ENSG00000048052 | HDAC9 | 1.264177895 | 2.52719E−06 |
| ENSG00000148053 | NTRK2 | 1.262128711 | 0.005455326 |
| ENSG00000106034 | CPED1 | 1.260821872 | 3.2394E−05 |
| ENSG00000106823 | ECM2 | 1.259370731 | 7.44669E−07 |
| ENSG00000188738 | FSIP2 | 1.257352706 | 0.010675808 |
| ENSG00000113209 | PCDHB5 | 1.256678528 | 0.000940712 |
| ENSG00000137558 | PI15 | 1.255925384 | 0.000297298 |
| ENSG00000196628 | TCF4 | 1.254329918 | 3.03846E−07 |
| ENSG00000115252 | PDE1A | 1.250018514 | 2.64199E−07 |
| ENSG00000072952 | MRVI1 | 1.249016956 | 7.43903E−07 |
| ENSG00000198829 | SUCNR1 | 1.248474774 | 0.000457552 |
| ENSG00000134184 | GSTM1 | 1.246483399 | 0.049619541 |
| ENSG00000133107 | TRPC4 | 1.245140196 | 1.38743E−05 |
| ENSG00000166482 | MFAP4 | 1.244718947 | 0.001246995 |
| ENSG00000154188 | ANGPT1 | 1.240410099 | 0.001977131 |
| ENSG00000133110 | POSTN | 1.240025355 | 0.00049143 |
| ENSG00000196569 | LAMA2 | 1.238282396 | 0.00013276 |
| ENSG00000116106 | EPHA4 | 1.235126812 | 0.002447196 |
| ENSG00000154263 | ABCA10 | 1.235000212 | 4.01602E−06 |
| ENSG00000164294 | GPX8 | 1.234317717 | 1.67227E−07 |
| ENSG00000110848 | CD69 | 1.233437962 | 0.005637431 |
| ENSG00000138135 | CH25H | 1.233435129 | 0.023392804 |
| ENSG00000173641 | HSPB7 | 1.233063449 | 9.60877E−05 |
| ENSG00000169946 | ZFPM2 | 1.23183164 | 1.27435E−06 |
| ENSG00000112852 | PCDHB2 | 1.231248693 | 0.001578874 |
| ENSG00000123570 | RAB9B | 1.22728599 | 4.39928E−07 |
| ENSG00000128606 | LRRC17 | 1.226245678 | 9.09664E−06 |
| ENSG00000129116 | PALLD | 1.225342682 | 1.55949E−07 |
| ENSG00000166450 | PRTG | 1.224488053 | 0.000598677 |
| ENSG00000139971 | C14orf37 | 1.224263327 | 6.65939E−06 |
| ENSG00000204442 | FAM155A | 1.222993304 | 0.018983556 |
| ENSG00000119865 | CNRIP1 | 1.219685116 | 3.34733E−07 |
| ENSG00000156103 | MMP16 | 1.218771751 | 2.31144E−07 |
| ENSG00000131080 | EDA2R | 1.217055712 | 0.000101826 |
| ENSG00000122877 | EGR2 | 1.216936582 | 6.88846E−06 |
| ENSG00000163453 | IGFBP7 | 1.216563719 | 2.72187E−06 |
| ENSG00000166432 | ZMAT1 | 1.216400013 | 4.23217E−06 |
| ENSG00000166086 | JAM3 | 1.21607949 | 3.00641E−07 |
| ENSG00000182263 | FIGN | 1.216012357 | 0.000149172 |
| ENSG00000107518 | ATRNL1 | 1.215303423 | 0.000179975 |
| ENSG00000078018 | MAP2 | 1.212839612 | 1.84683E−06 |
| ENSG00000184156 | KCNQ3 | 1.211113152 | 0.000813382 |
| ENSG00000196381 | ZNF781 | 1.210427458 | 0.000240218 |
| ENSG00000197776 | KLHDC1 | 1.209432681 | 5.69234E−07 |
| ENSG00000164161 | HHIP | 1.209376306 | 6.71049E−05 |
| ENSG00000171812 | COL8A2 | 1.209367078 | 1.0172E−05 |
| ENSG00000178033 | FAM26E | 1.20847607 | 7.80945E−08 |
| ENSG00000160181 | TFF2 | 1.20693478 | 0.007531691 |
| ENSG00000106571 | GLI3 | 1.206013848 | 4.88081E−06 |
| ENSG00000129009 | ISLR | 1.205882434 | 1.38743E−05 |
| ENSG00000135272 | MDFIC | 1.205668504 | 7.01323E−07 |
| ENSG00000164418 | GRIK2 | 1.203042047 | 0.006037303 |
| ENSG00000152580 | IGSF10 | 1.202815648 | 0.00626239 |
| ENSG00000152402 | GUCY1A2 | 1.202681424 | 1.27435E−06 |
| ENSG00000100625 | SIX4 | 1.202376393 | 0.007794779 |
| ENSG00000151617 | EDNRA | 1.198067738 | 7.19393E−06 |
| ENSG00000087245 | MMP2 | 1.197459695 | 9.80267E−05 |
| ENSG00000172915 | NBEA | 1.197191441 | 7.90995E−05 |
| ENSG00000188921 | PTPLAD2 | 1.196049296 | 3.32858E−06 |
| ENSG00000147650 | LRP12 | 1.194550854 | 6.66468E−06 |
| ENSG00000013297 | CLDN11 | 1.193131487 | 1.30641E−05 |
| ENSG00000176971 | FIBIN | 1.191387166 | 4.62169E−06 |
| ENSG00000196188 | CTSE | 1.19036335 | 0.02867178 |
| ENSG00000124813 | RUNX2 | 1.187351485 | 2.0362E−06 |
| ENSG00000172201 | ID4 | 1.185672386 | 9.67639E−05 |
| ENSG00000134874 | DZIP1 | 1.184217346 | 3.31914E−08 |
| ENSG00000143387 | CTSK | 1.183440751 | 2.76708E−07 |
| ENSG00000139174 | PRICKLE1 | 1.183240996 | 3.24469E−06 |
| ENSG00000134533 | RERG | 1.181677477 | 0.006782333 |
| ENSG00000060718 | COL11A1 | 1.181154156 | 0.000502238 |
| ENSG00000145147 | SLIT2 | 1.180183549 | 2.18991E−06 |
| ENSG00000153292 | GPR110 | 1.179913651 | 0.016985086 |
| ENSG00000131459 | GFPT2 | 1.177343428 | 3.92505E−05 |
| ENSG00000173391 | OLR1 | 1.176668514 | 0.000157527 |
| ENSG00000211448 | DIO2 | 1.176566161 | 0.002794342 |
| ENSG00000145908 | ZNF300 | 1.173849773 | 0.005884854 |
| ENSG00000078589 | P2RY10 | 1.173467703 | 0.001941456 |
| ENSG00000002746 | HECW1 | 1.172942469 | 0.00042753 |
| ENSG00000122707 | RECK | 1.170766842 | 6.92282E−07 |
| ENSG00000011201 | KAL1 | 1.169712647 | 3.73246E−06 |
| ENSG00000162692 | VCAM1 | 1.168255525 | 4.87131E−05 |
| ENSG00000138311 | ZNF365 | 1.164097151 | 0.000503182 |
| ENSG00000104447 | TRPS1 | 1.162299394 | 1.02767E−06 |
| ENSG00000171451 | DSEL | 1.161696195 | 5.38477E−06 |
| ENSG00000170017 | ALCAM | 1.160712896 | 5.43327E−07 |
| ENSG00000139926 | FRMD6 | 1.160391169 | 6.3079E−07 |
| ENSG00000073910 | FRY | 1.160163469 | 1.43319E−07 |
| ENSG00000137273 | FOXF2 | 1.157583315 | 0.000505777 |
| ENSG00000136541 | ERMN | 1.155176394 | 0.000102981 |
| ENSG00000182771 | GRID1 | 1.150150735 | 0.001014429 |
| ENSG00000171502 | COL24A1 | 1.149995428 | 0.000173828 |
| ENSG00000077264 | PAK3 | 1.146860321 | 0.00406611 |
| ENSG00000240038 | AMY2B | 1.145605746 | 2.51038E−06 |
| ENSG00000163430 | FSTL1 | 1.144209777 | 9.66921E−07 |
| ENSG00000058091 | CDK14 | 1.143535823 | 1.01886E−08 |
| ENSG00000164764 | SBSPON | 1.143172285 | 0.045433189 |
| ENSG00000146197 | SCUBE3 | 1.142794148 | 0.009794541 |
| ENSG00000115602 | IL1RL1 | 1.142365242 | 0.003792042 |
| ENSG00000090104 | RGS1 | 1.140354037 | 0.000269751 |
| ENSG00000149256 | TENM4 | 1.140085482 | 6.3029E−05 |
| ENSG00000138185 | ENTPD1 | 1.136272638 | 7.44236E−07 |
| ENSG00000137968 | SLC44A5 | 1.134600285 | 0.002631951 |
| ENSG00000144369 | FAM171B | 1.133531118 | 0.000171487 |
| ENSG00000111087 | GLI1 | 1.132278497 | 5.13596E−06 |
| ENSG00000124491 | F13A1 | 1.131386557 | 0.001904786 |
| ENSG00000269920 | RP11-690P14.4 | 1.131325905 | 0.000122922 |
| ENSG00000115468 | EFHD1 | 1.131079049 | 1.06566E−05 |
| ENSG00000139278 | GLIPR1 | 1.130826292 | 3.20069E−06 |
| ENSG00000178343 | SHISA3 | 1.130796631 | 0.000140736 |
| ENSG00000258227 | CLEC5A | 1.130639256 | 6.68719E−05 |
| ENSG00000166927 | MS4A7 | 1.129035171 | 0.000106242 |
| ENSG00000149212 | SESN3 | 1.127242924 | 3.33122E−05 |
| ENSG00000133392 | MYH11 | 1.122036671 | 0.000620067 |
| ENSG00000127863 | TNFRSF19 | 1.121417576 | 0.03330431 |
| ENSG00000120693 | SMAD9 | 1.120809741 | 0.01515157 |
| ENSG00000154654 | NCAM2 | 1.119563764 | 0.001355873 |
| ENSG00000117586 | TNFSF4 | 1.118211022 | 2.97567E−06 |
| ENSG00000183889 | AC138969.4 | 1.11819356 | 0.003137054 |
| ENSG00000115461 | IGFBP5 | 1.116692007 | 0.000368757 |
| ENSG00000162946 | DISC1 | 1.115736631 | 2.73984E−06 |
| ENSG00000254535 | PABPC4L | 1.115653577 | 7.85258E−05 |
| ENSG00000154262 | ABCA6 | 1.115587536 | 0.000547756 |
| ENSG00000154258 | ABCA9 | 1.115423601 | 0.00044798 |
| ENSG00000173088 | C10orf131 | 1.113938456 | 0.000278822 |
| ENSG00000107186 | MPDZ | 1.110675366 | 9.6024E−06 |
| ENSG00000204381 | LAYN | 1.106894012 | 1.03747E−05 |
| ENSG00000138735 | PDE5A | 1.106156211 | 1.3397E−06 |
| ENSG00000133104 | SPG20 | 1.104999415 | 8.38798E−07 |
| ENSG00000140092 | FBLN5 | 1.103920818 | 0.000591908 |
| ENSG00000115295 | CLIP4 | 1.103736605 | 1.49464E−05 |
| ENSG00000147138 | GPR174 | 1.103719128 | 0.004945204 |
| ENSG00000174740 | PABPC5 | 1.103343026 | 0.000227181 |
| ENSG00000196159 | FAT4 | 1.101801065 | 5.63573E−05 |
| ENSG00000116194 | ANGPTL1 | 1.101127894 | 0.006070983 |
| ENSG00000127990 | SGCE | 1.100578254 | 0.000718744 |
| ENSG00000122870 | BICC1 | 1.100576063 | 2.81243E−06 |
| ENSG00000185008 | ROB02 | 1.099265746 | 0.019037708 |
| ENSG00000213694 | S1PR3 | 1.09874409 | 3.48793E−05 |
| ENSG00000067798 | NAV3 | 1.098411466 | 0.000208293 |
| ENSG00000106624 | AEBP1 | 1.097916846 | 0.00045447 |
| ENSG00000185052 | SLC24A3 | 1.09641977 | 9.88467E−06 |
| ENSG00000155754 | ALS2CR11 | 1.095856973 | 0.000336063 |
| ENSG00000119681 | LTBP2 | 1.094933995 | 7.4972E−05 |
| ENSG00000101134 | DOK5 | 1.093841101 | 4.8433E−06 |
| ENSG00000136546 | SCN7A | 1.092355329 | 0.035256333 |
| ENSG00000170801 | HTRA3 | 1.092109737 | 0.000182185 |
| ENSG00000257242 | C12orf79 | 1.091539632 | 0.003039378 |
| ENSG00000154175 | ABI3BP | 1.089786874 | 0.000182124 |
| ENSG00000143195 | ILDR2 | 1.089285377 | 1.62346E−05 |
| ENSG00000198300 | PEG3 | 1.087321859 | 9.22214E−05 |
| ENSG00000152495 | CAMK4 | 1.084809673 | 0.000153038 |
| ENSG00000185070 | FLRT2 | 1.081974628 | 1.77411E−05 |
| ENSG00000164694 | FNDC1 | 1.081486749 | 0.000200727 |
| ENSG00000178776 | C5orf46 | 1.081247748 | 0.000128588 |
| ENSG00000180044 | C3orf80 | 1.080834073 | 0.000500175 |
| ENSG00000149534 | M54A2 | 1.080611922 | 0.000757529 |
| ENSG00000053328 | METTL24 | 1.079449984 | 0.000272257 |
| ENSG00000073712 | FERMT2 | 1.078621925 | 7.74666E−06 |
| ENSG00000176928 | GCNT4 | 1.075942338 | 3.80515E−05 |
| ENSG00000184408 | KCND2 | 1.075921601 | 0.000302824 |
| ENSG00000071205 | ARHGAP10 | 1.074797855 | 1.17758E−05 |
| ENSG00000154553 | PDLIM3 | 1.073477109 | 6.43903E−05 |
| ENSG00000171533 | MAP6 | 1.07252774 | 4.76448E−06 |
| ENSG00000079150 | FKBP7 | 1.072477835 | 2.45449E−08 |
| ENSG00000111860 | CEP85L | 1.071194982 | 2.54905E−05 |
| ENSG00000213949 | ITGA1 | 1.068171951 | 2.75625E−07 |
| ENSG00000206432 | TMEM200C | 1.068045023 | 0.000141934 |
| ENSG00000022267 | FHL1 | 1.067042498 | 4.62835E−05 |
| ENSG00000182732 | RGS6 | 1.065466272 | 0.015282834 |
| ENSG00000155886 | SLC24A2 | 1.064729282 | 0.032903108 |
| ENSG00000168772 | CXXC4 | 1.064636093 | 0.002123427 |
| ENSG00000105971 | CAV2 | 1.064015979 | 3.55749E−06 |
| ENSG00000198046 | ZNF667 | 1.062667648 | 1.58089E−05 |
| ENSG00000174946 | GPR171 | 1.062334847 | 0.034964856 |
| ENSG00000151067 | CACNA1C | 1.060045584 | 1.51394E−05 |
| ENSG00000183722 | LHFP | 1.059077805 | 5.41821E−06 |
| ENSG00000061918 | GUCY1B3 | 1.058644005 | 2.03083E−06 |
| ENSG00000183578 | TNFAIP8L3 | 1.0568722 | 0.00806722 |
| ENSG00000130176 | CNN1 | 1.055943534 | 3.11441E−05 |
| ENSG00000169508 | GPR183 | 1.055470175 | 0.004408157 |
| ENSG00000069667 | RORA | 1.054780767 | 0.000126 |
| ENSG00000163431 | LMOD1 | 1.054106458 | 1.47358E−05 |
| ENSG00000187955 | COL14A1 | 1.049628519 | 0.000253813 |
| ENSG00000184349 | EFNA5 | 1.049530306 | 0.000219329 |
| ENSG00000135046 | ANXA1 | 1.049165077 | 7.85401E−05 |
| ENSG00000124212 | PTGIS | 1.047840989 | 0.000371338 |
| ENSG00000120658 | ENOX1 | 1.044269556 | 0.000381683 |
| ENSG00000124205 | EDN3 | 1.043678212 | 0.029743438 |
| ENSG00000203778 | FAM229B | 1.043236628 | 0.000285701 |
| ENSG00000079257 | LXN | 1.041719528 | 2.97567E−06 |
| ENSG00000151388 | ADAMTS12 | 1.041086206 | 8.34369E−06 |
| ENSG00000188171 | ZNF626 | 1.040237517 | 0.000316508 |
| ENSG00000166147 | FBN1 | 1.038279431 | 4.43826E−06 |
| ENSG00000121297 | TSHZ3 | 1.038122506 | 1.06429E−06 |
| ENSG00000151320 | AKAP6 | 1.036943473 | 7.53394E−06 |
| ENSG00000149970 | CNKSR2 | 1.036454196 | 0.002094779 |
| ENSG00000140538 | NTRK3 | 1.035310447 | 0.004965763 |
| ENSG00000113657 | DPYSL3 | 1.035103238 | 4.08187E−05 |
| ENSG00000161281 | COX7A1 | 1.03502624 | 0.001537258 |
| ENSG00000198795 | ZNF521 | 1.033008485 | 6.55014E−05 |
| ENSG00000091879 | ANGPT2 | 1.032906302 | 8.72461E−05 |
| ENSG00000172817 | CYP7B1 | 1.031096543 | 0.000247574 |
| ENSG00000170681 | MURC | 1.031087133 | 4.44393E−05 |
| ENSG00000141431 | ASXL3 | 1.03100311 | 0.002626424 |
| ENSG00000175857 | GAPT | 1.030449253 | 0.000189649 |
| ENSG00000137831 | UACA | 1.02949176 | 9.33137E−09 |
| ENSG00000019549 | SNAI2 | 1.028571266 | 1.29449E−05 |
| ENSG00000175395 | ZNF25 | 1.028531193 | 3.31914E−08 |
| ENSG00000184304 | PRKD1 | 1.027828768 | 1.13227E−06 |
| ENSG00000042980 | ADAM28 | 1.027310134 | 2.37072E−05 |
| ENSG00000104332 | SFRP1 | 1.027009502 | 0.000811385 |
| ENSG00000185862 | EVI2B | 1.025232063 | 0.000259929 |
| ENSG00000179909 | ZNF154 | 1.024936412 | 2.34785E−06 |
| ENSG00000137941 | TTLL7 | 1.024753669 | 0.00048813 |
| ENSG00000158186 | MRAS | 1.023633593 | 4.30876E−06 |
| ENSG00000164107 | HAND2 | 1.023465446 | 0.000341507 |
| ENSG00000150076 | CCDC7 | 1.023359419 | 1.25833E−05 |
| ENSG00000079215 | SLC1A3 | 1.022467446 | 0.001579851 |
| ENSG00000180828 | BHLHE22 | 1.021515955 | 0.005523297 |
| ENSG00000170786 | SDR16C5 | 1.020395127 | 0.028803293 |
| ENSG00000144935 | TRPC1 | 1.019616324 | 8.26821E−06 |
| ENSG00000164116 | GUCY1A3 | 1.019118915 | 8.58316E−06 |
| ENSG00000147488 | ST18 | 1.01841513 | 0.000537016 |
| ENSG00000198846 | TOX | 1.015764922 | 0.010839842 |
| ENSG00000114251 | WNT5A | 1.01566104 | 0.000106591 |
| ENSG00000077092 | RARB | 1.015126049 | 4.34993E−06 |
| ENSG00000129675 | ARHGEF6 | 1.015044573 | 9.42125E−06 |
| ENSG00000181264 | TMEM136 | 1.014645604 | 4.0308E−05 |
| ENSG00000133063 | CHIT1 | 1.014438106 | 0.034967068 |
| ENSG00000181007 | ZFP82 | 1.013638458 | 0.001050926 |
| ENSG00000030419 | IKZF2 | 1.013441523 | 4.73393E−06 |
| ENSG00000165028 | NIPSNAP3B | 1.011960146 | 6.97902E−05 |
| ENSG00000165633 | VSTM4 | 1.011056541 | 3.27269E−06 |
| ENSG00000113580 | NR3C1 | 1.00921802 | 9.2765E−06 |
| ENSG00000162620 | LRRIQ3 | 1.007955325 | 0.001693044 |
| ENSG00000167757 | KLK11 | 1.007520278 | 0.03319787 |
| ENSG00000279342 | AP000866.1 | 1.007380732 | 6.58012E−07 |
| ENSG00000165072 | MAMDC2 | 1.0071817 | 0.026128001 |
| ENSG00000128739 | SNRPN | 1.006895383 | 0.00152501 |
| ENSG00000153253 | SCN3A | 1.006646736 | 0.02899193 |
| ENSG00000118526 | TCF21 | 1.003870849 | 0.000290825 |
| ENSG00000163563 | MNDA | 1.003220314 | 8.98979E−05 |
| ENSG00000196664 | TLR7 | 1.003111776 | 0.000586797 |
| ENSG00000050555 | LAMC3 | 1.001461135 | 0.015109089 |
| ENSG00000090376 | IRAK3 | 1.001220316 | 1.89957E−05 |
| ENSG00000185112 | FAM43A | −1.001751529 | 0.000376952 |
| ENSG00000174276 | ZNHIT2 | −1.00469317 | 9.76788E−07 |
| ENSG00000173825 | TIGD3 | −1.00548575 | 0.000430346 |
| ENSG00000188486 | H2AFX | −1.006974588 | 7.75283E−07 |
| ENSG00000161888 | SPC24 | −1.007462984 | 1.17683E−05 |
| ENSG00000103253 | HAGHL | −1.008338066 | 0.000354038 |
| ENSG00000100197 | CYP2D6 | −1.009743478 | 0.010379291 |
| ENSG00000015520 | NPC1L1 | −1.012482977 | 0.024809378 |
| ENSG00000092621 | PHGDH | −1.01807365 | 0.035234547 |
| ENSG00000204632 | HLA-G | −1.019711771 | 0.009831723 |
| ENSG00000009950 | MLXIPL | −1.022450426 | 0.002998485 |
| ENSG00000147813 | NAPRT | −1.022826621 | 1.2798E−06 |
| ENSG00000197785 | ATAD3A | −1.023578406 | 4.62542E−08 |
| ENSG00000076826 | CAMSAP3 | −1.024286378 | 1.14245E−07 |
| ENSG00000125872 | LRRN4 | −1.027034577 | 0.032550873 |
| ENSG00000245848 | CEBPA | −1.028184918 | 2.88143E−07 |
| ENSG00000261236 | BOP1 | −1.031422954 | 7.42402E−07 |
| ENSG00000170684 | ZNF296 | −1.03381677 | 7.16344E−06 |
| ENSG00000080854 | IGSF9B | −1.039776887 | 0.025947588 |
| ENSG00000100726 | TELO2 | −1.045077366 | 3.69366E−08 |
| ENSG00000066735 | KIF26A | −1.053439198 | 0.026280282 |
| ENSG00000149150 | SLC43A1 | −1.053473873 | 1.32362E−05 |
| ENSG00000169174 | PCSK9 | −1.054887434 | 0.007819461 |
| ENSG00000168993 | CPLX1 | −1.055120067 | 0.003115199 |
| ENSG00000172828 | CES3 | −1.055338797 | 0.004948623 |
| ENSG00000149599 | DUSP15 | −1.058032584 | 0.022689093 |
| ENSG00000186193 | SAPCD2 | −1.05890805 | 0.00011606 |
| ENSG00000174669 | SLC29A2 | −1.06077456 | 1.58682E−06 |
| ENSG00000132744 | ACY3 | −1.066735599 | 0.000490538 |
| ENSG00000176919 | C8G | −1.068239685 | 0.027915602 |
| ENSG00000169683 | LRRC45 | −1.069647186 | 3.52775E−08 |
| ENSG00000181790 | BAI1 | −1.071046307 | 0.003470875 |
| ENSG00000124249 | KCNK15 | −1.074427039 | 0.011369482 |
| ENSG00000132361 | CLUH | −1.076184095 | 5.99561E−08 |
| ENSG00000118160 | SLC8A2 | −1.08059558 | 0.010839842 |
| ENSG00000083807 | SLC27A5 | −1.080803954 | 0.004509569 |
| ENSG00000101412 | E2F1 | −1.083432202 | 3.46171E−06 |
| ENSG00000120885 | CLU | −1.084858207 | 0.038559395 |
| ENSG00000244731 | C4A | −1.088076152 | 0.039172855 |
| ENSG00000130881 | LRP3 | −1.089753794 | 4.0074E−05 |
| ENSG00000075702 | WDR62 | −1.090124394 | 2.74828E−06 |
| ENSG00000168061 | SAC3D1 | −1.090353943 | 5.12645E−06 |
| ENSG00000177602 | GSG2 | −1.090655414 | 0.000953296 |
| ENSG00000162062 | C16orf59 | −1.091490514 | 2.36561E−05 |
| ENSG00000104140 | RHOV | −1.091638933 | 0.001003411 |
| ENSG00000007216 | SLC13A2 | −1.093057698 | 0.048337224 |
| ENSG00000262814 | MRPL12 | −1.097740165 | 6.58012E−07 |
| ENSG00000155093 | PTPRN2 | −1.098512936 | 0.02196475 |
| ENSG00000166391 | MOGAT2 | −1.099004103 | 0.007180743 |
| ENSG00000137474 | MY07A | −1.100568191 | 0.000105143 |
| ENSG00000276043 | UHRF1 | −1.104503535 | 8.29192E−06 |
| ENSG00000176428 | VPS37D | −1.105348126 | 0.001507569 |
| ENSG00000159423 | ALDH4A1 | −1.10814098 | 0.001116356 |
| ENSG00000172782 | FADS6 | −1.110321794 | 0.047627443 |
| ENSG00000140534 | TICRR | −1.111545752 | 2.97061E−05 |
| ENSG00000160867 | FGFR4 | −1.111845165 | 1.31042E−06 |
| ENSG00000115718 | PROC | −1.112809745 | 0.043806321 |
| ENSG00000099769 | IGFALS | −1.113018281 | 0.038559395 |
| ENSG00000131188 | PRR7 | −1.114347592 | 9.94915E−07 |
| ENSG00000166840 | GLYATL1 | −1.121036655 | 0.028826854 |
| ENSG00000167700 | MFSD3 | −1.123728167 | 1.58464E−07 |
| ENSG00000164620 | RELL2 | −1.126376462 | 3.14734E−05 |
| ENSG00000162897 | FCAMR | −1.126687354 | 0.003931285 |
| ENSG00000177380 | PPFIA3 | −1.12968427 | 4.42898E−07 |
| ENSG00000125144 | MT1G | −1.13147587 | 0.030981806 |
| ENSG00000170482 | SLC23A1 | −1.132254448 | 0.005223893 |
| ENSG00000127564 | PKMYT1 | −1.132438141 | 1.43537E−06 |
| ENSG00000183971 | NPW | −1.133018221 | 0.004280082 |
| ENSG00000160949 | TONSL | −1.134113855 | 2.81623E−07 |
| ENSG00000135097 | MSI1 | −1.13420336 | 0.035623024 |
| ENSG00000160932 | LY6E | −1.135450546 | 5.82577E−05 |
| ENSG00000258429 | PDF | −1.136902431 | 1.3397E−06 |
| ENSG00000187017 | ESPN | −1.138239397 | 0.000101684 |
| ENSG00000167701 | GPT | −1.141315424 | 0.000173096 |
| ENSG00000167771 | RCOR2 | −1.141957361 | 0.001805997 |
| ENSG00000189410 | SH2D5 | −1.145190586 | 0.022606753 |
| ENSG00000169962 | TAS1R3 | −1.145928107 | 0.00728111 |
| ENSG00000277363 | SRCIN1 | −1.147730626 | 4.18251E−05 |
| ENSG00000204128 | C2orf72 | −1.148085566 | 0.001778856 |
| ENSG00000111249 | CUX2 | −1.148558393 | 0.032607905 |
| ENSG00000274267 | HIST1H3B | −1.155845751 | 6.14265E−05 |
| ENSG00000234602 | MCIDAS | −1.157274122 | 0.007699226 |
| ENSG00000185101 | ANO9 | −1.166208972 | 1.38743E−05 |
| ENSG00000178821 | TMEM52 | −1.166457683 | 0.001754164 |
| ENSG00000165716 | FAM69B | −1.168533327 | 0.007163727 |
| ENSG00000167900 | TK1 | −1.17018382 | 8.53148E−06 |
| ENSG00000099617 | EFNA2 | −1.170438905 | 0.00048183 |
| ENSG00000146670 | CDCA5 | −1.173617071 | 5.69234E−07 |
| ENSG00000136457 | CHAD | −1.182023544 | 0.023720863 |
| ENSG00000136883 | KIF12 | −1.186005175 | 5.18794E−05 |
| ENSG00000169710 | FASN | −1.190277713 | 1.25719E−07 |
| ENSG00000138030 | KHK | −1.191482192 | 3.86632E−05 |
| ENSG00000182327 | GLTPD2 | −1.196092546 | 0.016960088 |
| ENSG00000260220 | MGC50722 | −1.197201902 | 0.026962886 |
| ENSG00000168539 | CHRM1 | −1.198602912 | 0.022798841 |
| ENSG00000186185 | KIF18B | −1.20081172 | 1.97821E−06 |
| ENSG00000137825 | ITPKA | −1.201066297 | 0.000913212 |
| ENSG00000280789 | PAGR1 | −1.214839339 | 7.72049E−05 |
| ENSG00000173621 | LRFN4 | −1.214944868 | 1.55949E−07 |
| ENSG00000164061 | BSN | −1.215223715 | 0.025903361 |
| ENSG00000177238 | TRIM72 | −1.222382743 | 0.023849438 |
| ENSG00000167711 | SERPINF2 | −1.223629729 | 0.028145001 |
| ENSG00000141505 | ASGR1 | −1.225548184 | 0.045750614 |
| ENSG00000165644 | COMTD1 | −1.227602584 | 3.99847E−07 |
| ENSG00000173894 | CBX2 | −1.234452318 | 0.000678006 |
| ENSG00000181625 | SLX1B | −1.234995479 | 0.006225067 |
| ENSG00000171236 | LRG1 | −1.260424178 | 0.006121707 |
| ENSG00000214456 | PLIN5 | −1.264174479 | 0.013042149 |
| ENSG00000115290 | GRB14 | −1.267659636 | 0.026580911 |
| ENSG00000089820 | ARHGAP4 | −1.269593592 | 3.23199E−06 |
| ENSG00000102109 | PCSK1N | −1.270769881 | 0.029488509 |
| ENSG00000142748 | FCN3 | −1.272330535 | 0.020660974 |
| ENSG00000198732 | SMOC1 | −1.272802772 | 0.036222164 |
| ENSG00000187045 | TMPRSS6 | −1.27327971 | 0.016419753 |
| ENSG00000089558 | KCNH4 | −1.29307111 | 0.001229742 |
| ENSG00000139438 | FAM222A | −1.29359271 | 0.000272257 |
| ENSG00000181418 | DDN | −1.298959188 | 2.05807E−05 |
| ENSG00000273604 | C17orf96 | −1.299769213 | 1.93426E−05 |
| ENSG00000105409 | ATP1A3 | −1.301076665 | 0.017641118 |
| ENSG00000167676 | PLIN4 | −1.303896568 | 0.025372704 |
| ENSG00000160957 | RECQL4 | −1.304548948 | 8.38587E−07 |
| ENSG00000159650 | UROC1 | −1.306216441 | 0.019745439 |
| ENSG00000187486 | KCNJ11 | −1.309606656 | 2.88429E−05 |
| ENSG00000109758 | HGFAC | −1.313280001 | 0.045084644 |
| ENSG00000167992 | VWCE | −1.318194033 | 0.005077692 |
| ENSG00000127586 | CHTF18 | −1.319980205 | 6.94018E−09 |
| ENSG00000166823 | MESP1 | −1.323889019 | 0.001845967 |
| ENSG00000053108 | FSTL4 | −1.34822064 | 0.035955607 |
| ENSG00000123561 | SERPINA7 | −1.358842508 | 0.047539566 |
| ENSG00000167513 | CDT1 | −1.369477799 | 2.41385E−07 |
| ENSG00000160224 | AIRE | −1.372022713 | 0.030742055 |
| ENSG00000101057 | MYBL2 | −1.372454731 | 1.65526E−06 |
| ENSG00000174990 | CA5A | −1.38459883 | 0.009925098 |
| ENSG00000166183 | ASPG | −1.39727814 | 0.004509381 |
| ENSG00000197165 | SULT1A2 | −1.404041006 | 0.00014855 |
| ENSG00000173826 | KCNH6 | −1.418866587 | 0.024724912 |
| ENSG00000160716 | CHRNB2 | −1.42788262 | 0.031135519 |
| ENSG00000149927 | DOC2A | −1.440224607 | 0.016348954 |
| ENSG00000161944 | ASGR2 | −1.447757676 | 0.044823356 |
| ENSG00000131910 | NR0B2 | −1.449023932 | 0.000708453 |
| ENSG00000125462 | C1orf61 | −1.47103537 | 0.013708059 |
| ENSG00000145850 | TIMD4 | −1.4882556 | 0.006886844 |
| ENSG00000152669 | CCNO | −1.495196794 | 1.20302E−05 |
| ENSG00000008735 | MAPK8IP2 | −1.500321268 | 0.006921494 |
| ENSG00000180767 | CHST13 | −1.509067755 | 0.005363605 |
| ENSG00000156222 | SLC28A1 | −1.509136305 | 0.018472246 |
| ENSG00000162460 | TMEM82 | −1.530716421 | 0.003893738 |
| ENSG00000103723 | AP3B2 | −1.531296828 | 0.022096857 |
| ENSG00000188883 | KLRG2 | −1.53704878 | 0.018096135 |
| ENSG00000198535 | C2CD4A | −1.550299035 | 0.000264523 |
| ENSG00000131187 | F12 | −1.550770758 | 0.000598677 |
| ENSG00000167588 | GPD1 | −1.558848619 | 0.000851273 |
| ENSG00000169750 | RAC3 | −1.576133554 | 2.74832E−07 |
| ENSG00000259803 | SLC22A31 | −1.577099411 | 0.04584251 |
| ENSG00000114113 | RBP2 | −1.577684832 | 0.008239525 |
| ENSG00000277196 | PRODH | −1.578341259 | 0.003279393 |
| ENSG00000174672 | BRSK2 | −1.584357103 | 0.00110229 |
| ENSG00000156564 | LRFN2 | −1.586216269 | 0.013302955 |
| ENSG00000057593 | F7 | −1.588647623 | 0.019402994 |
| ENSG00000197444 | OGDHL | −1.591753059 | 0.007481313 |
| ENSG00000060566 | CREB3L3 | −1.655731801 | 0.010903719 |
| ENSG00000185686 | PRAME | −1.668077149 | 0.029594841 |
| ENSG00000112494 | UNC93A | −1.670260488 | 0.009379115 |
| ENSG00000141485 | SLC13A5 | −1.678473209 | 0.025092248 |
| ENSG00000128564 | VGF | −1.682031349 | 0.039393053 |
| ENSG00000107147 | KCNT1 | −1.687512018 | 0.006545909 |
| ENSG00000069482 | GAL | −1.707460098 | 0.009620975 |
| ENSG00000130054 | FAM155B | −1.727654652 | 0.014942749 |
| ENSG00000139269 | INHBE | −1.741155005 | 0.009647956 |
| ENSG00000172482 | AGXT | −1.749329793 | 0.026798512 |
| ENSG00000105707 | HPN | −1.794590272 | 0.007953985 |
| ENSG00000120054 | CPN1 | −1.812734659 | 0.015558969 |
| ENSG00000138308 | PLA2G12B | −1.832138485 | 0.001435233 |
| ENSG00000101204 | CHRNA4 | −1.858084234 | 0.004352226 |
| ENSG00000196604 | POTEF | −1.867424293 | 0.000555537 |
| ENSG00000143627 | PKLR | −1.868735021 | 0.002057997 |
| ENSG00000177984 | LCN15 | −1.96847946 | 0.043777543 |
| ENSG00000164362 | TERT | −2.116087505 | 1.40839E−06 |
| ENSG00000188176 | SMTNL2 | −2.218288559 | 0.000338971 |
| ENSG00000159217 | IGF2BP1 | −2.756799479 | 0.036354585 |

Tables 4A-C: Differentially expressed miRNAs across SNF clusters in 93 metastatic miRNA samples identified by the limma method. (A) Differentially expressed miRNAs (DEMs) between SNF1 versus SNF2 and 3. (B) DEMs between SNF2 versus SNF1 and 3. (C) DEMs between SNF3 versus SNF1 and 2. Log 2FC: estimate of the log 2 fold-change corresponding to the contrast. Adj.P.Val: Benjamini-Hochberg corrected P-value. Cutoff values for DEGs are log 2FC=±1 and adj.P.Val≤0.05.

TABLE 4A
Differentially expressed miRNAs between SNF1 vs. SNF2 and SNF3

| Probe Set Name | Transcript ID | log2FC | adj P-Val |
|---|---|---|---|
| MIMAT0000461_st | hsa-miR-195-5p | −1.016509129 | 5.74058E−06 |
| MIMAT0000423_st | hsa-miR-125b-5p | −1.101525081 | 0.000255317 |
| MIMAT0000451_st | hsa-miR-150-5p | −1.111931191 | 0.002189755 |
| MIMAT0000098_st | hsa-miR-100-5p | −1.15185586 | 5.93555E−05 |
| MIMAT0000097_st | hsa-miR-99a-5p | −1.37583683 | 0.000145126 |

TABLE 4B
Differentially expressed miRNAs between SNF2 vs. SNF1 and SNF3

| Probe Set Name | Transcript ID | log2FC | adj P-Val |
|---|---|---|---|
| MIMAT0000421_st | hsa-miR-122-5p | 2.240488686 | 0.003540262 |
| MIMAT0019041_st | hsa-miR-4505 | 1.298904707 | 2.5899E−08 |
| MIMAT0017990_st | hsa-miR-3613-5p | 1.18902498 | 2.63247E−06 |
| MIMAT0022967_st | hsa-miR-3620-5p | 1.115649419 | 3.92575E−07 |
| MIMAT0031002_st | hsa-miR-8075 | 1.080687514 | 8.01934E−07 |
| MIMAT0019044_st | hsa-miR-4507 | 1.075979828 | 2.73172E−06 |
| MIMAT0019069_st | hsa-miR-4530 | 1.051551622 | 3.22856E−07 |
| MIMAT0010313_st | hsa-miR-762 | 1.019279452 | 2.73172E−06 |
| MIMAT0022938_st | hsa-miR-937-5p | 1.011491477 | 5.9441E−06 |
| MIMAT0024616_st | hsa-miR-6132 | 1.002733507 | 1.39191E−07 |
| MIMAT0000075_st | hsa-miR-20a-5p | −1.028612526 | 8.36836E−06 |
| MIMAT0000078_st | hsa-miR-23a-3p | −1.112055891 | 1.11136E−08 |

TABLE 4C
Differentially expressed miRNAs between SNF3 vs. SNF1 and SNF2

| Probe Set Name | Transcript ID | log2FC | adj P-Val |
|---|---|---|---|
| MIMAT0000232_st | hsa-miR-199a-3p | 1.634106484 | 7.30981E−12 |
| MIMAT0004563_st | hsa-miR-199b-3p | 1.634106484 | 7.30981E−12 |
| MIMAT0000461_st | hsa-miR-195-5p | 1.42367983 | 6.10227E−12 |
| MIMAT0000231_st | hsa-miR-199a-5p | 1.37234542 | 8.05432E−10 |
| MIMAT0000417_st | hsa-miR-15b-5p | 1.207406832 | 3.95917E−09 |
| MIMAT0000418_st | hsa-miR-23b-3p | 1.158743828 | 8.45554E−10 |
| MIMAT0000078_st | hsa-miR-23a-3p | 1.104474733 | 5.56265E−10 |
| MIMAT0000098_st | hsa-miR-100-5p | 1.091350504 | 2.33107E−06 |
| MIMAT0000082_st | hsa-miR-26a-5p | 1.064950068 | 1.97806E−10 |
| MIMAT0027430_st | hsa-miR-6765-5p | −1.00825087 | 5.76113E−09 |
| MIMAT0019071_st | hsa-miR-4532 | −1.013090134 | 1.59281E−05 |
| MIMAT0016907_st | hsa-miR-4281 | −1.013807197 | 7.45102E−08 |
| MIMAT0028113_st | hsa-miR-7108-5p | −1.016242495 | 5.55427E−08 |
| MIMAT0015082_st | hsa-miR-3197 | −1.017126606 | 1.95996E−08 |
| MIMAT0027600_st | hsa-miR-6850-5p | −1.020909622 | 1.21182E−08 |
| MIMAT0027506_st | hsa-miR-6803-5p | −1.02336335 | 2.09619E−06 |
| MIMAT0027616_st | hsa-miR-6858-5p | −1.023785192 | 2.81438E−08 |
| MIMAT0018961_st | hsa-miR-4443 | −1.039247982 | 5.53513E−09 |
| MIMAT0019868_st | hsa-miR-4739 | −1.039721011 | 1.11781E−09 |
| MIMAT0015055_st | hsa-miR-3178 | −1.050761351 | 1.01112E−08 |
| MIMAT0023252_st | hsa-miR-5787 | −1.055718868 | 3.79141E−07 |
| MIMAT0019229_st | hsa-miR-3940-5p | −1.058608437 | 2.09139E−08 |
| MIMAT0005922_st | hsa-miR-1268a | −1.062230989 | 1.97806E−10 |
| MIMAT0022946_st | hsa-miR-1237-5p | −1.063146983 | 2.62774E−08 |
| MIMAT0003326_st | hsa-miR-663a | −1.065257671 | 5.68431E−09 |
| MIMAT0007347_st | hsa-miR-1469 | −1.068502669 | 4.80996E−09 |
| MIMAT0027510_st | hsa-miR-6805-5p | −1.071861119 | 1.66313E−08 |
| MIMAT0027472_st | hsa-miR-6786-5p | −1.073166356 | 2.3974E−08 |
| MIMAT0026486_st | hsa-miR-328-5p | −1.077373947 | 1.71028E−09 |
| MIMAT0027526_st | hsa-miR-6813-5p | −1.078466785 | 8.45554E−10 |
| MIMAT0004609_st | hsa-miR-149-3p | −1.081848255 | 2.21105E−09 |
| MIMAT0019756_st | hsa-miR-4674 | −1.090871453 | 5.538E−10 |
| MIMAT0019913_st | hsa-miR-4763-3p | −1.093332207 | 1.94757E−08 |
| MIMAT0005582_st | hsa-miR-1228-5p | −1.111569746 | 3.72512E−09 |
| MIMAT0019027_st | hsa-miR-4492 | −1.111808824 | 1.73716E−07 |
| MIMAT0027482_st | hsa-miR-6791-5p | −1.114303081 | 6.60831E−08 |
| MIMAT0019018_st | hsa-miR-4484 | −1.124098667 | 3.55167E−09 |
| MIMAT0024616_st | hsa-miR-6132 | −1.125184942 | 5.56265E−10 |
| MIMAT0019715_st | hsa-miR-4651 | −1.125360214 | 6.43746E−09 |
| MIMAT0023710_st | hsa-miR-6085 | −1.126514316 | 1.11781E−09 |
| MIMAT0024599_st | hsa-miR-6126 | −1.128707561 | 3.45588E−06 |
| MIMAT0027412_st | hsa-miR-6756-5p | −1.150459507 | 3.95917E−09 |
| MIMAT0027532_st | hsa-miR-6816-5p | −1.162047562 | 5.56265E−10 |
| MIMAT0010313_st | hsa-miR-762 | −1.162936804 | 1.05505E−08 |
| MIMAT0027458_st | hsa-miR-6779-5p | −1.190669994 | 1.85776E−09 |
| MIMAT0019044_st | hsa-miR-4507 | −1.193928349 | 2.3974E−08 |
| MIMAT0022967_st | hsa-miR-3620-5p | −1.200066769 | 5.70033E−09 |
| MIMAT0019077_st | hsa-miR-1587 | −1.20141414 | 5.538E−10 |
| MIMAT0019859_st | hsa-miR-4734 | −1.202687453 | 1.53243E−09 |
| MIMAT0022938_st | hsa-miR-937-5p | −1.214187024 | 1.05505E−08 |
| MIMAT0019041_st | hsa-miR-4505 | −1.30510348 | 1.03447E−09 |
| MIMAT0018929_st | hsa-miR-4417 | −1.346495056 | 3.31374E−07 |

Tables 5A-C: Ensemble of gene set enrichment analyses for hallmark mSigDB pathway signatures. Pathway enrichment or depletion (i.e., direction) was determined for each SNF cluster against the others (e.g., (SNF1−(SNF2+SNF3)/2)). The Hallmark Signature gene list was retrieved from Broad Institute's mSigDB. Twelve gene set enrichment algorithms (including GSVA, GAGE, PADOG, etc.) were used for analyses, and run independently for each set of gene lists. Results for SNF1 are set forth in Table 5A; results for SNF2 are test forth in Table 5B; and results for SNF3 are set forth in Table 5C. Raw P-values for a given pathway were combined across algorithms using Fisher's method and adjusted for multiple testing corrections by Bonferroni's method. Log 2 transformed fold-change (Log 2FC) was averaged in a similar fashion. A collective significance score proportional to combined P-values and average Log 2FC was generated and scaled from 0-100 to assess the degree of pathway enrichment or depletion relative to the inclusive set.

TABLE 5A
Ensemble of gene set enrichment for SNF1

| | SNF1 | | | |
|---|---|---|---|---|
| Hallmark Signature | Adj. P-value | Avg. Log2FC | Direction | Significance |
| HALLMARK_ADIPOGENESIS | 0.562602325 | 0.23191575 | + | 0.126012262 |
| HALLMARK_ALLOGRAFT_REJECTION | 6.75282E−63 | 0.553139857 | − | 82.1598301 |
| HALLMARK_ANDROGEN_RESPONSE | 0.953425282 | 0.250863329 | − | 0 |
| HALLMARK_ANGIOGENESIS | 9.00116E−24 | 0.436617029 | − | 24.03100939 |
| HALLMARK_APICAL_JUNCTION | 7.93541E−21 | 0.364378219 | − | 17.48860976 |
| HALLMARK_APICAL_SURFACE | 8.1046E−12 | 0.373536233 | − | 9.887230372 |
| HALLMARK_APOPTOSIS | 6.12844E−09 | 0.332983644 | − | 6.522077617 |
| HALLMARK_BILE_ACID_METABOLISM | 8.64164E−12 | 0.304744608 | − | 8.043789379 |
| HALLMARK_CHOLESTEROL_HOMEOSTASIS | 0.679276374 | 0.259427257 | − | 0.091697977 |
| HALLMARK_COAGULATION | 8.574E−79 | 0.484603535 | − | 90.38557752 |
| HALLMARK_COMPLEMENT | 4.87698E−28 | 0.395441688 | − | 25.79468901 |
| HALLMARK_DNA_REPAIR | 6.06118E−18 | 0.237931563 | + | 9.776315817 |
| HALLMARK_E2F_TARGETS | 2.35724E−38 | 0.387862836 | + | 34.86066987 |
| HALLMARK_EPITHELIAL_MESENCHYMAL_TRANSITION | 1.18976E−77 | 0.54410568 | − | 100 |
| HALLMARK_ESTROGEN_RESPONSE_EARLY | 3.29177E−08 | 0.303711199 | − | 5.417803144 |
| HALLMARK_ESTROGEN_RESPONSE_LATE | 5.77006E−18 | 0.353150484 | − | 14.53457159 |
| HALLMARK_FATTY_ACID_METABOLISM | 0.505352718 | 0.239834804 | + | 0.157448737 |
| HALLMARK_G2M_CHECKPOINT | 1.14089E−30 | 0.37450212 | + | 26.78246369 |
| HALLMARK_GLYCOLYSIS | 0.028472201 | 0.262976639 | + | 0.958795842 |
| HALLMARK_HEDGEHOG_SIGNALING | 4.35218E−10 | 0.402046073 | − | 8.980849187 |
| HALLMARK_HEME_METABOLISM | 0.633625016 | 0.249634839 | − | 0.105790836 |
| HALLMARK_HYPOXIA | 0.000531718 | 0.294139451 | − | 2.28891919 |
| HALLMARK_IL2_STAT5_SIGNALING | 2.20482E−13 | 0.381886699 | − | 11.5369519 |
| HALLMARK_IL6_JAK_STAT3_SIGNALING | 4.37758E−25 | 0.496155656 | − | 28.86637252 |
| HALLMARK_INFLAMMATORY_RESPONSE | 1.72579E−64 | 0.53516896 | − | 81.52660353 |
| HALLMARK_INTERFERON_ALPHA_RESPONSE | 2.56172E−21 | 0.464322514 | − | 22.83372197 |
| HALLMARK_INTERFERON_GAMMA_RESPONSE | 5.73495E−41 | 0.508877735 | − | 48.9195911 |
| HALLMARK_KRAS_SIGNALING_DN | 1.19638E−56 | 0.416757758 | − | 55.67706303 |
| HALLMARK_KRAS_SIGNALING_UP | 1.77104E−57 | 0.472465669 | − | 64.05768668 |
| HALLMARK_MITOTIC_SPINDLE | 2.35806E−09 | 0.274931735 | + | 5.655361679 |
| HALLMARK_MTORC1_SIGNALING | 1.11639E−12 | 0.256734732 | + | 7.319839135 |
| HALLMARK_MYC_TARGETS_V1 | 3.43273E−39 | 0.290461867 | + | 26.68402562 |
| HALLMARK_MYC_TARGETS_V2 | 6.0312E−22 | 0.277009724 | + | 14.03310559 |
| HALLMARK_MYOGENESIS | 4.50624E−31 | 0.387783334 | − | 28.10652706 |
| HALLMARK_NOTCH_SIGNALING | 9.31025E−07 | 0.33759945 | − | 4.852771165 |
| HALLMARK_OXIDATIVE_PHOSPHORYLATION | 8.77461E−09 | 0.178039061 | + | 3.415120409 |
| HALLMARK_P53_PATHWAY | 1.08052E−05 | 0.286922361 | − | 3.392519492 |
| HALLMARK_PANCREAS_BETA_CELLS | 1.01417E−08 | 0.459352744 | + | 8.761839682 |
| HALLMARK_PEROXISOME | 0.431746974 | 0.242997597 | + | 0.199384512 |
| HALLMARK_PI3K_AKT_MTOR_SIGNALING | 0.233835825 | 0.252230734 | + | 0.367943227 |
| HALLMARK_PROTEIN_SECRETION | 0.431746974 | 0.169764376 | + | 0.135553192 |
| HALLMARK_REACTIVE_OXIGEN_SPECIES_PATHWAY | 0.496910388 | 0.242744195 | + | 0.163753185 |
| HALLMARK_SPERMATOGENESIS | 4.5866E−15 | 0.342373456 | + | 11.71790297 |
| HALLMARK_TGF_BETA_SIGNALING | 0.001440397 | 0.23403223 | − | 1.576612534 |
| HALLMARK_TNFA_SIGNALING_VIA_NFKB | 2.31011E−16 | 0.371786122 | − | 13.87862487 |
| HALLMARK_UNFOLDED_PROTEIN_RESPONSE | 4.75471E−17 | 0.231061775 | + | 8.999777787 |
| HALLMARK_UV_RESPONSE_DN | 2.36277E−07 | 0.334683613 | − | 5.287018314 |
| HALLMARK_UV_RESPONSE_UP | 0.073726326 | 0.314916265 | − | 0.839686559 |
| HALLMARK_WNT_BETA_CATENIN_SIGNALING | 0.00030316 | 0.296203452 | − | 2.477772217 |
| HALLMARK_XENOBIOTIC_METABOLISM | 8.85403E−21 | 0.302492803 | − | 14.48187852 |

TABLE 5B
Ensemble of gene set enrichment for SNF2

| | SNF2 | | | |
|---|---|---|---|---|
| Hallmark Signature | Adj. P-value | Avg. Log2FC | Direction | Significance |
| HALLMARK_ADIPOGENESIS | 5.82382E−06 | 0.376237508 | + | 5.882317613 |
| HALLMARK_ALLOGRAFT_REJECTION | 1.45945E−40 | 0.543595115 | + | 72.99772011 |
| HALLMARK_ANDROGEN_RESPONSE | 2.99454E−12 | 0.370886686 | − | 13.73926065 |
| HALLMARK_ANGIOGENESIS | 0.00019338 | 0.411790376 | − | 4.381113101 |
| HALLMARK_APICAL_JUNCTION | 4.23961E−19 | 0.446257167 | + | 27.12128001 |
| HALLMARK_APICAL_SURFACE | 1.11488E−15 | 0.496384333 | + | 24.47343763 |
| HALLMARK_APOPTOSIS | 0.000198394 | 0.373000655 | + | 3.875844134 |
| HALLMARK_BILE_ACID_METABOLISM | 5.03525E−13 | 0.450256669 | + | 18.04634011 |
| HALLMARK_CHOLESTEROL_HOMEOSTASIS | 1.62885E−05 | 0.420630602 | + | 6.03405282 |
| HALLMARK_COAGULATION | 2.58606E−59 | 0.504790639 | + | 100 |
| HALLMARK_COMPLEMENT | 8.23511E−16 | 0.446198845 | + | 22.11507166 |
| HALLMARK_DNA_REPAIR | 3.14449E−07 | 0.368730815 | − | 7.342007266 |
| HALLMARK_E2F_TARGETS | 1.23697E−08 | 0.374919901 | − | 9.275439297 |
| HALLMARK_EPITHELIAL_MESENCHYMAL_TRANSITION | 1.9632E−21 | 0.407434289 | + | 27.93211451 |
| HALLMARK_ESTROGEN_RESPONSE_EARLY | 3.48034E−19 | 0.393898117 | + | 23.95655693 |
| HALLMARK_ESTROGEN_RESPONSE_LATE | 1.34211E−27 | 0.431906885 | + | 38.738538 |
| HALLMARK_FATTY_ACID_METABOLISM | 6.39585E−05 | 0.418958518 | − | 5.15824815 |
| HALLMARK_G2M_CHECKPOINT | 7.83336E−10 | 0.376504652 | − | 10.85654182 |
| HALLMARK_GLYCOLYSIS | 1.07007E−05 | 0.385285346 | + | 5.696741514 |
| HALLMARK_HEDGEHOG_SIGNALING | 0.063128326 | 0.342809773 | − | 0.569586763 |
| HALLMARK_HEME_METABOLISM | 1.91004E−08 | 0.416505838 | − | 10.12868816 |
| HALLMARK_HYPOXIA | 0.003124275 | 0.364832831 | + | 2.283534499 |
| HALLMARK_IL2_STAT5_SIGNALING | 2.10035E−05 | 0.406168105 | + | 5.645056887 |
| HALLMARK_IL6_JAK_STAT3_SIGNALING | 3.90097E−22 | 0.534823842 | + | 38.20549716 |
| HALLMARK_INFLAMMATORY_RESPONSE | 5.96656E−32 | 0.474979295 | + | 49.73266933 |
| HALLMARK_INTERFERON_ALPHA_RESPONSE | 2.06717E−21 | 0.544327528 | + | 37.55516943 |
| HALLMARK_INTERFERON_GAMMA_RESPONSE | 1.4102E−30 | 0.551942823 | + | 55.34125652 |
| HALLMARK_KRAS_SIGNALING_DN | 1.80809E−45 | 0.48049015 | + | 72.46554722 |
| HALLMARK_KRAS_SIGNALING_UP | 6.89314E−23 | 0.454194231 | + | 33.48581418 |
| HALLMARK_MITOTIC_SPINDLE | 4.86623E−17 | 0.40576277 | − | 21.73498143 |
| HALLMARK_MTORC1_SIGNALING | 2.36725E−09 | 0.359383083 | − | 9.736464696 |
| HALLMARK_MYC_TARGETS_V1 | 2.47646E−17 | 0.336329169 | − | 18.20962527 |
| HALLMARK_MYC_TARGETS_V2 | 0.071654539 | 0.34927155 | + | 0.530500529 |
| HALLMARK_MYOGENESIS | 5.11649E−22 | 0.466009232 | + | 32.99537081 |
| HALLMARK_NOTCH_SIGNALING | 0.127045562 | 0.27257069 | − | 0 |
| HALLMARK_OXIDATIVE_PHOSPHORYLATION | 4.53474E−05 | 0.312878012 | − | 3.800649111 |
| HALLMARK_P53_PATHWAY | 2.79313E−20 | 0.384211934 | + | 24.78207268 |
| HALLMARK_PANCREAS_BETA_CELLS | 1.32978E−08 | 0.514962394 | − | 12.99593374 |
| HALLMARK_PEROXISOME | 0.000540492 | 0.391346358 | + | 3.526670958 |
| HALLMARK_PI3K_AKT_MTOR_SIGNALING | 2.79466E−08 | 0.406359199 | − | 9.632656233 |
| HALLMARK_PROTEIN_SECRETION | 6.46486E−32 | 0.401763528 | − | 41.8905431 |
| HALLMARK_REACTIVE_OXIGEN_SPECIES_PATHWAY | 6.84591E−14 | 0.337891399 | + | 14.33325417 |
| HALLMARK_SPERMATOGENESIS | 8.98491E−25 | 0.399383156 | − | 31.91097763 |
| HALLMARK_TGF_BETA_SIGNALING | 3.39391E−07 | 0.358791161 | − | 7.081093476 |
| HALLMARK_TNFA_SIGNALING_VIA_NFKB | 5.51592E−08 | 0.401525725 | + | 9.103927568 |
| HALLMARK_UNFOLDED_PROTEIN_RESPONSE | 6.90293E−14 | 0.353336182 | − | 15.02213965 |
| HALLMARK_UV_RESPONSE_DN | 4.80807E−07 | 0.391600615 | − | 7.602798279 |
| HALLMARK_UV_RESPONSE_UP | 5.03525E−13 | 0.425384762 | + | 17.00347286 |
| HALLMARK_WNT_BETA_CATENIN_SIGNALING | 1.14962E−08 | 0.320004118 | + | 7.829567983 |
| HALLMARK_XENOBIOTIC_METABOLISM | 2.79972E−24 | 0.440206499 | + | 34.51707457 |

TABLE 5C
Ensemble of gene set enrichment for SNF3

| | SNF3 | | | |
|---|---|---|---|---|
| Hallmark Signature | Adj. P-value | Avg. Log2FC | Direction | Significance |
| HALLMARK_ADIPOGENESIS | 1.55818E−08 | 0.347062376 | + | 5.830276063 |
| HALLMARK_ALLOGRAFT_REJECTION | 1.54771E−30 | 0.409633949 | + | 26.66786146 |
| HALLMARK_ANDROGEN_RESPONSE | 5.58612E−15 | 0.352177023 | + | 10.89597364 |
| HALLMARK_ANGIOGENESIS | 3.45876E−24 | 0.598625543 | + | 30.68790239 |
| HALLMARK_APICAL_JUNCTION | 0.014311071 | 0.392496006 | + | 1.475421281 |
| HALLMARK_APICAL_SURFACE | 0.008557246 | 0.355582565 | − | 1.500278679 |
| HALLMARK_APOPTOSIS | 0.24028799 | 0.3370387 | + | 0.345621249 |
| HALLMARK_BILE_ACID_METABOLISM | 6.40651E−17 | 0.452607089 | − | 15.96124558 |
| HALLMARK_CHOLESTEROL_HOMEOSTASIS | 1.50612E−08 | 0.414956169 | − | 7.00617984 |
| HALLMARK_COAGULATION | 7.28876E−78 | 0.591799104 | + | 100 |
| HALLMARK_COMPLEMENT | 3.31628E−14 | 0.383671799 | + | 11.22954857 |
| HALLMARK_DNA_REPAIR | 7.09556E−18 | 0.296873524 | − | 11.05288138 |
| HALLMARK_E2F_TARGETS | 4.01343E−05 | 0.317359552 | − | 2.947776837 |
| HALLMARK_EPITHELIAL_MESENCHYMAL_TRANSITION | 2.98646E−64 | 0.5504785 | + | 76.57662828 |
| HALLMARK_ESTROGEN_RESPONSE_EARLY | 1.95684E−11 | 0.335674703 | − | 7.770914865 |
| HALLMARK_ESTROGEN_RESPONSE_LATE | 1.26415E−28 | 0.3670826 | − | 22.34674831 |
| HALLMARK_FATTY_ACID_METABOLISM | 0.026445455 | 0.316655267 | + | 0.98347634 |
| HALLMARK_G2M_CHECKPOINT | 0.004086786 | 0.346547757 | − | 1.703232442 |
| HALLMARK_GLYCOLYSIS | 2.14691E−06 | 0.307696685 | − | 3.712745619 |
| HALLMARK_HEDGEHOG_SIGNALING | 2.66912E−06 | 0.472783662 | + | 5.666836546 |
| HALLMARK_HEME_METABOLISM | 1.62438E−10 | 0.308692274 | + | 6.515026382 |
| HALLMARK_HYPOXIA | 0.025953316 | 0.326899469 | + | 1.024768452 |
| HALLMARK_IL2_STAT5_SIGNALING | 0.035655456 | 0.360532179 | + | 1.032675815 |
| HALLMARK_IL6_JAK_STAT3_SIGNALING | 0.001256043 | 0.355292971 | − | 2.148273749 |
| HALLMARK_INFLAMMATORY_RESPONSE | 1.44422E−30 | 0.389268775 | + | 25.36214016 |
| HALLMARK_INTERFERON_ALPHA_RESPONSE | 0.664951063 | 0.288462241 | − | 0 |
| HALLMARK_INTERFERON_GAMMA_RESPONSE | 0.059074297 | 0.324142705 | + | 0.761258006 |
| HALLMARK_KRAS_SIGNALING_DN | 4.17203E−55 | 0.411178484 | − | 48.92382364 |
| HALLMARK_KRAS_SIGNALING_UP | 5.55843E−49 | 0.444215624 | + | 46.8972376 |
| HALLMARK_MITOTIC_SPINDLE | 2.77845E−06 | 0.332372127 | − | 3.937846618 |
| HALLMARK_MTORC1_SIGNALING | 3.81536E−05 | 0.315587428 | − | 2.945903414 |
| HALLMARK_MYC_TARGETS_V1 | 0.03492292 | 0.23193917 | − | 0.628946187 |
| HALLMARK_MYC_TARGETS_V2 | 2.22275E−30 | 0.368170912 | − | 23.83027384 |
| HALLMARK_MYOGENESIS | 5.219E−14 | 0.417339246 | + | 12.04454127 |
| HALLMARK_NOTCH_SIGNALING | 1.45209E−05 | 0.34372808 | + | 3.534839252 |
| HALLMARK_OXIDATIVE_PHOSPHORYLATION | 1.45209E−05 | 0.237455231 | − | 2.407287582 |
| HALLMARK_P53_PATHWAY | 3.00444E−06 | 0.294412433 | − | 3.453380966 |
| HALLMARK_PANCREAS_BETA_CELLS | 2.51102E−10 | 0.418358872 | + | 8.695839717 |
| HALLMARK_PEROXISOME | 0.002008089 | 0.279801031 | − | 1.542951276 |
| HALLMARK_PI3K_AKT_MTOR_SIGNALING | 2.46179E−05 | 0.346054892 | + | 3.385539093 |
| HALLMARK_PROTEIN_SECRETION | 5.94544E−28 | 0.330636676 | + | 19.62936657 |
| HALLMARK_REACTIVE_OXIGEN_SPECIES_PATHWAY | 1.6742E−11 | 0.229941171 | − | 5.322020787 |
| HALLMARK_SPERMATOGENESIS | 0.00097818 | 0.327090841 | + | 2.046742764 |
| HALLMARK_TGF_BETA_SIGNALING | 3.32207E−13 | 0.340143129 | + | 9.196290718 |
| HALLMARK_TNFA_SIGNALING_VIA_NFKB | 0.047592797 | 0.324563372 | + | 0.829197421 |
| HALLMARK_UNFOLDED_PROTEIN_RESPONSE | 3.21878E−06 | 0.29786512 | + | 3.475645011 |
| HALLMARK_UV_RESPONSE_DN | 4.6735E−31 | 0.418429487 | + | 27.7200871 |
| HALLMARK_UV_RESPONSE_UP | 1.06469E−15 | 0.347575215 | − | 11.30086584 |
| HALLMARK_WNT_BETA_CATENIN_SIGNALING | 0.044318157 | 0.276535424 | − | 0.708681745 |
| HALLMARK_XENOBIOTIC_METABOLISM | 3.63495E−34 | 0.433604847 | − | 31.68602021 |

Table 6: Ensemble of gene set enrichment analyses for custom colorectal cancer pathways. Pathway enrichment or depletion (i.e., direction) was determined for each SNF against the others (e.g., (SNF1−(SNF2+SNF3)/2)). A compilation of pathways associated immunology, metabolism, canonical pathways, cancer signatures, and stromal infiltration estimates were retrieved from¹⁴. Twelve gene set enrichment algorithms (including GSVA, GAGE, PADOG, etc.) were used for analyses, and run independently for each set of gene lists. Raw P-values for a given pathway were combined across algorithms using Fisher's method and adjusted for multiple testing corrections by Bonferroni's method. Log 2 transformed fold-change (Log 2FC) was averaged in a similar fashion. A collective significance score proportional to combined p-values and average Log 2FC was generated and scaled from 0-100 to assess the degree of pathway enrichment or depletion relative to the inclusive set.

TABLE 6

| EGSEA Gene Set | Adj. P-value | Avg. Log2FC | Direction | Significance |
|---|---|---|---|---|
| | SNF1 | | | |
| ALANINE_ASPARTATE_GLUTAMATE_KEGG | 6.45E−03 | 0.3121 | + | 0.60 |
| AMINO_SUGAR_NUCLEO_METAB_KEGG | 5.02E−03 | 0.2700 | − | 0.55 |
| ARACHNOID_METAB_KEGG | 2.09E−36 | 0.4538 | − | 14.32 |
| CASPASE_BIOCARTA | 9.64E−01 | 0.2310 | + | 0.00 |
| CELL_CYCLE_BIOCARTA | 1.14E−08 | 0.4261 | + | 2.99 |
| CELL_CYCLE_PROTEASOME_REACTOME | 8.97E−27 | 0.3090 | + | 7.12 |
| CETUXIMAB_BENEFIT | 5.24E−08 | 1.0630 | + | 6.84 |
| COMPLEMENT_BIOCARTA | 4.85E−33 | 0.5989 | − | 17.12 |
| COMPLEMENT_COAG_KEGG | 1.45E−104 | 0.5228 | − | 48.01 |
| CRYPT_BASE | 1.46E−30 | 0.4270 | + | 11.27 |
| CRYPT_TOP | 1.49E−08 | 0.2952 | − | 2.04 |
| CSC_BATLLE | 4.74E−10 | 0.3773 | − | 3.11 |
| EMT_CORE_GENES | 3.74E−24 | 0.7653 | − | 15.86 |
| EPITH_LOBODA | 9.90E−01 | 0.2649 | − | 0.00 |
| FATTY_ACID_METAB_KEGG | 7.84E−01 | 0.1922 | + | 0.02 |
| FGF_ACT_CP | 8.71E−02 | 0.2797 | − | 0.26 |
| FOLFIRI_RESPONSE | 4.09E−06 | 0.3726 | − | 1.78 |
| FOLFOX_RESPONSE_DOWN | 2.29E−08 | 0.3564 | − | 2.41 |
| FRUTOSE_MANNOSE_METAB_KEGG | 9.90E−01 | 0.2460 | + | 0.00 |
| GALACTOSE_METAB_KEGG | 8.80E−01 | 0.2329 | − | 0.01 |
| GLUTAMINE_AMINO_ACID_GO_BP | 1.48E−01 | 0.2342 | + | 0.17 |
| GLUTAMINE_GO_BP | 3.09E−12 | 0.3239 | + | 3.30 |
| GLUTATHIONE_KEGG | 1.49E−01 | 0.2809 | − | 0.20 |
| GLYCEROPHOSPHOLIPID_GO_BP | 9.90E−01 | 0.2072 | − | 0.00 |
| GLYCEROPHOSPHOLIPID_METAB_KEGG | 9.64E−01 | 0.2738 | − | 0.00 |
| IGF1R_CP | 2.73E−01 | 0.1866 | + | 0.09 |
| IMMUNE_CD8MACRO_GALON | 6.33E−03 | 0.6922 | − | 1.35 |
| IMMUNE_ESTIMATE | 1.02E−97 | 0.6903 | − | 59.21 |
| IMMUNE_MDSC_ALBELDA | 2.47E−06 | 0.3828 | − | 1.90 |
| IMMUNE_MDSC_CERWENKA | 1.85E−08 | 1.0274 | − | 7.03 |
| IMMUNE_MSC | 3.10E−06 | 0.4365 | − | 2.13 |
| IMMUNE_NKC_BREAST | 1.03E−10 | 0.7383 | − | 6.52 |
| IMMUNE_RESP_GO_BP | 4.76E−95 | 0.5717 | − | 47.69 |
| IMMUNE_TH1_GALON | 1.52E−04 | 0.5974 | − | 2.02 |
| IMMUNE_TH17_GOUNARI | 2.92E−17 | 0.4639 | − | 6.78 |
| IMMUNE_THF_BREAST | 8.54E−07 | 0.5775 | − | 3.10 |
| IMMUNE_TREG_GALON | 6.08E−11 | 0.7164 | − | 6.47 |
| IMMUNE_TREG_LUCAS | 3.35E−06 | 0.4439 | + | 2.15 |
| IMMUNE_TREG_PASTILLE | 8.13E−11 | 0.5885 | − | 5.25 |
| INTEGRIN_BETA3_CP | 1.10E−21 | 0.4985 | − | 9.24 |
| INTEGRIN_BIOCARTA | 2.26E−03 | 0.1980 | − | 0.46 |
| JAK_STAT_GO_BP | 1.56E−04 | 0.3703 | − | 1.25 |
| JAK_STAT_KEGG | 1.94E−13 | 0.3674 | − | 4.13 |
| KEGG_CELL_CYCLE | 2.88E−22 | 0.3461 | + | 6.59 |
| KRAS_50_UP | 2.42E−41 | 0.6080 | − | 21.84 |
| LINOLEIC_METAB_KEGG | 2.12E−24 | 0.4010 | − | 8.40 |
| LYSOPHOSPHOLIPID_PID | 3.43E−08 | 0.2692 | − | 1.78 |
| MAPK_ACT_GO_BP | 2.97E−05 | 0.3023 | − | 1.21 |
| MAPK_KEGG | 3.63E−09 | 0.3242 | − | 2.42 |
| MATRIX_REMODEL_REACTOME | 4.51E−95 | 0.5268 | − | 43.96 |
| MESENCH_LOBODA | 8.97E−27 | 0.5880 | − | 13.55 |
| MYC_TARGETS_CP | 1.43E−24 | 0.2934 | + | 6.19 |
| MYC_TARGETS_ZELLER | 6.04E−26 | 0.3278 | + | 7.31 |
| NFKB_BIOCARTA | 1.16E−07 | 0.2644 | − | 1.62 |
| NITROGEN_METAB_KEGG | 1.52E−02 | 0.2839 | + | 0.46 |
| NOTCH_CP | 8.55E−01 | 0.2300 | + | 0.01 |
| NOTCH_REACTOME | 2.85E−01 | 0.2365 | + | 0.11 |
| NUCLEOTIDE_METAB_REACTOME | 3.43E−08 | 0.2753 | + | 1.82 |
| OXALIPLATIN_NCI60 | 6.42E−06 | 0.3554 | − | 1.63 |
| PD1_REACTOME | 7.10E−28 | 0.7587 | − | 18.22 |
| PENTOSE_GLUC_METAB_KEGG | 3.09E−12 | 0.3239 | + | 3.30 |
| PI3K_ACT_GO_BP | 9.90E−01 | 0.1734 | − | 0.00 |
| PI3K_ACT_REACTOME | 9.90E−01 | 0.1734 | − | 0.00 |
| PROTEASOME_KEGG | 1.25E−14 | 0.1688 | + | 2.08 |
| SERRATED_UP | 1.15E−01 | 0.2394 | + | 0.20 |
| SHH_CP | 2.99E−01 | 0.2535 | + | 0.12 |
| SHH_KEGG | 4.90E−22 | 0.4518 | − | 8.51 |
| SRC_ACT_BILD | 1.07E−01 | 0.2457 | + | 0.21 |
| SRC_ACT_BIOCARTA | 3.77E−12 | 0.3583 | + | 3.62 |
| STARCH_SUCROSE_METAB_KEGG | 1.71E−16 | 0.3235 | + | 4.51 |
| STROMAL_ESTIMATE | 9.72E−148 | 0.7691 | − | 100.00 |
| TGFB_1 | 2.61E−10 | 0.4074 | − | 3.45 |
| TGFB_2 | 5.95E−24 | 0.3303 | − | 6.78 |
| TGFB_CORE_GENES | 2.26E−01 | 0.2601 | − | 0.15 |
| TGFB_KEGG | 8.42E−02 | 0.3318 | + | 0.31 |
| TRANSLATION_RIBOS_REACTOME | 1.48E−18 | 0.2379 | + | 3.75 |
| TYROSINE_METAB_KEGG | 1.10E−08 | 0.2592 | + | 1.82 |
| VEGF_VEGFR_CP | 3.57E−07 | 0.3919 | − | 2.23 |
| VEGF_VEGFR_REACTOME | 4.11E−12 | 0.4352 | − | 4.38 |
| WNT_BIOCARTA | 5.10E−01 | 0.2460 | + | 0.06 |
| WNT_FLIER | 5.31E−13 | 0.2419 | + | 2.63 |
| WOUND_RESPONSE_GO_BP | 3.06E−88 | 0.5037 | − | 38.98 |
| | SNF2 | | | |
| ALANINE_ASPARTATE_GLUTAMATE_KEGG | 1.43E−03 | 0.5259 | + | 2.95 |
| AMINO_SUGAR_NUCLEO_METAB_KEGG | 5.45E−08 | 0.3783 | + | 5.43 |
| ARACHNOID_METAB_KEGG | 2.46E−29 | 0.5189 | + | 29.34 |
| CASPASE_BIOCARTA | 5.36E−03 | 0.3318 | + | 1.49 |
| CELL_CYCLE_BIOCARTA | 1.10E−01 | 0.3805 | − | 0.72 |
| CELL_CYCLE_PROTEASOME_REACTOME | 4.56E−11 | 0.3522 | − | 7.20 |
| CETUXIMAB_BENEFIT | 2.15E−08 | 1.1102 | − | 16.82 |
| COMPLEMENT_BIOCARTA | 3.20E−32 | 0.7351 | + | 45.76 |
| COMPLEMENT_COAG_KEGG | 1.11E−85 | 0.5955 | + | 100.00 |
| CRYPT_BASE | 2.53E−11 | 0.3928 | − | 8.23 |
| CRYPT_TOP | 2.53E−11 | 0.4178 | + | 8.75 |
| CSC_BATLLE | 3.13E−05 | 0.4805 | − | 4.28 |
| EMT_CORE_GENES | 5.87E−01 | 0.3254 | − | 0.15 |
| EPITH_LOBODA | 3.89E−10 | 0.5010 | + | 9.32 |
| FATTY_ACID_METAB_KEGG | 7.43E−02 | 0.4347 | + | 0.97 |
| FGF_ACT_CP | 3.34E−05 | 0.3901 | + | 3.45 |
| FOLFIRI_RESPONSE | 9.96E−01 | 0.2410 | − | 0.00 |
| FOLFOX_RESPONSE_DOWN | 3.49E−06 | 0.5442 | + | 5.87 |
| FRUTOSE_MANNOSE_METAB_KEGG | 3.61E−16 | 0.4445 | + | 13.57 |
| GALACTOSE_METAB_KEGG | 9.32E−11 | 0.3527 | + | 6.99 |
| GLUTAMINE_AMINO_ACID_GO_BP | 5.12E−02 | 0.5102 | + | 1.30 |
| GLUTAMINE_GO_BP | 1.75E−09 | 0.4108 | + | 7.11 |
| GLUTATHIONE_KEGG | 7.83E−05 | 0.3774 | + | 3.06 |
| GLYCEROPHOSPHOLIPID_GO_BP | 1.30E−04 | 0.4457 | + | 3.42 |
| GLYCEROPHOSPHOLIPID_METAB_KEGG | 1.02E−09 | 0.4612 | + | 8.19 |
| IGF1R_CP | 6.73E−07 | 0.3664 | − | 4.47 |
| IMMUNE_CD8MACRO_GALON | 3.89E−04 | 0.7371 | + | 4.97 |
| IMMUNE_ESTIMATE | 1.11E−52 | 0.5728 | + | 58.83 |
| IMMUNE_MDSC_ALBELDA | 1.41E−07 | 0.4591 | + | 6.22 |
| IMMUNE_MDSC_CERWENKA | 6.48E−07 | 0.7856 | + | 9.61 |
| IMMUNE_MSC | 4.72E−31 | 0.8030 | + | 48.14 |
| IMMUNE_NKC_BREAST | 2.71E−02 | 0.4827 | + | 1.49 |
| IMMUNE_RESP_GO_BP | 2.59E−62 | 0.5539 | + | 67.43 |
| IMMUNE_TH1_GALON | 2.81E−12 | 0.8923 | + | 20.37 |
| IMMUNE_TH17_GOUNARI | 3.57E−03 | 0.4321 | + | 2.09 |
| IMMUNE_THF_BREAST | 3.49E−06 | 0.6384 | + | 6.89 |
| IMMUNE_TREG_GALON | 1.46E−09 | 0.5948 | + | 10.39 |
| IMMUNE_TREG_LUCAS | 9.29E−12 | 0.4583 | − | 9.99 |
| IMMUNE_TREG_PASTILLE | 8.63E−02 | 0.2034 | + | 0.43 |
| INTEGRIN_BETA3_CP | 7.41E−06 | 0.4001 | + | 4.06 |
| INTEGRIN_BIOCARTA | 9.72E−17 | 0.4216 | + | 13.34 |
| JAK_STAT_GO_BP | 5.83E−03 | 0.4044 | + | 1.79 |
| JAK_STAT_KEGG | 4.05E−08 | 0.4469 | + | 6.53 |
| KEGG_CELL_CYCLE | 6.84E−13 | 0.4137 | − | 9.95 |
| KRAS_50_UP | 5.03E−25 | 0.5018 | + | 24.10 |
| LINOLEIC_METAB_KEGG | 1.76E−21 | 0.5265 | + | 21.60 |
| LYSOPHOSPHOLIPID_PID | 5.74E−22 | 0.4198 | + | 17.62 |
| MAPK_ACT_GO_BP | 2.04E−14 | 0.4992 | + | 13.51 |
| MAPK_KEGG | 3.38E−09 | 0.4381 | + | 7.33 |
| MATRIX_REMODEL_REACTOME | 8.79E−52 | 0.4832 | + | 48.76 |
| MESENCH_LOBODA | 1.82E−01 | 0.3600 | − | 0.53 |
| MYC_TARGETS_CP | 6.43E−04 | 0.3331 | − | 2.10 |
| MYC_TARGETS_ZELLER | 3.81E−10 | 0.3521 | − | 6.55 |
| NFKB_BIOCARTA | 1.36E−17 | 0.4104 | + | 13.68 |
| NITROGEN_METAB_KEGG | 4.79E−02 | 0.4037 | − | 1.05 |
| NOTCH_CP | 7.65E−08 | 0.3734 | − | 5.25 |
| NOTCH_REACTOME | 3.31E−06 | 0.3412 | − | 3.70 |
| NUCLEOTIDE_METAB_REACTOME | 2.26E−04 | 0.3892 | + | 2.80 |
| OXALIPLATIN_NCI60 | 4.97E−03 | 0.3641 | − | 1.66 |
| PD1_REACTOME | 7.31E−17 | 0.5907 | + | 18.84 |
| PENTOSE_GLUC_METAB_KEGG | 1.75E−09 | 0.4108 | + | 7.11 |
| PI3K_ACT_GO_BP | 4.09E−05 | 0.3884 | + | 3.37 |
| PI3K_ACT_REACTOME | 4.09E−05 | 0.3884 | + | 3.37 |
| PROTEASOME_KEGG | 8.41E−08 | 0.2689 | − | 3.76 |
| SERRATED_UP | 1.57E−08 | 0.3446 | − | 5.32 |
| SHH_CP | 1.48E−07 | 0.3289 | − | 4.44 |
| SHH_KEGG | 7.70E−07 | 0.3720 | + | 4.49 |
| SRC_ACT_BILD | 3.87E−17 | 0.4149 | − | 13.46 |
| SRC_ACT_BIOCARTA | 9.23E−02 | 0.3573 | + | 0.73 |
| STARCH_SUCROSE_METAB_KEGG | 1.35E−13 | 0.4711 | + | 11.98 |
| STROMAL_ESTIMATE | 1.81E−55 | 0.4198 | + | 45.42 |
| TGFB_1 | 6.58E−02 | 0.4142 | + | 0.97 |
| TGFB_2 | 5.14E−49 | 0.4962 | + | 47.36 |
| TGFB_CORE_GENES | 1.22E−07 | 0.4831 | − | 6.60 |
| TGFB_KEGG | 4.83E−15 | 0.4436 | − | 12.55 |
| TRANSLATION_RIBOS_REACTOME | 2.67E−09 | 0.3371 | − | 5.71 |
| TYROSINE_METAB_KEGG | 4.13E−08 | 0.4382 | + | 6.40 |
| VEGF_VEGFR_CP | 9.96E−01 | 0.2307 | − | 0.00 |
| VEGF_VEGFR_REACTOME | 9.96E−01 | 0.2565 | − | 0.00 |
| WNT_BIOCARTA | 3.13E−05 | 0.3676 | + | 3.27 |
| WNT_FLIER | 4.28E−01 | 0.3477 | − | 0.25 |
| WOUND_RESPONSE_GO_BP | 1.53E−64 | 0.5058 | + | 63.80 |
| ALANINE_ASPARTATE_GLUTAMATE_KEGG | 5.38E−08 | 0.5005 | − | 4.34 |
| AMINO_SUGAR_NUCLEO_METAB_KEGG | 8.96E−04 | 0.3264 | − | 1.19 |
| ARACHNOID_METAB_KEGG | 5.66E−31 | 0.4584 | − | 16.53 |
| CASPASE_BIOCARTA | 5.30E−02 | 0.2693 | + | 0.41 |
| CELL_CYCLE_BIOCARTA | 2.84E−03 | 0.3496 | − | 1.06 |
| CELL_CYCLE_PROTEASOME_REACTOME | 1.58E−02 | 0.2808 | − | 0.60 |
| CETUXIMAB_BENEFIT | 1.00E+00 | 0.1997 | − | 0.00 |
| COMPLEMENT_BIOCARTA | 2.71E−35 | 0.7185 | − | 29.61 |
| COMPLEMENT_COAG_KEGG | 6.88E−116 | 0.7284 | − | 100.00 |
| CRYPT_BASE | 7.28E−13 | 0.4123 | − | 5.97 |
| CRYPT_TOP | 7.38E−10 | 0.3274 | + | 3.56 |
| CSC_BATLLE | 2.09E−12 | 0.4522 | + | 6.30 |
| EMT_CORE_GENES | 3.86E−20 | 0.7973 | + | 18.45 |
| EPITH_LOBODA | 3.04E−10 | 0.4000 | − | 4.54 |
| FATTY_ACID_METAB_KEGG | 3.41E−03 | 0.4052 | − | 1.19 |
| FGF_ACT_CP | 8.54E−07 | 0.4215 | − | 3.05 |
| FOLFIRI_RESPONSE | 1.72E−05 | 0.4294 | + | 2.44 |
| FOLFOX_RESPONSE_DOWN | 8.63E−11 | 0.5447 | + | 6.53 |
| FRUTOSE_MANNOSE_METAB_KEGG | 4.90E−17 | 0.4120 | − | 8.01 |
| GALACTOSE_METAB_KEGG | 3.63E−08 | 0.3088 | − | 2.74 |
| GLUTAMINE_AMINO_ACID_GO_BP | 6.80E−07 | 0.5101 | − | 3.75 |
| GLUTAMINE_GO_BP | 8.45E−18 | 0.4575 | − | 9.31 |
| GLUTATHIONE_KEGG | 3.26E−03 | 0.2964 | − | 0.88 |
| GLYCEROPHOSPHOLIPID_GO_BP | 8.48E−03 | 0.3131 | − | 0.77 |
| GLYCEROPHOSPHOLIPID_METAB_KEGG | 3.23E−08 | 0.3738 | − | 3.34 |
| IGF1R_CP | 4.19E−04 | 0.2910 | + | 1.17 |
| IMMUNE_CD8MACRO_GALON | 1.00E+00 | 0.3206 | + | 0.00 |
| IMMUNE_ESTIMATE | 4.75E−31 | 0.4060 | + | 14.68 |
| IMMUNE_MDSC_ALBELDA | 8.41E−05 | 0.3353 | − | 1.63 |
| IMMUNE_MDSC_CERWENKA | 1.00E+00 | 0.3405 | + | 0.00 |
| IMMUNE_MSC | 4.59E−13 | 0.3666 | − | 5.39 |
| IMMUNE_NKC_BREAST | 2.42E−01 | 0.2869 | + | 0.21 |
| IMMUNE_RESP_GO_BP | 9.88E−59 | 0.4223 | + | 29.20 |
| IMMUNE_TH1_GALON | 6.41E−01 | 0.2950 | − | 0.07 |
| IMMUNE_TH17_GOUNARI | 5.66E−06 | 0.4089 | + | 2.56 |
| IMMUNE_THF_BREAST | 1.00E+00 | 0.3467 | + | 0.00 |
| IMMUNE_TREG_GALON | 1.00E+00 | 0.2405 | − | 0.00 |
| IMMUNE_TREG_LUCAS | 2.31E−07 | 0.4439 | + | 3.51 |
| IMMUNE_TREG_PASTILLE | 5.74E−02 | 0.4276 | + | 0.63 |
| INTEGRIN_BETA3_CP | 1.06E−13 | 0.4956 | + | 7.67 |
| INTEGRIN_BIOCARTA | 2.22E−08 | 0.3730 | − | 3.40 |
| JAK_STAT_GO_BP | 7.54E−01 | 0.3675 | − | 0.05 |
| JAK_STAT_KEGG | 4.76E−02 | 0.3342 | + | 0.53 |
| KEGG_CELL_CYCLE | 8.88E−02 | 0.3310 | − | 0.41 |
| KRAS_50_UP | 1.79E−44 | 0.6332 | + | 33.02 |
| LINOLEIC_METAB_KEGG | 8.13E−33 | 0.4636 | − | 17.73 |
| LYSOPHOSPHOLIPID_PID | 5.74E−04 | 0.3199 | − | 1.24 |
| MAPK_ACT_GO_BP | 2.43E−05 | 0.4404 | − | 2.42 |
| MAPK_KEGG | 2.11E−10 | 0.3684 | + | 4.25 |
| MATRIX_REMODEL_REACTOME | 1.01E−61 | 0.4717 | + | 34.30 |
| MESENCH_LOBODA | 3.07E−29 | 0.6368 | + | 21.64 |
| MYC_TARGETS_CP | 8.11E−09 | 0.3002 | − | 2.90 |
| MYC_TARGETS_ZELLER | 1.24E−02 | 0.2744 | − | 0.62 |
| NFKB_BIOCARTA | 6.61E−03 | 0.2917 | − | 0.76 |
| NITROGEN_METAB_KEGG | 1.36E−03 | 0.4234 | + | 1.45 |
| NOTCH_CP | 4.29E−05 | 0.2752 | + | 1.43 |
| NOTCH_REACTOME | 3.76E−06 | 0.3093 | + | 2.00 |
| NUCLEOTIDE_METAB_REACTOME | 2.11E−10 | 0.3340 | − | 3.85 |
| OXALIPLATIN_NCI60 | 5.48E−06 | 0.3886 | + | 2.44 |
| PD1_REACTOME | 1.00E−04 | 0.3976 | + | 1.90 |
| PENTOSE_GLUC_METAB_KEGG | 8.45E−18 | 0.4575 | − | 9.31 |
| PI3K_ACT_GO_BP | 1.38E−05 | 0.3182 | − | 1.84 |
| PI3K_ACT_REACTOME | 1.38E−05 | 0.3182 | − | 1.84 |
| PROTEASOME_KEGG | 1.48E−02 | 0.2122 | − | 0.46 |
| SERRATED_UP | 1.16E−16 | 0.3067 | + | 5.83 |
| SHH_CP | 3.58E−03 | 0.2572 | − | 0.75 |
| SHH_KEGG | 1.24E−11 | 0.3951 | + | 5.14 |
| SRC_ACT_BILD | 9.94E−14 | 0.3454 | + | 5.35 |
| SRC_ACT_BIOCARTA | 5.14E−15 | 0.4159 | − | 7.08 |
| STARCH_SUCROSE_METAB_KEGG | 3.00E−24 | 0.4139 | − | 11.61 |
| STROMAL_ESTIMATE | 9.00E−122 | 0.6830 | + | 98.55 |
| TGFB_1 | 1.64E−04 | 0.4405 | + | 1.99 |
| TGFB_2 | 1.44E−09 | 0.2866 | − | 3.02 |
| TGFB_CORE_GENES | 1.03E−13 | 0.4248 | + | 6.58 |
| TGFB_KEGG | 1.35E−21 | 0.4521 | + | 11.25 |
| TRANSLATION_RIBOS_REACTOME | 6.09E−01 | 0.2140 | + | 0.05 |
| TYROSINE_METAB_KEGG | 8.03E−17 | 0.4869 | − | 9.34 |
| VEGF_VEGFR_CP | 4.29E−05 | 0.3031 | + | 1.58 |
| VEGF_VEGFR_REACTOME | 6.99E−11 | 0.3939 | + | 4.77 |
| WNT_BIOCARTA | 8.48E−03 | 0.2987 | − | 0.74 |
| WNT_FLIER | 2.87E−13 | 0.3158 | − | 4.72 |
| WOUND_RESPONSE_GO_BP | 8.75E−83 | 0.5050 | + | 49.40 |

Table 7: Immune genes over-expressed in SNF2 metastases. Immune genes were extracted from the Hallmark signatures ‘inflammatory response’, ‘interferon alpha response’, and ‘interferon gamma response’, in addition to the custom gene sets ‘immune estimate’, ‘immune msc’, ‘immune response’, and ‘immune Th1’. Shown are differentially expressed genes in the comparison of SNF2 metastases to SNF1 and 3 metastases. Fold-change denotes ratio of SNF2 vs. SNF1+SNF3. P-value corrected for multiple comparisons using the Benjamini-Hochberg method.

TABLE 7

| Entrez ID | Gene Symbol | log2FC | adj P-Value |
|---|---|---|---|
| 101 | ADAM8 | 1.114151605 | 0.001613248 |
| 116071 | BATF2 | 1.050030827 | 0.000402744 |
| 684 | BST2 | 1.447305503 | 7.0543E−05 |
| 6352 | CCL5 | 1.330458386 | 0.007095402 |
| 929 | CD14 | 1.130725029 | 0.012587909 |
| 924 | CD7 | 1.267542763 | 0.032153144 |
| 3732 | CD82 | 1.023562283 | 3.13456E−05 |
| 10164 | CHST4 | 1.337259457 | 0.010577936 |
| 129607 | CMPK2 | 1.267895875 | 0.00020464 |
| 11151 | CORO1A | 1.197059439 | 0.003971877 |
| 1439 | CSF2RB | 1.217270252 | 0.000537403 |
| 1441 | CSF3R | 1.135318253 | 0.004176407 |
| 1521 | CTSW | 1.284888258 | 0.033477783 |
| 6373 | CXCL11 | 1.316957277 | 0.003212995 |
| 10148 | EBI3 | 1.096193201 | 0.033105492 |
| 10578 | GNLY | 1.493818884 | 0.004665499 |
| 85441 | HELZ2 | 1.03828577 | 0.000280215 |
| 3106 | HLA-B | 1.146355659 | 0.000498987 |
| 3107 | HLA-C | 1.021905249 | 0.000106004 |
| 3134 | HLA-F | 1.48934274 | 7.63496E−05 |
| 3135 | HLA-G | 1.426661079 | 0.016403862 |
| 3620 | IDO1 | 1.574024748 | 0.018152649 |
| 2537 | IFI6 | 1.29363079 | 0.00273504 |
| 3560 | IL2RB | 1.211399747 | 0.004000797 |
| 3665 | IRF7 | 1.127754457 | 7.13225E−05 |
| 9636 | ISG15 | 1.482687141 | 0.000641825 |
| 3695 | ITGB7 | 1.027118954 | 0.000731855 |
| 3965 | LGALS9 | 1.086883884 | 8.64875E−05 |
| 10859 | LILRB1 | 1.04319418 | 0.002360525 |
| 10288 | LILRB2 | 1.093481863 | 0.006651359 |
| 4050 | LTB | 1.391843525 | 0.0077572 |
| 4210 | MEFV | 1.50658012 | 0.000626788 |
| 931 | MS4A1 | 1.597053128 | 0.014568052 |
| 4599 | MX1 | 1.133975285 | 0.005273497 |
| 4689 | NCF4 | 1.013940329 | 0.00594171 |
| 150372 | NFAM1 | 1.022460225 | 0.000668619 |
| 4818 | NKG7 | 1.845375061 | 0.001205486 |
| 84166 | NLRC5 | 1.378670195 | 0.000117444 |
| 4939 | OAS2 | 1.18680124 | 0.003515069 |
| 8638 | OASL | 1.264199434 | 0.000440086 |
| 5133 | PDCD1 | 1.674556616 | 0.001721488 |
| 23533 | PIK3R5 | 1.013268448 | 0.0057499 |
| 5551 | PRF1 | 1.166608919 | 0.032817116 |
| 5699 | PSMB10 | 1.034307259 | 1.21519E−05 |
| 5698 | PSMB9 | 1.002183641 | 0.000622372 |
| 9051 | PSTPIP1 | 1.096234149 | 0.012609516 |
| 8698 | S1PR4 | 1.296702606 | 0.01465708 |
| 8578 | SCARF1 | 1.169639656 | 2.27411E−05 |
| 6398 | SECTM1 | 1.201216841 | 0.001565863 |
| 8651 | SOCS1 | 1.111619994 | 0.010405385 |
| 23166 | STAB1 | 1.006190485 | 0.001542236 |
| 6890 | TAP1 | 1.115449843 | 0.000228619 |
| 10312 | TCIRG1 | 1.191293713 | 2.12027E−05 |
| 7127 | TNFAIP2 | 1.024200168 | 0.003936091 |
| 8744 | TNFSF9 | 1.289096356 | 0.011517123 |
| 11274 | USP18 | 1.332873246 | 0.000168582 |
| 7454 | WAS | 1.080728587 | 0.003571432 |
| 54739 | XAF1 | 1.199077362 | 0.000854797 |
| 7535 | ZAP70 | 1.1775269 | 0.026150135 |
| 81030 | ZBP1 | 1.81033816 | 1.47166E−05 |

Table 8: Significantly mutated genes determined by MutSigCV. All variants that passed validation criteria in coding regions were categorized and tabulated to create an overall mutation type summary for each gene. n_syn=number of synonymous mutations; n_mis=number of missense mutations; n_lof=number of loss-of-function mutations; n_splice=number of splice junction mutations; n_indels_mis=number of inserts/deletions causing missense mutations; n_indels_lof=number of insertions/deletions causing loss-of-function mutations; num_unique=number of unique instances of a point mutation seen. MutSigCV v1.2 determined the probability of base level mutations within specific gene-level contexts given overall mutation rate, ratio of synonymous to non-synonymous mutation types, and other gene-levels factors including estimates of expression, replication rate, and chromatin state²¹. Raw P-values indicate the probability that the number of somatic mutations found within each gene is observed by chance with multiple testing corrections controlled by false discovery rate (FDR, q-value).

TABLE 8

| | | | | | | | | num_ | | FDR |
|---|---|---|---|---|---|---|---|---|---|---|
| Gene name | n_syn | n_mis | n_non | n_lof | n_splice | n_indels_mis | n_indels_lof | unique | P-value | (q-value) |
| KRAS | 0 | 22 | 0 | 0 | 0 | 0 | 0 | 4 | 0 | 0 |
| APC | 1 | 11 | 0 | 49 | 4 | 0 | 22 | 59 | 2.7E−15 | 2.5E−11 |
| TP53 | 0 | 32 | 0 | 7 | 0 | 1 | 6 | 32 | 4.2E−15 | 2.7E−11 |
| PIK3CA | 0 | 20 | 0 | 0 | 0 | 0 | 0 | 14 | 6.6E−09 | 3.1E−05 |
| FBXW7 | 0 | 7 | 0 | 2 | 0 | 0 | 0 | 9 | 4.5E−06 | 0.01681 |
| SOX9 | 0 | 0 | 0 | 3 | 0 | 1 | 4 | 7 | 6E−06 | 0.01889 |
| SMAD4 | 0 | 6 | 0 | 1 | 0 | 0 | 1 | 7 | 9.3E−06 | 0.02503 |
| NPPB | 0 | 3 | 0 | 0 | 0 | 0 | 0 | 3 | 0.00012 | 0.27423 |
| NRAS | 0 | 4 | 0 | 0 | 0 | 0 | 0 | 4 | 0.0002 | 0.4191 |
| TSC1 | 0 | 1 | 0 | 0 | 0 | 12 | 1 | 3 | 0.00033 | 0.62637 |
| RPL22 | 0 | 2 | 0 | 0 | 0 | 0 | 0 | 2 | 0.00052 | 0.88454 |
| KDR | 0 | 6 | 0 | 1 | 0 | 4 | 0 | 9 | 0.00061 | 0.95729 |
| HIST1H3I | 0 | 0 | 0 | 2 | 0 | 0 | 0 | 2 | 0.00094 | 1 |
| THSD7A | 0 | 8 | 0 | 0 | 0 | 0 | 0 | 8 | 0.00233 | 1 |
| CTNNA2 | 0 | 6 | 0 | 0 | 1 | 0 | 0 | 7 | 0.00269 | 1 |
| RNF2 | 0 | 2 | 0 | 1 | 0 | 0 | 0 | 3 | 0.00283 | 1 |
| RASA1 | 0 | 3 | 0 | 1 | 0 | 0 | 0 | 4 | 0.00496 | 1 |
| NUF2 | 0 | 3 | 0 | 0 | 0 | 0 | 0 | 3 | 0.00609 | 1 |
| CYP1A2 | 0 | 3 | 0 | 0 | 0 | 0 | 0 | 3 | 0.00612 | 1 |
| GPHN | 0 | 4 | 0 | 0 | 0 | 0 | 0 | 2 | 0.00717 | 1 |
| SOX10 | 0 | 3 | 0 | 0 | 0 | 0 | 0 | 3 | 0.0075 | 1 |
| CSMD3 | 4 | 20 | 0 | 1 | 1 | 0 | 1 | 27 | 0.00752 | 1 |
| TWSG1 | 0 | 2 | 0 | 0 | 0 | 0 | 0 | 2 | 0.00888 | 1 |
| NPM1 | 0 | 2 | 0 | 0 | 0 | 0 | 0 | 2 | 0.01038 | 1 |
| PAX3 | 0 | 4 | 0 | 0 | 0 | 0 | 0 | 4 | 0.01063 | 1 |
| RUNX1T1 | 0 | 5 | 0 | 3 | 0 | 0 | 0 | 7 | 0.0109 | 1 |
| FGF23 | 0 | 3 | 0 | 0 | 0 | 0 | 0 | 3 | 0.01166 | 1 |
| ESR1 | 0 | 5 | 0 | 0 | 0 | 0 | 0 | 4 | 0.01176 | 1 |
| RPN1 | 0 | 3 | 0 | 0 | 0 | 0 | 0 | 3 | 0.01194 | 1 |
| GNA13 | 1 | 2 | 0 | 1 | 0 | 0 | 0 | 4 | 0.01337 | 1 |
| SHCBP1 | 0 | 2 | 0 | 0 | 0 | 0 | 0 | 2 | 0.01427 | 1 |
| SOCS1 | 0 | 2 | 0 | 0 | 0 | 0 | 0 | 2 | 0.01553 | 1 |
| EZH2 | 0 | 5 | 0 | 0 | 0 | 0 | 0 | 5 | 0.0168 | 1 |
| SLCO1B1 | 0 | 2 | 0 | 0 | 0 | 0 | 0 | 2 | 0.01973 | 1 |
| FLG | 5 | 12 | 0 | 1 | 0 | 0 | 0 | 18 | 0.01985 | 1 |
| HIST1H1E | 1 | 2 | 0 | 0 | 0 | 0 | 0 | 3 | 0.02062 | 1 |
| PTEN | 0 | 2 | 0 | 1 | 0 | 0 | 0 | 3 | 0.02155 | 1 |
| SPATA17 | 0 | 1 | 0 | 1 | 0 | 0 | 0 | 2 | 0.02215 | 1 |
| CHN1 | 0 | 1 | 0 | 0 | 0 | 1 | 0 | 2 | 0.02263 | 1 |
| CDK12 | 0 | 3 | 0 | 0 | 0 | 0 | 1 | 4 | 0.02268 | 1 |
| EBF1 | 0 | 5 | 0 | 0 | 0 | 0 | 0 | 4 | 0.02279 | 1 |
| ARID2 | 0 | 4 | 0 | 1 | 0 | 0 | 0 | 5 | 0.02333 | 1 |
| SMAD3 | 0 | 3 | 0 | 0 | 0 | 0 | 0 | 3 | 0.02338 | 1 |
| MTRR | 0 | 3 | 0 | 0 | 0 | 0 | 1 | 4 | 0.02339 | 1 |
| EPHB1 | 0 | 4 | 0 | 0 | 0 | 0 | 1 | 4 | 0.02451 | 1 |
| BLID | 0 | 1 | 0 | 0 | 0 | 0 | 0 | 4 | 0.0249 | 1 |
| EP300 | 0 | 3 | 0 | 2 | 0 | 0 | 2 | 7 | 0.02613 | 1 |
| CDH11 | 0 | 5 | 0 | 0 | 0 | 0 | 0 | 5 | 0.02635 | 1 |
| BRIP1 | 0 | 4 | 0 | 0 | 0 | 0 | 0 | 4 | 0.02771 | 1 |
| SLC2A1 | 0 | 2 | 0 | 0 | 0 | 0 | 0 | 2 | 0.02786 | 1 |
| TP63 | 2 | 4 | 0 | 0 | 1 | 0 | 0 | 7 | 0.02843 | 1 |
| CCDC6 | 0 | 2 | 0 | 0 | 0 | 0 | 0 | 2 | 0.02905 | 1 |
| WT1 | 0 | 2 | 0 | 0 | 0 | 0 | 0 | 2 | 0.02993 | 1 |
| NAA16 | 0 | 2 | 0 | 0 | 0 | 0 | 1 | 3 | 0.03071 | 1 |
| PTGIS | 0 | 2 | 0 | 0 | 0 | 0 | 0 | 2 | 0.03085 | 1 |
| BCL11B | 0 | 4 | 0 | 0 | 0 | 0 | 0 | 4 | 0.03422 | 1 |
| H3F3B | 0 | 1 | 0 | 0 | 0 | 0 | 0 | 1 | 0.03444 | 1 |
| ANKRD37 | 0 | 1 | 0 | 0 | 0 | 0 | 0 | 1 | 0.03466 | 1 |
| FKBP1B | 0 | 1 | 0 | 0 | 0 | 0 | 0 | 1 | 0.0368 | 1 |
| NHP2 | 0 | 2 | 0 | 0 | 0 | 0 | 0 | 2 | 0.03717 | 1 |
| CYP2A6 | 0 | 2 | 0 | 0 | 0 | 0 | 0 | 2 | 0.03817 | 1 |
| INHBA | 0 | 3 | 0 | 0 | 0 | 0 | 0 | 3 | 0.03889 | 1 |
| ZNF331 | 0 | 5 | 0 | 0 | 0 | 0 | 0 | 5 | 0.04057 | 1 |
| SEMA3E | 1 | 4 | 0 | 0 | 1 | 0 | 0 | 6 | 0.04147 | 1 |
| HK2 | 2 | 2 | 0 | 2 | 0 | 0 | 0 | 5 | 0.0445 | 1 |
| PRSS1 | 0 | 1 | 0 | 0 | 0 | 0 | 0 | 1 | 0.04514 | 1 |
| NUDT3 | 0 | 1 | 0 | 0 | 0 | 0 | 0 | 1 | 0.04532 | 1 |
| LRP1B | 6 | 20 | 0 | 3 | 3 | 1 | 3 | 33 | 0.0471 | 1 |
| VTI1A | 0 | 0 | 0 | 1 | 0 | 0 | 0 | 1 | 0.0474 | 1 |
| HIST1H3B | 0 | 1 | 0 | 0 | 0 | 0 | 0 | 1 | 0.04747 | 1 |
| DOCK2 | 1 | 7 | 0 | 1 | 0 | 0 | 1 | 10 | 0.04843 | 1 |
| TCF7L2 | 0 | 2 | 0 | 6 | 1 | 0 | 2 | 11 | 0.04894 | 1 |
| WDR48 | 0 | 4 | 0 | 1 | 0 | 0 | 0 | 4 | 0.05144 | 1 |
| FGFR1OP2 | 0 | 0 | 0 | 1 | 0 | 0 | 0 | 1 | 0.05201 | 1 |
| BUB1B | 1 | 4 | 0 | 0 | 0 | 0 | 0 | 5 | 0.05216 | 1 |
| ACVR2A | 0 | 1 | 0 | 2 | 0 | 0 | 1 | 4 | 0.05637 | 1 |
| ZIM2 | 1 | 3 | 0 | 0 | 0 | 0 | 0 | 4 | 0.05666 | 1 |
| KLHL6 | 1 | 3 | 0 | 0 | 0 | 0 | 0 | 4 | 0.05694 | 1 |
| PPP2R1A | 0 | 1 | 0 | 1 | 0 | 0 | 0 | 2 | 0.0601 | 1 |
| INSL6 | 0 | 0 | 0 | 1 | 0 | 0 | 0 | 1 | 0.06116 | 1 |
| RPS10 | 0 | 0 | 0 | 1 | 0 | 0 | 0 | 1 | 0.06116 | 1 |
| JAZF1 | 0 | 2 | 0 | 0 | 0 | 0 | 0 | 2 | 0.06237 | 1 |
| MARK1 | 1 | 3 | 0 | 0 | 0 | 0 | 0 | 4 | 0.06297 | 1 |
| CYLD | 0 | 3 | 0 | 0 | 1 | 0 | 0 | 4 | 0.06322 | 1 |
| GALNT12 | 0 | 2 | 0 | 0 | 0 | 0 | 0 | 2 | 0.06372 | 1 |
| FMN2 | 1 | 4 | 0 | 1 | 0 | 1 | 0 | 7 | 0.06374 | 1 |
| TAF12 | 0 | 1 | 0 | 0 | 0 | 0 | 0 | 1 | 0.06397 | 1 |
| SPINK1 | 0 | 1 | 0 | 0 | 0 | 0 | 0 | 1 | 0.06432 | 1 |
| CTCF | 0 | 2 | 0 | 0 | 0 | 0 | 1 | 3 | 0.06449 | 1 |
| MAP3K7 | 1 | 2 | 0 | 0 | 0 | 0 | 0 | 3 | 0.06524 | 1 |
| FOXR2 | 0 | 1 | 0 | 0 | 0 | 0 | 0 | 1 | 0.06525 | 1 |
| NCOA4 | 0 | 2 | 0 | 0 | 0 | 0 | 0 | 2 | 0.06567 | 1 |
| XRCC5 | 0 | 2 | 0 | 0 | 0 | 0 | 0 | 2 | 0.07085 | 1 |
| TRAF5 | 0 | 3 | 0 | 0 | 0 | 0 | 0 | 3 | 0.07149 | 1 |
| BRAF | 0 | 3 | 0 | 0 | 0 | 0 | 0 | 2 | 0.07164 | 1 |
| ABCD2 | 0 | 5 | 0 | 0 | 0 | 0 | 0 | 5 | 0.07337 | 1 |
| CREB1 | 0 | 1 | 0 | 0 | 0 | 0 | 0 | 1 | 0.0746 | 1 |
| GREM1 | 0 | 1 | 0 | 0 | 0 | 0 | 0 | 1 | 0.07752 | 1 |
| SLC6A2 | 1 | 3 | 0 | 0 | 1 | 0 | 0 | 5 | 0.07753 | 1 |
| IRS2 | 0 | 6 | 0 | 0 | 0 | 2 | 0 | 7 | 0.07766 | 1 |
| DIS3L2 | 0 | 2 | 0 | 0 | 0 | 1 | 0 | 3 | 0.07784 | 1 |
| HSD17B7 | 0 | 1 | 0 | 0 | 0 | 0 | 0 | 1 | 0.07817 | 1 |
| RAF1 | 0 | 3 | 0 | 0 | 0 | 0 | 0 | 3 | 0.07861 | 1 |
| LPHN3 | 3 | 8 | 0 | 0 | 1 | 0 | 0 | 11 | 0.07872 | 1 |
| WDR36 | 0 | 2 | 0 | 0 | 0 | 0 | 1 | 3 | 0.07921 | 1 |
| PRKAR1A | 0 | 1 | 0 | 1 | 0 | 0 | 0 | 2 | 0.08113 | 1 |
| CSF3R | 0 | 1 | 0 | 1 | 0 | 0 | 0 | 2 | 0.08358 | 1 |
| MAGEA1 | 0 | 1 | 0 | 0 | 0 | 0 | 0 | 1 | 0.085381 | 1 |
| OPTN | 0 | 1 | 0 | 1 | 0 | 0 | 0 | 2 | 0.08753 | 1 |
| KLK2 | 0 | 0 | 0 | 1 | 0 | 0 | 0 | 1 | 0.08783 | 1 |
| THRAP3 | 1 | 2 | 0 | 0 | 0 | 0 | 0 | 3 | 0.08945 | 1 |
| DRD1 | 0 | 1 | 0 | 0 | 0 | 0 | 0 | 1 | 0.08951 | 1 |
| TFG | 0 | 2 | 0 | 0 | 0 | 0 | 0 | 2 | 0.08966 | 1 |
| B2M | 0 | 0 | 0 | 0 | 0 | 0 | 1 | 1 | 0.09012 | 1 |
| C11orf30 | 0 | 3 | 0 | 0 | 0 | 0 | 0 | 3 | 0.09037 | 1 |
| CSF1R | 1 | 3 | 0 | 0 | 0 | 0 | 0 | 3 | 0.09085 | 1 |
| CDH10 | 0 | 5 | 0 | 0 | 0 | 0 | 0 | 5 | 0.09135 | 1 |
| RALGDS | 0 | 1 | 0 | 1 | 0 | 0 | 0 | 2 | 0.09284 | 1 |
| HOXA3 | 0 | 1 | 0 | 0 | 0 | 0 | 0 | 1 | 0.09611 | 1 |
| PXDN | 4 | 7 | 0 | 0 | 0 | 0 | 0 | 10 | 0.09621 | 1 |
| CYP2C18 | 0 | 1 | 0 | 0 | 0 | 0 | 0 | 1 | 0.09824 | 1 |
| PRDM9 | 3 | 5 | 0 | 1 | 0 | 0 | 0 | 9 | 0.10326 | 1 |
| ITGA10 | 0 | 2 | 0 | 0 | 0 | 0 | 0 | 2 | 0.10338 | 1 |
| DNMT3A | 0 | 3 | 0 | 0 | 0 | 0 | 1 | 4 | 0.10375 | 1 |
| LPP | 0 | 2 | 0 | 0 | 0 | 0 | 0 | 2 | 0.10832 | 1 |
| SEMA3A | 0 | 2 | 0 | 0 | 0 | 0 | 0 | 2 | 0.11036 | 1 |
| LTF | 1 | 3 | 0 | 0 | 0 | 0 | 0 | 4 | 0.11134 | 1 |
| MSI2 | 0 | 1 | 0 | 0 | 0 | 0 | 0 | 1 | 0.11245 | 1 |
| MAP2K4 | 1 | 1 | 0 | 0 | 0 | 0 | 0 | 2 | 0.11325 | 1 |
| ZNF320 | 0 | 1 | 0 | 0 | 0 | 0 | 0 | 1 | 0.11338 | 1 |
| CSMD1 | 3 | 8 | 0 | 0 | 2 | 0 | 1 | 14 | 0.11491 | 1 |
| IL7R | 0 | 2 | 0 | 0 | 0 | 0 | 0 | 2 | 0.11984 | 1 |
| APOA1 | 0 | 1 | 0 | 0 | 0 | 0 | 0 | 1 | 0.12055 | 1 |
| CLTCL1 | 2 | 4 | 0 | 0 | 0 | 0 | 1 | 7 | 0.12229 | 1 |
| CCDC141 | 1 | 1 | 0 | 1 | 0 | 0 | 0 | 3 | 0.12235 | 1 |
| GOPC | 0 | 2 | 0 | 0 | 0 | 0 | 0 | 2 | 0.12248 | 1 |
| CXCR4 | 0 | 2 | 0 | 0 | 0 | 0 | 0 | 2 | 0.12339 | 1 |
| DNAH5 | 5 | 17 | 0 | 0 | 0 | 0 | 0 | 21 | 0.12421 | 1 |
| HOXD11 | 0 | 0 | 0 | 1 | 0 | 0 | 0 | 1 | 0.12442 | 1 |
| ABCG1 | 1 | 3 | 0 | 0 | 0 | 0 | 0 | 4 | 0.12739 | 1 |
| PTPN11 | 0 | 1 | 0 | 1 | 0 | 0 | 0 | 2 | 0.12857 | 1 |
| CA9 | 0 | 1 | 0 | 1 | 0 | 0 | 0 | 2 | 0.13143 | 1 |
| JAK1 | 0 | 2 | 0 | 0 | 0 | 0 | 0 | 2 | 0.13246 | 1 |
| ANTXR2 | 0 | 1 | 0 | 0 | 0 | 0 | 0 | 1 | 0.13305 | 1 |
| IKZF2 | 0 | 2 | 0 | 0 | 0 | 0 | 0 | 2 | 0.13412 | 1 |
| HIST1H4L | 0 | 1 | 0 | 0 | 0 | 0 | 0 | 1 | 0.13416 | 1 |
| KTN1 | 1 | 7 | 0 | 0 | 0 | 0 | 0 | 8 | 0.13604 | 1 |
| TAF1L | 2 | 3 | 0 | 1 | 0 | 0 | 0 | 6 | 0.13611 | 1 |
| SEC31A | 1 | 2 | 0 | 2 | 0 | 0 | 0 | 5 | 0.13624 | 1 |
| RPS7 | 0 | 1 | 0 | 0 | 0 | 0 | 0 | 1 | 0.13632 | 1 |
| CCL19 | 0 | 1 | 0 | 0 | 0 | 0 | 0 | 1 | 0.13742 | 1 |
| SLC38A6 | 0 | 1 | 0 | 0 | 1 | 0 | 0 | 2 | 0.13873 | 1 |
| GALNT3 | 0 | 0 | 0 | 1 | 0 | 0 | 0 | 1 | 0.13897 | 1 |
| HIST1H4F | 0 | 1 | 0 | 0 | 0 | 0 | 0 | 1 | 0.14096 | 1 |
| GLCCI1 | 0 | 1 | 0 | 0 | 0 | 0 | 0 | 1 | 0.14392 | 1 |
| FKBP5 | 0 | 2 | 0 | 0 | 0 | 0 | 0 | 2 | 0.14538 | 1 |
| ABCB1 | 0 | 5 | 0 | 0 | 0 | 0 | 0 | 5 | 0.14569 | 1 |
| PRSS37 | 0 | 1 | 0 | 0 | 1 | 0 | 0 | 2 | 0.14599 | 1 |
| FKBP9 | 0 | 1 | 0 | 0 | 0 | 0 | 0 | 1 | 0.14717 | 1 |
| SLC16A4 | 0 | 1 | 0 | 0 | 0 | 0 | 0 | 1 | 0.14736 | 1 |
| AFF1 | 0 | 3 | 0 | 1 | 0 | 0 | 0 | 3 | 0.14938 | 1 |
| RAD50 | 1 | 0 | 4 | 0 | 1 | 0 | 0 | 6 | 0.15015 | 1 |
| MAOA | 0 | 1 | 0 | 0 | 0 | 0 | 0 | 1 | 0.15035 | 1 |
| ROBO3 | 0 | 3 | 0 | 0 | 0 | 0 | 0 | 3 | 0.15035 | 1 |
| STAT4 | 0 | 2 | 0 | 0 | 0 | 0 | 0 | 2 | 0.15171 | 1 |
| CRBN | 0 | 2 | 0 | 0 | 0 | 0 | 0 | 2 | 0.15429 | 1 |
| ARID4A | 1 | 4 | 0 | 1 | 0 | 0 | 0 | 6 | 0.15491 | 1 |
| CYP2R1 | 0 | 2 | 0 | 1 | 0 | 0 | 0 | 3 | 0.15702 | 1 |
| CCDC63 | 0 | 2 | 0 | 0 | 0 | 0 | 0 | 2 | 0.16057 | 1 |
| IKZF3 | 0 | 2 | 0 | 0 | 0 | 0 | 0 | 2 | 0.16228 | 1 |
| PSIP1 | 0 | 1 | 0 | 0 | 0 | 0 | 0 | 1 | 0.16432 | 1 |
| ZNF490 | 1 | 1 | 0 | 0 | 0 | 0 | 0 | 2 | 0.16512 | 1 |
| CASP8 | 1 | 1 | 0 | 0 | 0 | 0 | 0 | 2 | 0.16541 | 1 |
| PAK7 | 0 | 3 | 0 | 0 | 0 | 0 | 0 | 3 | 0.16639 | 1 |
| NCOA3 | 0 | 2 | 0 | 0 | 0 | 0 | 0 | 2 | 0.16712 | 1 |
| MCCC1 | 0 | 1 | 0 | 0 | 0 | 0 | 0 | 1 | 0.169 | 1 |
| CD58 | 0 | 1 | 0 | 0 | 0 | 0 | 0 | 1 | 0.1694 | 1 |
| SUZ12 | 0 | 2 | 0 | 0 | 0 | 0 | 0 | 2 | 0.17011 | 1 |
| FRMPD1 | 0 | 4 | 0 | 0 | 0 | 0 | 0 | 4 | 0.1715 | 1 |
| GATA3 | 0 | 1 | 0 | 0 | 0 | 1 | 0 | 2 | 0.17195 | 1 |
| IL11 | 0 | 1 | 0 | 0 | 0 | 0 | 0 | 1 | 0.17335 | 1 |
| HIST1H3C | 0 | 1 | 0 | 0 | 0 | 0 | 0 | 1 | 0.17367 | 1 |
| ETV4 | 0 | 1 | 0 | 0 | 0 | 0 | 0 | 1 | 0.17572 | 1 |
| GATA2 | 0 | 1 | 0 | 0 | 0 | 0 | 0 | 1 | 0.17574 | 1 |
| HOXA9 | 0 | 1 | 0 | 0 | 0 | 0 | 0 | 1 | 0.17645 | 1 |
| SRC | 0 | 2 | 0 | 0 | 0 | 0 | 0 | 2 | 0.17816 | 1 |
| GPC3 | 0 | 2 | 0 | 0 | 0 | 0 | 0 | 2 | 0.18009 | 1 |
| NPAT | 0 | 3 | 0 | 1 | 0 | 0 | 0 | 4 | 0.18105 | 1 |
| SGK1 | 0 | 1 | 0 | 0 | 0 | 0 | 0 | 1 | 0.18136 | 1 |
| SHOC2 | 1 | 2 | 0 | 0 | 0 | 0 | 0 | 3 | 0.18329 | 1 |
| HSP90AB1 | 0 | 1 | 0 | 0 | 0 | 0 | 0 | 1 | 0.18408 | 1 |
| TGM7 | 0 | 2 | 0 | 0 | 0 | 0 | 0 | 2 | 0.18587 | 1 |
| EXO1 | 0 | 4 | 0 | 0 | 0 | 0 | 0 | 4 | 0.18695 | 1 |
| LTBP3 | 0 | 2 | 0 | 0 | 0 | 0 | 0 | 2 | 0.18713 | 1 |
| POT1 | 0 | 2 | 0 | 0 | 0 | 0 | 1 | 3 | 0.18791 | 1 |
| ROBO1 | 0 | 0 | 0 | 3 | 0 | 8 | 0 | 6 | 0.18853 | 1 |
| BCHE | 0 | 5 | 0 | 1 | 0 | 0 | 0 | 6 | 0.1903 | 1 |
| ARAF | 0 | 3 | 0 | 0 | 0 | 0 | 0 | 3 | 0.19112 | 1 |
| PIK3R1 | 0 | 3 | 0 | 2 | 0 | 1 | 0 | 6 | 0.19139 | 1 |
| CSF1 | 1 | 2 | 0 | 0 | 0 | 0 | 0 | 3 | 0.19189 | 1 |
| PSRC1 | 0 | 1 | 0 | 0 | 0 | 0 | 0 | 1 | 0.19209 | 1 |
| NR4A3 | 0 | 3 | 0 | 0 | 0 | 0 | 1 | 4 | 0.19487 | 1 |
| ARHGAP21 | 1 | 4 | 0 | 0 | 0 | 0 | 0 | 5 | 0.1949 | 1 |
| RRAGD | 0 | 1 | 0 | 0 | 0 | 0 | 0 | 1 | 0.19513 | 1 |
| PDCD1 | 0 | 1 | 0 | 0 | 0 | 0 | 0 | 1 | 0.19608 | 1 |
| SETBP1 | 1 | 6 | 0 | 0 | 0 | 0 | 0 | 7 | 0.19978 | 1 |
| RUNX1 | 0 | 2 | 0 | 0 | 0 | 0 | 0 | 2 | 0.2 | 1 |
| FANCC | 0 | 3 | 0 | 0 | 0 | 0 | 0 | 3 | 0.20029 | 1 |
| DNMT3B | 0 | 1 | 0 | 0 | 0 | 0 | 0 | 1 | 0.20041 | 1 |
| PCDHGA2 | 0 | 2 | 0 | 0 | 0 | 0 | 0 | 2 | 0.201 | 1 |
| COMT | 0 | 0 | 0 | 1 | 0 | 0 | 0 | 1 | 0.201 | 1 |
| ZFHX4 | 3 | 14 | 0 | 1 | 2 | 0 | 0 | 20 | 0.20245 | 1 |
| FAS | 0 | 1 | 0 | 0 | 0 | 0 | 0 | 1 | 0.20374 | 1 |
| RPS6KA2 | 0 | 1 | 0 | 1 | 1 | 0 | 0 | 3 | 0.20474 | 1 |
| TBXAS1 | 1 | 2 | 0 | 0 | 0 | 0 | 0 | 3 | 0.20486 | 1 |
| NTF3 | 0 | 0 | 0 | 1 | 0 | 0 | 0 | 1 | 0.20536 | 1 |
| FOXP1 | 0 | 1 | 0 | 1 | 0 | 0 | 0 | 2 | 0.20581 | 1 |
| NT5C2 | 0 | 2 | 0 | 0 | 0 | 0 | 0 | 2 | 0.20613 | 1 |
| CACNA1S | 1 | 3 | 0 | 0 | 0 | 1 | 0 | 5 | 0.20628 | 1 |
| SOS1 | 0 | 2 | 0 | 0 | 0 | 0 | 0 | 2 | 0.2069 | 1 |
| CHEK2 | 0 | 0 | 0 | 1 | 1 | 0 | 0 | 2 | 0.20824 | 1 |
| TOP1 | 0 | 1 | 0 | 0 | 2 | 0 | 0 | 3 | 0.20871 | 1 |
| NLRP7 | 1 | 3 | 0 | 0 | 0 | 1 | 0 | 5 | 0.20892 | 1 |
| HMCN1 | 1 | 15 | 0 | 3 | 1 | 0 | 0 | 19 | 0.2094 | 1 |
| FOXO3 | 1 | 0 | 0 | 0 | 0 | 0 | 1 | 2 | 0.21008 | 1 |
| NUP98 | 0 | 3 | 0 | 0 | 0 | 0 | 0 | 3 | 0.21024 | 1 |
| INPP4B | 0 | 1 | 0 | 0 | 0 | 0 | 0 | 1 | 0.21038 | 1 |
| SMUG1 | 0 | 1 | 0 | 0 | 0 | 0 | 0 | 1 | 0.21123 | 1 |
| TLR4 | 1 | 2 | 0 | 0 | 0 | 0 | 0 | 3 | 0.21202 | 1 |
| CDK4 | 0 | 0 | 0 | 1 | 0 | 0 | 0 | 1 | 0.21245 | 1 |
| AKT3 | 0 | 1 | 0 | 0 | 0 | 0 | 0 | 1 | 0.21261 | 1 |
| PLK2 | 0 | 2 | 0 | 0 | 0 | 0 | 0 | 2 | 0.21301 | 1 |
| CYP2C8 | 0 | 0 | 0 | 0 | 0 | 1 | 0 | 1 | 0.21387 | 1 |
| MTUS2 | 1 | 3 | 0 | 0 | 0 | 0 | 0 | 4 | 0.21493 | 1 |
| SLC47A2 | 0 | 0 | 0 | 1 | 0 | 0 | 0 | 1 | 0.21662 | 1 |
| PTPN3 | 0 | 2 | 0 | 0 | 0 | 0 | 0 | 2 | 0.21787 | 1 |
| MBD4 | 0 | 1 | 0 | 0 | 0 | 0 | 0 | 1 | 0.2181 | 1 |
| NTRK2 | 0 | 3 | 0 | 0 | 0 | 0 | 0 | 3 | 0.21818 | 1 |
| DDX10 | 0 | 2 | 0 | 0 | 0 | 0 | 0 | 2 | 0.21848 | 1 |
| SFRP1 | 0 | 1 | 0 | 0 | 0 | 0 | 0 | 1 | 0.21877 | 1 |
| MYB | 1 | 1 | 0 | 0 | 0 | 0 | 0 | 2 | 0.2192 | 1 |
| PTHLH | 0 | 1 | 0 | 0 | 0 | 0 | 0 | 1 | 0.2192 | 1 |
| FGFR1OP | 0 | 1 | 0 | 0 | 1 | 0 | 0 | 2 | 0.21955 | 1 |
| K1AA1524 | 0 | 0 | 0 | 1 | 0 | 0 | 0 | 1 | 0.22331 | 1 |
| STAT6 | 0 | 1 | 0 | 0 | 0 | 0 | 0 | 1 | 0.22534 | 1 |
| SLC22A1 | 1 | 1 | 0 | 0 | 0 | 0 | 0 | 2 | 0.22599 | 1 |
| EPCAM | 0 | 0 | 0 | 1 | 0 | 0 | 0 | 1 | 0.22776 | 1 |
| CIITA | 0 | 2 | 0 | 0 | 0 | 0 | 0 | 2 | 0.22896 | 1 |
| FGF3 | 0 | 1 | 0 | 0 | 0 | 0 | 0 | 1 | 0.23035 | 1 |
| IDH1 | 0 | 1 | 0 | 0 | 0 | 0 | 0 | 1 | 0.23269 | 1 |
| ICAM2 | 0 | 1 | 0 | 0 | 0 | 0 | 0 | 1 | 0.23291 | 1 |
| PSMA7 | 0 | 1 | 0 | 0 | 0 | 0 | 0 | 1 | 0.23321 | 1 |
| AFF2 | 2 | 4 | 0 | 0 | 0 | 0 | 0 | 6 | 0.23403 | 1 |
| EPAS1 | 0 | 1 | 0 | 0 | 0 | 0 | 0 | 1 | 0.23407 | 1 |
| UACA | 0 | 2 | 0 | 0 | 0 | 1 | 0 | 3 | 0.23507 | 1 |
| HIF1A | 0 | 1 | 0 | 1 | 0 | 0 | 0 | 2 | 0.2352 | 1 |
| MYSM1 | 0 | 1 | 0 | 0 | 0 | 0 | 0 | 1 | 0.23523 | 1 |
| CCND2 | 0 | 1 | 0 | 0 | 0 | 0 | 0 | 1 | 0.23837 | 1 |
| HTR1A | 0 | 2 | 0 | 0 | 0 | 0 | 0 | 2 | 0.23906 | 1 |
| MAX | 0 | 1 | 0 | 0 | 0 | 0 | 0 | 1 | 0.23933 | 1 |
| AXIN1 | 0 | 1 | 0 | 0 | 0 | 0 | 1 | 2 | 0.24218 | 1 |
| HOXB13 | 0 | 1 | 0 | 0 | 0 | 0 | 0 | 1 | 0.24487 | 1 |
| TRIM33 | 0 | 1 | 0 | 0 | 1 | 0 | 0 | 2 | 0.24518 | 1 |
| CIC | 2 | 2 | 0 | 0 | 0 | 0 | 0 | 4 | 0.2461 | 1 |
| CD79A | 0 | 1 | 0 | 0 | 0 | 0 | 0 | 1 | 0.24766 | 1 |
| YWHAE | 0 | 1 | 0 | 0 | 0 | 0 | 0 | 1 | 0.25035 | 1 |
| MSH6 | 0 | 8 | 0 | 0 | 0 | 0 | 0 | 6 | 0.24092 | 1 |
| HMGCR | 1 | 3 | 0 | 0 | 0 | 0 | 0 | 4 | 0.25093 | 1 |
| STX11 | 0 | 1 | 0 | 0 | 0 | 0 | 0 | 1 | 0.25323 | 1 |
| SLCO1A2 | 0 | 1 | 0 | 0 | 0 | 0 | 0 | 1 | 0.25335 | 1 |
| FCGR2B | 0 | 0 | 0 | 1 | 0 | 0 | 0 | 1 | 0.25434 | 1 |
| ABCA12 | 4 | 8 | 0 | 1 | 2 | 0 | 0 | 15 | 0.25652 | 1 |
| NDC80 | 0 | 1 | 0 | 0 | 0 | 0 | 0 | 1 | 0.25729 | 1 |
| AURKC | 0 | 1 | 0 | 0 | 0 | 0 | 0 | 1 | 0.25786 | 1 |
| PAX6 | 0 | 1 | 0 | 0 | 0 | 0 | 0 | 1 | 0.25849 | 1 |
| CEP57 | 0 | 1 | 0 | 0 | 0 | 0 | 0 | 1 | 0.261 | 1 |
| PRKDC | 1 | 7 | 0 | 1 | 4 | 0 | 0 | 13 | 0.2615 | 1 |
| PIK3CB | 0 | 2 | 0 | 0 | 0 | 0 | 0 | 2 | 0.26338 | 1 |
| SMARCB1 | 0 | 1 | 0 | 0 | 0 | 0 | 0 | 1 | 0.27128 | 1 |
| EAF2 | 0 | 0 | 0 | 1 | 0 | 0 | 0 | 1 | 0.27185 | 1 |
| CREB3L1 | 1 | 0 | 0 | 1 | 0 | 0 | 0 | 2 | 0.27399 | 1 |
| NTRK3 | 1 | 3 | 0 | 0 | 0 | 0 | 0 | 4 | 0.27585 | 1 |
| CASR | 0 | 2 | 0 | 0 | 0 | 0 | 0 | 2 | 0.27702 | 1 |
| ERBB2 | 0 | 3 | 0 | 0 | 0 | 0 | 0 | 3 | 0.28052 | 1 |
| RIMS2 | 1 | 10 | 0 | 0 | 0 | 0 | 0 | 11 | 0.28099 | 1 |
| PRDM1 | 1 | 2 | 0 | 0 | 1 | 0 | 0 | 4 | 0.28236 | 1 |
| CLTC | 1 | 2 | 0 | 0 | 0 | 0 | 0 | 3 | 0.28258 | 1 |
| ETV5 | 0 | 1 | 0 | 0 | 0 | 0 | 0 | 1 | 0.28279 | 1 |
| CDH1 | 1 | 4 | 0 | 0 | 0 | 0 | 0 | 5 | 0.2839 | 1 |
| NFKB1 | 1 | 2 | 0 | 0 | 0 | 0 | 0 | 3 | 0.28581 | 1 |
| IKBKB | 0 | 1 | 0 | 0 | 0 | 0 | 0 | 1 | 0.28589 | 1 |
| CAMTA1 | 0 | 2 | 0 | 0 | 0 | 0 | 0 | 2 | 0.28901 | 1 |
| ZNF384 | 0 | 1 | 0 | 0 | 0 | 0 | 0 | 1 | 0.28973 | 1 |
| TGFBR2 | 1 | 0 | 0 | 1 | 0 | 0 | 2 | 3 | 0.28993 | 1 |
| MSH3 | 0 | 2 | 0 | 0 | 0 | 1 | 0 | 3 | 0.29008 | 1 |
| ATP2B3 | 0 | 3 | 0 | 0 | 0 | 0 | 0 | 3 | 0.2912 | 1 |
| ACSL6 | 1 | 1 | 0 | 0 | 0 | 0 | 0 | 2 | 0.29274 | 1 |
| UGT1A1 | 0 | 1 | 0 | 0 | 0 | 0 | 0 | 1 | 0.29277 | 1 |
| PALB2 | 1 | 1 | 0 | 0 | 0 | 0 | 1 | 3 | 0.29307 | 1 |
| ITGB3 | 0 | 2 | 0 | 0 | 0 | 0 | 0 | 2 | 0.29406 | 1 |
| BCORL1 | 0 | 2 | 0 | 0 | 0 | 0 | 0 | 2 | 0.29488 | 1 |
| PCDH15 | 0 | 8 | 0 | 1 | 1 | 0 | 0 | 10 | 0.29552 | 1 |
| STAT3 | 0 | 1 | 0 | 0 | 0 | 0 | 0 | 1 | 0.29587 | 1 |
| MAML2 | 1 | 3 | 0 | 0 | 0 | 0 | 0 | 4 | 0.29652 | 1 |
| BCL9 | 1 | 1 | 0 | 0 | 0 | 0 | 2 | 4 | 0.29775 | 1 |
| JAK3 | 1 | 1 | 0 | 1 | 0 | 0 | 0 | 3 | 0.29807 | 1 |
| ARID5B | 2 | 2 | 0 | 0 | 0 | 2 | 0 | 6 | 0.2984 | 1 |
| CREBBP | 0 | 3 | 0 | 0 | 0 | 0 | 0 | 3 | 0.2986 | 1 |
| ZNF276 | 0 | 1 | 0 | 0 | 0 | 0 | 0 | 1 | 0.29952 | 1 |
| SETD2 | 1 | 3 | 0 | 1 | 0 | 0 | 0 | 5 | 0.3006 | 1 |
| FUBP1 | 0 | 0 | 0 | 0 | 0 | 0 | 2 | 2 | 0.30254 | 1 |
| MORC2 | 0 | 1 | 0 | 0 | 0 | 0 | 0 | 1 | 0.30353 | 1 |
| COL1A1 | 1 | 3 | 0 | 0 | 0 | 0 | 1 | 5 | 0.30376 | 1 |
| AIP | 0 | 1 | 0 | 0 | 0 | 0 | 0 | 1 | 0.30397 | 1 |
| PGAP3 | 0 | 1 | 0 | 0 | 0 | 0 | 0 | 1 | 0.30581 | 1 |
| MITF | 1 | 4 | 0 | 0 | 0 | 0 | 0 | 5 | 0.30665 | 1 |
| AFF3 | 1 | 2 | 0 | 0 | 0 | 0 | 0 | 3 | 0.30932 | 1 |
| STK11 | 0 | 1 | 0 | 0 | 0 | 0 | 0 | 1 | 0.31005 | 1 |
| SENP5 | 1 | 1 | 0 | 0 | 0 | 0 | 0 | 2 | 0.31257 | 1 |
| RANBP17 | 0 | 2 | 0 | 1 | 0 | 0 | 0 | 3 | 0.31413 | 1 |
| SARDH | 1 | 2 | 0 | 0 | 0 | 0 | 0 | 3 | 0.31629 | 1 |
| SPI1 | 0 | 1 | 0 | 0 | 0 | 0 | 0 | 1 | 0.31656 | 1 |
| GRIN2A | 2 | 6 | 0 | 1 | 0 | 0 | 0 | 9 | 0.31702 | 1 |
| TNKS | 0 | 2 | 0 | 1 | 0 | 0 | 0 | 3 | 0.31855 | 1 |
| IKBKE | 0 | 2 | 0 | 0 | 0 | 0 | 0 | 2 | 0.32182 | 1 |
| FANCM | 1 | 3 | 0 | 0 | 0 | 1 | 0 | 3 | 0.32313 | 1 |
| NUP214 | 1 | 3 | 0 | 0 | 0 | 0 | 0 | 4 | 0.32475 | 1 |
| GEN1 | 1 | 1 | 0 | 1 | 0 | 0 | 0 | 3 | 0.32572 | 1 |
| SF3B1 | 1 | 2 | 0 | 0 | 0 | 0 | 0 | 3 | 0.32623 | 1 |
| PPM1D | 0 | 1 | 0 | 0 | 0 | 0 | 0 | 1 | 0.32628 | 1 |
| UTP11L | 0 | 1 | 0 | 0 | 0 | 0 | 0 | 1 | 0.32704 | 1 |
| EED | 0 | 1 | 0 | 0 | 1 | 0 | 0 | 2 | 0.32736 | 1 |
| PAX8 | 0 | 1 | 0 | 0 | 0 | 0 | 0 | 1 | 0.32783 | 1 |
| PMS1 | 0 | 0 | 0 | 1 | 0 | 0 | 1 | 2 | 0.32983 | 1 |
| GUCY1A2 | 0 | 1 | 0 | 0 | 0 | 0 | 0 | 1 | 0.33104 | 1 |
| NELL1 | 1 | 4 | 0 | 1 | 0 | 0 | 0 | 6 | 0.33173 | 1 |
| KDM5A | 1 | 2 | 0 | 0 | 0 | 0 | 0 | 3 | 0.33264 | 1 |
| PCSK7 | 1 | 3 | 0 | 0 | 0 | 0 | 0 | 4 | 0.33303 | 1 |
| TET3 | 1 | 2 | 0 | 0 | 0 | 0 | 0 | 3 | 0.33334 | 1 |
| SETDB1 | 0 | 2 | 0 | 0 | 1 | 0 | 1 | 4 | 0.3365 | 1 |
| PGR | 1 | 1 | 0 | 1 | 1 | 0 | 0 | 4 | 0.3377 | 1 |
| LRP6 | 0 | 2 | 0 | 0 | 0 | 0 | 0 | 2 | 0.3386 | 1 |
| PLCG2 | 0 | 1 | 0 | 0 | 2 | 0 | 0 | 3 | 0.33903 | 1 |
| UIMC1 | 0 | 1 | 0 | 1 | 0 | 0 | 0 | 2 | 0.34019 | 1 |
| RHBDF2 | 2 | 3 | 0 | 0 | 0 | 0 | 0 | 5 | 0.34321 | 1 |
| FOXL2 | 1 | 1 | 0 | 0 | 0 | 0 | 0 | 2 | 0.344 | 1 |
| NALCN | 2 | 4 | 0 | 1 | 3 | 0 | 1 | 11 | 0.34534 | 1 |
| WRN | 0 | 3 | 0 | 0 | 0 | 0 | 0 | 3 | 0.34603 | 1 |
| CCDC18 | 0 | 2 | 0 | 0 | 0 | 0 | 0 | 2 | 0.34637 | 1 |
| GFI1 | 0 | 1 | 0 | 0 | 0 | 0 | 0 | 1 | 0.3465 | 1 |
| CARD11 | 2 | 3 | 0 | 0 | 1 | 0 | 0 | 6 | 0.34708 | 1 |
| WAS | 0 | 1 | 0 | 0 | 0 | 0 | 0 | 1 | 0.34743 | 1 |
| GOT1 | 1 | 1 | 0 | 0 | 0 | 0 | 0 | 2 | 0.34764 | 1 |
| SLCO1C1 | 1 | 4 | 0 | 0 | 0 | 0 | 1 | 6 | 0.34823 | 1 |
| BLM | 0 | 0 | 0 | 1 | 0 | 0 | 0 | 1 | 0.35544 | 1 |
| DCC | 1 | 2 | 0 | 0 | 1 | 0 | 0 | 4 | 0.35633 | 1 |
| BAI3 | 1 | 6 | 0 | 0 | 1 | 0 | 0 | 8 | 0.35722 | 1 |
| PTK2 | 1 | 2 | 0 | 0 | 0 | 0 | 0 | 3 | 0.3578 | 1 |
| ITK | 0 | 1 | 0 | 0 | 0 | 0 | 0 | 1 | 0.35866 | 1 |
| NPHS2 | 0 | 1 | 0 | 0 | 1 | 0 | 0 | 2 | 0.36118 | 1 |
| GABRA1 | 1 | 1 | 0 | 0 | 0 | 0 | 0 | 2 | 0.36162 | 1 |
| ALDH2 | 1 | 1 | 0 | 0 | 0 | 0 | 0 | 2 | 0.36293 | 1 |
| ERG | 0 | 1 | 0 | 0 | 0 | 0 | 0 | 1 | 0.36446 | 1 |
| MSN | 0 | 1 | 0 | 0 | 0 | 0 | 0 | 1 | 0.36569 | 1 |
| PCLO | 2 | 8 | 0 | 2 | 0 | 1 | 0 | 13 | 0.36714 | 1 |
| ITGA3 | 0 | 3 | 0 | 0 | 0 | 0 | 0 | 3 | 0.36749 | 1 |
| ACSL3 | 0 | 1 | 0 | 0 | 0 | 0 | 0 | 1 | 0.36867 | 1 |
| YTHDF2 | 0 | 2 | 0 | 0 | 0 | 0 | 0 | 2 | 0.37224 | 1 |
| TUBB6 | 0 | 1 | 0 | 0 | 0 | 0 | 0 | 1 | 0.37345 | 1 |
| BTK | 0 | 1 | 0 | 0 | 0 | 0 | 0 | 1 | 0.37599 | 1 |
| AHRR | 0 | 1 | 0 | 0 | 0 | 0 | 0 | 1 | 0.37729 | 1 |
| BDNF | 0 | 1 | 0 | 0 | 0 | 0 | 0 | 1 | 0.3779 | 1 |
| ZNF217 | 0 | 2 | 0 | 0 | 0 | 0 | 0 | 2 | 0.37868 | 1 |
| GNAS | 0 | 3 | 0 | 0 | 0 | 0 | 0 | 3 | 0.37877 | 1 |
| REL | 0 | 0 | 0 | 1 | 0 | 0 | 0 | 1 | 0.37926 | 1 |
| TCF4 | 0 | 1 | 0 | 0 | 0 | 0 | 0 | 1 | 0.3811 | 1 |
| RNASEL | 0 | 1 | 0 | 0 | 0 | 0 | 0 | 1 | 0.38309 | 1 |
| FOXO1 | 0 | 1 | 0 | 0 | 0 | 0 | 0 | 1 | 0.38541 | 1 |
| CENPF | 1 | 8 | 0 | 0 | 0 | 0 | 0 | 9 | 0.38686 | 1 |
| PLK1 | 0 | 1 | 0 | 0 | 0 | 0 | 0 | 1 | 0.38752 | 1 |
| TCF | 0 | 1 | 0 | 0 | 0 | 0 | 0 | 1 | 0.39154 | 1 |
| PIK3C2B | 0 | 2 | 0 | 0 | 0 | 0 | 1 | 3 | 0.39182 | 1 |
| HTR2A | 0 | 1 | 0 | 0 | 0 | 0 | 0 | 1 | 0.39215 | 1 |
| USP9X | 0 | 3 | 0 | 0 | 0 | 0 | 1 | 4 | 0.39319 | 1 |
| PRRX1 | 0 | 1 | 0 | 0 | 0 | 0 | 0 | 1 | 0.39486 | 1 |
| AXIN2 | 0 | 0 | 0 | 0 | 0 | 0 | 1 | 1 | 0.39541 | 1 |
| RABEP1 | 0 | 1 | 0 | 0 | 0 | 0 | 1 | 2 | 0.39646 | 1 |
| BARD1 | 0 | 1 | 0 | 0 | 0 | 0 | 0 | 1 | 0.39698 | 1 |
| SAMD9 | 0 | 4 | 0 | 1 | 0 | 0 | 0 | 5 | 0.39763 | 1 |
| CDH6 | 0 | 2 | 0 | 0 | 1 | 0 | 0 | 3 | 0.39965 | 1 |
| PFKP | 1 | 2 | 0 | 0 | 0 | 0 | 0 | 3 | 0.39979 | 1 |
| PAX7 | 0 | 1 | 0 | 0 | 0 | 0 | 0 | 1 | 0.40359 | 1 |
| SMOX | 0 | 1 | 0 | 0 | 0 | 0 | 0 | 1 | 0.40577 | 1 |
| SCN5A | 4 | 7 | 0 | 1 | 1 | 0 | 0 | 12 | 0.40615 | 1 |
| CACNB2 | 0 | 0 | 0 | 1 | 0 | 1 | 0 | 2 | 0.40866 | 1 |
| HDAC1 | 0 | 0 | 0 | 1 | 0 | 0 | 0 | 1 | 0.40885 | 1 |
| PLCB4 | 0 | 2 | 0 | 0 | 1 | 0 | 0 | 3 | 0.40903 | 1 |
| NOTCH2 | 1 | 2 | 0 | 1 | 0 | 1 | 0 | 5 | 0.41057 | 1 |
| SMAD2 | 0 | 1 | 0 | 0 | 0 | 0 | 0 | 1 | 0.41589 | 1 |
| MAP3K8 | 0 | 1 | 0 | 0 | 0 | 0 | 0 | 1 | 0.4207 | 1 |
| CDC73 | 0 | 1 | 0 | 0 | 0 | 0 | 0 | 1 | 0.42116 | 1 |
| FIP1L1 | 0 | 1 | 0 | 0 | 0 | 0 | 0 | 1 | 0.42249 | 1 |
| CASC5 | 0 | 2 | 0 | 0 | 1 | 0 | 0 | 3 | 0.42324 | 1 |
| ARNT | 0 | 1 | 0 | 0 | 0 | 0 | 0 | 1 | 0.42415 | 1 |
| RTEL1 | 0 | 2 | 0 | 0 | 0 | 0 | 0 | 2 | 0.42486 | 1 |
| SLC47A1 | 0 | 1 | 0 | 0 | 0 | 0 | 0 | 1 | 0.42574 | 1 |
| MLH1 | 0 | 3 | 0 | 1 | 0 | 0 | 0 | 4 | 0.4263 | 1 |
| EXT2 | 0 | 1 | 0 | 0 | 0 | 0 | 0 | 1 | 0.42683 | 1 |
| ROBO2 | 1 | 3 | 0 | 0 | 0 | 0 | 0 | 4 | 0.4275 | 1 |
| KIF5B | 1 | 1 | 0 | 0 | 0 | 0 | 0 | 2 | 0.42768 | 1 |
| SLC22A2 | 0 | 1 | 0 | 0 | 0 | 0 | 0 | 1 | 0.42794 | 1 |
| TOP2A | 0 | 1 | 0 | 0 | 0 | 0 | 0 | 1 | 0.43285 | 1 |
| PTK2B | 1 | 1 | 0 | 0 | 1 | 0 | 0 | 3 | 0.433 | 1 |
| FOXA1 | 0 | 1 | 0 | 0 | 0 | 0 | 0 | 1 | 0.43406 | 1 |
| KLHL14 | 0 | 1 | 0 | 0 | 0 | 0 | 0 | 1 | 0.43414 | 1 |
| ATR | 0 | 2 | 0 | 0 | 0 | 1 | 0 | 3 | 0.43663 | 1 |
| HNF1A | 0 | 1 | 0 | 0 | 0 | 0 | 0 | 1 | 0.43666 | 1 |
| PDPK1 | 1 | 1 | 0 | 0 | 0 | 0 | 0 | 2 | 0.43697 | 1 |
| CYP2B6 | 1 | 1 | 0 | 0 | 0 | 0 | 0 | 2 | 0.43745 | 1 |
| FANCA | 0 | 1 | 0 | 0 | 0 | 0 | 0 | 1 | 0.4382 | 1 |
| MET | 1 | 1 | 0 | 0 | 0 | 0 | 0 | 2 | 0.43946 | 1 |
| CBL | 0 | 1 | 0 | 0 | 0 | 0 | 0 | 1 | 0.4398 | 1 |
| BAP1 | 0 | 1 | 0 | 0 | 0 | 0 | 0 | 1 | 0.44006 | 1 |
| CAPRIN1 | 0 | 1 | 0 | 0 | 0 | 0 | 0 | 1 | 0.44058 | 1 |
| STAG2 | 0 | 1 | 0 | 0 | 0 | 0 | 0 | 1 | 0.44079 | 1 |
| MAFB | 0 | 1 | 0 | 0 | 0 | 0 | 0 | 1 | 0.44344 | 1 |
| USP6 | 1 | 2 | 0 | 0 | 1 | 0 | 0 | 4 | 0.44369 | 1 |
| NF2 | 0 | 1 | 0 | 0 | 0 | 0 | 0 | 1 | 0.4446 | 1 |
| SLC15A2 | 0 | 1 | 0 | 0 | 0 | 0 | 0 | 1 | 0.44884 | 1 |
| RNF43 | 0 | 1 | 0 | 0 | 0 | 0 | 0 | 1 | 0.45078 | 1 |
| CRTC3 | 0 | 1 | 0 | 0 | 0 | 0 | 0 | 1 | 0.45188 | 1 |
| SLCO2B1 | 1 | 1 | 0 | 0 | 0 | 0 | 0 | 2 | 0.45194 | 1 |
| BCR | 0 | 3 | 0 | 0 | 0 | 0 | 0 | 3 | 0.45379 | 1 |
| BRCA2 | 2 | 12 | 0 | 0 | 0 | 0 | 0 | 14 | 0.45384 | 1 |
| SLC34A2 | 1 | 1 | 0 | 0 | 0 | 0 | 0 | 2 | 0.45502 | 1 |
| PDGFRA | 1 | 1 | 0 | 1 | 1 | 0 | 0 | 4 | 0.45506 | 1 |
| NFL2L2 | 0 | 1 | 0 | 0 | 0 | 0 | 0 | 1 | 0.45598 | 1 |
| ARHGEF11 | 1 | 2 | 0 | 0 | 0 | 0 | 0 | 3 | 0.45732 | 1 |
| SMARCA4 | 0 | 2 | 0 | 0 | 0 | 0 | 0 | 2 | 0.45803 | 1 |
| AKT1 | 0 | 1 | 0 | 0 | 0 | 0 | 0 | 1 | 0.45826 | 1 |
| SLIT2 | 1 | 2 | 0 | 0 | 1 | 0 | 0 | 4 | 0.4588 | 1 |
| ITGB2 | 0 | 1 | 0 | 0 | 1 | 0 | 0 | 2 | 0.45918 | 1 |
| MBD1 | 0 | 1 | 0 | 0 | 1 | 0 | 0 | 2 | 0.45926 | 1 |
| PLCG1 | 2 | 2 | 0 | 1 | 0 | 0 | 0 | 5 | 0.46207 | 1 |
| TAF15 | 0 | 1 | 0 | 0 | 0 | 0 | 0 | 1 | 0.46761 | 1 |
| LRRTM4 | 1 | 1 | 0 | 0 | 0 | 0 | 0 | 2 | 0.46844 | 1 |
| TNFAIP3 | 0 | 1 | 0 | 0 | 0 | 0 | 0 | 1 | 0.46867 | 1 |
| DSP | 0 | 4 | 0 | 0 | 0 | 0 | 0 | 3 | 0.46868 | 1 |
| STIL | 1 | 0 | 0 | 1 | 0 | 0 | 0 | 2 | 0.4694 | 1 |
| NSD1 | 2 | 4 | 0 | 0 | 0 | 0 | 0 | 6 | 0.46969 | 1 |
| WBSCR17 | 2 | 4 | 0 | 0 | 0 | 0 | 0 | 6 | 0.4722 | 1 |
| ABCC2 | 0 | 3 | 0 | 1 | 1 | 0 | 0 | 5 | 0.47328 | 1 |
| LAMP1 | 1 | 1 | 0 | 0 | 0 | 0 | 0 | 2 | 0.47412 | 1 |
| ABCC5 | 1 | 2 | 0 | 0 | 0 | 0 | 0 | 3 | 0.47537 | 1 |
| HECW1 | 3 | 3 | 0 | 0 | 0 | 0 | 0 | 6 | 0.47542 | 1 |
| NCOA2 | 0 | 4 | 0 | 0 | 0 | 0 | 0 | 4 | 0.4758 | 1 |
| DTX1 | 0 | 1 | 0 | 0 | 1 | 0 | 0 | 2 | 0.47635 | 1 |
| PHLPP2 | 2 | 0 | 0 | 0 | 0 | 0 | 1 | 2 | 0.47656 | 1 |
| DBH | 0 | 1 | 0 | 0 | 1 | 0 | 0 | 2 | 0.47675 | 1 |
| TETI | 0 | 2 | 0 | 1 | 0 | 0 | 0 | 3 | 0.47755 | 1 |
| PTPRS | 5 | 4 | 0 | 0 | 0 | 1 | 0 | 10 | 0.4777 | 1 |
| KIAA1549 | 2 | 2 | 0 | 0 | 0 | 0 | 0 | 4 | 0.48389 | 1 |
| MRE11A | 0 | 1 | 0 | 0 | 0 | 0 | 0 | 1 | 0.48749 | 1 |
| SRGAP3 | 1 | 3 | 0 | 1 | 0 | 0 | 0 | 5 | 0.4901 | 1 |
| DDR2 | 1 | 1 | 0 | 0 | 0 | 0 | 0 | 2 | 0.49459 | 1 |
| ATP10D | 2 | 5 | 0 | 0 | 0 | 0 | 1 | 8 | 0.49469 | 1 |
| IL21R | 0 | 2 | 0 | 0 | 0 | 0 | 0 | 2 | 0.49632 | 1 |
| SLCO1B3 | 0 | 3 | 0 | 0 | 0 | 0 | 0 | 3 | 0.49653 | 1 |
| OBSCN | 7 | 22 | 0 | 0 | 1 | 1 | 1 | 32 | 0.50023 | 1 |
| PCDHB6 | 2 | 1 | 0 | 0 | 0 | 0 | 0 | 3 | 0.50367 | 1 |
| PYGL | 1 | 1 | 0 | 0 | 0 | 0 | 0 | 2 | 0.50479 | 1 |
| JAKMIP2 | 0 | 3 | 0 | 1 | 0 | 0 | 0 | 4 | 0.50785 | 1 |
| RIPK1 | 1 | 1 | 0 | 1 | 0 | 0 | 0 | 3 | 0.50966 | 1 |
| AXL | 1 | 4 | 0 | 1 | 0 | 0 | 0 | 6 | 0.50979 | 1 |
| BCL6 | 0 | 2 | 0 | 0 | 0 | 0 | 0 | 2 | 0.51026 | 1 |
| SF3B2 | 0 | 1 | 0 | 0 | 0 | 0 | 0 | 1 | 0.51151 | 1 |
| GOLGA5 | 0 | 1 | 0 | 0 | 0 | 0 | 0 | 1 | 0.51413 | 1 |
| ZMYM2 | 2 | 1 | 0 | 0 | 0 | 0 | 0 | 3 | 0.52388 | 1 |
| CYP1B1 | 1 | 1 | 0 | 0 | 0 | 0 | 0 | 2 | 0.5252 | 1 |
| EPC1 | 0 | 1 | 0 | 0 | 0 | 0 | 0 | 1 | 0.52668 | 1 |
| FOXP2 | 0 | 3 | 0 | 0 | 0 | 0 | 0 | 3 | 0.5292 | 1 |
| NEBL | 1 | 1 | 0 | 0 | 0 | 1 | 0 | 3 | 0.52942 | 1 |
| ANKRD26 | 0 | 2 | 0 | 2 | 1 | 1 | 0 | 6 | 0.53263 | 1 |
| NBN | 1 | 3 | 0 | 0 | 0 | 0 | 0 | 4 | 0.53298 | 1 |
| CUL1 | 0 | 1 | 0 | 0 | 0 | 0 | 0 | 1 | 0.53369 | 1 |
| ELAC2 | 0 | 1 | 0 | 0 | 1 | 0 | 0 | 2 | 0.53435 | 1 |
| MALT1 | 1 | 1 | 0 | 0 | 0 | 0 | 1 | 3 | 0.53482 | 1 |
| DDX3X | 0 | 1 | 0 | 0 | 0 | 0 | 0 | 1 | 0.53722 | 1 |
| ERCC3 | 1 | 2 | 0 | 0 | 0 | 0 | 0 | 3 | 0.53957 | 1 |
| CDH6 | 1 | 5 | 0 | 0 | 0 | 0 | 0 | 5 | 0.53984 | 1 |
| NCOR1 | 1 | 2 | 0 | 1 | 1 | 0 | 0 | 5 | 0.54422 | 1 |
| ERCC4 | 1 | 0 | 0 | 1 | 0 | 0 | 0 | 2 | 0.54453 | 1 |
| MKI67 | 2 | 10 | 0 | 1 | 0 | 0 | 0 | 13 | 0.5746 | 1 |
| XPC | 1 | 1 | 0 | 0 | 0 | 0 | 0 | 2 | 0.54955 | 1 |
| CDH2 | 0 | 3 | 0 | 0 | 0 | 0 | 0 | 3 | 0.55277 | 1 |
| COLEC12 | 0 | 2 | 0 | 0 | 0 | 0 | 0 | 2 | 0.55486 | 1 |
| BMPR1A | 1 | 2 | 0 | 0 | 0 | 0 | 0 | 3 | 0.55526 | 1 |
| FH | 1 | 1 | 0 | 0 | 0 | 0 | 0 | 2 | 0.55558 | 1 |
| BRD3 | 1 | 1 | 0 | 0 | 0 | 0 | 0 | 2 | 0.55736 | 1 |
| XPO1 | 0 | 1 | 0 | 0 | 0 | 0 | 0 | 1 | 0.55899 | 1 |
| UNC13D | 0 | 0 | 0 | 1 | 0 | 1 | 0 | 2 | 0.56097 | 1 |
| FGFR3 | 1 | 1 | 0 | 0 | 0 | 0 | 0 | 2 | 0.5615 | 1 |
| ANTXR1 | 0 | 1 | 0 | 0 | 0 | 0 | 0 | 1 | 0.56165 | 1 |
| KEAP1 | 1 | 1 | 0 | 0 | 0 | 0 | 0 | 2 | 0.56244 | 1 |
| PIK3CG | 2 | 3 | 0 | 0 | 0 | 0 | 0 | 5 | 0.56341 | 1 |
| RBM15 | 0 | 2 | 0 | 0 | 0 | 0 | 0 | 2 | 0.56352 | 1 |
| EPHB2 | 0 | 1 | 0 | 0 | 0 | 0 | 0 | 1 | 0.56514 | 1 |
| COL1A2 | 2 | 1 | 0 | 0 | 1 | 0 | 0 | 4 | 0.5678 | 1 |
| FGF14 | 1 | 1 | 0 | 0 | 0 | 0 | 0 | 2 | 0.57328 | 1 |
| RNF213 | 2 | 10 | 0 | 0 | 1 | 0 | 0 | 13 | 0.57376 | 1 |
| NRP1 | 0 | 2 | 0 | 0 | 0 | 0 | 0 | 2 | 0.57622 | 1 |
| HAS2 | 0 | 1 | 0 | 0 | 0 | 0 | 0 | 1 | 0.57713 | 1 |
| MLH3 | 0 | 1 | 0 | 0 | 0 | 0 | 0 | 1 | 0.57795 | 1 |
| ACE | 0 | 2 | 0 | 0 | 0 | 0 | 0 | 2 | 0.58057 | 1 |
| BRD4 | 2 | 2 | 0 | 0 | 0 | 0 | 0 | 4 | 0.5876 | 1 |
| RB1 | 0 | 1 | 0 | 2 | 0 | 0 | 0 | 3 | 0.58993 | 1 |
| MYBL2 | 0 | 1 | 0 | 0 | 1 | 0 | 0 | 2 | 0.59065 | 1 |
| DICER1 | 0 | 2 | 0 | 0 | 0 | 0 | 0 | 2 | 0.59491 | 1 |
| ERCC2 | 0 | 1 | 0 | 0 | 0 | 0 | 0 | 1 | 0.59499 | 1 |
| SMO | 1 | 1 | 0 | 0 | 0 | 0 | 0 | 2 | 0.59778 | 1 |
| BCL11A | 0 | 1 | 0 | 0 | 0 | 0 | 0 | 1 | 0.60039 | 1 |
| JAK2 | 0 | 2 | 0 | 0 | 0 | 0 | 0 | 2 | 0.60061 | 1 |
| MED12 | 1 | 3 | 0 | 0 | 0 | 0 | 0 | 4 | 0.60339 | 1 |
| CACNA1C | 1 | 2 | 0 | 1 | 1 | 0 | 0 | 5 | 0.60443 | 1 |
| ABCB11 | 0 | 1 | 0 | 0 | 0 | 0 | 0 | 1 | 0.60572 | 1 |
| EPHA5 | 1 | 3 | 0 | 0 | 0 | 0 | 0 | 4 | 0.60734 | 1 |
| THBS1 | 0 | 0 | 0 | 1 | 0 | 0 | 0 | 1 | 0.60782 | 1 |
| PLXNA1 | 3 | 5 | 0 | 0 | 0 | 0 | 0 | 8 | 0.60896 | 1 |
| PTPRT | 2 | 4 | 0 | 1 | 0 | 0 | 0 | 7 | 0.61012 | 1 |
| COL11A1 | 1 | 4 | 0 | 1 | 2 | 0 | 1 | 9 | 0.61069 | 1 |
| NLRP1 | 0 | 0 | 0 | 1 | 0 | 0 | 0 | 1 | 0.61133 | 1 |
| ANLN | 0 | 0 | 0 | 1 | 0 | 0 | 0 | 1 | 0.61661 | 1 |
| LIFR | 1 | 3 | 0 | 0 | 0 | 0 | 0 | 4 | 0.61924 | 1 |
| GAS7 | 0 | 1 | 0 | 0 | 0 | 0 | 0 | 1 | 0.61964 | 1 |
| ASXLI | 3 | 1 | 0 | 0 | 0 | 0 | 0 | 4 | 0.6221 | 1 |
| ZSCAN20 | 1 | 2 | 0 | 0 | 0 | 0 | 0 | 3 | 0.62315 | 1 |
| FAT2 | 1 | 6 | 0 | 0 | 0 | 0 | 0 | 7 | 0.62355 | 1 |
| SMC1A | 0 | 1 | 0 | 0 | 0 | 0 | 0 | 1 | 0.62361 | 1 |
| DMD | 0 | 6 | 0 | 3 | 1 | 0 | 0 | 10 | 0.62567 | 1 |
| EXT1 | 0 | 3 | 0 | 1 | 0 | 0 | 0 | 4 | 0.62571 | 1 |
| MYCN | 0 | 1 | 0 | 0 | 0 | 0 | 0 | 1 | 0.63058 | 1 |
| PHLDB1 | 0 | 1 | 0 | 0 | 0 | 0 | 0 | 1 | 0.63211 | 1 |
| CES1 | 2 | 3 | 0 | 0 | 0 | 0 | 0 | 5 | 0.63224 | 1 |
| ERC1 | 1 | 2 | 0 | 0 | 0 | 0 | 0 | 3 | 0.63564 | 1 |
| MLLT3 | 1 | 1 | 0 | 1 | 0 | 0 | 0 | 3 | 0.63749 | 1 |
| PIK3R2 | 2 | 1 | 0 | 0 | 0 | 0 | 0 | 3 | 0.63915 | 1 |
| ALOX5 | 1 | 1 | 0 | 0 | 0 | 0 | 0 | 2 | 0.64058 | 1 |
| RARA | 2 | 2 | 0 | 0 | 0 | 0 | 0 | 4 | 0.64211 | 1 |
| STK36 | 1 | 1 | 0 | 0 | 0 | 0 | 0 | 2 | 0.643 | 1 |
| PLEKHG5 | 1 | 1 | 0 | 0 | 0 | 0 | 0 | 2 | 0.64339 | 1 |
| FLT3 | 1 | 1 | 0 | 0 | 1 | 0 | 1 | 4 | 0.65141 | 1 |
| NF1 | 1 | 6 | 0 | 0 | 1 | 1 | 0 | 9 | 0.65407 | 1 |
| ARHGEF12 | 0 | 0 | 0 | 1 | 1 | 0 | 0 | 2 | 0.65428 | 1 |
| TSC2 | 1 | 1 | 0 | 0 | 1 | 0 | 0 | 3 | 0.65455 | 1 |
| FAT3 | 6 | 10 | 0 | 0 | 0 | 0 | 0 | 16 | 0.65699 | 1 |
| DNMT1 | 1 | 2 | 0 | 0 | 0 | 0 | 0 | 3 | 0.65996 | 1 |
| NIN | 0 | 1 | 0 | 0 | 0 | 0 | 0 | 1 | 0.66257 | 1 |
| PDGFRB | 0 | 1 | 0 | 0 | 0 | 0 | 0 | 1 | 0.66312 | 1 |
| ALK | 3 | 4 | 0 | 0 | 3 | 0 | 0 | 9 | 0.66451 | 1 |
| PTCH2 | 2 | 1 | 0 | 0 | 0 | 0 | 0 | 3 | 0.66865 | 1 |
| RICTOR | 0 | 1 | 0 | 1 | 1 | 0 | 0 | 3 | 0.66879 | 1 |
| WHSC1 | 0 | 1 | 0 | 0 | 1 | 0 | 0 | 2 | 0.67667 | 1 |
| SEMA5B | 2 | 2 | 0 | 0 | 0 | 0 | 0 | 4 | 0.67712 | 1 |
| ERBB3 | 0 | 1 | 0 | 0 | 0 | 0 | 0 | 1 | 0.67983 | 1 |
| RIF1 | 2 | 6 | 0 | 0 | 0 | 0 | 0 | 8 | 0.68191 | 1 |
| GLI3 | 3 | 6 | 0 | 0 | 0 | 0 | 0 | 9 | 0.68506 | 1 |
| NPR1 | 1 | 0 | 0 | 1 | 0 | 0 | 0 | 2 | 0.68695 | 1 |
| FLT1 | 2 | 3 | 0 | 0 | 0 | 0 | 0 | 5 | 0.6899 | 1 |
| GOLGA4 | 0 | 2 | 0 | 0 | 0 | 0 | 1 | 3 | 0.69049 | 1 |
| SPEN | 2 | 3 | 0 | 1 | 0 | 0 | 1 | 7 | 0.69605 | 1 |
| MED1 | 0 | 1 | 0 | 0 | 0 | 0 | 0 | 1 | 0.69607 | 1 |
| HDAC4 | 2 | 1 | 0 | 0 | 0 | 0 | 0 | 3 | 0.69737 | 1 |
| PEAR1 | 2 | 2 | 0 | 0 | 0 | 0 | 0 | 4 | 0.69762 | 1 |
| MST1R | 1 | 1 | 0 | 0 | 0 | 0 | 0 | 2 | 0.70452 | 1 |
| KIT | 1 | 4 | 0 | 0 | 0 | 0 | 0 | 5 | 0.70577 | 1 |
| NCOA1 | 0 | 1 | 0 | 0 | 0 | 2 | 0 | 2 | 0.71084 | 1 |
| PTCH1 | 0 | 1 | 0 | 0 | 1 | 0 | 0 | 2 | 0.71534 | 1 |
| SMC3 | 0 | 1 | 0 | 0 | 1 | 0 | 0 | 2 | 0.71567 | 1 |
| UBR5 | 1 | 1 | 0 | 0 | 0 | 0 | 0 | 2 | 0.71685 | 1 |
| HIP1 | 1 | 0 | 0 | 1 | 1 | 0 | 0 | 3 | 0.71809 | 1 |
| ERCC5 | 1 | 2 | 0 | 1 | 0 | 0 | 0 | 4 | 0.71841 | 1 |
| ZBTB16 | 2 | 2 | 0 | 0 | 1 | 0 | 1 | 6 | 0.71983 | 1 |
| NCOR2 | 1 | 3 | 0 | 0 | 0 | 0 | 0 | 4 | 0.72473 | 1 |
| NR3C1 | 0 | 1 | 0 | 0 | 0 | 0 | 0 | 1 | 0.72588 | 1 |
| INSR | 1 | 2 | 0 | 0 | 0 | 0 | 0 | 3 | 0.7281 | 1 |
| NDRG1 | 1 | 1 | 0 | 0 | 0 | 0 | 0 | 2 | 0.72947 | 1 |
| MYO18A | 3 | 1 | 0 | 0 | 1 | 0 | 0 | 5 | 0.73563 | 1 |
| PKHD1 | 3 | 10 | 0 | 0 | 0 | 0 | 0 | 13 | 0.7378 | 1 |
| STAT5A | 0 | 1 | 0 | 0 | 0 | 0 | 0 | 1 | 0.74112 | 1 |
| PDE4DIP | 4 | 9 | 0 | 2 | 1 | 0 | 0 | 16 | 0.7431 | 1 |
| PIK3CD | 1 | 1 | 0 | 0 | 1 | 0 | 0 | 3 | 0.74804 | 1 |
| MAP3K1 | 2 | 2 | 0 | 1 | 2 | 0 | 0 | 7 | 0.75112 | 1 |
| KIF1B | 0 | 2 | 0 | 0 | 0 | 0 | 0 | 2 | 0.75594 | 1 |
| ADAMTS20 | 3 | 4 | 0 | 0 | 0 | 0 | 0 | 7 | 0.75809 | 1 |
| XIRP2 | 7 | 15 | 0 | 0 | 0 | 0 | 0 | 22 | 0.7609 | 1 |
| LRRFIP1 | 1 | 1 | 0 | 0 | 0 | 0 | 0 | 2 | 0.76522 | 1 |
| PTGS2 | 1 | 2 | 0 | 0 | 0 | 0 | 0 | 3 | 0.76652 | 1 |
| MYH9 | 4 | 2 | 0 | 0 | 0 | 0 | 0 | 6 | 0.76717 | 1 |
| ZNF521 | 1 | 1 | 0 | 0 | 0 | 0 | 0 | 2 | 0.76959 | 1 |
| RIPK4 | 2 | 1 | 0 | 0 | 0 | 0 | 0 | 3 | 0.77303 | 1 |
| WRAP53 | 2 | 1 | 0 | 0 | 0 | 0 | 0 | 3 | 0.77524 | 1 |
| GRM8 | 0 | 2 | 0 | 0 | 0 | 0 | 0 | 2 | 0.77532 | 1 |
| TERT | 2 | 1 | 0 | 0 | 0 | 0 | 0 | 3 | 0.77924 | 1 |
| DOT1L | 0 | 2 | 0 | 0 | 0 | 0 | 0 | 2 | 0.78239 | 1 |
| FAT4 | 5 | 9 | 0 | 0 | 0 | 0 | 0 | 14 | 0.78626 | 1 |
| RET | 1 | 2 | 0 | 0 | 0 | 0 | 0 | 3 | 0.78774 | 1 |
| APOB | 4 | 5 | 0 | 2 | 0 | 0 | 1 | 12 | 0.79118 | 1 |
| SRRM2 | 2 | 4 | 0 | 1 | 0 | 0 | 1 | 8 | 0.79925 | 1 |
| MTR | 1 | 2 | 0 | 0 | 0 | 0 | 0 | 3 | 0.80002 | 1 |
| FAT1 | 4 | 8 | 0 | 1 | 0 | 0 | 0 | 13 | 0.80428 | 1 |
| ITPKB | 2 | 1 | 0 | 0 | 0 | 0 | 0 | 3 | 0.80509 | 1 |
| RAD21 | 2 | 2 | 0 | 0 | 0 | 0 | 0 | 4 | 0.80568 | 1 |
| NOTCH3 | 1 | 2 | 0 | 0 | 0 | 0 | 0 | 3 | 0.80664 | 1 |
| MECOM | 2 | 0 | 0 | 0 | 0 | 0 | 1 | 3 | 0.80729 | 1 |
| TET2 | 1 | 1 | 0 | 0 | 0 | 0 | 0 | 2 | 0.81506 | 1 |
| BRCA1 | 1 | 1 | 0 | 2 | 0 | 0 | 0 | 4 | 0.82338 | 1 |
| AKAP12 | 1 | 2 | 0 | 1 | 0 | 0 | 0 | 4 | 0.83113 | 1 |
| NTRK1 | 2 | 2 | 0 | 0 | 0 | 0 | 0 | 4 | 0.83151 | 1 |
| ATRX | 1 | 3 | 0 | 0 | 0 | 0 | 0 | 4 | 0.83899 | 1 |
| ITGA9 | 3 | 2 | 0 | 0 | 0 | 0 | 0 | 5 | 0.84206 | 1 |
| EPHA7 | 2 | 4 | 0 | 1 | 0 | 0 | 0 | 7 | 0.84363 | 1 |
| MYOM2 | 0 | 1 | 0 | 0 | 0 | 0 | 0 | 1 | 0.84404 | 1 |
| EVPL | 0 | 1 | 0 | 0 | 0 | 0 | 1 | 2 | 0.84507 | 1 |
| RPTOR | 2 | 1 | 0 | 0 | 0 | 0 | 0 | 3 | 0.84992 | 1 |
| POLE | 2 | 4 | 0 | 0 | 0 | 0 | 0 | 6 | 0.85235 | 1 |
| DPYD | 2 | 4 | 0 | 0 | 0 | 0 | 0 | 6 | 0.8554 | 1 |
| PTGS1 | 0 | 1 | 0 | 1 | 0 | 0 | 0 | 2 | 0.85895 | 1 |
| PTPRD | 2 | 2 | 0 | 0 | 0 | 0 | 0 | 4 | 0.86366 | 1 |
| TAF1 | 0 | 1 | 0 | 0 | 2 | 0 | 0 | 3 | 0.87126 | 1 |
| FN1 | 4 | 4 | 0 | 0 | 1 | 2 | 0 | 11 | 0.87225 | 1 |
| CBLB | 2 | 3 | 0 | 2 | 0 | 0 | 0 | 7 | 0.87889 | 1 |
| MAGI1 | 0 | 3 | 0 | 0 | 0 | 0 | 0 | 3 | 0.8893 | 1 |
| CTNNA1 | 2 | 1 | 0 | 0 | 1 | 0 | 0 | 4 | 0.89081 | 1 |
| EPHA3 | 1 | 1 | 0 | 0 | 1 | 0 | 0 | 3 | 0.89152 | 1 |
| CLCA2 | 3 | 2 | 0 | 0 | 0 | 0 | 0 | 5 | 0.89364 | 1 |
| LRP5 | 2 | 1 | 0 | 0 | 1 | 0 | 0 | 4 | 0.90452 | 1 |
| SUFU | 1 | 1 | 0 | 0 | 0 | 0 | 0 | 2 | 0.90698 | 1 |
| CACNA2D1 | 2 | 2 | 0 | 0 | 0 | 1 | 0 | 5 | 0.91361 | 1 |
| ERBB4 | 0 | 0 | 0 | 1 | 0 | 0 | 0 | 1 | 0.91636 | 1 |
| FLT4 | 4 | 3 | 0 | 0 | 0 | 0 | 0 | 7 | 0.91808 | 1 |
| ROS1 | 2 | 3 | 0 | 0 | 1 | 0 | 0 | 6 | 0.91814 | 1 |
| USP42 | 1 | 2 | 0 | 0 | 0 | 0 | 0 | 3 | 0.92166 | 1 |
| MUC16 | 12 | 31 | 0 | 2 | 0 | 1 | 0 | 46 | 0.92619 | 1 |
| ARHGEF7 | 2 | 2 | 0 | 0 | 0 | 0 | 0 | 4 | 0.92895 | 1 |
| ARID1A | 1 | 2 | 0 | 2 | 0 | 0 | 1 | 6 | 0.9312 | 1 |
| MLLT4 | 2 | 0 | 0 | 0 | 1 | 0 | 0 | 3 | 0.9318 | 1 |
| PRF1 | 2 | 3 | 0 | 0 | 0 | 0 | 0 | 5 | 0.93429 | 1 |
| SYNE1 | 11 | 27 | 0 | 3 | 0 | 3 | 0 | 43 | 0.03909 | 1 |
| MTOR | 2 | 2 | 0 | 0 | 0 | 0 | 0 | 4 | 0.034469 | 1 |
| TRIM24 | 0 | 1 | 0 | 1 | 0 | 0 | 0 | 2 | 0.034714 | 1 |
| HOXC13 | 1 | 1 | 0 | 0 | 0 | 0 | 0 | 2 | 0.035026 | 1 |
| PCDH9 | 3 | 10 | 0 | 2 | 0 | 0 | 0 | 15 | 0.035126 | 1 |
| MXRA5 | 5 | 4 | 0 | 0 | 0 | 0 | 0 | 9 | 0.035246 | 1 |
| ATP10A | 3 | 4 | 0 | 0 | 1 | 0 | 0 | 8 | 0.035282 | 1 |
| ZNF423 | 2 | 3 | 0 | 0 | 0 | 0 | 0 | 5 | 0.036271 | 1 |
| IGF2R | 1 | 0 | 0 | 1 | 0 | 0 | 0 | 2 | 0.036893 | 1 |
| FASN | 0 | 3 | 0 | 0 | 1 | 0 | 0 | 4 | 0.037462 | 1 |
| PTPRC | 4 | 3 | 0 | 0 | 0 | 0 | 1 | 8 | 0.09762 | 1 |
| DAB2IP | 2 | 2 | 0 | 0 | 0 | 0 | 0 | 4 | 0.97809 | 1 |
| MYH11 | 3 | 1 | 0 | 0 | 0 | 0 | 0 | 4 | 0.98944 | 1 |
| MAP2K2 | 2 | 1 | 0 | 0 | 0 | 0 | 0 | 3 | 0.99099 | 1 |
| CYP3A4 | 1 | 1 | 0 | 0 | 0 | 0 | 0 | 2 | 0.9935 | 1 |
| ATM | 0 | 8 | 0 | 2 | 1 | 0 | 0 | 11 | 0.99384 | 1 |
| SDHA | 3 | 3 | 0 | 0 | 0 | 0 | 0 | 6 | 0.99544 | 1 |
| AR | 4 | 5 | 0 | 0 | 0 | 0 | 0 | 9 | 0.99653 | 1 |
| GPR98 | 2 | 13 | 0 | 0 | 2 | 1 | 1 | 19 | 0.99704 | 1 |
| TAL2 | 1 | 1 | 0 | 0 | 0 | 0 | 0 | 2 | 0.99913 | 1 |
| POR | 1 | 1 | 0 | 0 | 0 | 0 | 0 | 2 | 0.99932 | 1 |
| PRDM16 | 2 | 1 | 0 | 0 | 0 | 0 | 0 | 3 | 0.99978 | 1 |
| EP400 | 7 | 6 | 0 | 1 | 0 | 0 | 0 | 12 | 0.99997 | 1 |
| SIGLEC10 | 3 | 1 | 0 | 0 | 0 | 0 | 0 | 4 | 0.99999 | 1 |
| NUMA1 | 0 | 5 | 0 | 0 | 0 | 0 | 0 | 5 | 0.99999 | 1 |
| MKL1 | 4 | 2 | 0 | 0 | 0 | 0 | 0 | 6 | 1 | 1 |
| TRRAP | 4 | 7 | 0 | 0 | 1 | 0 | 0 | 10 | 1 | 1 |
| ARID3B | 4 | 1 | 0 | 0 | 0 | 0 | 0 | 5 | 1 | 1 |
| ABCG2 | 1 | 0 | 0 | 0 | 0 | 0 | 0 | 1 | 1 | 1 |
| ABL1 | 3 | 0 | 0 | 0 | 0 | 0 | 0 | 3 | 1 | 1 |
| ACVR1B | 1 | 0 | 0 | 0 | 0 | 1 | 0 | 2 | 1 | 1 |
| ADAMTSL3 | 3 | 0 | 0 | 0 | 0 | 0 | 0 | 3 | 1 | 1 |
| ADD3 | 1 | 0 | 0 | 0 | 0 | 0 | 0 | 1 | 1 | 1 |
| AFF4 | 0 | 0 | 0 | 0 | 1 | 0 | 0 | 1 | 1 | 1 |
| AHR | 1 | 0 | 0 | 0 | 0 | 0 | 0 | 1 | 1 | 1 |
| AIM2 | 0 | 0 | 0 | 0 | 2 | 0 | 0 | 2 | 1 | 1 |
| ARHGAP26 | 0 | 0 | 0 | 0 | 0 | 0 | 1 | 1 | 1 | 1 |
| ARID4B | 2 | 0 | 0 | 0 | 0 | 0 | 0 | 2 | 1 | 1 |
| AURKB | 1 | 0 | 0 | 0 | 0 | 0 | 0 | 1 | 1 | 1 |
| BCL2A1 | 1 | 0 | 0 | 0 | 0 | 0 | 0 | 1 | 1 | 1 |
| BCOR | 1 | 0 | 0 | 0 | 0 | 0 | 0 | 1 | 1 | 1 |
| BNIP3 | 0 | 0 | 0 | 0 | 1 | 0 | 0 | 1 | 1 | 1 |
| CCND3 | 0 | 0 | 0 | 0 | 1 | 0 | 0 | 1 | 1 | 1 |
| CD274 | 1 | 0 | 0 | 0 | 0 | 0 | 0 | 1 | 1 | 1 |
| CD36 | 1 | 0 | 0 | 0 | 0 | 0 | 0 | 1 | 1 | 1 |
| CD44 | 1 | 0 | 0 | 0 | 0 | 0 | 0 | 1 | 1 | 1 |
| CD8A | 2 | 0 | 0 | 0 | 0 | 0 | 0 | 2 | 1 | 1 |
| CDKN2A | 0 | 0 | 0 | 1 | 0 | 0 | 0 | 1 | 1 | 1 |
| CDKN3 | 1 | 0 | 0 | 0 | 0 | 0 | 0 | 1 | 1 | 1 |
| CDX2 | 0 | 0 | 0 | 0 | 0 | 1 | 0 | 1 | 1 | 1 |
| CHCHD7 | 0 | 1 | 0 | 0 | 0 | 0 | 0 | 1 | 1 | 1 |
| CHD1 | 1 | 0 | 0 | 0 | 1 | 0 | 0 | 2 | 1 | 1 |
| CTNNA3 | 4 | 0 | 0 | 0 | 0 | 0 | 0 | 4 | 1 | 1 |
| CUX1 | 2 | 0 | 0 | 0 | 0 | 0 | 0 | 2 | 1 | 1 |
| DDX41 | 0 | 0 | 0 | 0 | 0 | 0 | 1 | 1 | 1 | 1 |
| DDX5 | 1 | 0 | 0 | 0 | 0 | 0 | 0 | 1 | 1 | 1 |
| DDX6 | 1 | 0 | 0 | 0 | 0 | 0 | 0 | 1 | 1 | 1 |
| DRD2 | 2 | 0 | 0 | 0 | 0 | 0 | 0 | 2 | 1 | 1 |
| EIF4A2 | 0 | 0 | 0 | 0 | 1 | 0 | 0 | 1 | 1 | 1 |
| ELK4 | 0 | 1 | 0 | 0 | 0 | 0 | 0 | 1 | 1 | 1 |
| ELN | 0 | 0 | 0 | 0 | 0 | 1 | 0 | 1 | 1 | 1 |
| EPHA10 | 1 | 0 | 0 | 0 | 0 | 0 | 0 | 1 | 1 | 1 |
| EPHA2 | 1 | 0 | 0 | 0 | 0 | 0 | 0 | 1 | 1 | 1 |
| ETS1 | 1 | 0 | 0 | 0 | 0 | 0 | 0 | 1 | 1 | 1 |
| FADD | 1 | 0 | 0 | 0 | 0 | 0 | 0 | 1 | 1 | 1 |
| FANCB | 0 | 0 | 0 | 0 | 1 | 0 | 0 | 1 | 1 | 1 |
| FANCD2 | 0 | 0 | 0 | 0 | 1 | 0 | 3 | 2 | 1 | 1 |
| FANCE | 2 | 0 | 0 | 0 | 0 | 0 | 0 | 2 | 1 | 1 |
| FANCF | 1 | 0 | 0 | 0 | 0 | 0 | 0 | 1 | 1 | 1 |
| FANCI | 1 | 0 | 0 | 0 | 0 | 0 | 0 | 1 | 1 | 1 |
| FBXO11 | 1 | 0 | 0 | 0 | 1 | 0 | 0 | 2 | 1 | 1 |
| FGFR1 | 1 | 0 | 0 | 0 | 0 | 0 | 0 | 1 | 1 | 1 |
| FGFR2 | 2 | 0 | 0 | 0 | 0 | 0 | 0 | 2 | 1 | 1 |
| FGFR4 | 1 | 0 | 0 | 0 | 0 | 0 | 0 | 1 | 1 | 1 |
| FUS | 1 | 0 | 0 | 0 | 0 | 0 | 0 | 1 | 1 | 1 |
| FZR1 | 0 | 0 | 0 | 0 | 1 | 0 | 0 | 1 | 1 | 1 |
| GBGT1 | 0 | 0 | 0 | 0 | 0 | 1 | 0 | 1 | 1 | 1 |
| GIT2 | 0 | 0 | 0 | 0 | 1 | 0 | 0 | 1 | 1 | 1 |
| GLMN | 1 | 0 | 0 | 0 | 0 | 4 | 0 | 2 | 1 | 1 |
| GMPS | 1 | 0 | 0 | 0 | 1 | 0 | 0 | 2 | 1 | 1 |
| GNA11 | 1 | 0 | 0 | 0 | 0 | 0 | 0 | 1 | 1 | 1 |
| GRK4 | 2 | 0 | 0 | 0 | 0 | 0 | 0 | 2 | 1 | 1 |
| GSK3B | 0 | 0 | 0 | 0 | 0 | 0 | 1 | 1 | 1 | 1 |
| HAX1 | 1 | 0 | 0 | 0 | 0 | 0 | 0 | 1 | 1 | 1 |
| HEXIM1 | 1 | 0 | 0 | 0 | 0 | 0 | 0 | 1 | 1 | 1 |
| HIST1H4I | 1 | 0 | 0 | 0 | 0 | 0 | 0 | 1 | 1 | 1 |
| HRAS | 1 | 0 | 0 | 0 | 0 | 0 | 0 | 1 | 1 | 1 |
| IKZF1 | 3 | 0 | 0 | 0 | 0 | 0 | 0 | 3 | 1 | 1 |
| JUN | 1 | 0 | 0 | 0 | 0 | 0 | 0 | 1 | 1 | 1 |
| KCNH2 | 1 | 0 | 0 | 0 | 0 | 0 | 0 | 1 | 1 | 1 |
| KDM5C | 1 | 0 | 0 | 0 | 1 | 0 | 0 | 2 | 1 | 1 |
| KDM6A | 0 | 0 | 0 | 0 | 0 | 0 | 2 | 1 | 1 | 1 |
| KL | 0 | 0 | 0 | 0 | 0 | 0 | 1 | 1 | 1 | 1 |
| KLF4 | 1 | 0 | 0 | 0 | 0 | 0 | 0 | 1 | 1 | 1 |
| LHFP | 2 | 0 | 0 | 0 | 1 | 0 | 0 | 3 | 1 | 1 |
| LMO1 | 1 | 0 | 0 | 0 | 0 | 0 | 0 | 1 | 1 | 1 |
| LTBP2 | 1 | 0 | 0 | 0 | 0 | 0 | 0 | 1 | 1 | 1 |
| LTK | 1 | 0 | 0 | 0 | 0 | 0 | 1 | 2 | 1 | 1 |
| MARK4 | 0 | 0 | 0 | 0 | 1 | 0 | 0 | 1 | 1 | 1 |
| MC1R | 2 | 0 | 0 | 0 | 0 | 0 | 0 | 2 | 1 | 1 |
| MDM4 | 2 | 1 | 0 | 0 | 0 | 0 | 0 | 3 | 1 | 1 |
| MEF2D | 1 | 0 | 0 | 0 | 0 | 0 | 0 | 1 | 1 | 1 |
| MEN1 | 1 | 0 | 0 | 0 | 0 | 0 | 0 | 1 | 1 | 1 |
| MLLT1 | 1 | 0 | 0 | 0 | 0 | 0 | 0 | 1 | 1 | 1 |
| MLLT10 | 1 | 0 | 0 | 0 | 0 | 0 | 0 | 1 | 1 | 1 |
| MLLT6 | 1 | 0 | 0 | 0 | 0 | 0 | 0 | 1 | 1 | 1 |
| MMP2 | 1 | 0 | 0 | 0 | 0 | 0 | 0 | 1 | 1 | 1 |
| MRPL3 | 1 | 0 | 0 | 0 | 0 | 0 | 0 | 1 | 1 | 1 |
| MSR1 | 1 | 0 | 0 | 0 | 1 | 0 | 0 | 2 | 1 | 1 |
| NFIB | 0 | 2 | 0 | 0 | 0 | 0 | 0 | 2 | 1 | 1 |
| NFKB2 | 1 | 0 | 0 | 0 | 0 | 0 | 0 | 1 | 1 | 1 |
| NOTCH1 | 3 | 2 | 0 | 1 | 0 | 0 | 0 | 6 | 1 | 1 |
| P4HA2 | 1 | 0 | 0 | 0 | 0 | 0 | 0 | 1 | 1 | 1 |
| PARP1 | 1 | 0 | 0 | 0 | 0 | 0 | 0 | 1 | 1 | 1 |
| PAX5 | 0 | 0 | 0 | 0 | 2 | 0 | 0 | 2 | 1 | 1 |
| PBRM1 | 1 | 0 | 0 | 0 | 0 | 0 | 0 | 1 | 1 | 1 |
| PEG3 | 1 | 0 | 0 | 0 | 0 | 0 | 0 | 1 | 1 | 1 |
| PER1 | 1 | 0 | 0 | 0 | 0 | 1 | 0 | 2 | 1 | 1 |
| PHOX2B | 1 | 0 | 0 | 0 | 0 | 0 | 0 | 1 | 1 | 1 |
| PIM1 | 1 | 1 | 0 | 0 | 0 | 0 | 0 | 1 | 1 | 1 |
| PML | 2 | 0 | 0 | 0 | 0 | 0 | 0 | 2 | 1 | 1 |
| PMS2 | 1 | 0 | 0 | 0 | 0 | 0 | 0 | 1 | 1 | 1 |
| PTPRJ | 2 | 0 | 0 | 0 | 0 | 0 | 0 | 2 | 1 | 1 |
| PXN | 0 | 1 | 0 | 0 | 0 | 0 | 0 | 1 | 1 | 1 |
| RAD51 | 1 | 0 | 0 | 0 | 0 | 0 | 0 | 1 | 1 | 1 |
| RGS20 | 2 | 0 | 0 | 0 | 0 | 0 | 0 | 2 | 1 | 1 |
| RRM1 | 0 | 0 | 0 | 0 | 1 | 0 | 0 | 1 | 1 | 1 |
| SBF2 | 1 | 0 | 0 | 0 | 0 | 0 | 1 | 2 | 1 | 1 |
| SFXN4 | 0 | 0 | 0 | 0 | 1 | 0 | 0 | 1 | 1 | 1 |
| SLC16A1 | 0 | 2 | 0 | 0 | 0 | 0 | 0 | 1 | 1 | 1 |
| SLC22A3 | 1 | 0 | 0 | 0 | 0 | 0 | 0 | 1 | 1 | 1 |
| SLC38A3 | 0 | 1 | 0 | 0 | 0 | 0 | 0 | 1 | 1 | 1 |
| SLC6A3 | 0 | 0 | 0 | 0 | 1 | 0 | 0 | 1 | 1 | 1 |
| SLC9A3R1 | 0 | 0 | 0 | 0 | 1 | 0 | 0 | 1 | 1 | 1 |
| SMAD6 | 1 | 0 | 0 | 0 | 0 | 0 | 0 | 1 | 1 | 1 |
| SNX2 | 1 | 0 | 0 | 0 | 0 | 0 | 0 | 1 | 1 | 1 |
| SNX29 | 2 | 0 | 0 | 0 | 0 | 0 | 0 | 1 | 1 | 1 |
| SOCS3 | 1 | 0 | 0 | 0 | 0 | 0 | 0 | 1 | 1 | 1 |
| STAT1 | 2 | 0 | 0 | 0 | 0 | 0 | 0 | 2 | 1 | 1 |
| STAT5B | 2 | 0 | 0 | 0 | 0 | 0 | 0 | 2 | 1 | 1 |
| STXBP2 | 0 | 0 | 0 | 0 | 1 | 0 | 0 | 1 | 1 | 1 |
| SYK | 1 | 0 | 0 | 0 | 0 | 0 | 0 | 1 | 1 | 1 |
| T | 0 | 1 | 0 | 0 | 0 | 0 | 0 | 1 | 1 | 1 |
| TBX22 | 0 | 0 | 0 | 0 | 1 | 0 | 0 | 1 | 1 | 1 |
| TCF12 | 2 | 0 | 0 | 0 | 0 | 0 | 0 | 1 | 1 | 1 |
| TCL1A | 1 | 0 | 0 | 0 | 0 | 0 | 0 | 1 | 1 | 1 |
| TFRC | 1 | 0 | 0 | 0 | 0 | 0 | 0 | 1 | 1 | 1 |
| TNFRSF13B | 1 | 0 | 0 | 0 | 0 | 0 | 0 | 1 | 1 | 1 |
| TNKS2 | 3 | 0 | 0 | 0 | 0 | 0 | 0 | 3 | 1 | 1 |
| TPM3 | 1 | 0 | 0 | 0 | 0 | 0 | 0 | 1 | 1 | 1 |
| TRAF7 | 2 | 1 | 0 | 0 | 0 | 0 | 0 | 3 | 1 | 1 |
| TRIP11 | 0 | 0 | 0 | 0 | 1 | 0 | 0 | 1 | 1 | 1 |
| TSHR | 0 | 0 | 0 | 0 | 1 | 0 | 0 | 1 | 1 | 1 |
| TYK2 | 2 | 0 | 0 | 0 | 0 | 0 | 0 | 2 | 1 | 1 |
| VKORC1 | 1 | 0 | 0 | 0 | 0 | 0 | 0 | 1 | 1 | 1 |
| ZMIZ1 | 2 | 0 | 0 | 0 | 0 | 0 | 0 | 2 | 1 | 1 |
| SEPT2 | 0 | 1 | 0 | 0 | 0 | 0 | 0 | 1 | 1 | 1 |
| SEPT6 | 0 | 1 | 0 | 1 | 0 | 0 | 0 | 2 | 1 | 1 |
| SEPT9 | 0 | 1 | 0 | 0 | 1 | 0 | 0 | 2 | 1 | 1 |
| ACKR3 | 2 | 0 | 0 | 0 | 0 | 0 | 0 | 2 | 1 | 1 |
| AMER1 | 0 | 3 | 0 | 2 | 0 | 0 | 0 | 5 | 1 | 1 |
| ARHGEF26 | 1 | 2 | 0 | 0 | 0 | 0 | 0 | 3 | 1 | 1 |
| BIVM-ERCC5 | 1 | 2 | 0 | 1 | 0 | 0 | 0 | 4 | 1 | 1 |
| C12orf79 | 0 | 0 | 0 | 0 | 1 | 0 | 0 | 1 | 1 | 1 |
| CNTRL | 0 | 1 | 0 | 0 | 0 | 0 | 0 | 1 | 1 | 1 |
| DEPTOR | 0 | 1 | 0 | 0 | 0 | 0 | 0 | 1 | 1 | 1 |
| DROSHA | 2 | 2 | 0 | 0 | 0 | 2 | 1 | 6 | 1 | 1 |
| ENKD1 | 0 | 1 | 0 | 0 | 0 | 0 | 0 | 1 | 1 | 1 |
| KAT6A | 0 | 2 | 0 | 0 | 0 | 0 | 0 | 2 | 1 | 1 |
| KAT6B | 1 | 1 | 0 | 0 | 0 | 0 | 0 | 2 | 1 | 1 |
| KHDC3L | 0 | 0 | 0 | 0 | 1 | 0 | 0 | 1 | 1 | 1 |
| KMT2A | 1 | 3 | 0 | 0 | 0 | 0 | 0 | 4 | 1 | 1 |
| KMT2B | 1 | 4 | 0 | 0 | 0 | 0 | 0 | 5 | 1 | 1 |
| KMT2C | 0 | 7 | 0 | 0 | 0 | 0 | 1 | 9 | 1 | 1 |
| KMT2D | 2 | 4 | 0 | 1 | 0 | 0 | 0 | 6 | 1 | 1 |
| LOC100129083 | 0 | 0 | 0 | 1 | 0 | 0 | 0 | 1 | 1 | 1 |
| LRIF1 | 0 | 2 | 0 | 0 | 0 | 0 | 1 | 3 | 1 | 1 |
| MEF2BNB-MEF2B | 0 | 1 | 0 | 0 | 0 | 0 | 0 | 1 | 1 | 1 |
| MRGBP | 1 | 0 | 0 | 0 | 0 | 0 | 0 | 1 | 1 | 1 |
| NOA1 | 0 | 1 | 0 | 0 | 0 | 0 | 0 | 1 | 1 | 1 |
| NUTM1 | 0 | 1 | 0 | 0 | 0 | 0 | 0 | 1 | 1 | 1 |
| RPS10-NUDT3 | 0 | 1 | 0 | 1 | 0 | 0 | 0 | 2 | 1 | 1 |
| SLCO1B7 | 0 | 2 | 0 | 0 | 0 | 0 | 0 | 2 | 1 | 1 |
| SLX4 | 1 | 2 | 0 | 0 | 0 | 0 | 0 | 3 | 1 | 1 |
| SPECC1 | 0 | 3 | 0 | 0 | 0 | 0 | 0 | 3 | 1 | 1 |
| SRSF2 | 0 | 1 | 0 | 0 | 0 | 0 | 0 | 1 | 1 | 1 |
| SRSF3 | 0 | 1 | 0 | 0 | 0 | 0 | 0 | 1 | 1 | 1 |

Table 9: Genomic alterations unique to each SNF subtype. Differentially enriched mutations and gene-level copy number variations are presented. Analysis of gene-level copy number variations was performed for those genes identified by TCGA in primary colorectal cancers²⁵. Overall, analyses were performed for genomic aberrations with at least 20% frequency in at least one SNF subtype. Statistical significance was determined using Fisher's exact tests between each SNF group versus the remaining two SNF groups.

TABLE 9

| Genomic | | | | | |
|---|---|---|---|---|---|
| Aberration | SNF1 | SNF2 | SNF3 | Comparison | P-value |
| NOTCH1 mutation | 23.1% | 0.0% | 0.0% | SNF1v2,3 | 0.0232 |
| PIK3C2B mutation | 23.1% | 0.0% | 0.0% | SNF1v2,3 | 0.0232 |
| CDK12 mutation | 0.0% | 20.0% | 0.0% | SNF2v1,3 | 0.0369 |
| EBF1 mutation | 0.0% | 20.0% | 0.0% | SNF2v1,3 | 0.0369 |
| NRAS mutation | 0.0% | 20.0% | 0.0% | SNF2v1,3 | 0.0369 |
| DMD mutation | 0.0% | 26.7% | 6.7% | SNF2v1,3 | 0.0428 |
| VEGFA | 46.2% | 6.7% | 40.0% | SNF2v1,3 | 0.017 |
| amplification | | | | | |
| SMAD3 mutation | 0.0% | 0.0% | 20.0% | SNF3v1,2 | 0.0369 |

Tables 10A-C: Table 10A lists genes whose expression is analyzed in a classification model for identifying SNF2 metastases. The difference in gene expression (“Log 2FC” column) between SNF2 metastases as compared to SNF1 and SNF3 metastases is shown, along with the function or pathway associated with each gene. Table 10B lists the genes in Table 10A that are expressed at a significantly higher level in SNF2 metastases than in SNF1 and SNF3 metastases. Table 10C lists the genes in Table 10A that are expressed at a significantly lower level in SNF2 metastases than in SNF1 and SNF3 metastases.

TABLE 10A

| | Gene | log2FC | |
|---|---|---|---|
| ENSEMBL ID | Symbol | (SNF2 vs. SNF1, 3) | Function/Pathway |
| ENSG00000082438 | COBLL1 | −0.800881317 | Actin binding |
| ENSG00000170417 | TMEM182 | −1.045618291 | Adipogenesis; myogenesis |
| ENSG00000176720 | BOK | 1.690960733 | Apoptosis; Development |
| ENSG00000156171 | DRAM2 | −0.851968947 | Autophagy |
| ENSG00000140941 | MAP1LC3B | −0.735698571 | Autophagy |
| ENSG00000129467 | ADCY4 | 1.220343115 | cAMP formation |
| ENSG00000076662 | ICAM3 | 1.368014118 | Cell adhesion |
| ENSG00000164162 | ANAPC10 | −0.746014397 | Cell cycle |
| ENSG00000107443 | CCNJ | −0.890337582 | Cell cycle |
| ENSG00000132466 | ANKRD17 | −0.533617194 | Cell differentiation |
| ENSG00000129474 | AJUBA | 1.046456583 | Cell proliferation and |
| | | | differentiation |
| ENSG00000105518 | TMEM205 | 0.879144662 | Cisplatin resistance |
| ENSG00000134318 | ROCK2 | −1.026390625 | Cytokinesis; focal adhesions; |
| | | | cytoskeleton |
| ENSG00000116127 | ALMS1 | −0.723286687 | Cytoskeleton |
| ENSG00000120860 | CCDC53 | −0.761708434 | Cytoskeleton; Endosome |
| ENSG00000089094 | KDM2B | 0.758836161 | Demethylation |
| ENSG00000144468 | RHBDD1 | −0.537760383 | Endopeptidase |
| ENSG00000179044 | EXOC3L1 | 1.566820255 | Exocytosis |
| ENSG00000181031 | RPH3AL | 0.988775688 | Exocytosis |
| ENSG00000080573 | COL5A3 | 1.413644796 | Extracellular matrix formation |
| ENSG00000205864 | KRTAP5-6 | 1.987671868 | Extracellular matrix formation |
| ENSG00000183153 | GJD3 | 2.478515517 | Gap junctions |
| ENSG00000138604 | GLCE | −1.04250676 | Glycosaminoglycan metabolism |
| ENSG00000115806 | GORASP2 | −0.428318392 | Golgi function |
| ENSG00000165801 | ARHGEF40 | 0.997555327 | G-protein receptor signaling |
| ENSG00000065135 | GNAI3 | −0.757257708 | G-protein receptor signaling |
| ENSG00000172380 | GNG12 | −0.836333361 | G-protein receptor signaling |
| ENSG00000132518 | GUCY2D | 2.393664098 | G-protein receptor signaling |
| ENSG00000189410 | SH2D5 | 2.394773121 | G-protein receptor signaling |
| ENSG00000170647 | TMEM133 | −1.262797743 | G-protein receptor signaling |
| ENSG00000158062 | UBXN11 | 0.948861211 | G-protein receptor signaling |
| ENSG00000162694 | EXTL2 | −0.92833322 | Heparin modification |
| ENSG00000181045 | SLC26A11 | 1.048138867 | Homeostasis; electrolyte |
| | | | balance |
| ENSG00000177663 | IL17RA | 0.571310231 | Immune response |
| ENSG00000155099 | TMEM55A | −0.841200023 | Inositol phosphate metabolism |
| ENSG00000125629 | INSIG2 | −0.868043988 | Lipid metabolism |
| ENSG00000144182 | LIPT1 | −1.120043984 | Lipid metabolism |
| ENSG00000110921 | MVK | 1.023529085 | Lipid metabolism |
| ENSG00000182378 | PLCXD1 | 1.44049891 | Lipid metabolism |
| ENSG00000167705 | RILP | 1.346192582 | Lysosome function |
| ENSG00000162600 | OMA1 | −0.979926283 | Metalloprotease |
| ENSG00000168906 | MAT2A | −0.571058603 | Methionine adenosyltransferase |
| | | | activity |
| ENSG00000198130 | HIBCH | −0.782578708 | Mitochondrial function; viral |
| | | | infection |
| ENSG00000148339 | SLC25A25 | 1.081771899 | Mitochondrial transport |
| ENSG00000068796 | KIF2A | −0.865443147 | Mitosis |
| ENSG00000175470 | PPP2R2D | 0.704123126 | Mitosis |
| ENSG00000100968 | NFATC4 | 1.330059279 | Motility |
| ENSG00000118965 | WDR35 | −1.127339428 | Motility; Sonic Hedgehog |
| | | | signaling |
| ENSG00000186654 | PRR5 | 0.869634036 | mTOR signaling |
| ENSG00000173786 | CNP | 0.776165238 | Nucleic acid binding |
| ENSG00000121058 | COIL | −0.705992325 | Organelle formation |
| ENSG00000204160 | ZDHHC18 | 0.82799159 | Palmitoyltransferase |
| ENSG00000090975 | PITPNM2 | 1.075064848 | Phosphatidylinositol transfer |
| ENSG00000227345 | PARG | −0.512288313 | Poly (ADP-ribose) catabolism |
| ENSG00000141161 | UNC45B | 2.270982748 | Protein chaperone |
| ENSG00000240344 | PPIL3 | −0.86000924 | Protein folding |
| ENSG00000178904 | DPY19L3 | −0.805965771 | Protein modification |
| ENSG00000121350 | PYROXD1 | −0.982117531 | Protein modification |
| ENSG00000155304 | HSPA13 | −0.97308995 | Protein processing |
| ENSG00000107902 | LHPP | 1.035307778 | Purine metabolism |
| ENSG00000222014 | RAB6C\|WTH3DI | −2.099593453 | RAS signaling |
| ENSG00000010017 | RANBP9 | −0.714562753 | RAS signaling |
| ENSG00000023572 | GLRX2 | −0.859346451 | Redox signaling |
| ENSG00000130244 | FAM98C | 0.893820425 | RNA binding |
| ENSG00000180917 | CMTR2 | −0.875904403 | RNA modification |
| ENSG00000185716 | C16orf521\|LOC102725138 | −0.949150607 | Sonic hedgehog signaling |
| ENSG00000203965 | EFCAB7 | −1.089559098 | Sonic hedgehog signaling |
| ENSG00000124380 | SNRNP27 | −0.627397765 | Splicing |
| ENSG00000112305 | SMAP1 | −0.624975625 | Stromal cell activation |
| ENSG00000164506 | STXBP5 | −0.974501473 | Synaptic vesicles |
| ENSG00000121989 | ACVR2A | −1.051537003 | TGF-beta signaling |
| ENSG00000223802 | GDF1\|CERS1 | 1.844243212 | TGF-beta signaling |
| ENSG00000157933 | SKI | 0.696032727 | TGF-beta signaling |
| ENSG00000136603 | SKIL | −1.090266877 | TGF-beta signaling |
| ENSG00000179348 | GATA2 | 1.867742578 | Transcription; immune signaling |
| ENSG00000160741 | CRTC2 | 0.605701789 | Transcriptional regulation |
| ENSG00000141979 | CTD-3222D19.2 (Clone-based | −2.076024446 | Transcriptional regulation |
| | (Vega) gene) | | |
| ENSG00000198746 | GPATCH3 | 0.827826305 | Transcriptional regulation |
| ENSG00000119669 | IRF2BPL | 1.115703855 | Transcriptional regulation |
| ENSG00000140044 | JDP2 | 1.005345222 | Transcriptional regulation |
| ENSG00000116044 | NFE2L2 | −0.592450472 | Transcriptional regulation |
| ENSG00000080298 | RFX3 | −0.869749954 | Transcriptional regulation |
| ENSG00000085721 | RRN3 | −0.702609197 | Transcriptional regulation |
| ENSG00000111832 | RWDD1 | −0.796074992 | Transcriptional regulation |
| ENSG00000087266 | SH3BP2 | 0.755613904 | Transcriptional regulation |
| ENSG00000115750 | TAF1B | −0.804267724 | Transcriptional regulation |
| ENSG00000184517 | ZFP1 | −0.709320083 | Transcriptional regulation |
| ENSG00000213096 | ZNF254 | −1.210045897 | Transcriptional regulation |
| ENSG00000181315 | ZNF322 | −0.803566951 | Transcriptional regulation |
| ENSG00000083838 | ZNF446 | 0.9935166 | Transcriptional regulation |
| ENSG00000132677 | RHBG | 2.904694676 | Transmembrane protein transport |
| ENSG00000075790 | BCAP29 | −1.010689151 | Transport |
| ENSG00000138036 | DYNC2LI1 | −0.889841056 | Transport |
| ENSG00000068697 | LAPTM4A | −0.632250511 | Transport |
| ENSG00000174327 | SLC16A13 | 1.083425693 | Transport |
| ENSG00000196950 | SLC39A10 | −0.96271217 | Transport |
| ENSG00000167703 | SLC43A2 | 1.086260232 | Transport |
| ENSG00000167645 | YIF1B | 1.033798947 | Transport |
| ENSG00000130222 | GADD45G | 2.063438246 | Tumor suppressor; cell cycle; |
| | | | apoptosis; senescence |
| ENSG00000145569 | FAM105A | −1.454434258 | Ubiquitin modification |
| ENSG00000151116 | UEVLD | −0.758631616 | Ubiquitin modification |
| ENSG00000182670 | TTC3 | −0.815325423 | Ubiquitin modification; Akt |
| | | | signaling |
| ENSG00000175104 | TRAF6 | −0.672175695 | Ubiquitin modification; immune |
| | | | response |
| ENSG00000077232 | DNAJC10 | −0.75940466 | Unfolded protein binding |
| ENSG00000110011 | DNAJC4 | 0.839114239 | Unfolded protein binding |
| ENSG00000137720 | C11orf1 | −1.041150318 | Unknown |
| ENSG00000159761 | C16orf86 | 1.728334231 | Unknown |
| ENSG00000108021 | FAM208B | −0.607079753 | Unknown |
| ENSG00000059122 | FLYWCH1 | 1.023031667 | Unknown |
| ENSG00000113638 | TTC33 | −1.011428566 | Unknown |
| ENSG00000146574 | CCZ1B | −1.044675474 | Vesicle transport |
| ENSG00000280433 | LOC102724200 | −1.459202933 | Vesicle transport |
| ENSG00000111906 | HDDC2 | −0.933634846 | Viral infection |

TABLE 10B

| | Gene | log2FC |
|---|---|---|
| ENSEMBL ID | Symbol | (SNF2 vs. SNF1, 3) |
| ENSG00000176720 | BOK | 1.690960733 |
| ENSG00000129467 | ADCY4 | 1.220343115 |
| ENSG00000076662 | ICAM3 | 1.368014118 |
| ENSG00000129474 | AJUBA | 1.046456583 |
| ENSG00000105518 | TMEM205 | 0.879144662 |
| ENSG00000089094 | KDM2B | 0.758836161 |
| ENSG00000179044 | EXOC3L1 | 1.566820255 |
| ENSG00000181031 | RPH3AL | 0.988775688 |
| ENSG00000080573 | COL5A3 | 1.413644796 |
| ENSG00000205864 | KRTAP5-6 | 1.987671868 |
| ENSG00000183153 | GJD3 | 2.478515517 |
| ENSG00000165801 | ARHGEF40 | 0.997555327 |
| ENSG00000132518 | GUCY2D | 2.393664098 |
| ENSG00000189410 | SH2D5 | 2.394773121 |
| ENSG00000158062 | UBXN11 | 0.948861211 |
| ENSG00000181045 | SLC26A11 | 1.048138867 |
| ENSG00000177663 | IL17RA | 0.571310231 |
| ENSG00000110921 | MVK | 1.023529085 |
| ENSG00000182378 | PLCXD1 | 1.44049891 |
| ENSG00000167705 | RILP | 1.346192582 |
| ENSG00000148339 | SLC25A25 | 1.081771899 |
| ENSG00000175470 | PPP2R2D | 0.704123126 |
| ENSG00000100968 | NFATC4 | 1.330059279 |
| ENSG00000186654 | PRR5 | 0.869634036 |
| ENSG00000173786 | CNP | 0.776165238 |
| ENSG00000204160 | ZDHHC18 | 0.82799159 |
| ENSG00000090975 | PITPNM2 | 1.075064848 |
| ENSG00000141161 | UNC45B | 2.270982748 |
| ENSG00000107902 | LHPP | 1.035307778 |
| ENSG00000130244 | FAM98C | 0.893820425 |
| ENSG00000223802 | GDF1\|CERS1 | 1.844243212 |
| ENSG00000157933 | SKI | 0.696032727 |
| ENSG00000179348 | GATA2 | 1.867742578 |
| ENSG00000160741 | CRTC2 | 0.605701789 |
| ENSG00000198746 | GPATCH3 | 0.827826305 |
| ENSG00000119669 | IRF2BPL | 1.115703855 |
| ENSG00000140044 | JDP2 | 1.005345222 |
| ENSG00000087266 | SH3BP2 | 0.755613904 |
| ENSG00000083838 | ZNF446 | 0.9935166 |
| ENSG00000132677 | RHBG | 2.904694676 |
| ENSG00000174327 | SLC16A13 | 1.083425693 |
| ENSG00000167703 | SLC43A2 | 1.086260232 |
| ENSG00000167645 | YIF1B | 1.033798947 |
| ENSG00000130222 | GADD45G | 2.063438246 |
| ENSG00000110011 | DNAJC4 | 0.839114239 |
| ENSG00000159761 | C16orf86 | 1.728334231 |
| ENSG00000059122 | FLYWCH1 | 1.023031667 |

TABLE 10C

| | Gene | log2FC |
|---|---|---|
| ENSEMBL ID | Symbol | (SNF2 vs. SNF1, 3) |
| ENSG00000082438 | COBLL1 | −0.800881317 |
| ENSG00000170417 | TMEM182 | −1.045618291 |
| ENSG00000156171 | DRAM2 | −0.851968947 |
| ENSG00000140941 | MAP1LC3B | −0.735698571 |
| ENSG00000164162 | ANAPC10 | −0.746014397 |
| ENSG00000107443 | CCNJ | −0.890337582 |
| ENSG00000132466 | ANKRD17 | −0.533617194 |
| ENSG00000134318 | ROCK2 | −1.026390625 |
| ENSG00000116127 | ALMS1 | −0.723286687 |
| ENSG00000120860 | CCDC53 | −0.761708434 |
| ENSG00000144468 | RHBDD1 | −0.537760383 |
| ENSG00000138604 | GLCE | −1.04250676 |
| ENSG00000115806 | GORASP2 | −0.428318392 |
| ENSG00000065135 | GNAI3 | −0.757257708 |
| ENSG00000172380 | GNG12 | −0.836333361 |
| ENSG00000170647 | TMEM133 | −1.262797743 |
| ENSG00000162694 | EXTL2 | −0.92833322 |
| ENSG00000155099 | TMEM55A | −0.841200023 |
| ENSG00000125629 | INSIG2 | −0.868043988 |
| ENSG00000144182 | LIPT1 | −1.120043984 |
| ENSG00000162600 | OMA1 | −0.979926283 |
| ENSG00000168906 | MAT2A | −0.571058603 |
| ENSG00000198130 | HIBCH | −0.782578708 |
| ENSG00000068796 | KIF2A | −0.865443147 |
| ENSG00000118965 | WDR35 | −1.127339428 |
| ENSG00000121058 | COIL | −0.705992325 |
| ENSG00000227345 | PARG | −0.512288313 |
| ENSG00000240344 | PPIL3 | −0.86000924 |
| ENSG00000178904 | DPY19L3 | −0.805965771 |
| ENSG00000121350 | PYROXD1 | −0.982117531 |
| ENSG00000155304 | HSPA13 | −0.97308995 |
| ENSG00000222014 | RAB6C\|WTH3DI | −2.099593453 |
| ENSG00000010017 | RANBP9 | −0.714562753 |
| ENSG00000023572 | GLRX2 | −0.859346451 |
| ENSG00000180917 | CMTR2 | −0.875904403 |
| ENSG00000185716 | C16orf52\|LOC102725138 | −0.949150607 |
| ENSG00000203965 | EFCAB7 | −1.089559098 |
| ENSG00000124380 | SNRNP27 | −0.627397765 |
| ENSG00000112305 | SMAP1 | −0.624975625 |
| ENSG00000164506 | STXBP5 | −0.974501473 |
| ENSG00000121989 | ACVR2A | −1.051537003 |
| ENSG00000136603 | SKIL | −1.090266877 |
| ENSG00000141979 | CTD-3222D19.2 (Clone- | −2.076024446 |
| | based (Vega) gene) | |
| ENSG00000116044 | NFE2L2 | −0.592450472 |
| ENSG00000080298 | RFX3 | −0.869749954 |
| ENSG00000085721 | RRN3 | −0.702609197 |
| ENSG00000111832 | RWDD1 | −0.796074992 |
| ENSG00000115750 | TAF1B | −0.804267724 |
| ENSG00000184517 | ZFP1 | −0.709320083 |
| ENSG00000213096 | ZNF254 | −1.210045897 |
| ENSG00000181315 | ZNF322 | −0.803566951 |
| ENSG00000075790 | BCAP29 | −1.010689151 |
| ENSG00000138036 | DYNC2LI1 | −0.889841056 |
| ENSG00000068697 | LAPTM4A | −0.632250511 |
| ENSG00000196950 | SLC39A10 | −0.96271217 |
| ENSG00000145569 | FAM105A | −1.454434258 |
| ENSG00000151116 | UEVLD | −0.758631616 |
| ENSG00000182670 | TTC3 | −0.815325423 |
| ENSG00000175104 | TRAF6 | −0.672175695 |
| ENSG00000077232 | DNAJC10 | −0.75940466 |
| ENSG00000137720 | C11orf1 | −1.041150318 |
| ENSG00000108021 | FAM208B | −0.607079753 |
| ENSG00000113638 | TTC33 | −1.011428566 |
| ENSG00000146574 | CCZ1B | −1.044675474 |
| ENSG00000280433 | LOC102724200 | −1.459202933 |
| ENSG00000111906 | HDDC2 | −0.933634846 |

Tables 11A-C: Table 11A lists miRNAs whose expression is analyzed in a classification model for identifying SNF2 metastases. The difference in miRNA expression (“Log 2FC” column) between SNF2 metastases as compared to SNF1 and SNF3 metastases is shown. Table 11B lists the miRNAs in Table 11A that are expressed at a significantly higher level in SNF2 metastases than in SNF1 and SNF3 metastases. Table 11C lists the miRNAs in Table 11A that are expressed at a significantly lower levels in SNF2 metastases than in SNF1 and SNF3 metastases.

TABLE 11A

| | Transcript | log2FC |
|---|---|---|
| Probe Set Name | ID | (SNF2 vs. SNF1, 3) |
| MIMAT0000418_st | hsa-miR-23b-3p | −0.973911144 |
| MIMAT0000103_st | hsa-miR-106a-5p | −0.940091903 |
| MIMAT0000065_st | hsa-let-7d-5p | −0.855624818 |
| MIMAT0001080_st | hsa-miR-196b-5p | −0.766240429 |
| MIMAT0000264_st | hsa-miR-203a | −0.763078462 |
| MIMAT0000086_st | hsa-miR-29a-3p | −0.735033964 |
| MIMAT0000222_st | hsa-miR-192-5p | −0.714440376 |
| MIMAT0022259_st | hsa-miR-5100 | −0.69282742 |
| MIMAT0000066_st | hsa-let-7e-5p | −0.672530325 |
| MIMAT0000259_st | hsa-miR-182-5p | −0.648299131 |
| MIMAT0000093_st | hsa-miR-93-5p | −0.644062486 |
| MIMAT0000318_st | hsa-miR-200b-3p | −0.641440676 |
| MIMAT0000076_st | hsa-miR-21-5p | −0.636319688 |
| MIMAT0000101_st | hsa-miR-103a-3p | −0.635289418 |
| MIMAT0000080_st | hsa-miR-24-3p | −0.599049459 |
| MIMAT0000691_st | hsa-miR-130b-3p | −0.549958054 |
| MIMAT0000278_st | hsa-miR-221-3p | −0.527703234 |
| MIMAT0000243_st | hsa-miR-148a-3p | −0.445186581 |
| MIMAT0000415_st | hsa-let-7i-5p | −0.407014793 |
| MIMAT0000071_st | hsa-miR-17-3p | −0.307974314 |
| MIMAT0001620_st | hsa-miR-200a-5p | −0.260909679 |
| MIMAT0018967_st | hsa-miR-4448 | −0.253668944 |
| MIMAT0018179_st | hsa-miR-3907 | 0.172170872 |
| MIMAT0027492_st | hsa-miR-6796-5p | 0.180916714 |
| MIMAT0027423_st | hsa-miR-6761-3p | 0.181240609 |
| MIMAT0022979_st | hsa-miR-4750-3p | 0.191503329 |
| MIMAT0007882_st | hsa-miR-1909-5p | 0.223357116 |
| MIMAT0026741_st | hsa-miR-1251-3p | 0.258013283 |
| MIMAT0000242_st | hsa-miR-129-5p | 0.27251568 |
| MIMAT0015070_st | hsa-miR-3188 | 0.27589605 |
| MIMAT0019809_st | hsa-miR-4708-5p | 0.281033806 |
| MIMAT0018065_st | hsa-miR-3646 | 0.284592893 |
| MIMAT0003240_st | hsa-miR-575 | 0.303532074 |
| MIMAT0019835_st | hsa-miR-4721 | 0.306997951 |
| MIMAT0004976_st | hsa-miR-933 | 0.322809789 |
| MIMAT0006765_st | hsa-miR-1825 | 0.347249747 |
| MIMAT0017352_st | hsa-miR-2277-5p | 0.350295367 |
| MIMAT0004561_st | hsa-miR-187-5p | 0.366473454 |
| MIMAT0019068_st | hsa-miR-4529-3p | 0.368761744 |
| MIMAT0027431_st | hsa-miR-6765-3p | 0.371166068 |
| MIMAT0030430_st | hsa-miR-7855-5p | 0.379288897 |
| MIMAT0028117_st | hsa-miR-7110-5p | 0.432829251 |
| MIMAT0003270_st | hsa-miR-602 | 0.460879594 |
| MIMAT0005951_st | hsa-miR-1307-3p | 0.465359964 |
| MIMAT0022742_st | hsa-miR-1273g-3p | 0.511636188 |
| MIMAT0015052_st | hsa-miR-3175 | 0.537970962 |
| MIMAT0004614_st | hsa-miR-193a-5p | 0.557948308 |
| MIMAT0005867_st | hsa-miR-663b | 0.75513205 |
| MIMAT0019745_st | hsa-miR-4668-5p | 0.814588736 |
| MIMAT0005939_st | hsa-miR-1281 | 0.866951101 |
| MIMAT0015055_st | hsa-miR-3178 | 1.047590393 |
| MIMAT0019069_st | hsa-miR-4530 | 1.280186233 |
| MIMAT0000421_st | hsa-miR-122-5p | 2.12222278 |

TABLE 11B

| | Transcript | log2FC |
|---|---|---|
| Probe Set Name | ID | (SNF2 vs. SNF1, 3) |
| MIMAT0018179_st | hsa-miR-3907 | 0.172170872 |
| MIMAT0027492_st | hsa-miR-6796-5p | 0.180916714 |
| MIMAT0027423_st | hsa-miR-6761-3p | 0.181240609 |
| MIMAT0022979_st | hsa-miR-4750-3p | 0.191503329 |
| MIMAT0007882_st | hsa-miR-1909-5p | 0.223357116 |
| MIMAT0026741_st | hsa-miR-1251-3p | 0.258013283 |
| MIMAT0000242_st | hsa-miR-129-5p | 0.27251568 |
| MIMAT0015070_st | hsa-miR-3188 | 0.27589605 |
| MIMAT0019809_st | hsa-miR-4708-5p | 0.281033806 |
| MIMAT0018065_st | hsa-miR-3646 | 0.284592893 |
| MIMAT0003240_st | hsa-miR-575 | 0.303532074 |
| MIMAT0019835_st | hsa-miR-4721 | 0.306997951 |
| MIMAT0004976_st | hsa-miR-933 | 0.322809789 |
| MIMAT0006765_st | hsa-miR-1825 | 0.347249747 |
| MIMAT0017352_st | hsa-miR-2277-5p | 0.350295367 |
| MIMAT0004561_st | hsa-miR-187-5p | 0.366473454 |
| MIMAT0019068_st | hsa-miR-4529-3p | 0.368761744 |
| MIMAT0027431_st | hsa-miR-6765-3p | 0.371166068 |
| MIMAT0030430_st | hsa-miR-7855-5p | 0.379288897 |
| MIMAT0028117_st | hsa-miR-7110-5p | 0.432829251 |
| MIMAT0003270_st | hsa-miR-602 | 0.460879594 |
| MIMAT0005951_st | hsa-miR-1307-3p | 0.465359964 |
| MIMAT0022742_st | hsa-miR-1273g-3p | 0.511636188 |
| MIMAT0015052_st | hsa-miR-3175 | 0.537970962 |
| MIMAT0004614_st | hsa-miR-193a-5p | 0.557948308 |
| MIMAT0005867_st | hsa-miR-663b | 0.75513205 |
| MIMAT0019745_st | hsa-miR-4668-5p | 0.814588736 |
| MIMAT0005939_st | hsa-miR-1281 | 0.866951101 |
| MIMAT0015055_st | hsa-miR-3178 | 1.047590393 |
| MIMAT0019069_st | hsa-miR-4530 | 1.280186233 |
| MIMAT0000421_st | hsa-miR-122-5p | 2.12222278 |

TABLE 11C

| | Transcript | log2FC |
|---|---|---|
| Probe Set Name | ID | (SNF2 vs. SNF1, 3) |
| MIMAT0000418_st | hsa-miR-23b-3p | −0.973911144 |
| MIMAT0000103_st | hsa-miR-106a-5p | −0.940091903 |
| MIMAT0000065_st | hsa-let-7d-5p | −0.855624818 |
| MIMAT0001080_st | hsa-miR-196b-5p | −0.766240429 |
| MIMAT0000264_st | hsa-miR-203a | −0.763078462 |
| MIMAT0000086_st | hsa-miR-29a-3p | −0.735033964 |
| MIMAT0000222_st | hsa-miR-192-5p | −0.714440376 |
| MIMAT0022259_st | hsa-miR-5100 | −0.69282742 |
| MIMAT0000066_st | hsa-let-7e-5p | −0.672530325 |
| MIMAT0000259_st | hsa-miR-182-5p | −0.648299131 |
| MIMAT0000093_st | hsa-miR-93-5p | −0.644062486 |
| MIMAT0000318_st | hsa-miR-200b-3p | −0.641440676 |
| MIMAT0000076_st | hsa-miR-21-5p | −0.636319688 |
| MIMAT0000101_st | hsa-miR-103a-3p | −0.635289418 |
| MIMAT0000080_st | hsa-miR-24-3p | −0.599049459 |
| MIMAT0000691_st | hsa-miR-130b-3p | −0.549958054 |
| MIMAT0000278_st | hsa-miR-221-3p | −0.527703234 |
| MIMAT0000243_st | hsa-miR-148a-3p | −0.445186581 |
| MIMAT0000415_st | hsa-let-7i-5p | −0.407014793 |
| MIMAT0000071_st | hsa-miR-17-3p | −0.307974314 |
| MIMAT0001620_st | hsa-miR-200a-5p | −0.260909679 |
| MIMAT0018967_st | hsa-miR-4448 | −0.253668944 |

### REFERENCES

1 Hellman S, Weichselbaum R R. Oligometastases. J Clin Oncol 1995; 13:8-10.
2. Weichselbaum R R, Hellman S. Oligometastases revisited. Nat Rev Clin Oncol 2011; 8:378-82.
3. Palma D A, Salama J K, Lo S S, et al. The oligometastatic state—separating truth from wishful thinking. Nat Rev Clin Oncol 2014; 11:549-57.
4. Treasure T. Oligometastatic cancer: an entity, a useful concept, or a therapeutic opportunity? J R Soc Med 2012; 105:242-6.
5. Mehta N, Mauer A M, Hellman S, et al. Analysis of further disease progression in metastatic non-small cell lung cancer: implications for locoregional treatment. Int J Oncol 2004; 25:1677-83.
6. Hong J C, Salama J K. The expanding role of stereotactic body radiation therapy in oligometastatic solid tumors: What do we know and where are we going? Cancer Treat Rev 2017; 52:22-32.
7. Tosoian J J, Gorin M A, Ross A E, Pienta K J, Tran P T, Schaeffer E M. Oligometastatic prostate cancer: definitions, clinical outcomes, and treatment considerations. Nat Rev Urol 2017; 14:15-25.
8. Loh J, Davis I D, Martin J M, Siva S. Extracranial oligometastatic renal cell carcinoma: current management and future directions. Future Oncol 2014; 10:761-74.
9. Fong Y, Fortner J, Sun R L, Brennan M F, Blumgart L H. Clinical score for predicting recurrence after hepatic resection for metastatic colorectal cancer: analysis of 1001 consecutive cases. Ann Surg 1999; 230:309-18; discussion 18-21.
10. Tomlinson J S, Jarnagin W R, DeMatteo R P, et al. Actual 10-year survival after resection of colorectal liver metastases defines cure. J Clin Oncol 2007; 25:4575-80.
11. Kadri S, Long B C, Mujacic I, et al. Clinical Validation of a Next-Generation Sequencing Genomic Oncology Panel via Cross-Platform Benchmarking against Established Amplicon Sequencing Assays. J Mol Diagn 2017; 19:43-56.
12. Mann C D, Metcalfe M S, Leopardi L N, Maddern G J. The clinical risk score: emerging as a reliable preoperative prognostic index in hepatectomy for colorectal metastases. Arch Surg 2004; 139:1168-72.
13. Ivanecz A, Potrc S, Horvat M, Jagric T, Gadzijev E. The validity of clinical risk score for patients undergoing liver resection for colorectal metastases. Hepatogastroenterology 2009; 56:1452-8.
14. Perou C M, Sorlie T, Eisen M B, et al. Molecular portraits of human breast tumours. Nature 2000; 406:747-52.
15. Choi W, Porten S, Kim S, et al. Identification of distinct basal and luminal subtypes of muscle-invasive bladder cancer with different sensitivities to frontline chemotherapy. Cancer cell 2014; 25:152-65.
16. Guinney J, Dienstmann R, Wang X, et al. The consensus molecular subtypes of colorectal cancer. Nat Med 2015; 21:1350-6.
17. Dienstmann R, Vermeulen L, Guinney J, Kopetz S, Tejpar S, Tabernero J. Consensus molecular subtypes and the evolution of precision medicine in colorectal cancer. Nat Rev Cancer 2017; 17:268.
18. Cancer Genome Atlas N. Comprehensive molecular characterization of human colon and rectal cancer. Nature 2012; 487:330-7.
19. Cantini L, Isella C, Petti C, et al. MicroRNA-mRNA interactions underlying colorectal cancer molecular subtypes. Nature communications 2015; 6:8878.
20. Uppal A, Wightman S C, Mallon S, et al. 14q32-encoded microRNAs mediate an oligometastatic phenotype. Oncotarget 2015; 6:3540-52.
21. Lussier Y A, Xing H R, Salama J K, et al. MicroRNA expression characterizes oligometastasis(es). PloS one; 6:e28650.
22. Dvinge H, Git A, Graf S, et al. The shaping and functional consequences of the microRNA landscape in breast cancer. Nature 2013; 497:378-82.
23. Wang B, Mezlini A M, Demir F, et al. Similarity network fusion for aggregating data types on a genomic scale. Nat Methods 2014; 11:333-7.
24. Alhamdoosh M, Ng M, Wilson N J, et al. Combining multiple tools outperforms individual methods in gene set enrichment analyses. Bioinformatics 2017; 33:414-24.
25. Mlecnik B, Bindea G, Angell H K, et al. Integrative Analyses of Colorectal Cancer Show Immunoscore Is a Stronger Predictor of Patient Survival Than Microsatellite Instability. Immunity 2016; 44:698-711.
26. Vilar E, Gruber S B. Microsatellite instability in colorectal cancer—the stable evidence. Nat Rev Clin Oncol 2010; 7:153-62.
27. Rooney M S, Shukla S A, Wu C J, Getz G, Hacohen N. Molecular and genetic properties of tumors associated with local immune cytolytic activity. Cell 2015; 160:48-61.
28. Sveen A, Loes I M, Alagaratnam S, et al. Intra-patient Inter-metastatic Genetic
Heterogeneity in Colorectal Cancer as a Key Determinant of Survival after Curative Liver Resection. PLoS Genet 2016; 12:e1006225.
29. Lee S Y, Haq F, Kim D, et al. Comparative genomic analysis of primary and synchronous metastatic colorectal cancers. PloS one 2014; 9:e90459.
30. Koebel C M, Vermi W, Swann J B, et al. Adaptive immunity maintains occult cancer in an equilibrium state. Nature 2007; 450:903-7.
31. Dunn G P, Old L J, Schreiber R D. The immunobiology of cancer immunosurveillance and immunoediting. Immunity 2004; 21:137-48.
32. Chiang A C, Massague J. Molecular basis of metastasis. The New England journal of medicine 2008; 359:2814-23.
33. Vanharanta S, Massague J. Origins of metastatic traits. Cancer cell 2013; 24:410-21.
34. Massague J, Obenauf A C. Metastatic colonization by circulating tumour cells. Nature 2016; 529:298-306.
35. Lambert A W, Pattabiraman D R, Weinberg R A. Emerging Biological Principles of Metastasis. Cell 2017; 168:670-91.
36. Valastyan S, Weinberg R A. Tumor metastasis: molecular insights and evolving paradigms. Cell 2011; 147:275-92.
37. Fong Y, Fortner J, Sun R L, Brennan M F, Blumgart L H. Clinical score for predicting recurrence after hepatic resection for metastatic colorectal cancer: analysis of 1001 consecutive cases. Ann Surg 1999; 230:309-18; discussion 18-21.
38. Dobin A, Davis C A, Schlesinger F, et al. STAR: ultrafast universal RNA-seq aligner. Bioinformatics 2013; 29:15-21.
39. Tarasov A, Vilella A J, Cuppen E, Nijman I J, Prins P. Sambamba: fast processing of NGS alignment formats. Bioinformatics 2015; 31:2032-4.
40. Liao Y, Smyth G K, Shi W. The Subread aligner: fast, accurate and scalable read mapping by seed-and-vote. Nucleic acids research 2013; 41:e108.
41. Robinson M D, McCarthy D J, Smyth G K. edgeR: a Bioconductor package for differential expression analysis of digital gene expression data. Bioinformatics 2010; 26:139-40.
42. Ritchie M E, Phipson B, Wu D, et al. limma powers differential expression analyses for RNA-sequencing and microarray studies. Nucleic acids research 2015; 43:e47.
43. Leek J T, Johnson W E, Parker H S, Jaffe A E, Storey J D. The sva package for removing batch effects and other unwanted variation in high-throughput experiments. Bioinformatics 2012; 28:882-3.
44. Wu D, Hu Y, Tong S, Williams B R, Smyth G K, Gantier M P. The use of miRNA microarrays for the analysis of cancer samples with global miRNA decrease. Rna 2013; 19:876-88.
45. Johnson W E, Li C, Rabinovic A. Adjusting batch effects in microarray expression data using empirical Bayes methods. Biostatistics 2007; 8:118-27.
46. Senbabaoglu Y, Michailidis G, Li J Z. Critical limitations of consensus clustering in class discovery. Scientific reports 2014; 4:6207.
47. Wang B, Mezlini A M, Demir F, et al. Similarity network fusion for aggregating data types on a genomic scale. Nat Methods 2014; 11:333-7.
48. Alhamdoosh M, Ng M, Wilson N J, et al. Combining multiple tools outperforms individual methods in gene set enrichment analyses. Bioinformatics 2017; 33:414-24.
49. Liberzon A, Subramanian A, Pinchback R, Thorvaldsdottir H, Tamayo P, Mesirov J P. Molecular signatures database (MSigDB) 3.0. Bioinformatics 2011; 27:1739-40.
50. Guinney J, Dienstmann R, Wang X, et al. The consensus molecular subtypes of colorectal cancer. Nat Med 2015; 21:1350-6.
51. Bindea G, Mlecnik B, Tosolini M, et al. Spatiotemporal dynamics of intratumoral immune cells reveal the immune landscape in human cancer. Immunity 2013; 39:782-95.
52. Tibshirani R, Hastie T, Narasimhan B, Chu G. Diagnosis of multiple cancer types by shrunken centroids of gene expression. Proceedings of the National Academy of Sciences of the United States of America 2002; 99:6567-72.
53. Du P, Kibbe W A, Lin S M. lumi: a pipeline for processing Illumina microarray. Bioinformatics 2008; 24:1547-8.
54. Kadri S, Long B C, Mujacic I, et al. Clinical Validation of a Next-Generation Sequencing Genomic Oncology Panel via Cross-Platform Benchmarking against Established Amplicon Sequencing Assays. J Mol Diagn 2017; 19:43-56.
55. Costello M, Pugh T J, Fennell T J, et al. Discovery and characterization of artifactual mutations in deep coverage targeted capture sequencing data due to oxidative DNA damage during sample preparation. Nucleic acids research 2013; 41:e67.
56. Ramos A H, Lichtenstein L, Gupta M, et al. Oncotator: cancer variant annotation tool. Hum Mutat 2015; 36:E2423-9.
57. Lawrence M S, Stojanov P, Polak P, et al. Mutational heterogeneity in cancer and the search for new cancer-associated genes. Nature 2013; 499:214-8.
58. Reva B, Antipin Y, Sander C. Predicting the functional impact of protein mutations: application to cancer genomics. Nucleic acids research 2011; 39:e118.
59. Landrum M J, Lee J M, Riley G R, et al. ClinVar: public archive of relationships among sequence variation and human phenotype. Nucleic acids research 2014; 42:D980-5.
60. Talevich E, Shain A H, Botton T, Bastian B C. CNVkit: Genome-Wide Copy Number Detection and Visualization from Targeted DNA Sequencing. PLoS Comput Biol 2016; 12:e1004873.
61. Cancer Genome Atlas N. Comprehensive molecular characterization of human colon and rectal cancer. Nature 2012; 487:330-7.

1. A method comprising measuring expression levels of one or more genes listed in Table 10A or one or more miRNAs listed in Table 11A in a sample comprising tissue from a metastasis from a primary cancer tumor.

2. The method of claim 1, wherein the metastasis is a liver metastasis and the primary cancer tumor is a colorectal cancer tumor.

3. (canceled)

4. The method of claim 1, wherein the expression levels of at least 5 of the genes listed in Table 10A are measured.

5. The method of claim 1, wherein the expression levels of at least 5 of the miRNAs listed in Table 11A are measured.

6. The method of claim 1, wherein the expression levels of one or more genes listed in Table 10A and one or more miRNAs listed in Table 11A are measured.

7. (canceled)

8. The method of claim 1, wherein the expression levels of the one or more genes or one or more miRNAs are within a predetermined amount of a mean expression level in metastases of a cohort of patients having an oligometastatic phenotype, a mean expression level in metastases of a cohort of patients who are likely to be healed without the administration of systemic cancer therapy, or a mean expression level in metastases of a cohort of patients having a mean ten-year overall survival expectation that is at least 60%.

9. The method of claim 8, wherein the cohort of patients comprises a representative sample of patients having an oligometastatic phenotype.

10. (canceled)

11. The method of claim 1, wherein:
(A) the expression levels of one or more genes listed in Table 10A or one or more miRNAs listed in Table 11A deviate by a predetermined amount from the mean expression levels of the one or more genes or the one or more miRNAs in metastases of a cohort of metastatic colorectal cancer patients having a mean ten-year overall survival expectation that is less than 50%; or
(b) the expression levels of one or more genes listed in Table 10B are higher by a predetermined amount than the mean expression level of the one or more genes in metastases of said cohort; or
(c) the measured expression levels of one or more genes listed in Table 10C are lower by a predetermined amount than the mean expression level of the one or more genes in metastases of said cohort; or
(d) the measured expression levels of one or more miRNAs listed in Table 11B are higher by a predetermined amount than the mean expression level of the one or more miRNAs in metastases of said cohort; or
(e) the measured expression levels of one or more miRNAs listed in Table 11C are lower by a predetermined amount than the mean expression level of the one or more miRNAs in metastases of said cohort.

12.-15. (canceled)

16. The method of claim 11, wherein said cohort comprises a representative sample of metastatic colorectal cancer patients having a mean ten-year overall survival expectation that is less than 50%.

17. (canceled)

18. The method of claim 1, further comprising calculating a clinical risk score for the patient.

19.-22. (canceled)

24. A method of treating metastatic cancer in a patient, the method comprising administering to the patient a local cancer therapy without administering systemic cancer therapy or administering to the patient an immunotherapy, wherein the patient has been determined to have a metastasis having expression levels of one or more genes listed in Table 10A or one or more miRNAs listed in Table 11B that are within a predetermined amount of the mean expression level of the one or more genes or miRNAs in metastases of a cohort of metastatic cancer patients having a mean overall ten-year survival expectation that is at least 60%.

25. The method of claim 24, wherein the patient has been determined to have a metastasis having expression levels of at least 5 genes listed in Table 10A and/or at least 5 miRNAs listed in Table 11A that are within a predetermined amount of the mean expression level of the one or more genes or miRNAs in metastases of a cohort of metastatic cancer patients having a mean overall ten-year survival expectation that is at least 60%.

26. The method of claim 24, wherein the patient has been determined to have an mRNA and/or miRNA expression profile indicating an oligometastatic phenotype based on the expression of one or more genes listed in Table 10A and the expression of one or more genes listed in Table 11A.

27.-30. (canceled)

31. The method of, wherein the metastasis is a liver metastasis of a colorectal cancer.

32.-42. (canceled)

43. A method comprising evaluating expression levels of multiple mRNA and/or miRNA species in a sample comprising tissue from a liver metastasis of a patient that has metastatic colorectal cancer to identify the patient as belonging to a first group of patients or a second group of patients, wherein:
(a) the first group has one or more of the following characteristics:
(i) a mean ten-year overall survival expectation of at least 60%;
(ii) a mean ten-year overall survival expectation that is higher than that for patients outside of the first group;
(iii) a likelihood of experiencing metastatic recurrence after hepatic resection that is lower than the likelihood for patients outside of the first group;
(iv) a likelihood of being successfully treated without systemic cancer treatments that is higher than the likelihood for patients outside of the first group; and
(v) a likelihood of being successfully treated with immune checkpoint therapy that is higher than the likelihood for patients outside of the first group; and
(b) the second group has one or more of the following characteristics:
(i) a mean ten-year overall survival expectation of less than 50%;
(ii) a mean ten-year overall survival expectation that is lower than that for patients outside of the second group;
(iii) a likelihood of experiencing metastatic recurrence after hepatic resection that is higher than for patients outside of the second group;
(iv) a likelihood of being successfully treated without systemic cancer treatments that is lower than the likelihood for patients outside of the second group;
(v) a likelihood of being successfully treated with immune checkpoint therapy that is lower than the likelihood for patients outside of the second group; and
(vi) a likelihood of being successfully treated with DNA damaging cancer therapy that is higher than the likelihood for patients outside of the second group.

44. The method of claim 43, wherein the expression levels of the multiple mRNA species comprise expression levels of transcripts of one or more genes listed in Table 10A.

45. The method of claim 43, wherein the expression levels of the multiple miRNA species comprise expression levels of one or more of the miRNAs listed in Table 11A.

46. The method of claim 44, wherein the patient is identified as belonging to the first group of patients if the expression levels of one or more genes listed in Table 10A is within a predetermined amount of a reference expression level of the one or more genes or if the expression levels of one or more miRNAs listed in Table 11A is within a predetermined amount of a reference expression level of the one or more miRNAs.

47. (canceled)

48. The method of claim 43, wherein step (b) comprises using a classifier that has been trained to identify an RNA expression pattern associated with the first group of patients, wherein the classifier evaluates the expression levels of at least 5 genes listed in Table 10A and at least 5 of miRNAs listed in Table 11A.

49.-50. (canceled)

51. The method of claim 43, further comprising: (a) administering an immune checkpoint therapy or a local treatment of liver metastases unaccompanied by systemic cancer treatment to a patient identified as belonging to the first group or (b) administering a DNA damaging cancer therapy to a patient identified as belonging to the second group of patients.

52. (canceled)

53. (canceled)
